(19) United States
(12) Patent Application Publication
(10) Pub. No.: US 2025/0011392 A1
(43) Pub. Date: Jan. 9, 2025

(54) COLLAGEN COMPOSITIONS AND METHODS OF USE THEREOF

(71) Applicant: Modern Meadow, Inc., Nutley, NJ (US)

(72) Inventors: Lixin DAI, Nutley, NJ (US); Dale L HANDLIN, Jr., Nutley, NJ (US); Stanley LUE, Nutley, NJ (US); Monique DE LEEUW, Nutley, NJ (US); Dan DUEPPEN, Nutley, NJ (US); Dave WILLIAMSON, Nutley, NJ (US); Isabelle HANSENNE-CERVANTES, Nutley, NJ (US); Brenna RAUW, Nutley, NJ (US)

(21) Appl. No.: 18/557,244

(22) PCT Filed: Apr. 29, 2022
(86) PCT No.: PCT/US2022/027016

§ 371 Date: Oct. 25, 2023

Related U.S. Application Data
(63) Continuation-in-part of application No. PCT/US2021/030180, filed on Apr. 30, 2021.
(60) Provisional application No. 63/278,849, filed on Nov. 12, 2021.
(60) Provisional application No. 63/278,827, filed on Nov. 12, 2021.
(60) Provisional application No. 63/209,745, filed on Jun. 11, 2021.

(51) Int. Cl. C07K 14/78; A61K 8/65; A61K 38/00; A61Q 19/00; C12N 15/81

(52) U.S. Cl. CPC C07K 14/78; A61K 8/65; A61Q 19/00; C12N 15/815; A61K 38/00

(57) ABSTRACT

The present disclosure relates to a recombinant collagen, peptide fragments thereof, and sequence variants thereof. A novel Pichia pastoris strain for producing the recombinant collagen, fragments thereof, and sequence variants thereof, and methods of using the recombinant collagen, fragments, and sequence variants thereof are also provided. The present disclosure fulfills a need for environmentally friendly and safe alternatives for skincare.

### REFERENCE TO SEQUENCE LISTING SUBMITTED ELECTRONICALLY

The content of the electronically submitted sequence listing in ASCII text file (Name: 4431_082PC04_Seglisting_ST25; Size: 841,608; and Date of Creation: Apr. 27, 2022) filed with the application is incorporated herein by reference in its entirety.

### BACKGROUND

Collagen is one of the most important proteins in the human body, and is present in connective tissue such as cartilage, bones, tendons, ligaments, and skin, and it is the major protein in the extra-cellular matrix of human cells. “Recombinant collagen” refers to the family of at least 28 distinct naturally occurring collagen types prepared using recombinant techniques.

There are many known uses for collagen. In the cosmetics and skincare industry, for example, skincare compositions that include collagen can be used to combat the effects of aging and environmental stress on the appearance, elasticity, and thickness of skin. For example, ageing and environmental factors can lead to dermatological conditions including, but not limited to fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, and rhinophyma. Although there are numerous skincare products on the market to improve skin appearance, many consumers are hesitant to use chemically-synthesized products they perceive as being environmentally unfriendly or otherwise unsafe.

### BRIEF SUMMARY

In some embodiments, the present disclosure provides a recombinant collagen fragment having a molecular weight of about 50 kDa and a sequence identity of at least about 85% to the amino acid sequence set forth in SEQ ID NO: 986. In some embodiments, the recombinant collagen fragment is unhydroxylated. In some embodiments, the recombinant collagen fragment is hydroxylated. In some embodiments, collagen fragment has the amino acid sequence set forth in SEQ ID NO: 986.

In some embodiments, the present disclosure provides a sequence variant of a recombinant collagen fragment having a molecular weight of about 50 kDa and a sequence identity of at least about 85% to the amino acid sequence set forth in SEQ ID NO: 986, wherein the sequence variant comprises the amino acid sequence set forth in any one of SEQ ID NOs: 987-1015. In some embodiments, the sequence variant is unhydroxylated. In some embodiments, the sequence variant is hydroxylated.

In some embodiments, the present disclosure provides a composition comprising a recombinant collagen fragment having a molecular weight of about 50 kDa and a sequence identity of at least about 85% to the amino acid sequence set forth in SEQ ID NO: 986. In some embodiments, the present disclosure provides a composition comprising a sequence variant of a recombinant collagen fragment having a molecular weight of about 50 kDa and a sequence identity of at least about 85% to the amino acid sequence set forth in SEQ ID NO: 986, wherein the sequence variant comprises the amino acid sequence set forth in any one of SEQ ID NOs: 987-1015. In some embodiments, the composition further comprises one or more peptides formed from the hydrolyzation of the collagen fragment having the amino acid sequence set forth in SEQ ID NO: 986. In some embodiments, at least one of the one or more peptides formed from the hydrolyzation of the collagen fragment having the amino acid sequence set forth in SEQ ID NO: 986 has an amino acid sequence according to one of SEQ ID NOs: 2-972. In some embodiments, the composition further comprises a pharmaceutically acceptable or cosmetically acceptable excipient.

In some embodiments, the present disclosure provides a method of producing comprising a recombinant collagen fragment having a molecular weight of about 50 kDa and a sequence identity of at least about 85% to the amino acid sequence set forth in SEQ ID NO: 986, comprising producing the recombinant collagen fragment in a genetically engineered strain of yeast. In some embodiments, the yeast is Pichia pastoris. In some embodiments, the yeast has been transformed with a plasmid comprising the nucleic acid sequence set forth in SEQ ID NO: 973. In some embodiments, the yeast is a yeast that has been transformed with a plasmid comprising the nucleic acid sequence set forth in SEQ ID NO: 974.

In some embodiments, the method comprises: (i) fermenting a genetically engineered yeast in a fermentation broth; (ii) recovering from the fermentation broth recombinant collagen fragments secreted by the genetically engineered yeast; and (iii) optionally, purifying the recombinant collagen fragments. In some embodiments, the method further comprises hydroxylating the recombinant collagen fragment ex vivo.

In some embodiments, the present disclosure provides a method of producing the sequence variant disclosed herein, comprising producing the recombinant collagen fragment in a genetically engineered strain of yeast. In some embodiments, the yeast is Pichia pastoris. In some embodiments, the yeast has been transformed with a plasmid comprising the nucleic acid sequence set forth in any one of SEQ ID NO: 1045-1073.

In some embodiments, the method comprises: (i) fermenting a genetically engineered yeast in a fermentation broth; (ii) recovering from the fermentation broth recombinant collagen fragment sequence variants secreted by the genetically engineered yeast; and (iii) optionally, purifying the recombinant collagen fragments.

In some embodiments, the method further comprises hydroxylating the recombinant collagen sequence variant ex vivo.

In some embodiments, the present disclosure provides a strain of yeast genetically engineered to produce the recombinant collagen fragment described herein, wherein the strain of yeast comprises a vector comprising a DNA sequence encoding the recombinant collagen.

In some embodiments, the present disclosure provides a strain of yeast genetically engineered to produce the sequence variants described herein, wherein the strain of yeast comprises a vector comprising a DNA sequence encoding the variant.

The strain of yeast of claim 24, wherein the vector comprises a nucleic acid sequence comprising the DNA sequence set forth in SEQ ID NO: 973. In some embodiments, the strain of yeast further comprises a second vector comprising a nucleic acid sequence comprising the DNA sequence set forth in SEQ ID NO: 974. In some embodiments, the vector comprises a nucleic acid sequence comprising the DNA sequence set forth in any one of SEQ ID NO: 1045-1073. In some embodiments, the strain of yeast described herein is a Pichia pastoris.

In some embodiments, the present disclosure provides a method of treating a dermatological condition comprising administering an effective amount of the recombinant collagen fragment having a molecular weight of about 50 kDa and a sequence identity of at least about 85% to the amino acid sequence set forth in SEQ ID NO: 986, described herein, or the sequence variants thereof, to a subject in need thereof.

In some embodiments, the present disclosure provides a method of treating a dermatological condition comprising administering an effective amount of the composition described herein to a subject in need thereof. In some embodiments, the dermatological condition comprises fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma. In some embodiments, the composition is topically administered to an area of skin. In some embodiments, the area of skin is selected from the group consisting of a facial surface, scalp, neck, ears, shoulders, chest (including breasts and/or the decolletage), arms, hands, legs, stomach, buttocks, groin, back, feet, and combinations thereof.

In some embodiments, the present disclosure provides a method of increasing collagen production in cells, comprising administering an effective amount of the recombinant collagen fragment having a molecular weight of about 50 kDa and a sequence identity of at least about 85% to the amino acid sequence set forth in SEQ ID NO: 986, described herein, or the sequence variants thereof, to the cells. In some embodiments, the method increases the production of Type I collagen. In some embodiments, the cells are fibroblasts. In some embodiments, the cells are cultured cells. In some embodiments, the fragment or variant is formulated in a composition. In some embodiments, the fragment has an amino acid sequence set forth in SEQ ID NO: 986. In some embodiments, the sequence variant has an amino acid sequence set forth in any one of SEQ ID NOs: 987-1015.

In some embodiments, the present disclosure provides a skincare product comprising the composition described herein, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

In some embodiments, the present disclosure provides a method of treating a wound in a human subject in need thereof, the method comprising applying the composition described herein to the wound on the subject, wherein applying the recombinant collagen fragment induces the production of human Type I collagen, human Type III collagen, or a combination thereof. In some embodiments, the collagen fragment is topically applied to the wound.

In some embodiments, the present disclosure provides a recombinant collagen fragment having a molecular weight of about 50 kDa and a sequence identity of at least about 85% to the amino acid sequence set forth in SEQ ID NO: 1 or SEQ ID NO: 986. In some embodiments, the recombinant collagen fragment can be unhydroxylated. In some embodiments, the recombinant collagen fragment can be hydroxylated. In some embodiments, the collagen fragment can have the amino acid sequence set forth in SEQ ID NO: 1 or SEQ ID NO: 986. In some embodiments, the present disclosure provides a recombinant collagen fragment comprising an amino acid sequence according to any one of SEQ ID NOs: 2-972.

In some embodiments, the present disclosure provides a composition comprising a recombinant collagen fragment described herein. In some embodiments, the composition further comprises one or more peptides formed from hydrolysis of the collagen fragment having the amino acid sequence set forth in SEQ ID NO: 1 or from hydrolysis of the collagen fragment having the amino acid sequence set forth in SEQ ID NO: 986.

In some embodiments of the present disclosure, at least one of the one or more peptides formed from hydrolysis of the collagen fragment having the amino acid sequence set forth in SEQ ID NO: 1 has an amino acid sequence according to one of SEQ ID NOs: 2-972. In some embodiments of the present disclosure, at least one of the one or more peptides formed from hydrolysis of the collagen fragment having the amino acid sequence set forth in SEQ ID NO: 986 has an amino acid sequence according to one of SEQ ID NOs: 2-972. In some embodiments, the composition further comprises a pharmaceutically acceptable or cosmetically acceptable excipient.

In some embodiments, the present disclosure provides a method of producing the recombinant collagen fragment comprising producing the recombinant collagen fragment in a genetically engineered strain of yeast. In some embodiments, the yeast can be Pichia pastoris. In some embodiments, the yeast can be transformed with a plasmid comprising the nucleic acid sequence set forth in SEQ ID NO: 973. In some embodiments, the yeast can be a yeast that has been further transformed with a plasmid comprising the nucleic acid sequence set forth in SEQ ID NO: 974. In some embodiments, the method comprises: (i) fermenting a genetically engineered yeast in a fermentation broth; (ii) recovering from the fermentation broth recombinant collagen fragments secreted by the genetically engineered yeast; and (iii) optionally, purifying the recombinant collagen fragments. In some embodiments, the method further comprises hydroxylating the recombinant collagen fragments ex vivo.

In some embodiments, the present disclosure provides a strain of yeast genetically engineered to produce the recombinant collagen fragment described herein, wherein the strain of yeast comprises a vector comprising a DNA sequence encoding the recombinant collagen. In some embodiments, the vector comprises a nucleic acid sequence comprising the DNA sequence set forth in SEQ ID NO: 973. In some embodiments, the strain of yeast described herein further comprises a second vector comprising a nucleic acid sequence comprising the DNA sequence set forth in SEQ ID NO: 974. In some embodiments, the strain of yeast can be Pichia pastoris.

In some embodiments, the present disclosure provides a method of treating a dermatological condition comprising administering an effective amount of the recombinant collagen fragment described herein to a subject in need thereof. In some embodiments, the present disclosure provides a method of treating a dermatological condition comprising administering an effective amount of the composition disclosed herein to a subject in need thereof. In some embodiments, the dermatological condition can be fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma. In some embodiments, the composition can be topically administered to an area of skin. In some embodiments, the area of skin can be selected from the group consisting of a facial surface, scalp, neck, ears, shoulders, chest (including breasts and/or the decolletage), arms, hands, legs, stomach, buttocks, groin, back, feet, and combinations thereof.

In some embodiments, the present disclosure provides a method of increasing collagen production in cells, comprising administering an effective amount of the recombinant collagen fragment described herein to the cells. In some embodiments, the method can increase the production of Type I collagen. In some embodiments, the method can increase the production of Type III collagen. In some embodiments, the cells can be fibroblasts. In some embodiments, the cells can be cultured cells.

In some embodiments of the methods disclosed herein, the fragment can be formulated in a composition. In some embodiments, the fragment can have an amino acid sequence according to SEQ ID NO: 1, or SEQ ID NO: 986.

In some embodiments, the present disclosure provides a skincare product comprising the composition described herein, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

In some embodiments the present disclosure provides methods of treating a wound in a human subject in need thereof, the method comprising applying a composition comprising a recombinant collagen fragment disclosed herein to the wound on the subject, wherein applying the recombinant collagen fragment induces the production of human Type I collagen, human Type III collagen, or a combination thereof. In certain embodiments of these methods, the collagen fragment is topically applied to the wound.

### BRIEF DESCRIPTION OF THE FIGURES

FIG. 1 shows the vector diagram of Vector A, a plasmid encoding a 50 kDa fragment of human collagen III, with zeocin resistance.

FIG. 2 shows the vector diagram of Vector B, a plasmid encoding a 50 kDa fragment of human collagen III, N-acetyl transferase, and beta-lactamase.

FIG. 3A shows the biomass density curve of a yeast culture expressing a 50 kDa fragment of human collagen, as detected by optical density measurements at 600 nm. The X-axis is fermentation time in hours. The Y-axis is the biomass density.

FIG. 3B shows the biomass density curve of a yeast culture expressing a 50 kDa fragment of human collagen, as detected by wet cell weight measurements. The X-axis is fermentation time in hours. The Y-axis is the wet cell weight.

FIG. 3C shows the glycerol concentration of a yeast culture expressing a 50 kDa fragment of human collagen, relative to the fermentation time of the culture. The X-axis is fermentation time in hours. The Y-axis is the glycerol concentration.

FIG. 3D shows the carbon dioxide evolution rate (CER) of a yeast culture expressing a 50 kDa fragment of human collagen, relative to the fermentation time of the culture. The X-axis is fermentation time in hours. The Y-axis is the CER.

FIG. 4 shows the abundance of various-sized peptides generated from hydrolyzation of a 50 kDa fragment of human collagen, as detected by mass spectrometry analysis. The analysis showed that a range of peptides appears after incubating one week at room temperature, and that the peptides present were equivalent after one and three weeks of incubation.

FIG. 5 shows a flow chart of the process to purify a unhydroxylated 50 kDa fragment of human collagen.

FIG. 6 shows a flow chart of the process to purify a hydroxylated 50 kDa fragment of human collagen.

FIG. 7 shows the percent recovery of a 50 kDa fragment of human collagen during individual steps of its purification process, and overall during all of the steps up to those individual steps.

FIG. 8 shows the percent hydroxylation of a 50 kDa fragment of human collagen achieved over time during an ex vivo hydroxylation reaction.

FIG. 9A and FIG. 9B show thermograms of 50 kDa collagen before and after hydroxylation, respectively. Hydroxylated 50 kDa collagen builds rheology and has improved thermal stability post hydroxylation.

FIG. 10 shows a circular dichroism spectra of hydroxylated 50 kDa collagen, which exhibits the signature profile expected for helical collagen.

FIG. 11A and FIG. 11B show the results of an MTT assay to measure the effects of a different collagen preparations, at a range of concentrations, on the viability of fibroblasts. None of the collagens were observed to impact cell viability.

FIG. 12A and FIG. 12B show the results of a type I collagen assay to detect the effects of different collagen and collagen fragment preparations to impact type I collagen synthesis in treated primary human dermal fibroblasts.

FIG. 13A and FIG. 13B show the results of a type III collagen assay to detect the effects of different collagen and collagen fragment preparations to impact type III collagen synthesis in treated primary human dermal fibroblasts.

FIG. 14 shows the measured solubility and character of collagen and collagen fragment solutions.

FIG. 15A and FIG. 15B show the results of an EpiOcular test conducted on Human Recombinant Collagen III and a 50 kDa fragment of human collagen

FIG. 16A provides the statistical result for each attribute objectively graded for Formulations 1 and 2.

FIGS. 16B and 16C provide the self-assessment questions and results for Formulations 1 and 2.

FIG. 16D shows the statistical results the levels of collagen within the skin both before and after 6 weeks of application of either Formulation 1 or 2.

FIGS. 17A-17H show the results of an MTT assay to measure the effects of a different collagen variant preparations, at a range of concentrations, on the viability of fibroblasts.

FIGS. 18A-18H show the results of a type I collagen assay to detect the effects of different collagen variant preparations to impact type I collagen synthesis in treated primary human dermal fibroblasts.

FIGS. 19A-19H show the results of a type III collagen assay to detect the effects of different collagen variant preparations to impact type III collagen synthesis in treated primary human dermal fibroblasts.

### DETAILED DESCRIPTION

### Definitions

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used herein, the term “about” used with numerical values means “within 10% of the stated value,” unless expressly noted otherwise. For example, “about 5% by weight” means from 4.5% by weight to 5.5% by weight.

The term “modified,” as applied to the collagen fragments disclosed herein, refers to collagen fragments comprising an amino acid sequence that is at least 70%, 80%, 90%, 95%, or 99% identical or similar to the amino acid sequence of a biologically active molecule. In some embodiments, the modified collagen fragment comprises an amino acid sequence that is at least 75%, 80%, 85%, 86%, 87%, 88%, 89%, 90%, 91%, 92%, 93%, 94%, 95%, 96%, 97%, 98%, or 99% identical to the amino acid sequence of a native or previously engineered sequence. The modified sequence can comprise additions, deletions, substitutions, or a combination thereof to the amino acid sequence of a native or previously engineered molecule. For example, a modified collagen fragment can incorporate or delete 1, 2, 3, 4, 5, 6, 7, 8, 9, 10 or more amino acid residues compared to a native collagen sequence. Such selections can be made to modify the looseness or tightness of a recombinant collagen. The degree of hydroxylation of collagen correlates with the looseness or tightness of the collagen triple helix. A modified collagen fragment can also include chemical modifications to a polypeptide, such as crosslinks between cysteine residues, or hydroxylated or glycosylated residues. As used herein, the terms “variant” and “sequence variant” refer to a polypeptide sequence that is about 75% to about 99% identical to the amino acid sequence set forth in SEQ ID NO: 986.

The terms “pharmaceutically acceptable” and “cosmetically acceptable,” as applied to carriers, excipients, or stabilizers that may be used in the compositions described herein, refer to carriers, excipients, or stabilizers that are nontoxic to recipients at the dosages and concentrations employed.

As used herein, the term “tissue repair” refers to the restoration of tissue architecture and function after an injury in the context of the healing of damaged tissue. Tissue regeneration refers to a type of healing in which new growth restores portions of damaged tissue to a normal state.

### A. Collagen

The term “collagen” refers to any one of the known collagen types, including collagen types I through XX described below, as well as to any other collagens, whether natural, synthetic, semi-synthetic, or recombinant. The term collagen includes collagen, collagen fragments, collagen-like proteins, triple helical collagen, alpha chains, monomers, gelatin, trimers and combinations thereof. It includes all of the collagens, modified collagens and collagen-like proteins described herein. The term also encompasses procollagens and collagen-like proteins or collagenous proteins comprising the motif (Gly-X-Y)n where n is an integer. It encompasses molecules of collagen and collagen-like proteins, trimers of collagen molecules, fibrils of collagen, and fibers of collagen fibrils. It also refers to chemically, enzymatically or recombinantly-modified collagens or collagen-like molecules that can be fibrillated as well as fragments of collagen, collagen-like molecules and collagenous molecules capable of assembling into a nanofiber. Recombinant collagen molecules whether native or engineered will generally comprise a repeated -(Gly-X-Y)n- sequence.

As used herein, collagen is a generic term for a family of at least 28 distinct collagen types. Various distinct collagen types have been identified in a range of species, including bovine, ovine, porcine, chicken, marine, plant, and human collagens. Animal skin is typically Type I collagen, although other types of collagen can be used in forming leather including type III collagen. The term “collagen” encompasses unprocessed (e.g., procollagens) as well as post-translationally modified and proteolyzed collagens having a triple helical structure. Type I collagen is the major fibrillar collagen of bone and skin comprising approximately 80-90% of an organism's total collagen. Type I collagen is the major structural macromolecule present in the extracellular matrix of multicellular organisms and comprises approximately 20% of total protein mass. Type I collagen is a heterotrimeric molecule comprising two α1(I) chains and one α2(I) chain, encoded by the COL1A1 and COL1A2 genes, respectively. In vivo assembly of Type I collagen fibrils, fibers, and fiber bundles takes place during development and provides mechanical support to the tissue while allowing for cellular motility and nutrient transport. Other collagen types are less abundant than type I collagen and exhibit different distribution patterns. Type III collagen is a major fibrillar collagen found in skin and vascular tissues. Type III collagen is a homotrimeric collagen comprising three identical α1(III) chains encoded by the COL3A1 gene.

### B. Recombinant Collagen and Recombinant Collagen Fragments

As used herein, the term “recombinant collagen” refers to the family of at least 28 distinct naturally occurring collagen types including, but not limited to collagen types I through XXVIII, prepared using recombinant techniques.

In some embodiments, the recombinant collagen described herein is a recombinant collagen fragment. A recombinant collagen fragment can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n).

Exemplary collagen sequences from which fragments can be derived include amino acid sequences of Col1A1, Col1A2, and Col3A1, such as those described by Accession Nos. P02461.4 (SEQ ID NO: 982; human Col3A1) (www.ncbi.nlm.nih.gov/protein/124056490), NP_001029211.1 (SEQ ID NO: 978; bovine Col1A1) (www.ncbi.nlm.nih.gov/protein/77404252), NP_776945.1 (SEQ ID NO: 979; bovine Col1A2) (www.ncbi.nlm.nih.gov/protein/27806257) and NP_001070299.1 (SEQ ID NO: 980; bovine Col3A1) (www.ncbi.nlm.nih.gov/protein/116003881), which are incorporated herein by reference.

A gene encoding a collagen can be truncated or otherwise modified to add or remove sequences, for example to encode the collagen fragments disclosed herein. In addition, gene modifications can be made to customize the size of a polynucleotide or vector, to target the expressed protein to the endoplasmic reticulum or other cellular or extracellular compartment, or to control the length of an encoded protein. Modifications can be made to polynucleotides encoding collagen fragments. For example, a polynucleotide coding sequence for a collagen or collagen fragment can be modified to encode a protein that is at least 70, 80, 90, 95, 96, 97, 98, or 100% identical or similar to a known amino acid sequence. Such modifications can include codon-modifying or codon-optimizing a polynucleotide encoding a collagen fragment.

In some embodiments, the collagen fragment disclosed herein can have a molecular weight from about 40 kDa to about 60 kDa. In some embodiments, the collagen fragment can have a molecular weight of about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, or about 60 kDa. In a particular embodiment, the collagen fragment can have a molecular weight of about 50 kDa.

In some embodiments, the collagen fragment described herein can have an amino acid chain length from about 350 amino acids to about 600 amino acids and can overlap with the amino acid sequence of SEQ ID NO: 1 or SEQ ID NO: 986. In some embodiments, the overlapping collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the collagen fragment described herein can have an amino acid sequence according to SEQ ID NO: 1 or SEQ ID NO: 986. In some embodiments, the collagen fragment can have at least about 70%, at least about 75%, at least about 80%, about 85%, at least about 87.5%, at least about 90%, at least about 92.5%, at least about 95%, at least about 97.5%, at least about 98%, at least about 99% or 100% sequence identity, or similarity to SEQ ID NO: 1. In some embodiments, the collagen fragment can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to SEQ ID NO: 986.

The amino acid sequence of SEQ ID NO: 1 is:
DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPP.
The amino acid sequence of SEQ ID NO: 986 is:
DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG.

In some embodiments, the collagen fragment described herein can have an amino acid chain length from about 350 amino acids to about 600 amino acids and can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1 or SEQ ID NO: 986. In some embodiments, the such a collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the recombinant collagen can comprise a hydrolysis product of a collagen fragment, wherein the hydrolysis product can have a sequence that is a portion of SEQ ID NO: 1 or SEQ ID NO: 986. In some embodiments, the hydrolysis product can have a sequence according to one of SEQ ID NOs: 2-972.

In some embodiments, the lysine, proline, or lysine and proline residues present in the collagen fragment are not hydroxylated. In other embodiments, the collagen fragment described herein can be hydroxylated. In some embodiments, at least 1%, 2%, 3%, 4%, 5%, 10%, 15%, 20%, 30%, 40%, 50%, 60%, 70%, 80%, 90%, 95% or 100% (or any intermediate value or subrange) of the lysine, proline, or lysine and proline residues in the recombinant collagen fragment can be hydroxylated. Hydroxylating collagen can build rheology and improve thermal stability of a collagen molecule or fragment. Hydroxylated collagen and hydroxylated collagen fragments are also resistant to high concentration pepsin digestion, for example at a pepsin:total protein ratio of 1:25 to 1:1.

The degree of hydroxylation of proline, lysine or proline and lysine residues in a collagen fragment can be estimated by determining the melting temperature of a hydrated collagen, such as a hydrogel, and comparing the melting point of the hydrogel to a “control” collagen fragment having a known content of hydroxylated amino acid residues. Collagen melting temperatures can range from 25-40° C. with more highly hydroxylated collagens generally having higher melting temperatures.

In some embodiments, the collagen fragment described herein can have an amino acid sequence as set forth below in Table 1 or Table 2. In some embodiments, the collagen fragment can be a collagen fragment sequence variant having the amino acid Table 1.

TABLE 1

| SEQ | |
|---|---|
| ID | |
| NO. | Sequence |
| 1 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPP |
| 2 | AAGARGNDGARGSDGQPGPPGPPGTA |
| 3 | AAGARGNDGARGSDGQPGPPGPPGTAGFPGSPGAK |
| 4 | AAGARGNDGARGSDGQPGPPGPPGTAGFPGSPGAKGEVGPA |
| 5 | AAGEPGRDGVPGGPGMRGMPG |
| 6 | AAGEPGRDGVPGGPGMRGMPGSPGGPG |
| 7 | AAGERGAPGFRGPAGPN |
| 8 | AGAQGPPGPPGIN |
| 9 | AGAQGPPGPPGINGSPGGKGEMGPAGIPGAPGLM |
| 10 | AGARGNDGARGSDGQPGPPGPPGTA |
| 11 | AGARGNDGARGSDGQPGPPGPPGTAGFPGSPGAKGEVGPA |
| 12 | AGEPGRDGVPGGPGMRGMPGSPGGPG |
| 13 | AGERGAPGFRGPAGPN |
| 14 | AGIPGAPGLM |
| 15 | AGIPGAPGLMGARGPPGPAGANGAPGLR |
| 16 | AGIPGFPGMKGH |
| 17 | AGIPGFPGMKGHRG |
| 18 | AGIPGFPGMKGHRGF |
| 19 | AGIPGFPGMKGHRGFDGRN |
| 20 | AGIPGFPGMKGHRGFDGRNGEKGETGAPGLK |
| 21 | AGIPGFPGMKGHRGFDGRNGEKGETGAPGLKGEN |
| 22 | AGKDGESGRPGRPGERGLPGPPGIK |
| 23 | AGKDGESGRPGRPGERGLPGPPGIKGPA |
| 24 | AGPPGPPGPPGTS |
| 25 | AGPPGPPGPPGTSGH |
| 26 | AGPSGPPGPPGAIGPS |
| 27 | AGPSGPPGPPGAIGPSGPAGKDGESGRPGRPGERGLPGPPGIK |
| 28 | AGSPGSNGAPGQRGEPGPQGHAGAQGPPGPPGINGSPGGKGEMGPAGIPGAPGLM |
| 29 | AGYPGPAGPPGPPGPPGTS |
| 30 | AGYPGPAGPPGPPGPPGTSGHPGSPGSPGYQGPPGEPGQAGPSGPPGPPGAIGPS |
| 31 | AIGPSGPAGKDGESGRPGRPGERGLPGPPGIK |
| 32 | AKGEVGPAGSPGSNGAPGQRGEPGPQG |
| 33 | AKGEVGPAGSPGSNGAPGQRGEPGPQGHAGAQGPPGPPGIN |
| 34 | ANGAPGLRGGAGEPGKNGAKGEPGPR |
| 35 | ANGLPGAAGERGAPGFRGPAGPN |
| 36 | APGERGRPGLPGAAGAR |
| 37 | APGERGRPGLPGAAGARGNDGARGSDGQPGPPGPPGTA |
| 38 | APGLKGENGLPGEN |
| 39 | APGLKGENGLPGENGAPGPMGPR |
| 40 | APGLMGARGPPGPA |
| 41 | APGLMGARGPPGPAGAN |
| 42 | APGLMGARGPPGPAGANGAPGLR |
| 43 | APGQRGEPGPQGHAGAQGPPGPPGIN |
| 44 | AQGPPGPPGIN |
| 45 | AQGPPGPPGINGSPG |
| 46 | AQGPPGPPGINGSPGGK |
| 47 | AQGPPGPPGINGSPGGKGEMGPA |
| 48 | AQGPPGPPGINGSPGGKGEMGPAGIPGAPGLMGAR |
| 49 | ARGNDGARGSDGQPGPPGPPGTA |
| 50 | ARGNDGARGSDGQPGPPGPPGTAGF |
| 51 | ARGNDGARGSDGQPGPPGPPGTAGFPGSPGAKGEVGPA |
| 52 | ARGNDGARGSDGQPGPPGPPGTAGFPGSPGAKGEVGPAGSPGSN |
| 53 | ARGPPGPAGANGAPGLR |
| 54 | ARGPPGPAGANGAPGLRGGAGEPGKN |
| 55 | ARGSDGQPGPPGPPGTA |
| 56 | ARGSDGQPGPPGPPGTAGFPGSPGAK |
| 57 | DAGAPGAPGGKGDAGAPGERGPP |
| 58 | DGAPGKNGERGGPGGPGPQGPPG |
| 59 | DGAPGKNGERGGPGGPGPQGPPGKN |
| 60 | DGAPGKNGERGGPGGPGPQGPPGKNGE |
| 61 | DGAPGKNGERGGPGGPGPQGPPGKNGETG |
| 62 | DGAPGKNGERGGPGGPGPQGPPGKNGETGP |
| 63 | DGAPGKNGERGGPGGPGPQGPPGKNGETGPQ |
| 64 | DGAPGKNGERGGPGGPGPQGPPGKNGETGPQG |
| 65 | DGAPGKNGERGGPGGPGPQGPPGKNGETGPQGPPG |
| 66 | DGAPGKNGERGGPGGPGPQGPPGKNGETGPQGPPGP |
| 67 | DGAPGKNGERGGPGGPGPQGPPGKNGETGPQGPPGPTG |
| 68 | DGAPGKNGERGGPGGPGPQGPPGKNGETGPQGPPGPTGPG |
| 69 | DGAPGKNGERGGPGGPGPQGPPGKNGETGPQGPPGPTGPGG |
| 70 | DGARGSDGQPGPPGPPG |
| 71 | DGARGSDGQPGPPGPPGTA |
| 72 | DGARGSDGQPGPPGPPGTAG |
| 73 | DGARGSDGQPGPPGPPGTAGF |
| 74 | DGARGSDGQPGPPGPPGTAGFPG |
| 75 | DGARGSDGQPGPPGPPGTAGFPGSPGAK |
| 76 | DGARGSDGQPGPPGPPGTAGFPGSPGAKG |
| 77 | DGARGSDGQPGPPGPPGTAGFPGSPGAKGEVGPA |
| 78 | DGARGSDGQPGPPGPPGTAGFPGSPGAKGEVGPAG |
| 79 | DGARGSDGQPGPPGPPGTAGFPGSPGAKGEVGPAGSPG |
| 80 | DGARGSDGQPGPPGPPGTAGFPGSPGAKGEVGPAGSPGSN |
| 81 | DGARGSDGQPGPPGPPGTAGFPGSPGAKGEVGPAGSPGSNGAPGQRGEPGPQ |
| 82 | DGESGRPGRPGERGLPGPPGIK |
| 83 | DGESGRPGRPGERGLPGPPGIKGPA |
| 84 | DGKPGPPGSQGESGRPGPPGPSGPRGQPGVM |
| 85 | DGQPGPPGPPGTA |
| 86 | DGRNGEKGETGAPGLK |
| 87 | DGRNGEKGETGAPGLKGENGLPGEN |
| 88 | DGRNGEKGETGAPGLKGENGLPGENGAPGPMGPR |
| 89 | DGRNGEKGETGAPGLKGENGLPGENGAPGPMGPRGAPGERGRPGLPGAA |
| 90 | DGRNGEKGETGAPGLKGENGLPGENGAPGPMGPRGAPGERGRPGLPGAAGAR |
| 91 | EKGETGAPGLKGENGLPGENGAPGPMGPR |
| 92 | EMGPAGIPGAPGLMGAR |
| 93 | ENGLPGENGAPGPMGPR |
| 94 | ENGLPGENGAPGPMGPRGAPGERGRPGLPGAAGAR |
| 95 | EPGQAGPSGPPGPPGAIGPS |
| 96 | ERGLPGPPGIKGPA |
| 97 | ESGRPGPPGPSGPRGQPGVM |
| 98 | ESGRPGPPGPSGPRGQPGVMGFPGPKGN |
| 99 | ESGRPGRPGERGLPGPPGIK |
| 100 | ETGAPGLKGENGLPGEN |
| 101 | ETGAPGLKGENGLPGENGAPGPMGPR |
| 102 | ETGPQGPPGPTGPGGD |
| 103 | EVGPAGSPGSNGAPGQRGEPGPQ |
| 104 | EVGPAGSPGSNGAPGQRGEPGPQGH |
| 105 | EVGPAGSPGSNGAPGQRGEPGPQGHAG |
| 106 | EVGPAGSPGSNGAPGQRGEPGPQGHAGAQ |
| 107 | EVGPAGSPGSNGAPGQRGEPGPQGHAGAQGPPGPPGIN |
| 108 | EVGPAGSPGSNGAPGQRGEPGPQGHAGAQGPPGPPGINGSPGGK |
| 109 | FDGRNGEKGETGAPGLKGEN |
| 110 | FDGRNGEKGETGAPGLKGENGLPGEN |
| 111 | FDGRNGEKGETGAPGLKGENGLPGENGAPGPMGPR |
| 112 | FPGSPGAKGEVGPA |
| 113 | FPGSPGAKGEVGPAGSPGSN |
| 114 | GAAGARGNDGARGSDGQPGPPGPP |
| 115 | GAAGARGNDGARGSDGQPGPPGPPGTA |
| 116 | GAAGARGNDGARGSDGQPGPPGPPGTAGFPGSPGAK |
| 117 | GAAGARGNDGARGSDGQPGPPGPPGTAGFPGSPGAKGEVGPA |
| 118 | GAAGEPGRDGVPGGPGMRGMPG |
| 119 | GAAGEPGRDGVPGGPGMRGMPGSPGGPG |
| 120 | GAAGERGAPGFRGPAGPN |
| 121 | GAIGPSGPAGKDGESGRPGRPGER |
| 122 | GAIGPSGPAGKDGESGRPGRPGERGLPGPP |
| 123 | GAIGPSGPAGKDGESGRPGRPGERGLPGPPGIK |
| 124 | GAKGEPGPRGERGEAGIPGVPG |
| 125 | GAKGEPGPRGERGEAGIPGVPGAK |
| 126 | GAKGEPGPRGERGEAGIPGVPGAKG |
| 127 | GAKGEPGPRGERGEAGIPGVPGAKGEDGK |
| 128 | GAKGEPGPRGERGEAGIPGVPGAKGEDGKDG |
| 129 | GAKGEPGPRGERGEAGIPGVPGAKGEDGKDGS |
| 130 | GAKGEPGPRGERGEAGIPGVPGAKGEDGKDGSPGEPGAN |
| 131 | GAKGEVGPAGSPGSNGAPGQRGEPGPQ |
| 132 | GAKGEVGPAGSPGSNGAPGQRGEPGPQG |
| 133 | GAKGEVGPAGSPGSNGAPGQRGEPGPQGH |
| 134 | GAKGEVGPAGSPGSNGAPGQRGEPGPQGHAGAQ |
| 135 | GAKGEVGPAGSPGSNGAPGQRGEPGPQGHAGAQGPPGPPGIN |
| 136 | GANGAPGLR |
| 137 | GANGAPGLRGGAGEPGKN |
| 138 | GANGAPGLRGGAGEPGKNGAK |
| 139 | GANGAPGLRGGAGEPGKNGAKGEPGPR |
| 140 | GANGAPGLRGGAGEPGKNGAKGEPGPRG |
| 141 | GANGAPGLRGGAGEPGKNGAKGEPGPRGER |
| 142 | GANGAPGLRGGAGEPGKNGAKGEPGPRGERG |
| 143 | GANGAPGLRGGAGEPGKNGAKGEPGPRGERGEAG |
| 144 | GAPGERGRPGLPGAA |
| 145 | GAPGERGRPGLPGAAG |
| 146 | GAPGERGRPGLPGAAGA |
| 147 | GAPGERGRPGLPGAAGAR |
| 148 | GAPGERGRPGLPGAAGARGN |
| 149 | GAPGERGRPGLPGAAGARGND |
| 150 | GAPGERGRPGLPGAAGARGNDG |
| 151 | GAPGERGRPGLPGAAGARGNDGA |
| 152 | GAPGERGRPGLPGAAGARGNDGAR |
| 153 | GAPGERGRPGLPGAAGARGNDGARG |
| 154 | GAPGERGRPGLPGAAGARGNDGARGS |
| 155 | GAPGERGRPGLPGAAGARGNDGARGSD |
| 156 | GAPGERGRPGLPGAAGARGNDGARGSDG |
| 157 | GAPGERGRPGLPGAAGARGNDGARGSDGQ |
| 158 | GAPGERGRPGLPGAAGARGNDGARGSDGQPG |
| 159 | GAPGERGRPGLPGAAGARGNDGARGSDGQPGP |
| 160 | GAPGERGRPGLPGAAGARGNDGARGSDGQPGPPG |
| 161 | GAPGERGRPGLPGAAGARGNDGARGSDGQPGPPGP |
| 162 | GAPGERGRPGLPGAAGARGNDGARGSDGQPGPPGPPG |
| 163 | GAPGERGRPGLPGAAGARGNDGARGSDGQPGPPGPPGTA |
| 164 | GAPGERGRPGLPGAAGARGNDGARGSDGQPGPPGPPGTAGFPGSPGAK |
| 165 | GAPGERGRPGLPGAAGARGNDGARGSDGQPGPPGPPGTAGFPGSPGAKGEVG |
| 166 | GAPGERGRPGLPGAAGARGNDGARGSDGQPGPPGPPGTAGFPGSPGAKGEVGPA |
| 167 | GAPGERGRPGLPGAAGARGNDGARGSDGQPGPPGPPGTAGFPGSPGAKGEVGPAG |
| 168 | GAPGLKGENGLPGENGAPGPMGPR |
| 169 | GAPGLMGARGPPGPA |
| 170 | GAPGLMGARGPPGPAGAN |
| 171 | GAPGLMGARGPPGPAGANGAP |
| 172 | GAPGLMGARGPPGPAGANGAPGLR |
| 173 | GAPGLMGARGPPGPAGANGAPGLRGGAGEPGKN |
| 174 | GAPGLRGGAGEPGKNGAKGEPGPRGERGEA |
| 175 | GAPGPAGPRGAAGEPGRDGVPG |
| 176 | GAPGPAGPRGAAGEPGRDGVPGGPGMRGMPG |
| 177 | GAPGPMGPRGAPGERGRPGLP |
| 178 | GAPGPMGPRGAPGERGRPGLPG |
| 179 | GAPGPMGPRGAPGERGRPGLPGAA |
| 180 | GAPGPMGPRGAPGERGRPGLPGAAG |
| 181 | GAPGPMGPRGAPGERGRPGLPGAAGA |
| 182 | GAPGPMGPRGAPGERGRPGLPGAAGAR |
| 183 | GAPGPMGPRGAPGERGRPGLPGAAGARGN |
| 184 | GAPGPMGPRGAPGERGRPGLPGAAGARGNDGAR |
| 185 | GAPGPMGPRGAPGERGRPGLPGAAGARGNDGARG |
| 186 | GAPGPMGPRGAPGERGRPGLPGAAGARGNDGARGSDGQPGPPGPPGTA |
| 187 | GAPGQRGEPGPQGHAGAQGPPGPP |
| 188 | GAPGQRGEPGPQGHAGAQGPPGPPG |
| 189 | GAPGQRGEPGPQGHAGAQGPPGPPGIN |
| 190 | GAPGQRGEPGPQGHAGAQGPPGPPGING |
| 191 | GAPGQRGEPGPQGHAGAQGPPGPPGINGS |
| 192 | GAPGQRGEPGPQGHAGAQGPPGPPGINGSPG |
| 193 | GAPGQRGEPGPQGHAGAQGPPGPPGINGSPGG |
| 194 | GAPGQRGEPGPQGHAGAQGPPGPPGINGSPGGK |
| 195 | GAPGQRGEPGPQGHAGAQGPPGPPGINGSPGGKGEM |
| 196 | GAPGQRGEPGPQGHAGAQGPPGPPGINGSPGGKGEMG |
| 197 | GAPGQRGEPGPQGHAGAQGPPGPPGINGSPGGKGEMGPA |
| 198 | GAPGQRGEPGPQGHAGAQGPPGPPGINGSPGGKGEMGPAGIPGAPGLM |
| 199 | GAPGQRGEPGPQGHAGAQGPPGPPGINGSPGGKGEMGPAGIPGAPGLMG |
| 200 | GAPGQRGEPGPQGHAGAQGPPGPPGINGSPGGKGEMGPAGIPGAPGLMGA |
| 201 | GAPGQRGEPGPQGHAGAQGPPGPPGINGSPGGKGEMGPAGIPGAPGLMGAR |
| 202 | GAQGPPGPPGIN |
| 203 | GARGNDGARGSDGQPGPPGPP |
| 204 | GARGNDGARGSDGQPGPPGPPGTA |
| 205 | GARGNDGARGSDGQPGPPGPPGTAGFPG |
| 206 | GARGNDGARGSDGQPGPPGPPGTAGFPGSPGA |
| 207 | GARGNDGARGSDGQPGPPGPPGTAGFPGSPGAK |
| 208 | GARGNDGARGSDGQPGPPGPPGTAGFPGSPGAKGEVGPA |
| 209 | GARGNDGARGSDGQPGPPGPPGTAGFPGSPGAKGEVGPAG |
| 210 | GARGNDGARGSDGQPGPPGPPGTAGFPGSPGAKGEVGPAGSPG |
| 211 | GARGNDGARGSDGQPGPPGPPGTAGFPGSPGAKGEVGPAGSPGSN |
| 212 | GARGPPGPAGAN |
| 213 | GARGPPGPAGANGAPGL |
| 214 | GARGPPGPAGANGAPGLR |
| 215 | GARGPPGPAGANGAPGLRG |
| 216 | GARGPPGPAGANGAPGLRGG |
| 217 | GARGPPGPAGANGAPGLRGGA |
| 218 | GARGPPGPAGANGAPGLRGGAG |
| 219 | GARGPPGPAGANGAPGLRGGAGE |
| 220 | GARGPPGPAGANGAPGLRGGAGEPG |
| 221 | GARGPPGPAGANGAPGLRGGAGEPGKN |
| 222 | GARGPPGPAGANGAPGLRGGAGEPGKNG |
| 223 | GARGPPGPAGANGAPGLRGGAGEPGKNGA |
| 224 | GARGPPGPAGANGAPGLRGGAGEPGKNGAK |
| 225 | GARGPPGPAGANGAPGLRGGAGEPGKNGAKGE |
| 226 | GARGPPGPAGANGAPGLRGGAGEPGKNGAKGEPG |
| 227 | GARGPPGPAGANGAPGLRGGAGEPGKNGAKGEPGPR |
| 228 | GARGPPGPAGANGAPGLRGGAGEPGKNGAKGEPGPRGER |
| 229 | GARGSDGQPGPPGPPGTA |
| 230 | GARGSDGQPGPPGPPGTAGFPGSPGAK |
| 231 | GARGSDGQPGPPGPPGTAGFPGSPGAKGEVGPA |
| 232 | GDKGDTGPPGPQGLQGLPGTGGPPGEN |
| 233 | GEKGETGAPGLK |
| 234 | GEKGETGAPGLKGENGLPGEN |
| 235 | GEKGETGAPGLKGENGLPGENGAPGPM |
| 236 | GEKGETGAPGLKGENGLPGENGAPGPMGPR |
| 237 | GEKGETGAPGLKGENGLPGENGAPGPMGPRGAPGERGRPGLPGAAGAR |
| 238 | GEMGPAGIPGAPGLM |
| 239 | GEMGPAGIPGAPGLMGAR |
| 240 | GEMGPAGIPGAPGLMGARGPPGPA |
| 241 | GEMGPAGIPGAPGLMGARGPPGPAGAN |
| 242 | GEMGPAGIPGAPGLMGARGPPGPAGANGAPGLR |
| 243 | GEMGPAGIPGAPGLMGARGPPGPAGANGAPGLRGGAGEPGKN |
| 244 | GENGLPGENGAPGPM |
| 245 | GENGLPGENGAPGPMGPR |
| 246 | GENGLPGENGAPGPMGPRG |
| 247 | GENGLPGENGAPGPMGPRGAPG |
| 248 | GENGLPGENGAPGPMGPRGAPGER |
| 249 | GENGLPGENGAPGPMGPRGAPGERG |
| 250 | GENGLPGENGAPGPMGPRGAPGERGR |
| 251 | GENGLPGENGAPGPMGPRGAPGERGRPGLPGAA |
| 252 | GENGLPGENGAPGPMGPRGAPGERGRPGLPGAAGA |
| 253 | GENGLPGENGAPGPMGPRGAPGERGRPGLPGAAGAR |
| 254 | GENGLPGENGAPGPMGPRGAPGERGRPGLPGAAGARGN |
| 255 | GEPGANGLPGAAGERGAPGFRGPAGPN |
| 256 | GEPGPKGDAGAPGAPGGKGDAGAPG |
| 257 | GEPGPKGDAGAPGAPGGKGDAGAPGE |
| 258 | GEPGPQGHAGAQGPPGPPGIN |
| 259 | GEPGPQGHAGAQGPPGPPGINGSPGGKGEMGPA |
| 260 | GEPGPQGHAGAQGPPGPPGINGSPGGKGEMGPAGIPGAPGLM |
| 261 | GEPGPRGERGEAGIPGVPGAKGEDGKDG |
| 262 | GEPGQAGPSGPPGPPGAIGPS |
| 263 | GEPGQAGPSGPPGPPGAIGPSGPAGKD |
| 264 | GEPGRDGVPGGPGMRGMPGSPGGPG |
| 265 | GERGEAGIPGVPGAKG |
| 266 | GERGEAGIPGVPGAKGEDGKDG |
| 267 | GERGEAGIPGVPGAKGEDGKDGSPGEPGAN |
| 268 | GERGGPGGPGPQGPPGK |
| 269 | GERGGPGGPGPQGPPGKN |
| 270 | GERGGPGGPGPQGPPGKNG |
| 271 | GERGGPGGPGPQGPPGKNGETGP |
| 272 | GERGGPGGPGPQGPPGKNGETGPQ |
| 273 | GERGGPGGPGPQGPPGKNGETGPQG |
| 274 | GERGGPGGPGPQGPPGKNGETGPQGPP |
| 275 | GERGGPGGPGPQGPPGKNGETGPQGPPG |
| 276 | GERGGPGGPGPQGPPGKNGETGPQGPPGP |
| 277 | GERGGPGGPGPQGPPGKNGETGPQGPPGPT |
| 278 | GERGGPGGPGPQGPPGKNGETGPQGPPGPTG |
| 279 | GERGGPGGPGPQGPPGKNGETGPQGPPGPTGPG |
| 280 | GERGGPGGPGPQGPPGKNGETGPQGPPGPTGPGG |
| 281 | GERGGPGGPGPQGPPGKNGETGPQGPPGPTGPGGD |
| 282 | GERGGPGGPGPQGPPGKNGETGPQGPPGPTGPGGDK |
| 283 | GERGGPGGPGPQGPPGKNGETGPQGPPGPTGPGGDKG |
| 284 | GERGGPGGPGPQGPPGKNGETGPQGPPGPTGPGGDKGD |
| 285 | GERGGPGGPGPQGPPGKNGETGPQGPPGPTGPGGDKGDTG |
| 286 | GERGGPGGPGPQGPPGKNGETGPQGPPGPTGPGGDKGDTGPPG |
| 287 | GERGGPGGPGPQGPPGKNGETGPQGPPGPTGPGGDKGDTGPPGPQ |
| 288 | GERGGPGGPGPQGPPGKNGETGPQGPPGPTGPGGDKGDTGPPGPQG |
| 289 | GERGGPGGPGPQGPPGKNGETGPQGPPGPTGPGGDKGDTGPPGPQGLQ |
| 290 | GERGGPGGPGPQGPPGKNGETGPQGPPGPTGPGGDKGDTGPPGPQGLQG |
| 291 | GERGLPGPPGIK |
| 292 | GERGLPGPPGIKGPA |
| 293 | GERGLPGPPGIKGPAGIPGFPGMK |
| 294 | GESGRPGPPGPSGPRGQPGVM |
| 295 | GESGRPGPPGPSGPRGQPGVMGFPGPKGN |
| 296 | GESGRPGRPGERGLPGPPGIK |
| 297 | GESGRPGRPGERGLPGPPGIKG |
| 298 | GESGRPGRPGERGLPGPPGIKGPA |
| 299 | GESGRPGRPGERGLPGPPGIKGPAGIPGFPGMK |
| 300 | GETGAPGLKGENGLPGEN |
| 301 | GETGAPGLKGENGLPGENGAPGPMGPR |
| 302 | GETGAPGLKGENGLPGENGAPGPMGPRGAPGERGRPGLPGAAGAR |
| 303 | GETGAPGLKGENGLPGENGAPGPMGPRGAPGERGRPGLPGAAGARGN |
| 304 | GETGPQGPPGPTGPG |
| 305 | GETGPQGPPGPTGPGGD |
| 306 | GETGPQGPPGPTGPGGDKGDTG |
| 307 | GETGPQGPPGPTGPGGDKGDTGPPG |
| 308 | GETGPQGPPGPTGPGGDKGDTGPPGPQ |
| 309 | GETGPQGPPGPTGPGGDKGDTGPPGPQG |
| 310 | GEVGPAGSPGSNGAPG |
| 311 | GEVGPAGSPGSNGAPGQ |
| 312 | GEVGPAGSPGSNGAPGQRGE |
| 313 | GEVGPAGSPGSNGAPGQRGEP |
| 314 | GEVGPAGSPGSNGAPGQRGEPG |
| 315 | GEVGPAGSPGSNGAPGQRGEPGP |
| 316 | GEVGPAGSPGSNGAPGQRGEPGPQ |
| 317 | GEVGPAGSPGSNGAPGQRGEPGPQG |
| 318 | GEVGPAGSPGSNGAPGQRGEPGPQGH |
| 319 | GEVGPAGSPGSNGAPGQRGEPGPQGHA |
| 320 | GEVGPAGSPGSNGAPGQRGEPGPQGHAG |
| 321 | GEVGPAGSPGSNGAPGQRGEPGPQGHAGA |
| 322 | GEVGPAGSPGSNGAPGQRGEPGPQGHAGAQ |
| 323 | GEVGPAGSPGSNGAPGQRGEPGPQGHAGAQG |
| 324 | GEVGPAGSPGSNGAPGQRGEPGPQGHAGAQGP |
| 325 | GEVGPAGSPGSNGAPGQRGEPGPQGHAGAQGPP |
| 326 | GEVGPAGSPGSNGAPGQRGEPGPQGHAGAQGPPG |
| 327 | GEVGPAGSPGSNGAPGQRGEPGPQGHAGAQGPPGP |
| 328 | GEVGPAGSPGSNGAPGQRGEPGPQGHAGAQGPPGPP |
| 329 | GEVGPAGSPGSNGAPGQRGEPGPQGHAGAQGPPGPPG |
| 330 | GEVGPAGSPGSNGAPGQRGEPGPQGHAGAQGPPGPPGIN |
| 331 | GEVGPAGSPGSNGAPGQRGEPGPQGHAGAQGPPGPPGING |
| 332 | GEVGPAGSPGSNGAPGQRGEPGPQGHAGAQGPPGPPGINGS |
| 333 | GEVGPAGSPGSNGAPGQRGEPGPQGHAGAQGPPGPPGINGSPG |
| 334 | GEVGPAGSPGSNGAPGQRGEPGPQGHAGAQGPPGPPGINGSPGG |
| 335 | GEVGPAGSPGSNGAPGQRGEPGPQGHAGAQGPPGPPGINGSPGGK |
| 336 | GEVGPAGSPGSNGAPGQRGEPGPQGHAGAQGPPGPPGINGSPGGKG |
| 337 | GEVGPAGSPGSNGAPGQRGEPGPQGHAGAQGPPGPPGINGSPGGKGEM |
| 338 | GEVGPAGSPGSNGAPGQRGEPGPQGHAGAQGPPGPPGINGSPGGKGEMG |
| 339 | GEVGPAGSPGSNGAPGQRGEPGPQGHAGAQGPPGPPGINGSPGGKGEMGPA |
| 340 | GEVGPAGSPGSNGAPGQRGEPGPQGHAGAQGPPGPPGINGSPGGKGEMGPAG |
| 341 | GFDGRNGEKGETGAPGLK |
| 342 | GFDGRNGEKGETGAPGLKG |
| 343 | GFDGRNGEKGETGAPGLKGEN |
| 344 | GFDGRNGEKGETGAPGLKGENG |
| 345 | GFDGRNGEKGETGAPGLKGENGLPGEN |
| 346 | GFDGRNGEKGETGAPGLKGENGLPGENGAPGPMGPR |
| 347 | GFDGRNGEKGETGAPGLKGENGLPGENGAPGPMGPRGAPGERGRPGLPGAA |
| 348 | GFPGMKGHRGFDGRNGEKGETGAPGLK |
| 349 | GFPGMKGHRGFDGRNGEKGETGAPGLKGEN |
| 350 | GFPGPKGNDGAPGKN |
| 351 | GFPGPKGNDGAPGKNGER |
| 352 | GFPGPKGNDGAPGKNGERG |
| 353 | GFPGPKGNDGAPGKNGERGG |
| 354 | GFPGPKGNDGAPGKNGERGGP |
| 355 | GFPGPKGNDGAPGKNGERGGPG |
| 356 | GFPGPKGNDGAPGKNGERGGPGG |
| 357 | GFPGPKGNDGAPGKNGERGGPGGP |
| 358 | GFPGPKGNDGAPGKNGERGGPGGPG |
| 359 | GFPGPKGNDGAPGKNGERGGPGGPGP |
| 360 | GFPGPKGNDGAPGKNGERGGPGGPGPQ |
| 361 | GFPGPKGNDGAPGKNGERGGPGGPGPQG |
| 362 | GFPGPKGNDGAPGKNGERGGPGGPGPQGP |
| 363 | GFPGPKGNDGAPGKNGERGGPGGPGPQGPP |
| 364 | GFPGPKGNDGAPGKNGERGGPGGPGPQGPPG |
| 365 | GFPGPKGNDGAPGKNGERGGPGGPGPQGPPGK |
| 366 | GFPGPKGNDGAPGKNGERGGPGGPGPQGPPGKN |
| 367 | GFPGPKGNDGAPGKNGERGGPGGPGPQGPPGKNG |
| 368 | GFPGPKGNDGAPGKNGERGGPGGPGPQGPPGKNGE |
| 369 | GFPGPKGNDGAPGKNGERGGPGGPGPQGPPGKNGET |
| 370 | GFPGPKGNDGAPGKNGERGGPGGPGPQGPPGKNGETG |
| 371 | GFPGPKGNDGAPGKNGERGGPGGPGPQGPPGKNGETGP |
| 372 | GFPGPKGNDGAPGKNGERGGPGGPGPQGPPGKNGETGPQ |
| 373 | GFPGPKGNDGAPGKNGERGGPGGPGPQGPPGKNGETGPQG |
| 374 | GFPGPKGNDGAPGKNGERGGPGGPGPQGPPGKNGETGPQGPPG |
| 375 | GFPGPKGNDGAPGKNGERGGPGGPGPQGPPGKNGETGPQGPPGPTG |
| 376 | GFPGPKGNDGAPGKNGERGGPGGPGPQGPPGKNGETGPQGPPGPTGPGGDK |
| 377 | GFPGSPGAKGEVG |
| 378 | GFPGSPGAKGEVGP |
| 379 | GFPGSPGAKGEVGPA |
| 380 | GFPGSPGAKGEVGPAG |
| 381 | GFPGSPGAKGEVGPAGS |
| 382 | GFPGSPGAKGEVGPAGSPG |
| 383 | GFPGSPGAKGEVGPAGSPGSN |
| 384 | GFPGSPGAKGEVGPAGSPGSNG |
| 385 | GFPGSPGAKGEVGPAGSPGSNGA |
| 386 | GFPGSPGAKGEVGPAGSPGSNGAPG |
| 387 | GFPGSPGAKGEVGPAGSPGSNGAPGQR |
| 388 | GFPGSPGAKGEVGPAGSPGSNGAPGQRG |
| 389 | GFPGSPGAKGEVGPAGSPGSNGAPGQRGE |
| 390 | GFPGSPGAKGEVGPAGSPGSNGAPGQRGEP |
| 391 | GFPGSPGAKGEVGPAGSPGSNGAPGQRGEPG |
| 392 | GFPGSPGAKGEVGPAGSPGSNGAPGQRGEPGPQ |
| 393 | GFPGSPGAKGEVGPAGSPGSNGAPGQRGEPGPQG |
| 394 | GFPGSPGAKGEVGPAGSPGSNGAPGQRGEPGPQGH |
| 395 | GFPGSPGAKGEVGPAGSPGSNGAPGQRGEPGPQGHA |
| 396 | GFPGSPGAKGEVGPAGSPGSNGAPGQRGEPGPQGHAG |
| 397 | GFPGSPGAKGEVGPAGSPGSNGAPGQRGEPGPQGHAGA |
| 398 | GFPGSPGAKGEVGPAGSPGSNGAPGQRGEPGPQGHAGAQ |
| 399 | GFPGSPGAKGEVGPAGSPGSNGAPGQRGEPGPQGHAGAQG |
| 400 | GFPGSPGAKGEVGPAGSPGSNGAPGQRGEPGPQGHAGAQGPP |
| 401 | GFPGSPGAKGEVGPAGSPGSNGAPGQRGEPGPQGHAGAQGPPG |
| 402 | GFPGSPGAKGEVGPAGSPGSNGAPGQRGEPGPQGHAGAQGPPGPPG |
| 403 | GFPGSPGAKGEVGPAGSPGSNGAPGQRGEPGPQGHAGAQGPPGPPGIN |
| 404 | GFPGSPGAKGEVGPAGSPGSNGAPGQRGEPGPQGHAGAQGPPGPPGING |
| 405 | GFPGSPGAKGEVGPAGSPGSNGAPGQRGEPGPQGHAGAQGPPGPPGINGSPG |
| 406 | GFPGSPGAKGEVGPAGSPGSNGAPGQRGEPGPQGHAGAQGPPGPPGINGSPGGK |
| 407 | GFRGPAGPNGIPGEKGPAGERGAPGPA |
| 408 | GGAGEPGKNGAKGEPGPR |
| 409 | GGAGEPGKNGAKGEPGPRGERGEAGIP |
| 410 | GGAGEPGKNGAKGEPGPRGERGEAGIPG |
| 411 | GGKGEMGPAGIPGAPGLM |
| 412 | GGKGEMGPAGIPGAPGLMGA |
| 413 | GGKGEMGPAGIPGAPGLMGAR |
| 414 | GGPGGPGPQGPPGKNGETGPQGPPGPTGPGGD |
| 415 | GGPGMRGMPGSPGGPGSDGKPGPPGSQGESGRPGPPGPSGPRGQPGVM |
| 416 | GGPGSDGKPGPPGSQGESGRPGPPG |
| 417 | GGPGSDGKPGPPGSQGESGRPGPPGPS |
| 418 | GGPGSDGKPGPPGSQGESGRPGPPGPSGPR |
| 419 | GGPGSDGKPGPPGSQGESGRPGPPGPSGPRGQ |
| 420 | GGPGSDGKPGPPGSQGESGRPGPPGPSGPRGQPGVM |
| 421 | GHAGAQGPPGPPGIN |
| 422 | GHAGAQGPPGPPGING |
| 423 | GHAGAQGPPGPPGINGSPG |
| 424 | GHAGAQGPPGPPGINGSPGGK |
| 425 | GHAGAQGPPGPPGINGSPGGKGEMG |
| 426 | GHAGAQGPPGPPGINGSPGGKGEMGPA |
| 427 | GHAGAQGPPGPPGINGSPGGKGEMGPAGIPGAPGLM |
| 428 | GHAGAQGPPGPPGINGSPGGKGEMGPAGIPGAPGLMG |
| 429 | GHAGAQGPPGPPGINGSPGGKGEMGPAGIPGAPGLMGA |
| 430 | GHAGAQGPPGPPGINGSPGGKGEMGPAGIPGAPGLMGAR |
| 431 | GHAGAQGPPGPPGINGSPGGKGEMGPAGIPGAPGLMGARGPPGPA |
| 432 | GHAGAQGPPGPPGINGSPGGKGEMGPAGIPGAPGLMGARGPPGPAGAN |
| 433 | GHAGAQGPPGPPGINGSPGGKGEMGPAGIPGAPGLMGARGPPGPAGANGAPGLR |
| 434 | GHPGSPGSPGYQGPPGEPG |
| 435 | GHPGSPGSPGYQGPPGEPGQA |
| 436 | GHPGSPGSPGYQGPPGEPGQAG |
| 437 | GHPGSPGSPGYQGPPGEPGQAGPS |
| 438 | GHPGSPGSPGYQGPPGEPGQAGPSGPPGPPGAIGPS |
| 439 | GHPGSPGSPGYQGPPGEPGQAGPSGPPGPPGAIGPSGPA |
| 440 | GHPGSPGSPGYQGPPGEPGQAGPSGPPGPPGAIGPSGPAGKDGESGRPGRPGER |
| 441 | GHRGFDGRNGEKGETGAPGLK |
| 442 | GHRGFDGRNGEKGETGAPGLKGEN |
| 443 | GHRGFDGRNGEKGETGAPGLKGENG |
| 444 | GHRGFDGRNGEKGETGAPGLKGENGLPGEN |
| 445 | GHRGFDGRNGEKGETGAPGLKGENGLPGENGAPGPMGPR |
| 446 | GHRGFDGRNGEKGETGAPGLKGENGLPGENGAPGPMGPRGAPGER |
| 447 | GIKGPAGIP |
| 448 | GIKGPAGIPGFPG |
| 449 | GIKGPAGIPGFPGMKG |
| 450 | GIPGAPGLM |
| 451 | GIPGAPGLMG |
| 452 | GIPGAPGLMGA |
| 453 | GIPGAPGLMGAR |
| 454 | GIPGAPGLMGARGPPG |
| 455 | GIPGAPGLMGARGPPGPA |
| 456 | GIPGAPGLMGARGPPGPAG |
| 457 | GIPGAPGLMGARGPPGPAGAN |
| 458 | GIPGAPGLMGARGPPGPAGANGAPGLR |
| 459 | GIPGAPGLMGARGPPGPAGANGAPGLRG |
| 460 | GIPGAPGLMGARGPPGPAGANGAPGLRGGAGEPGKN |
| 461 | GIPGEKGPAGERGAPGPA |
| 462 | GIPGEKGPAGERGAPGPAG |
| 463 | GIPGEKGPAGERGAPGPAGPR |
| 464 | GIPGEKGPAGERGAPGPAGPRG |
| 465 | GIPGEKGPAGERGAPGPAGPRGA |
| 466 | GIPGEKGPAGERGAPGPAGPRGAA |
| 467 | GIPGEKGPAGERGAPGPAGPRGAAG |
| 468 | GIPGEKGPAGERGAPGPAGPRGAAGEPG |
| 469 | GIPGEKGPAGERGAPGPAGPRGAAGEPGR |
| 470 | GIPGEKGPAGERGAPGPAGPRGAAGEPGRD |
| 471 | GIPGEKGPAGERGAPGPAGPRGAAGEPGRDGVPG |
| 472 | GIPGEKGPAGERGAPGPAGPRGAAGEPGRDGVPGG |
| 473 | GIPGEKGPAGERGAPGPAGPRGAAGEPGRDGVPGGPG |
| 474 | GIPGEKGPAGERGAPGPAGPRGAAGEPGRDGVPGGPGMR |
| 475 | GIPGFPGMKGH |
| 476 | GIPGFPGMKGHR |
| 477 | GIPGFPGMKGHRGF |
| 478 | GIPGFPGMKGHRGFD |
| 479 | GIPGFPGMKGHRGFDGR |
| 480 | GIPGFPGMKGHRGFDGRN |
| 481 | GIPGFPGMKGHRGFDGRNGEK |
| 482 | GIPGFPGMKGHRGFDGRNGEKG |
| 483 | GIPGFPGMKGHRGFDGRNGEKGETGAPGLK |
| 484 | GIPGFPGMKGHRGFDGRNGEKGETGAPGLKGEN |
| 485 | GIPGFPGMKGHRGFDGRNGEKGETGAPGLKGENGLPGEN |
| 486 | GIPGFPGMKGHRGFDGRNGEKGETGAPGLKGENGLPGENGAPGPMGPR |
| 487 | GKDGESGRPGRPGERGLPGPPGIK |
| 488 | GKDGESGRPGRPGERGLPGPPGIKGPA |
| 489 | GKDGESGRPGRPGERGLPGPPGIKGPAGIPGFPGMK |
| 490 | GKDGSPGEPGANGLPGA |
| 491 | GKDGSPGEPGANGLPGAAGERGAPG |
| 492 | GKDGSPGEPGANGLPGAAGERGAPGFRGPAGPN |
| 493 | GKGEMGPAGIPGAPGLM |
| 494 | GKGEMGPAGIPGAPGLMG |
| 495 | GKGEMGPAGIPGAPGLMGA |
| 496 | GKGEMGPAGIPGAPGLMGAR |
| 497 | GKGEMGPAGIPGAPGLMGARGPPGPA |
| 498 | GKGEMGPAGIPGAPGLMGARGPPGPAGAN |
| 499 | GKGEMGPAGIPGAPGLMGARGPPGPAGANGAPGLR |
| 500 | GKPGEPGPKGDAGAPGAPGGKGDAGAPG |
| 501 | GKPGEPGPKGDAGAPGAPGGKGDAGAPGE |
| 502 | GKPGEPGPKGDAGAPGAPGGKGDAGAPGER |
| 503 | GKPGEPGPKGDAGAPGAPGGKGDAGAPGERG |
| 504 | GKPGEPGPKGDAGAPGAPGGKGDAGAPGERGPP |
| 505 | GKPGPPGSQGESGRPGPPGPSGPR |
| 506 | GKPGPPGSQGESGRPGPPGPSGPRGQ |
| 507 | GKPGPPGSQGESGRPGPPGPSGPRGQPGVM |
| 508 | GLAGYPGPAGPPGPPGPPGTS |
| 509 | GLAGYPGPAGPPGPPGPPGTSGHPGSPGSPGYQGPPGEPGQAGPSGPPGPPGAIGPS |
| 510 | GLKGENGLPGENGAPGPMGPR |
| 511 | GLMGARGPPGPAGANGAPGLR |
| 512 | GLPGAAGARGNDGARGSDGQPGPPGPPGTA |
| 513 | GLPGAAGARGNDGARGSDGQPGPPGPPGTAGFPGSPGAKGEVGPA |
| 514 | GLPGAAGERGAPGF |
| 515 | GLPGAAGERGAPGFR |
| 516 | GLPGAAGERGAPGFRG |
| 517 | GLPGAAGERGAPGFRGPAG |
| 518 | GLPGAAGERGAPGFRGPAGPN |
| 519 | GLPGAAGERGAPGFRGPAGPNGIPGEKG |
| 520 | GLPGAAGERGAPGFRGPAGPNGIPGEKGPAGER |
| 521 | GLPGAAGERGAPGFRGPAGPNGIPGEKGPAGERGAPG |
| 522 | GLPGAAGERGAPGFRGPAGPNGIPGEKGPAGERGAPGPAGPR |
| 523 | GLPGENGAPGPM |
| 524 | GLPGENGAPGPMG |
| 525 | GLPGENGAPGPMGPR |
| 526 | GLPGENGAPGPMGPRG |
| 527 | GLPGENGAPGPMGPRGAPG |
| 528 | GLPGENGAPGPMGPRGAPGERGR |
| 529 | GLPGENGAPGPMGPRGAPGERGRPG |
| 530 | GLPGENGAPGPMGPRGAPGERGRPGLPGAAGAR |
| 531 | GLPGENGAPGPMGPRGAPGERGRPGLPGAAGARGN |
| 532 | GLPGENGAPGPMGPRGAPGERGRPGLPGAAGARGNDGARG |
| 533 | GLPGENGAPGPMGPRGAPGERGRPGLPGAAGARGNDGARGSDGQ |
| 534 | GLPGENGAPGPMGPRGAPGERGRPGLPGAAGARGNDGARGSDGQPGPPGPPGTA |
| 535 | GLPGPPGIK |
| 536 | GLPGPPGIKG |
| 537 | GLPGPPGIKGP |
| 538 | GLPGPPGIKGPA |
| 539 | GLPGPPGIKGPAG |
| 540 | GLPGPPGIKGPAGIP |
| 541 | GLPGPPGIKGPAGIPG |
| 542 | GLPGPPGIKGPAGIPGF |
| 543 | GLPGPPGIKGPAGIPGFPGM |
| 544 | GLPGPPGIKGPAGIPGFPGMK |
| 545 | GLPGPPGIKGPAGIPGFPGMKG |
| 546 | GLPGPPGIKGPAGIPGFPGMKGH |
| 547 | GLPGPPGIKGPAGIPGFPGMKGHR |
| 548 | GLPGPPGIKGPAGIPGFPGMKGHRGF |
| 549 | GLPGTGGPPGEN |
| 550 | GLRGGAGEPGKNGAKGEPGPRGERGEAGIP |
| 551 | GMPGSPGGPGSDGKPGPPG |
| 552 | GMPGSPGGPGSDGKPGPPGSQ |
| 553 | GMPGSPGGPGSDGKPGPPGSQG |
| 554 | GMPGSPGGPGSDGKPGPPGSQGESGRPGPPGPSGPR |
| 555 | GMPGSPGGPGSDGKPGPPGSQGESGRPGPPGPSGPRGQPGVM |
| 556 | GMRGMPGSPGGPGSDGKPGPPGSQGESGRPGPPGPSGPRGQPGVM |
| 557 | GNDGAPGKNGERGGPGGPGPQGPPGKN |
| 558 | GNDGAPGKNGERGGPGGPGPQGPPGKNGETGPQG |
| 559 | GNDGAPGKNGERGGPGGPGPQGPPGKNGETGPQGPPGP |
| 560 | GNDGARGSDGQPGPPGPPGTA |
| 561 | GNDGARGSDGQPGPPGPPGTAG |
| 562 | GNDGARGSDGQPGPPGPPGTAGF |
| 563 | GNDGARGSDGQPGPPGPPGTAGFPG |
| 564 | GNDGARGSDGQPGPPGPPGTAGFPGS |
| 565 | GNDGARGSDGQPGPPGPPGTAGFPGSPGAK |
| 566 | GNDGARGSDGQPGPPGPPGTAGFPGSPGAKG |
| 567 | GNDGARGSDGQPGPPGPPGTAGFPGSPGAKGEVG |
| 568 | GNDGARGSDGQPGPPGPPGTAGFPGSPGAKGEVGPA |
| 569 | GNDGARGSDGQPGPPGPPGTAGFPGSPGAKGEVGPAG |
| 570 | GNDGARGSDGQPGPPGPPGTAGFPGSPGAKGEVGPAGS |
| 571 | GNDGARGSDGQPGPPGPPGTAGFPGSPGAKGEVGPAGSPG |
| 572 | GNDGARGSDGQPGPPGPPGTAGFPGSPGAKGEVGPAGSPGSN |
| 573 | GNDGARGSDGQPGPPGPPGTAGFPGSPGAKGEVGPAGSPGSNGAPGQR |
| 574 | GNDGARGSDGQPGPPGPPGTAGFPGSPGAKGEVGPAGSPGSNGAPGQRGE |
| 575 | GNDGARGSDGQPGPPGPPGTAGFPGSPGAKGEVGPAGSPGSNGAPGQRGEPGPQ |
| 576 | GNDGARGSDGQPGPPGPPGTAGFPGSPGAKGEVGPAGSPGSNGAPGQRGEPGPQG |
| 577 | GPAGANGAPGLRGGAGEPG |
| 578 | GPAGIPGAPGLM |
| 579 | GPAGIPGAPGLMG |
| 580 | GPAGIPGAPGLMGA |
| 581 | GPAGIPGAPGLMGAR |
| 582 | GPAGIPGAPGLMGARGPPGPAGANGAPGLR |
| 583 | GPAGIPGFP |
| 584 | GPAGIPGFPG |
| 585 | GPAGIPGFPGM |
| 586 | GPAGIPGFPGMK |
| 587 | GPAGIPGFPGMKG |
| 588 | GPAGIPGFPGMKGH |
| 589 | GPAGIPGFPGMKGHR |
| 590 | GPAGIPGFPGMKGHRG |
| 591 | GPAGIPGFPGMKGHRGF |
| 592 | GPAGIPGFPGMKGHRGFD |
| 593 | GPAGIPGFPGMKGHRGFDG |
| 594 | GPAGIPGFPGMKGHRGFDGR |
| 595 | GPAGIPGFPGMKGHRGFDGRN |
| 596 | GPAGIPGFPGMKGHRGFDGRNG |
| 597 | GPAGIPGFPGMKGHRGFDGRNGE |
| 598 | GPAGIPGFPGMKGHRGFDGRNGEK |
| 599 | GPAGIPGFPGMKGHRGFDGRNGEKG |
| 600 | GPAGIPGFPGMKGHRGFDGRNGEKGE |
| 601 | GPAGIPGFPGMKGHRGFDGRNGEKGET |
| 602 | GPAGIPGFPGMKGHRGFDGRNGEKGETG |
| 603 | GPAGIPGFPGMKGHRGFDGRNGEKGETGA |
| 604 | GPAGIPGFPGMKGHRGFDGRNGEKGETGAP |
| 605 | GPAGIPGFPGMKGHRGFDGRNGEKGETGAPG |
| 606 | GPAGIPGFPGMKGHRGFDGRNGEKGETGAPGL |
| 607 | GPAGIPGFPGMKGHRGFDGRNGEKGETGAPGLK |
| 608 | GPAGIPGFPGMKGHRGFDGRNGEKGETGAPGLKG |
| 609 | GPAGIPGFPGMKGHRGFDGRNGEKGETGAPGLKGEN |
| 610 | GPAGIPGFPGMKGHRGFDGRNGEKGETGAPGLKGENGLPG |
| 611 | GPAGIPGFPGMKGHRGFDGRNGEKGETGAPGLKGENGLPGEN |
| 612 | GPAGIPGFPGMKGHRGFDGRNGEKGETGAPGLKGENGLPGENGAPGPM |
| 613 | GPAGIPGFPGMKGHRGFDGRNGEKGETGAPGLKGENGLPGENGAPGPMGPR |
| 614 | GPAGKDGESGRPGRPGERGLPGPP |
| 615 | GPAGKDGESGRPGRPGERGLPGPPG |
| 616 | GPAGKDGESGRPGRPGERGLPGPPGIK |
| 617 | GPAGKDGESGRPGRPGERGLPGPPGIKGPA |
| 618 | GPAGKDGESGRPGRPGERGLPGPPGIKGPAGIPGFPGMK |
| 619 | GPAGPNGIPGEKGPAGERGAPGPAGPR |
| 620 | GPAGPPGPPGPP |
| 621 | GPGMRGMPGSPGGPGSDGKPGPPG |
| 622 | GPGMRGMPGSPGGPGSDGKPGPPGSQGESGRPGPPGPSGPRGQPGVM |
| 623 | GPGSDGKPGPPGSQGESGRPGPPG |
| 624 | GPGSDGKPGPPGSQGESGRPGPPGPSG |
| 625 | GPGSDGKPGPPGSQGESGRPGPPGPSGPR |
| 626 | GPGSDGKPGPPGSQGESGRPGPPGPSGPRGQPGVM |
| 627 | GPGSDGKPGPPGSQGESGRPGPPGPSGPRGQPGVMGFPGPKGN |
| 628 | GPMGPRGAPGERGRPGLPGAA |
| 629 | GPMGPRGAPGERGRPGLPGAAGAR |
| 630 | GPNGIPGEKGPAGERGAPGPA |
| 631 | GPPGAIGPSGPAGKDGESGRPGRPGER |
| 632 | GPPGAIGPSGPAGKDGESGRPGRPGERGLPGPPGIK |
| 633 | GPPGEPGQAGPSGPPGPPG |
| 634 | GPPGEPGQAGPSGPPGPPGAIGPS |
| 635 | GPPGEPGQAGPSGPPGPPGAIGPSGPAGKD |
| 636 | GPPGEPGQAGPSGPPGPPGAIGPSGPAGKDGESGRPGRPGERGLPGPPGIK |
| 637 | GPPGIKGPAGIPGFPGMK |
| 638 | GPPGINGSPGGKGEMGPAGIPGAPGLM |
| 639 | GPPGPAGANGAPGLR |
| 640 | GPPGPAGANGAPGLRG |
| 641 | GPPGPAGANGAPGLRGG |
| 642 | GPPGPAGANGAPGLRGGA |
| 643 | GPPGPAGANGAPGLRGGAG |
| 644 | GPPGPAGANGAPGLRGGAGE |
| 645 | GPPGPAGANGAPGLRGGAGEP |
| 646 | GPPGPAGANGAPGLRGGAGEPG |
| 647 | GPPGPAGANGAPGLRGGAGEPGK |
| 648 | GPPGPAGANGAPGLRGGAGEPGKN |
| 649 | GPPGPAGANGAPGLRGGAGEPGKNG |
| 650 | GPPGPAGANGAPGLRGGAGEPGKNGA |
| 651 | GPPGPAGANGAPGLRGGAGEPGKNGAK |
| 652 | GPPGPAGANGAPGLRGGAGEPGKNGAKG |
| 653 | GPPGPAGANGAPGLRGGAGEPGKNGAKGE |
| 654 | GPPGPAGANGAPGLRGGAGEPGKNGAKGEPG |
| 655 | GPPGPAGANGAPGLRGGAGEPGKNGAKGEPGPR |
| 656 | GPPGPAGANGAPGLRGGAGEPGKNGAKGEPGPRG |
| 657 | GPPGPAGANGAPGLRGGAGEPGKNGAKGEPGPRGE |
| 658 | GPPGPAGANGAPGLRGGAGEPGKNGAKGEPGPRGER |
| 659 | GPPGPAGANGAPGLRGGAGEPGKNGAKGEPGPRGERG |
| 660 | GPPGPAGANGAPGLRGGAGEPGKNGAKGEPGPRGERGE |
| 661 | GPPGPAGANGAPGLRGGAGEPGKNGAKGEPGPRGERGEA |
| 662 | GPPGPPGAIGPS |
| 663 | GPPGPPGAIGPSGPA |
| 664 | GPPGPPGAIGPSGPAGKDGESGRPGRPGER |
| 665 | GPPGPPGAIGPSGPAGKDGESGRPGRPGERGLPGPPGIK |
| 666 | GPPGPPGINGSPGGKGEMGPA |
| 667 | GPPGPPGINGSPGGKGEMGPAGIPGAPGLM |
| 668 | GPPGPPGINGSPGGKGEMGPAGIPGAPGLMGA |
| 669 | GPPGPPGINGSPGGKGEMGPAGIPGAPGLMGAR |
| 670 | GPPGPPGINGSPGGKGEMGPAGIPGAPGLMGARGPPGPAGANGAPGLR |
| 671 | GPPGPPGPPGTS |
| 672 | GPPGPPGPPGTSGHPG |
| 673 | GPPGPPGPPGTSGHPGSPGSPGYQGPPGEPGQAGPSGPPGPPGAIGPS |
| 674 | GPPGPPGTAGFPGSPGAK |
| 675 | GPPGPPGTAGFPGSPGAKGEVGPA |
| 676 | GPPGPSGPRGQPGVM |
| 677 | GPPGPSGPRGQPGVMGFPGPKGNDGAPGKN |
| 678 | GPPGSQGESGRPGPPGPSGPRGQPGVM |
| 679 | GPPGTAGFPGSPGAKGEVGPA |
| 680 | GPQGHAGAQGPPGPPGIN |
| 681 | GPRGAAGEPGRDGVPGGPGMRGMPG |
| 682 | GPRGAPGERGRPGLPGAA |
| 683 | GPRGAPGERGRPGLPGAAGA |
| 684 | GPRGAPGERGRPGLPGAAGAR |
| 685 | GPRGAPGERGRPGLPGAAGARGN |
| 686 | GPRGAPGERGRPGLPGAAGARGNDGARG |
| 687 | GPRGAPGERGRPGLPGAAGARGNDGARGSDGQPGPPGPPGTA |
| 688 | GPRGQPGVMGFPGPKGN |
| 689 | GPRGQPGVMGFPGPKGNDGAPGKN |
| 690 | GPSGPAGKDGESGRPGRPGERGLPGPPGIK |
| 691 | GPSGPAGKDGESGRPGRPGERGLPGPPGIKGPA |
| 692 | GPSGPPGPPGAIGPS |
| 693 | GPSGPRGQPGVMGFPGPKGNDGAPGKN |
| 694 | GQAGPSGPPGPPGAIGPS |
| 695 | GQPGPPGPPGTA |
| 696 | GQPGPPGPPGTAGFPGSPGAK |
| 697 | GQPGPPGPPGTAGFPGSPGAKGEVGPA |
| 698 | GQPGVMGFPGPK |
| 699 | GQPGVMGFPGPKGN |
| 700 | GQPGVMGFPGPKGNDGAPGKN |
| 701 | GQRGEPGPQGHAGAQGPPGPPGIN |
| 702 | GRDGVPGGPGMRGMPGSPGGPG |
| 703 | GRDGVPGGPGMRGMPGSPGGPGSDGKPGPPGSQGESGRPGPPGPSGPRGQPGVM |
| 704 | GRNGEKGETGAPGLKGENGLPGEN |
| 705 | GRNGEKGETGAPGLKGENGLPGENGAPGPMGPR |
| 706 | GRPGERGLPGPPGIK |
| 707 | GRPGERGLPGPPGIKGPA |
| 708 | GRPGLPGAAGAR |
| 709 | GRPGLPGAAGARGNDGARGSDGQPGPPGPPGTA |
| 710 | GRPGLPGAAGARGNDGARGSDGQPGPPGPPGTAGFPGSPGAK |
| 711 | GRPGLPGAAGARGNDGARGSDGQPGPPGPPGTAGFPGSPGAKGEVGPA |
| 712 | GRPGPPGPSGPRGQPGVM |
| 713 | GRPGRPGERGLPGPPGIK |
| 714 | GRPGRPGERGLPGPPGIKGPA |
| 715 | GSDGKPGPPGSQGESGRPGPPGPSGPRGQPGVM |
| 716 | GSDGQPGPPGPPGTA |
| 717 | GSDGQPGPPGPPGTAGFPGSPGAK |
| 718 | GSDGQPGPPGPPGTAGFPGSPGAKGEVGPA |
| 719 | GSNGAPGQRGEPGPQGHAGAQGPPGPPGIN |
| 720 | GSPGEPGANGLPGAAGERGAPGFRGPAGPN |
| 721 | GSPGGKGEMGPAGIPGAPG |
| 722 | GSPGGKGEMGPAGIPGAPGLM |
| 723 | GSPGGKGEMGPAGIPGAPGLMG |
| 724 | GSPGGKGEMGPAGIPGAPGLMGA |
| 725 | GSPGGKGEMGPAGIPGAPGLMGAR |
| 726 | GSPGGKGEMGPAGIPGAPGLMGARG |
| 727 | GSPGGKGEMGPAGIPGAPGLMGARGPPG |
| 728 | GSPGGKGEMGPAGIPGAPGLMGARGPPGPA |
| 729 | GSPGGKGEMGPAGIPGAPGLMGARGPPGPAG |
| 730 | GSPGGKGEMGPAGIPGAPGLMGARGPPGPAGAN |
| 731 | GSPGGKGEMGPAGIPGAPGLMGARGPPGPAGANGAPGLR |
| 732 | GSPGGKGEMGPAGIPGAPGLMGARGPPGPAGANGAPGLRGGAGEPGKN |
| 733 | GSPGGPGSDGKPGPPGSQGESGRPGPPGPSGPRGQPGVM |
| 734 | GSPGSNGAPGQRGEPGPQ |
| 735 | GSPGSNGAPGQRGEPGPQGHAGAQ |
| 736 | GSPGSNGAPGQRGEPGPQGHAGAQGP |
| 737 | GSPGSNGAPGQRGEPGPQGHAGAQGPP |
| 738 | GSPGSNGAPGQRGEPGPQGHAGAQGPPG |
| 739 | GSPGSNGAPGQRGEPGPQGHAGAQGPPGP |
| 740 | GSPGSNGAPGQRGEPGPQGHAGAQGPPGPP |
| 741 | GSPGSNGAPGQRGEPGPQGHAGAQGPPGPPG |
| 742 | GSPGSNGAPGQRGEPGPQGHAGAQGPPGPPGIN |
| 743 | GSPGSNGAPGQRGEPGPQGHAGAQGPPGPPGING |
| 744 | GSPGSNGAPGQRGEPGPQGHAGAQGPPGPPGINGS |
| 745 | GSPGSNGAPGQRGEPGPQGHAGAQGPPGPPGINGSPG |
| 746 | GSPGSNGAPGQRGEPGPQGHAGAQGPPGPPGINGSPGG |
| 747 | GSPGSNGAPGQRGEPGPQGHAGAQGPPGPPGINGSPGGK |
| 748 | GSPGSNGAPGQRGEPGPQGHAGAQGPPGPPGINGSPGGKG |
| 749 | GSPGSNGAPGQRGEPGPQGHAGAQGPPGPPGINGSPGGKGEM |
| 750 | GSPGSNGAPGQRGEPGPQGHAGAQGPPGPPGINGSPGGKGEMG |
| 751 | GSPGSNGAPGQRGEPGPQGHAGAQGPPGPPGINGSPGGKGEMGPA |
| 752 | GSPGSNGAPGQRGEPGPQGHAGAQGPPGPPGINGSPGGKGEMGPAGIPGAPGLM |
| 753 | GSPGSNGAPGQRGEPGPQGHAGAQGPPGPPGINGSPGGKGEMGPAGIPGAPGLMG |
| 754 | GSPGSNGAPGQRGEPGPQGHAGAQGPPGPPGINGSPGGKGEMGPAGIPGAPGLMGA |
| 755 | GSQGESGRPGPPGPSGPRGQPGVM |
| 756 | GTAGFPGSPGAKGEVGPA |
| 757 | GVPGGPGMRGMPGSPGGPGSDGKPGPPGSQ |
| 758 | GVPGGPGMRGMPGSPGGPGSDGKPGPPGSQGESGRPGPPGPSGPRGQPGVM |
| 759 | GYPGPAGPPGPPGPPGT |
| 760 | GYPGPAGPPGPPGPPGTS |
| 761 | GYPGPAGPPGPPGPPGTSGH |
| 762 | GYPGPAGPPGPPGPPGTSGHPG |
| 763 | GYPGPAGPPGPPGPPGTSGHPGSPGSPGYQGPPGEPGQAGPSGPPGPPGAIGPS |
| 764 | HAGAQGPPGPPGIN |
| 765 | HAGAQGPPGPPGINGSPG |
| 766 | HAGAQGPPGPPGINGSPGGKGEMG |
| 767 | HAGAQGPPGPPGINGSPGGKGEMGPA |
| 768 | HAGAQGPPGPPGINGSPGGKGEMGPAGIPGAPGLMGAR |
| 769 | HPGSPGSPGYQGPPGEPGQAGPSGPPGPPGAIGPS |
| 770 | HRGFDGRNGEKGETGAPGLKGEN |
| 771 | HRGFDGRNGEKGETGAPGLKGENGLPGENGAPGPMGPR |
| 772 | IGPSGPAGKDGESGRPGRPGERGLPGPPGIK |
| 773 | IKGPAGIPGFPG |
| 774 | IKGPAGIPGFPGMK |
| 775 | IPGAPGLMGARGPPGPAGANGAPGLR |
| 776 | IPGFPGMKGHRGFDGRN |
| 777 | KDGESGRPGRPGERGLPGPPGIK |
| 778 | KDGESGRPGRPGERGLPGPPGIKGPA |
| 779 | KDGSPGEPGANGLPGAAGERGAPGFRGPAGPN |
| 780 | KGENGLPGENGAPGPMGPR |
| 781 | KGPAGIPGFPGMK |
| 782 | KPGPPGSQGESGRPGPPGPSGPR |
| 783 | KPGPPGSQGESGRPGPPGPSGPRGQPGVM |
| 784 | LKGENGLPGENGAPGPMGPR |
| 785 | LMGARGPPGPAGANGAPGLR |
| 786 | LPGAAGARGNDGARGSDGQPGPPGPPGTA |
| 787 | LPGAAGERGAPGFRGPAGPN |
| 788 | LPGPPGIK |
| 789 | LPGPPGIKGPA |
| 790 | LPGPPGIKGPAGIPGFPGMK |
| 791 | MGARGPPGPAGANGAPGLR |
| 792 | MGARGPPGPAGANGAPGLRGGAGEPGKN |
| 793 | MGPAGIPGAPGLMGARGPPGPAGANGAPGLR |
| 794 | MGPRGAPGERGRPGLPGAAGAR |
| 795 | MPGSPGGPGSDGKPGPPGSQGESGRPGPPGPSGPRGQPGVM |
| 796 | MRGMPGSPGGPGSDGKPGPPGSQGESGRPGPPGPSGPRGQPGVM |
| 797 | NDGARGSDGQPGPPGPPGTA |
| 798 | NDGARGSDGQPGPPGPPGTAGFPGSPGAKGEVGPA |
| 799 | NGAPGPMGPRGAPGE |
| 800 | NGAPGPMGPRGAPGERGRPGLPGAAGAR |
| 801 | NGEKGETGAPGLKGENGLPGENGAPGPMGPR |
| 802 | NGETGPQGPPGPTGPGGD |
| 803 | NGLPGENGAPGPMGPR |
| 804 | NGSPGGKGEMGPAGIPGAPGLM |
| 805 | NGSPGGKGEMGPAGIPGAPGLMGARGPPGPAGANGAPGLR |
| 806 | PAGIPGAPGLM |
| 807 | PAGIPGAPGLMG |
| 808 | PAGIPGFPGMK |
| 809 | PAGIPGFPGMKG |
| 810 | PAGIPGFPGMKGHR |
| 811 | PAGIPGFPGMKGHRG |
| 812 | PAGIPGFPGMKGHRGF |
| 813 | PAGIPGFPGMKGHRGFDG |
| 814 | PAGIPGFPGMKGHRGFDGRN |
| 815 | PAGKDGESGRPGRPGERGLPGPPGIK |
| 816 | PGAAGERGAPGFRGPAGPN |
| 817 | PGANGLPGAAGERGAP |
| 818 | PGAPGLMGARGPPGPAGANGAPGLR |
| 819 | PGEPGPKGDAGAPGAPGGKGDAGAPGE |
| 820 | PGERGLPGPPGIK |
| 821 | PGERGRPGLPGAAGAR |
| 822 | PGFPGMKGH |
| 823 | PGFPGMKGHR |
| 824 | PGFPGMKGHRGF |
| 825 | PGFPGMKGHRGFDGR |
| 826 | PGLMGARGPPGPAGANGAPGL |
| 827 | PGLPGAAGARGNDGARGSDGQPGPPGPPGTAGFPGSPGAKG |
| 828 | PGPAGANGAPGLRGGAGEPGKN |
| 829 | PGPAGPPGPPGPPG |
| 830 | PGPAGPPGPPGPPGTS |
| 831 | PGPAGPPGPPGPPGTSG |
| 832 | PGPAGPPGPPGPPGTSGH |
| 833 | PGPAGPPGPPGPPGTSGHPGSPGSPGYQGPPGEPGQAGPSGP |
| 834 | PGPAGPPGPPGPPGTSGHPGSPGSPGYQGPPGEPGQAGPSGPPGPPGAIGPSGPAGK |
| 835 | PGPMGPRGAPGERGRPGLPGAAGAR |
| 836 | PGPPGAIGPS |
| 837 | PGPPGIKGPAGIPGFPGMK |
| 838 | PGPPGPPGTAGFPGSPGAK |
| 839 | PGPPGPSGPRGQPGVM |
| 840 | PGRPGERGLPGPPGIK |
| 841 | PGSDGKPGPPGSQGESGRPGPPGPSGPRGQPGVM |
| 842 | PGSNGAPGQRGEPGPQGHAGAQGPPGPPGINGS |
| 843 | PPGAIGPSGPAGKDGESGRPGRPGER |
| 844 | PPGAIGPSGPAGKDGESGRPGRPGERGLPGPPGIK |
| 845 | PPGEPGQAGPSGPPGPPGAIGPS |
| 846 | PPGEPGQAGPSGPPGPPGAIGPSGPAGKD |
| 847 | PPGIKGPAGIPGFPGMK |
| 848 | PPGINGSPGGKGEMGPAGIPGAPGLM |
| 849 | PPGPAGANGAPGLR |
| 850 | PPGPPGAIGPS |
| 851 | PPGPPGAIGPSGPAGKDGESGRPGRPGERGLPGPPGIK |
| 852 | PPGPPGINGSPGGKGEMGPAGIPGAPGLM |
| 853 | PPGPPGTAGFPGSPGAKGEVGPA |
| 854 | PPGSQGESGRPGPPGPSGPRGQPGVM |
| 855 | PPGTAGFPGSPGAKGEVGPA |
| 856 | PQGHAGAQGPPGPPGIN |
| 857 | PSGPAGKDGESGRPGRPGERGLPGPPGIK |
| 858 | PSGPPGPPGAIGPS |
| 859 | QAGPSGPPGPPGAIGPS |
| 860 | QGPPGEPGQAGPSGPPGPPGAIGPSGPAGKD |
| 861 | QRGEPGPQGHAGAQGPPGPPGIN |
| 862 | RGFDGRNGEKGETGAPGLKGEN |
| 863 | RGLPGPPGIK |
| 864 | RGLPGPPGIKGPA |
| 865 | RGLPGPPGIKGPAGIPGFPGMK |
| 866 | RGMPGSPGGPGSDGKPGPPGSQGESGRPGPPGPSGPRGQPGVM |
| 867 | RGNDGARGSDGQPGPPGPPGTA |
| 868 | RGNDGARGSDGQPGPPGPPGTAGFPGSPGAK |
| 869 | RGNDGARGSDGQPGPPGPPGTAGFPGSPGAKGEVGPA |
| 870 | RGPPGPAGANGAPGL |
| 871 | RGPPGPAGANGAPGLR |
| 872 | RGPPGPAGANGAPGLRG |
| 873 | RGPPGPAGANGAPGLRGGAGEPGKN |
| 874 | RGPPGPAGANGAPGLRGGAGEPGKNGAK |
| 875 | RGSDGQPGPPGPPGTAGFPGSPGAK |
| 876 | RGSDGQPGPPGPPGTAGFPGSPGAKGEVGPA |
| 877 | RPGERGLPGPPGIK |
| 878 | RPGERGLPGPPGIKGPA |
| 879 | RPGLPGAAGARGNDGARGSDGQPGPPGP |
| 880 | RPGLPGAAGARGNDGARGSDGQPGPPGPPGTA |
| 881 | RPGPPGPSGPRGQPGVM |
| 882 | RPGRPGERGLPGPPGIK |
| 883 | RPGRPGERGLPGPPGIKGPA |
| 884 | SDGKPGPPGSQGESGRPGPPGPS |
| 885 | SDGKPGPPGSQGESGRPGPPGPSG |
| 886 | SDGKPGPPGSQGESGRPGPPGPSGPR |
| 887 | SDGKPGPPGSQGESGRPGPPGPSGPRG |
| 888 | SDGKPGPPGSQGESGRPGPPGPSGPRGQPG |
| 889 | SDGKPGPPGSQGESGRPGPPGPSGPRGQPGVM |
| 890 | SDGKPGPPGSQGESGRPGPPGPSGPRGQPGVMGFPGPKGN |
| 891 | SDGQPGPPGPPGTA |
| 892 | SDGQPGPPGPPGTAGFPGSPGAK |
| 893 | SDGQPGPPGPPGTAGFPGSPGAKGEVGPA |
| 894 | SGPAGKDGESGRPGRPGERGLPGPPGIK |
| 895 | SGPPGPPGAIGPS |
| 896 | SGPPGPPGAIGPSGPAGKD |
| 897 | SGPPGPPGAIGPSGPAGKDGESGRPGRPGER |
| 898 | SGPPGPPGAIGPSGPAGKDGESGRPGRPGERGLPGPPGIK |
| 899 | SGPPGPPGAIGPSGPAGKDGESGRPGRPGERGLPGPPGIKGPA |
| 900 | SGPRGQPGVMGFPGPKGN |
| 901 | SGRPGPPGPSGPRGQPGVM |
| 902 | SGRPGPPGPSGPRGQPGVMGFPGPKGN |
| 903 | SGRPGRPGERGLPGPP |
| 904 | SGRPGRPGERGLPGPPG |
| 905 | SGRPGRPGERGLPGPPGIK |
| 906 | SGRPGRPGERGLPGPPGIKGPA |
| 907 | SNGAPGQRGEPGPQGHAGAQGPPGPPGIN |
| 908 | SNGAPGQRGEPGPQGHAGAQGPPGPPGINGSPGGK |
| 909 | SNGAPGQRGEPGPQGHAGAQGPPGPPGINGSPGGKGEMGPA |
| 910 | SPGAKGEVGPAGSPGSNGAPG |
| 911 | SPGAKGEVGPAGSPGSNGAPGQ |
| 912 | SPGAKGEVGPAGSPGSNGAPGQRGE |
| 913 | SPGAKGEVGPAGSPGSNGAPGQRGEPG |
| 914 | SPGAKGEVGPAGSPGSNGAPGQRGEPGPQ |
| 915 | SPGAKGEVGPAGSPGSNGAPGQRGEPGPQG |
| 916 | SPGAKGEVGPAGSPGSNGAPGQRGEPGPQGH |
| 917 | SPGAKGEVGPAGSPGSNGAPGQRGEPGPQGHAG |
| 918 | SPGAKGEVGPAGSPGSNGAPGQRGEPGPQGHAGAQGPPGPPGIN |
| 919 | SPGEPGANGLPGAAGERGAPGFRGPAGPN |
| 920 | SPGGKGEMGPAGIPGAPGLM |
| 921 | SPGGKGEMGPAGIPGAPGLMGA |
| 922 | SPGGKGEMGPAGIPGAPGLMGAR |
| 923 | SPGGKGEMGPAGIPGAPGLMGARGPPGPAGAN |
| 924 | SPGGPGSDGKPGPPGSQGESGRPGPPG |
| 925 | SPGGPGSDGKPGPPGSQGESGRPGPPGP |
| 926 | SPGGPGSDGKPGPPGSQGESGRPGPPGPS |
| 927 | SPGGPGSDGKPGPPGSQGESGRPGPPGPSGPR |
| 928 | SPGGPGSDGKPGPPGSQGESGRPGPPGPSGPRG |
| 929 | SPGGPGSDGKPGPPGSQGESGRPGPPGPSGPRGQPGVM |
| 930 | SPGGPGSDGKPGPPGSQGESGRPGPPGPSGPRGQPGVMGFPGPKGN |
| 931 | SPGSNGAPGQRGEPGPQGHAGAQGPPG |
| 932 | SPGSNGAPGQRGEPGPQGHAGAQGPPGPPGIN |
| 933 | SPGSNGAPGQRGEPGPQGHAGAQGPPGPPGINGSPG |
| 934 | SPGSNGAPGQRGEPGPQGHAGAQGPPGPPGINGSPGGK |
| 935 | SPGSNGAPGQRGEPGPQGHAGAQGPPGPPGINGSPGGKGEMG |
| 936 | SPGSNGAPGQRGEPGPQGHAGAQGPPGPPGINGSPGGKGEMGPA |
| 937 | SPGSPGYQGPPGEPGQAGPSGPPGPPGAIGPS |
| 938 | SPGYQGPPGEPGQAGPSGPPGPPGAIGPS |
| 939 | SQGESGRPGPPGPSGPRGQPGVM |
| 940 | SQGESGRPGPPGPSGPRGQPGVMGFPGPKGN |
| 941 | TAGFPGSPGAKGEVGPA |
| 942 | TAGFPGSPGAKGEVGPAG |
| 943 | TAGFPGSPGAKGEVGPAGSPG |
| 944 | TAGFPGSPGAKGEVGPAGSPGSN |
| 945 | TGAPGLKGENGLPGENGAPGPMGPR |
| 946 | TGPQGPPGPTGPGGD |
| 947 | VGPAGSPGSNGAPGQRGEPGPQG |
| 948 | GHPGSPGSPGYQGPPGEPGQAGPSGPPGPPGAIGPSGPAGKD |
| 949 | GNDGARGSDGQPGPPGPPGTAGFPGSPGAKGEVGP |
| 950 | GFPGPKGNDGAPGKNGERGGPGGPGPQGPPGKNGETGPQGPPGPTGPGGD |
| 951 | GSPGSNGAPGQRGEPGPQGHAGAQGPPGPPGINGSPGGKGEMGPAG |
| 952 | GNDGARGSDGQPGPPGPPGTAGFPGSPGAKGE |
| 953 | GNDGARGSDGQPGPPGPPGTAGFPGSPGAKGEVGPAGSPGSNG |
| 954 | PGPKGNDGAPGKNGERGGPGGPGPQGPPGKN |
| 955 | GTSGHPGSPGSPGYQGPPGEPGQAGPSGPPGPPGAIGPS |
| 956 | GHPGSPGSPGYQGPPGEPGQAGPSGPPGPPGAIGPSGPAGKDGES |
| 957 | GNDGARGSDGQPGPPGPPGTAGFPGSPGAKGEVGPAGSPGSNGAPGQ |
| 958 | PGSPGAKGEVGPA |
| 959 | GAPGLRGGAGEPGKN |
| 960 | GIPGAPGLMGARGPPGPAGA |
| 961 | GFPGSPGAKGEVGPAGSPGSNGAPGQ |
| 962 | PGPKGNDGAPGKN |
| 963 | GEVGPAGSPGSNGAPGQR |

TABLE 2

| SEQ ID | |
|---|---|
| NO. | Sequences |
| 29 | AGYPGPAGPPGPPGPPGTS |
| 30 | AGYPGPAGPPGPPGPPGTSGHPGSPGSPGYQGPPGEPGQAGPSGPPGPPGAIGPS |
| 53 | ARGPPGPAGANGAPGLR |
| 75 | DGARGSDGQPGPPGPPGTAGFPGSPGAK |
| 77 | DGARGSDGQPGPPGPPGTAGFPGSPGAKGEVGPA |
| 86 | DGRNGEKGETGAPGLK |
| 86 | DGRNGEKGETGAPGLK |
| 88 | DGRNGEKGETGAPGLKGENGLPGENGAPGPMGPR |
| 89 | DGRNGEKGETGAPGLKGENGLPGENGAPGPMGPRGAPGERGRPGLPGAA |
| 137 | GANGAPGLRGGAGEPGKN |
| 163 | GAPGERGRPGLPGAAGARGNDGARGSDGQPGPPGPPGTA |
| 164 | GAPGERGRPGLPGAAGARGNDGARGSDGQPGPPGPPGTAGFPGSPGAK |
| 166 | GAPGERGRPGLPGAAGARGNDGARGSDGQPGPPGPPGTAGFPGSPGAKGEVGPA |
| 189 | GAPGQRGEPGPQGHAGAQGPPGPPGIN |
| 197 | GAPGQRGEPGPQGHAGAQGPPGPPGINGSPGGKGEMGPA |
| 204 | GARGNDGARGSDGQPGPPGPPGTA |
| 207 | GARGNDGARGSDGQPGPPGPPGTAGFPGSPGAK |
| 208 | GARGNDGARGSDGQPGPPGPPGTAGFPGSPGAKGEVGPA |
| 211 | GARGNDGARGSDGQPGPPGPPGTAGFPGSPGAKGEVGPAGSPGSN |
| 212 | GARGPPGPAGAN |
| 214 | GARGPPGPAGANGAPGLR |
| 221 | GARGPPGPAGANGAPGLRGGAGEPGKN |
| 224 | GARGPPGPAGANGAPGLRGGAGEPGKNGAK |
| 229 | GARGSDGQPGPPGPPGTA |
| 231 | GARGSDGQPGPPGPPGTAGFPGSPGAKGEVGPA |
| 236 | GEKGETGAPGLKGENGLPGENGAPGPMGPR |
| 238 | GEMGPAGIPGAPGLM |
| 239 | GEMGPAGIPGAPGLMGAR |
| 241 | GEMGPAGIPGAPGLMGARGPPGPAGAN |
| 245 | GENGLPGENGAPGPMGPR |
| 258 | GEPGPQGHAGAQGPPGPPGIN |
| 262 | GEPGQAGPSGPPGPPGAIGPS |
| 269 | GERGGPGGPGPQGPPGKN |
| 271 | GERGGPGGPGPQGPPGKNGETGP |
| 272 | GERGGPGGPGPQGPPGKNGETGPQ |
| 273 | GERGGPGGPGPQGPPGKNGETGPQG |
| 275 | GERGGPGGPGPQGPPGKNGETGPQGPPG |
| 278 | GERGGPGGPGPQGPPGKNGETGPQGPPGPTG |
| 279 | GERGGPGGPGPQGPPGKNGETGPQGPPGPTGPG |
| 280 | GERGGPGGPGPQGPPGKNGETGPQGPPGPTGPGG |
| 282 | GERGGPGGPGPQGPPGKNGETGPQGPPGPTGPGGDK |
| 283 | GERGGPGGPGPQGPPGKNGETGPQGPPGPTGPGGDKG |
| 284 | GERGGPGGPGPQGPPGKNGETGPQGPPGPTGPGGDKGD |
| 285 | GERGGPGGPGPQGPPGKNGETGPQGPPGPTGPGGDKGDTG |
| 289 | GERGGPGGPGPQGPPGKNGETGPQGPPGPTGPGGDKGDTGPPGPQGLQ |
| 316 | GEVGPAGSPGSNGAPGQRGEPGPQ |
| 317 | GEVGPAGSPGSNGAPGQRGEPGPQG |
| 322 | GEVGPAGSPGSNGAPGQRGEPGPQGHAGAQ |
| 323 | GEVGPAGSPGSNGAPGQRGEPGPQGHAGAQG |
| 326 | GEVGPAGSPGSNGAPGQRGEPGPQGHAGAQGPPG |
| 330 | GEVGPAGSPGSNGAPGQRGEPGPQGHAGAQGPPGPPGIN |
| 339 | GEVGPAGSPGSNGAPGQRGEPGPQGHAGAQGPPGPPGINGSPGGKGEMGPA |
| 341 | GFDGRNGEKGETGAPGLK |
| 346 | GFDGRNGEKGETGAPGLKGENGLPGENGAPGPMGPR |
| 350 | GFPGPKGNDGAPGKN |
| 351 | GFPGPKGNDGAPGKNGER |
| 356 | GFPGPKGNDGAPGKNGERGGPGG |
| 360 | GFPGPKGNDGAPGKNGERGGPGGPGPQ |
| 364 | GFPGPKGNDGAPGKNGERGGPGGPGPQGPPG |
| 366 | GFPGPKGNDGAPGKNGERGGPGGPGPQGPPGKN |
| 368 | GFPGPKGNDGAPGKNGERGGPGGPGPQGPPGKNGE |
| 370 | GFPGPKGNDGAPGKNGERGGPGGPGPQGPPGKNGETG |
| 372 | GFPGPKGNDGAPGKNGERGGPGGPGPQGPPGKNGETGPQ |
| 373 | GFPGPKGNDGAPGKNGERGGPGGPGPQGPPGKNGETGPQG |
| 374 | GFPGPKGNDGAPGKNGERGGPGGPGPQGPPGKNGETGPQGPPG |
| 375 | GFPGPKGNDGAPGKNGERGGPGGPGPQGPPGKNGETGPQGPPGPTG |
| 379 | GFPGSPGAKGEVGPA |
| 382 | GFPGSPGAKGEVGPAGSPG |
| 383 | GFPGSPGAKGEVGPAGSPGSN |
| 384 | GFPGSPGAKGEVGPAGSPGSNG |
| 385 | GFPGSPGAKGEVGPAGSPGSNGA |
| 387 | GFPGSPGAKGEVGPAGSPGSNGAPGQR |
| 390 | GFPGSPGAKGEVGPAGSPGSNGAPGQRGEP |
| 391 | GFPGSPGAKGEVGPAGSPGSNGAPGQRGEPG |
| 392 | GFPGSPGAKGEVGPAGSPGSNGAPGQRGEPGPQ |
| 398 | GFPGSPGAKGEVGPAGSPGSNGAPGQRGEPGPQGHAGAQ |
| 399 | GFPGSPGAKGEVGPAGSPGSNGAPGQRGEPGPQGHAGAQG |
| 403 | GFPGSPGAKGEVGPAGSPGSNGAPGQRGEPGPQGHAGAQGPPGPPGIN |
| 406 | GFPGSPGAKGEVGPAGSPGSNGAPGQRGEPGPQGHAGAQGPPGPPGINGSPGGK |
| 420 | GGPGSDGKPGPPGSQGESGRPGPPGPSGPRGQPGVM |
| 426 | GHAGAQGPPGPPGINGSPGGKGEMGPA |
| 432 | GHAGAQGPPGPPGINGSPGGKGEMGPAGIPGAPGLMGARGPPGPAGAN |
| 433 | GHAGAQGPPGPPGINGSPGGKGEMGPAGIPGAPGLMGARGPPGPAGANGAPGLR |
| 435 | GHPGSPGSPGYQGPPGEPGQA |
| 437 | GHPGSPGSPGYQGPPGEPGQAGPS |
| 438 | GHPGSPGSPGYQGPPGEPGQAGPSGPPGPPGAIGPS |
| 439 | GHPGSPGSPGYQGPPGEPGQAGPSGPPGPPGAIGPSGPA |
| 441 | GHRGFDGRNGEKGETGAPGLK |
| 445 | GHRGFDGRNGEKGETGAPGLKGENGLPGENGAPGPMGPR |
| 452 | GIPGAPGLMGA |
| 457 | GIPGAPGLMGARGPPGPAGAN |
| 493 | GKGEMGPAGIPGAPGLM |
| 508 | GLAGYPGPAGPPGPPGPPGTS |
| 509 | GLAGYPGPAGPPGPPGPPGTSGHPGSPGSPGYQGPPGEPGQAGPSGPPGPPGAIGPS |
| 511 | GLMGARGPPGPAGANGAPGLR |
| 538 | GLPGPPGIKGPA |
| 544 | GLPGPPGIKGPAGIPGFPGMK |
| 547 | GLPGPPGIKGPAGIPGFPGMKGHR |
| 560 | GNDGARGSDGQPGPPGPPGTA |
| 562 | GNDGARGSDGQPGPPGPPGTAGF |
| 563 | GNDGARGSDGQPGPPGPPGTAGFPG |
| 565 | GNDGARGSDGQPGPPGPPGTAGFPGSPGAK |
| 566 | GNDGARGSDGQPGPPGPPGTAGFPGSPGAKG |
| 567 | GNDGARGSDGQPGPPGPPGTAGFPGSPGAKGEVG |
| 568 | GNDGARGSDGQPGPPGPPGTAGFPGSPGAKGEVGPA |
| 572 | GNDGARGSDGQPGPPGPPGTAGFPGSPGAKGEVGPAGSPGSN |
| 573 | GNDGARGSDGQPGPPGPPGTAGFPGSPGAKGEVGPAGSPGSNGAPGQR |
| 574 | GNDGARGSDGQPGPPGPPGTAGFPGSPGAKGEVGPAGSPGSNGAPGQRGE |
| 575 | GNDGARGSDGQPGPPGPPGTAGFPGSPGAKGEVGPAGSPGSNGAPGQRGEPGPQ |
| 578 | GPAGIPGAPGLM |
| 583 | GPAGIPGFP |
| 586 | GPAGIPGFPGMK |
| 587 | GPAGIPGFPGMKG |
| 588 | GPAGIPGFPGMKGH |
| 589 | GPAGIPGFPGMKGHR |
| 591 | GPAGIPGFPGMKGHRGF |
| 607 | GPAGIPGFPGMKGHRGFDGRNGEKGETGAPGLK |
| 609 | GPAGIPGFPGMKGHRGFDGRNGEKGETGAPGLKGEN |
| 626 | GPGSDGKPGPPGSQGESGRPGPPGPSGPRGQPGVM |
| 634 | GPPGEPGQAGPSGPPGPPGAIGPS |
| 635 | GPPGEPGQAGPSGPPGPPGAIGPSGPAGKD |
| 639 | GPPGPAGANGAPGLR |
| 648 | GPPGPAGANGAPGLRGGAGEPGKN |
| 662 | GPPGPPGAIGPS |
| 666 | GPPGPPGINGSPGGKGEMGPA |
| 667 | GPPGPPGINGSPGGKGEMGPAGIPGAPGLM |
| 673 | GPPGPPGPPGTSGHPGSPGSPGYQGPPGEPGQAGPSGPPGPPGAIGPS |
| 675 | GPPGPPGTAGFPGSPGAKGEVGPA |
| 678 | GPPGSQGESGRPGPPGPSGPRGQPGVM |
| 679 | GPPGTAGFPGSPGAKGEVGPA |
| 709 | GRPGLPGAAGARGNDGARGSDGQPGPPGPPGTA |
| 717 | GSDGQPGPPGPPGTAGFPGSPGAK |
| 718 | GSDGQPGPPGPPGTAGFPGSPGAKGEVGPA |
| 722 | GSPGGKGEMGPAGIPGAPGLM |
| 724 | GSPGGKGEMGPAGIPGAPGLMGA |
| 725 | GSPGGKGEMGPAGIPGAPGLMGAR |
| 728 | GSPGGKGEMGPAGIPGAPGLMGARGPPGPA |
| 730 | GSPGGKGEMGPAGIPGAPGLMGARGPPGPAGAN |
| 734 | GSPGSNGAPGQRGEPGPQ |
| 739 | GSPGSNGAPGQRGEPGPQGHAGAQGPPGP |
| 741 | GSPGSNGAPGQRGEPGPQGHAGAQGPPGPPG |
| 742 | GSPGSNGAPGQRGEPGPQGHAGAQGPPGPPGIN |
| 743 | GSPGSNGAPGQRGEPGPQGHAGAQGPPGPPGING |
| 745 | GSPGSNGAPGQRGEPGPQGHAGAQGPPGPPGINGSPG |
| 747 | GSPGSNGAPGQRGEPGPQGHAGAQGPPGPPGINGSPGGK |
| 750 | GSPGSNGAPGQRGEPGPQGHAGAQGPPGPPGINGSPGGKGEMG |
| 751 | GSPGSNGAPGQRGEPGPQGHAGAQGPPGPPGINGSPGGKGEMGPA |
| 752 | GSPGSNGAPGQRGEPGPQGHAGAQGPPGPPGINGSPGGKGEMGPAGIPGAPGLM |
| 754 | GSPGSNGAPGQRGEPGPQGHAGAQGPPGPPGINGSPGGKGEMGPAGIPGAPGLMGA |
| 756 | GTAGFPGSPGAKGEVGPA |
| 760 | GYPGPAGPPGPPGPPGTS |
| 763 | GYPGPAGPPGPPGPPGTSGHPGSPGSPGYQGPPGEPGQAGPSGPPGPPGAIGPS |
| 791 | MGARGPPGPAGANGAPGLR |
| 871 | RGPPGPAGANGAPGLR |
| 889 | SDGKPGPPGSQGESGRPGPPGPSGPRGQPGVM |
| 893 | SDGQPGPPGPPGTAGFPGSPGAKGEVGPA |
| 895 | SGPPGPPGAIGPS |
| 920 | SPGGKGEMGPAGIPGAPGLM |
| 937 | SPGSPGYQGPPGEPGQAGPSGPPGPPGAIGPS |
| 939 | SQGESGRPGPPGPSGPRGQPGVM |
| 948 | GHPGSPGSPGYQGPPGEPGQAGPSGPPGPPGAIGPSGPAGKD |
| 964 | GYPGPAGPPGPPGPPGTSGHPGSPGSPGYQGPPGEPGQAGPSGPPGPPGAIGPSGPA |
| 965 | GEKGETGAPGLKGENGLPGENGAPGPMGPRGAPGERGRPGLPGAA |
| 966 | GGAGEPGKNGAKGEPGPRGERGEAGIPGVPGAKGEDGKDG |
| 967 | GGAGEPGKNGAKGEPGPRGERGEAGIPGVPGAKGEDGKDGSPGEPGAN |
| 968 | GHRGFDGRNGEKGETGAPGLKGENGLPGENGAPGPM |
| 969 | DGQPGPPGPPGTAGFPGSPGAKGEVGPA |
| 970 | ARGSDGQPGPPGPPGTAGFPGSPGAKGEVGPA |
| 971 | GNDGARGSDGQPGPPGPPGTAGFPGSPG |
| 972 | GNDGARGSDGQPGPPGPPG |

In some embodiments, the collagen fragment sequence variant described herein can have an amino acid sequence provided below in Table 4. In some embodiments, the collagen fragment sequence variant described herein can have an amino acid sequence that is encoded by a nucleic acid sequence set forth below in Table 5. The relationship between the amino acid sequence, the nucleic acid sequence encoding the amino acid sequence, and the sequence similarity to SEQ ID NO: 986 is provided below in Table 3.

TABLE 3

| | | % Protein Sequence |
|---|---|---|
| AA SEQ | DNA SEQ | similarity to SEQ ID |
| ID NO. | ID NO: | NO: 986 |
| 987 | 1016 | 100% |
| 988 | 1017 | 92% |
| 989 | 1018 | 90% |
| 990 | 1019 | 98% |
| 991 | 1020 | 90% |
| 992 | 1021 | 96% |
| 993 | 1022 | 90% |
| 994 | 1023 | 100% |
| 995 | 1024 | 77% |
| 996 | 1025 | 77% |
| 997 | 1026 | 68% |
| 998 | 1027 | 99% |
| 999 | 1028 | 92% |
| 1000 | 1029 | 94% |
| 1001 | 1030 | 90% |
| 1002 | 1031 | 99% |
| 1003 | 1032 | 93% |
| 1004 | 1033 | 90% |
| 1005 | 1034 | 99% |
| 1006 | 1035 | 96% |
| 1007 | 1036 | 91% |
| 1008 | 1037 | 90% |
| 1009 | 1038 | 100% |
| 1010 | 1039 | 98% |
| 1011 | 1040 | 98% |
| 1012 | 1041 | 96% |
| 1013 | 1042 | 90% |
| 1014 | 1043 | 91% |
| 1015 | 1044 | 99% |

TABLE 4

| SEQ ID | |
|---|---|
| NO. | Sequences |
| 987 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 988 | DVKSGVAVGGLAGYPGPAGAPGPPGPPGASGHPGSPGAPGYQGPPGAPGQAGPSGAP |
| | GPPGAIGASGPAGKDGASGRPGRPGERGLPGPPGAKGPAGIPGAPGMKGHRGFDGRN |
| | GEKGATGAPGLKGANGLPGENGAPGPMGPRGAPGERGRPGAPGAAGARGADGARGSD |
| | GAPGPPGPPGAAGFPGSPGAKGEVGPAGAPGSNGAPGARGEPGPQGAAGAQGPPGAP |
| | GINGSPGAKGEMGPAGAPGAPGLMGARGPPGPAGANGAPGLRGAAGEPGKNGAKGEP |
| | GPRGARGEAGIPGAPGAKGEDGADGSPGEPGANGLPGAAGARGAPGFRGAAGPNGIP |
| | GAKGPAGERGAPGPAGPRGAAGEPGRDGAPGGPGMRGAPGSPGGPGADGKPGPPGAQ |
| | GESGRPGAPGPSGPRGAPGVMGFPGAKGNDGAPGANGERGGPGAPGPQGPPGANGET |
| | GPQGAPGPTGPGGAKGDTGPPGAQGLQGLPGAGGPPGENGAPGEPGPKGAAGAPGAP |
| | GAKGDAGAPGERGPPAIAGIGGEKAGGFAPYYG |
| 989 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGEPGLMGPRGLPGSPGGPGPAGKEGPVGLP |
| | GIDGRPGPIGPAGARGEPGVMGFPGPKGNDGDPGKNGDKGHAGLAGARGAPGPDGET |
| | GAQGPPGPQGVQGGKGEQGPAGPPGFQGLPGTGGPAGEVGKPGEQGLHGEFGLPGPA |
| | GPRGERGAPGERGPPAIAGIGGEKAGGFAPYYG |
| 990 | DVKSGVAVGGLAGYPGPAGPPGPPGPPGTSGHPGSPGSPGYQGPPGEPGQAGPSGPP |
| | GPPGAIGPSGPAGKRGESGRPGRPGERGLPGPPGIKGPAGIPGFPGMKGHRGFRGRN |
| | GEKGETGAPGLKGENGLPGENGAPGPMGPRGAPGERGRPGLPGAAGARGNRGARGSD |
| | GQPGPPGPPGTAGFPGSPGAKGRVGPAGSPGSNGAPGQRGEPGPQGHAGAQGPPGPP |
| | GINGSPGGKGEMGPAGIPGAPGLMGARGPPGPAGANGAPGLRGGAGRPGKNGAKGEP |
| | GPRGERGRAGIPGVPGAKGERGKDGSPGEPGANGLPGAAGERGAPGFRGPAGPNGIP |
| | GEKGPAGERGAPGPAGPRGAAGEPGRRGVPGGPGMRGMPGSPGGPGSRGKPGPPGSQ |
| | GESGRPGPPGPSGPRGQPGVMGFPGPKGNDGAPGKNGERGGPGGPGPQGPPGKNGET |
| | GPQGPPGPTGPGGRKGDTGPPGPQGLQGLPGTGGPPGENGKPGRPGPKGDAGAPGAP |
| | GGKGRAGAPGERGPPAIAGIGGEKAGGFAPYYG |
| 991 | DVKSGVAVGGLAGYPGPAGPPGPPGPPGASGHPGSPGSPGYQGPPGEPGQAGPAGP |
| | PGPPGAIGPSGPAGKDGESGRPGRPGERGLPGPPGIKGPAGIPGFPGMKGHRGFDGR |
| | NGEKGEAGAPGLKGENGLPGENGAPGPMGPRGAPGERGRPGLPGAAGARGNDGARGS |
| | DGQPGPPGPPGAAGFPGAPGAKGEVGPAGSPGSNGAPGQRGEPGPQGHAGAQGPPGP |
| | PGINGSPGGKGEMGPAGIPGAPGLMGARGPPGPAGANGAPGLRGGAGEPGKNGAKGE |
| | PGPRGERGEAGIPGVPGAKGEDGKDGSPGEPGANGLPGAAGERGAPGFRGPAGPNGI |
| | PGEKGPAGERGAPGPAGPRGAAGEPGRDGVPGGPGMRGMPGSPGGPGSDGKPGPPGA |
| | QGESGRPGPPGPSGPRGQPGVMGFPGPKGNDGAPGKNGERGGPGGPGPQGPPGKNGE |
| | TGPQGPPGPTGPGGDKGDTGPPGPQGLQGLPGTGGPPGENGKPGEPGPKGDAGAPGA |
| | PGGKGDAGAPGERGPPAIAGIGGEKAGGFAPYYG |
| 992 | DVKSGVAVGGLAGYPGPAGPPGPPGPPGNSGHPGSPGSPGYQGPPGEPGQAGPNGPP |
| | GPPGAIGPSGPAGKDGESGRPGRPGERGLPGPPGIKGPAGIPGFPGMKGHRGFDGRN |
| | GEKGENGAPGLKGENGLPGENGAPGPMGPRGAPGERGRPGLPGAAGARGNDGARGSD |
| | GQPGPPGPPGNAGFPGNPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGNQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 993 | DVKSGVAVGGLAGYPGPAGAPGPPGPPGASGHPGSPGAPGYQGPPGAPGQAGPSGAP |
| | GPPGAIGASGPAGKDGASGRPGRPGERGLPGPPGAKGPAGIPGAPGMKGHRGFDGRN |
| | GEKGATGAPGLKGANGLPGENGAPGPMGPRGAPGERGRPGAPGAAGARGADGARGSD |
| | GAPGPPGPPGAAGFPGSPGAKGEVGPAGAPGSNGAPGARGEPGPQGAAGAQGPPGAP |
| | GINGSPGAKGEMGPAGAPGAPGLMGARGPPGPAGANGAPGLRGAAGEPGKNGAKGEP |
| | GPRGARGEAGIPGAPGAKGEDGADGSPGEPGANGLPGAAGARGAPGFRGAAGPNGIP |
| | GAKGPAGERGAPGPAGPRGAAGEPGRDGAPGGPGMRGAPGSPGGPGADGKPGPPGAQ |
| | GESGRPGAPGPSGPRGAPGVMGFPGAKGNDGAPGANGERGGPGAPGPQGPPGANGET |
| | GPQGAPGPTGPGGAKGDTGPPGAQGLQGLPGAGGPPGENGAPGEPGPKGAAGAPGAP |
| | GAKGDAGAPGERGPPAIAGIGGEKAGGFAPYYG |
| 994 | DSYDVKSGVAVGGLAGYPGPAGPPGPPGPPGTSGHPGSPGSPGYQGPPGEPGQAGPS |
| | GPPGPPGAIGPSGPAGKDGESGRPGRPGERGLPGPPGIKGPAGIPGFPGMKGHRGFD |
| | GRNGEKGETGAPGLKGENGLPGENGAPGPMGPRGAPGERGRPGLPGAAGARGNDGAR |
| | GSDGQPGPPGPPGTAGFPGSPGAKGEVGPAGSPGSNGAPGQRGEPGPQGHAGAQGPP |
| | GPPGINGSPGGKGEMGPAGIPGAPGLMGARGPPGPAGANGAPGLRGGAGEPGKNGAK |
| | GEPGPRGERGEAGIPGVPGAKGEDGKDGSPGEPGANGLPGAAGERGAPGFRGPAGPN |
| | GIPGEKGPAGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPGPQGPPGKN |
| | GETGPQGPPGPTGPGGDKGDTGPPGPQGLQGLPGTGGPPGENGKPGEPGPKGDAGAP |
| | GAPGGKGDAGAPGERGPPAIAGIGGEKAGGFAPYYG |
| 995 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGAIGPSGPAGKDGESGRPGRPGERGLPGPPGIKGPAGIPGFPGMKGHR |
| | GFDGRNGEKGETGAPGLKGENGLPGENGAPGPMGPRGAPGERGRPGLPGAAGARGND |
| | GARGSDGQPGPPGPPAIAGIGGEKAGGFAPYYG |
| 996 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGAIGPSGPAGKDGESGRPGRPGERGLPGPPGIKGPAGIPGFPGMKGHR |
| | GFDGRNGEKGETGAPGLKGENGLPGENGAPGPMGPRGAPGERGRPGLPGAAGARGND |
| | GARGSDGQPGPPGPPAIAGIGGEKAGGFAPYYG |
| 997 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGAIGPSGPAGKDGESGRPGRPGERGLPGPPGIKGPAGIPGFPGMKGHR |
| | GFDGRNGEKGETGAPGLKGENGLPGENGAPGPMGPRGAPGERGRPGLPGAAGARGND |
| | GARGSDGQPGPPGPPGAIGPSGPAGKDGESGRPGRPGERGLPGPPGIKGPAGIPGFP |
| | GMKGHRGFDGRNGEKGETGAPGLKGENGLPGENGAPGPMGPRGAPGERGRPGLPGAA |
| | GARGNDGARGSDGQPGPPGPPAIAGIGGEKAGGFAPYYG |
| 998 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGHRGFPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 999 | DVKSGVAVGGLAGYPGPAGPPGPPGPPGTSGHPGSPGSPGYQGPPGEPGQAGPSGPP |
| | GPPGAIGPSGPAGKDGESGRPGRPGAAGLPGPPGIKGPAGIPGFPGMKGHRGFDGRN |
| | GEKGETGAPGLKGENGLPGAAGAPGPMGPRGAPGAAGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGPLVLRQLLVLVEKRPAVSLHIMVNQEDVRMPFA |
| 1000 | DVKSGVAVGGLAGYPGPAGPPGPPGPPGTSGHPGSPGSPGYQGPPGEPGQAGPSGPP |
| | GPPGAIGPSGPAGKDGESGKPGRPGERGLPGPPGIKGPAGIPGFPGMKGHRGFDGKN |
| | GEKGETGAPGLKGENGLPGENGAPGPMGPKGAPGERGKPGLPGAAGAKGNDGAKGSD |
| | GQPGPPGPPGTAGFPGSPGAKGEVGPAGSPGSNGAPGQKGEPGPQGHAGAQGPPGPP |
| | GINGSPGGKGEMGPAGIPGAPGLMGAKGPPGPAGANGAPGLKGGAGEPGKNGAKGEP |
| | GPKGEKGEAGIPGVPGAKGEDGKDGSPGEPGANGLPGAAGEKGAPGFKGPAGPNGIP |
| | GEKGPAGEKGAPGPAGPKGAAGEPGKDGVPGGPGMKGMPGSPGGPGSDGKPGPPGSQ |
| | GESGKPGPPGPSGPKGQPGVMGFPGPKGNDGAPGKNGEKGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 1001 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGAVGAKGEAGPQGPRGSEGPQGVRGEPGPPGPAGAAGPAGPP |
| | GADGQPGAKGAMGAPGIAGAPGFPGARGPPGPQGAGGPPGPKGGAGEPGAPGAKGDT |
| | GAKGERGPVGVQGPPGPAGEEGKRGARGEPGPTGLPGPPGERGGPGFRGFPGADGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 1002 | DVKSGVAVGGIAGYPGPAGPPGPPGPPGTSGHPGSPGSPGYQGPPGEPGQAGPSGPP |
| | GPPGALGPSGPAGKDGESGRPGRPGERGVPGPPGVKGPAGLPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGELGPAGSPGSNGAPGQRGEPGPQGHAGAQGPPGPP |
| | GLNGSPGGKGEMGPAGVPGAPGIMGARGPPGPAGANGAPGIRGGAGEPGKNGAKGEP |
| | GPRGERGEAGLPGLPGAKGEDGKDGSPGEPGANGVPGAAGERGAPGFRGPAGPNGLP |
| | GEKGPAGERGAPGPAGPRGAAGEPGRDGIPGGPGMRGMPGSPGGPGSDGKPGPPGSQ |
| | GESGRPGPPGPSGPRGQPGLMGFPGPKGNDGAPGKNGERGGPGGPGPQGPPGKNGET |
| | GPQGPPGPTGPGGDKGDTGPPGPQGVQGLPGTGGPPGENGKPGEPGPKGDAGAPGAP |
| | GGKGDAGAPGERGPPAIAGIGGEKAGGFAPYYG |
| 1003 | DVKSGVAVGGLAGYPGPAGPPGPPGPPGAAGHPGAPGAPGYQGPPGEPGQAGPAGPP |
| | GPPGAIGPAGPAGKDGEAGRPGRPGERGLPGPPGIKGPAGIPGFPGMKGHRGFDGRD |
| | GEKGEAGAPGLKGEDGLPGENGAPGPMGPRGAPGERGRPGLPGAAGARGDDGARGAD |
| | GQPGPPGPPGAAGFPGAPGAKGEVGPAGAPGADGAPGQRGEPGPQGHAGAQGPPGPP |
| | GIDGAPGGKGEMGPAGIPGAPGLMGARGPPGPAGADGAPGLRGGAGEPGKDGAKGEP |
| | GPRGERGEAGIPGVPGAKGEDGKDGAPGEPGADGLPGAAGERGAPGFRGPAGPDGIP |
| | GEKGPAGERGAPGPAGPRGAAGEPGRDGVPGGPGMRGMPGAPGGPGADGKPGPPGAQ |
| | GEAGRPGPPGPAGPRGQPGVMGFPGPKGDDGAPGKDGERGGPGGPGPQGPPGKDGEA |
| | GPQGPPGPAGPGGDKGDAGPPGPQGLQGLPGAGGPPGEDGKPGEPGPKGDAGAPGAP |
| | GGKGDAGAPGERGPPAIAGIGGEKAGGFAPYYG |
| 1004 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIEPGRPEGEKGGGRGGAPGAPGRGAGNGEGARPPPLMGGAG |
| | GNKAGPLAAPIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 1005 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIEPGRPEGEKGGGRGGAPGAPGRGAGNGEGARPPPLMGGAG |
| | GNKAGPLAAPIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 1006 | DVKSGVAVGGLAGYPGPAGPPGPPGPPGTSGHPGSPGSPGYQGPPGEPGQAGPSGPP |
| | GPPGAIGPSGPAGKRGESGRPGRPGERGLPGPPGIKGPAGIPGFPGMKGHRGFRGRN |
| | GEKGRTGAPGLKGENGLPGENGAPGPMGPRGAPGERGRPGLPGAAGARGNRGARGSR |
| | GQPGPPGPPGTAGFPGSPGAKGRVGPAGSPGSNGAPGQRGRPGPQGHAGAQGPPGPP |
| | GINGSPGGKGRMGPAGIPGAPGLMGARGPPGPAGANGAPGLRGGAGRPGKNGAKGRP |
| | GPRGERGRAGIPGVPGAKGRRGKRGSPGEPGANGLPGAAGERGAPGFRGPAGPNGIP |
| | GEKGPAGERGAPGPAGPRGAAGRPGRRGVPGGPGMRGMPGSPGGPGSRGKPGPPGSQ |
| | GESGRPGPPGPSGPRGQPGVMGFPGPKGNRGAPGKNGRRGGPGGPGPQGPPGKNGET |
| | GPQGPPGPTGPGGRKGRTGPPGPQGLQGLPGTGGPPGENGKPGRPGPKGDAGAPGAP |
| | GGKGRAGAPGERGPPAIAGIGGEKAGGFAPYYG |
| 1007 | DVKSGVAVGGMGGYPGPAGPPGPPGPPGVSGHPGAPGAPGYQGPPGEPGQAGPAGPP |
| | GPPGAMGPAGPAGKDGESGRPGRPGERGFPGPPGIKGPAGMPGFPGMKGHRGFDGRN |
| | GEKGDTGAPGLKGENGLPGENGAPGPMGPRGAPGERGRPGLPGAAGARGNDGARGSD |
| | GQPGPPGPPGTAGFPGSPGAKGEVGPAGAPGPNGAPGQRGEPGLQGQAGAPGPPGPP |
| | GINGSPGGKGEMGPAGIPGAPGLIGARGPPGPPGANGVPGQRGAAGEPGKNGAKGDP |
| | GARGERGEAGIPGIAGPKGEDGKDGSPGEPGANGLPGAPGERGPPGFRGAPGANGIP |
| | GEKGPPGERGGPGPAGPRGVAGEPGRDGLPGGPGLRGIPGSPGGPGSDGKPGPPGSQ |
| | GESGRPGPPAHLVQGVSLVSWVSPVQRVMTEHRVKMVNVVAQVVQDCPVQQEKTVRQ |
| | VLKDLQDLPVLQEIREMQDRQDLLDCKVCLVQEALPEKMVSLESQDQKAMSEPQVFP |
| | EVRETPVLRVSVVLRQLLVLVEKRPAVSLHIMVNQEDVRMPFA |
| 1008 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGVA |
| | GPKGPAGERGAPGPAGPKGAAGEAGRPGEAGLPGAKGMPGSPGGPGPDGKPGPPGPA |
| | GQDGRPGPPGPPGARGQAGVMGFPGPKGAAGEPGKAGERGVPGPPGAVGPAGKDGEA |
| | GAQGPPGPAGPAGERGEQGPAGPQGFQGLPGPAGPPGEAGKPGEQGVPGDLGAPGAP |
| | GARGERGAPGERGPPAIAGIGGEKAGGFAPYYG |
| 1009 | DSYDVKSGVAVGGLAGYPGPAGPPGPPGPPGTSGHPGSPGSPGYQGPPGEPGQAGPS |
| | GPPGPPGAIGPSGPAGKDGESGRPGRPGERGLPGPPGIKGPAGIPGFPGMKGHRGFD |
| | GRNGEKGETGAPGLKGENGLPGENGAPGPMGPRGAPGERGRPGLPGAAGARGNDGAR |
| | GSDGQPGPPGPPGTAGFPGSPGAKGEVGPAGSPGSNGAPGQRGEPGPQGHAGAQGPP |
| | GPPGINGSPGGKGEMGPAGIPGAPGLMGARGPPGPAGANGAPGLRGGAGEPGKNGAK |
| | GEPGPRGERGEAGIPGVPGAKGEDGKDGSPGEPGANGLPGAAGERGAPGFRGPAGPN |
| | GIPGEKGPAGERGAPGPAGPRGAAGEPGRDGVPGGPGMRGMPGSPGGPGSDGKPGPP |
| | GSQGESGRPGPPGPSGPRGQPGVMGFPGPKGNDGAPGKNGERGGPGGPGPQGPPGKN |
| | GETGPQGPPGPTGPGGDKGDTGPPGPQGLQGLPGTGGPPGENGKPGEPGPKGDAGAP |
| | GAPGGKGDAGAPGERGPP |
| 1010 | DVKSGVAVGKTTKSGLAGYPGPAGPPGPPGPPGTSGHPGSPGSPGYQGPPGEPGQAG |
| | PSGPPGPPGAIGPSGPAGKDGESGRPGRPGERGLPGPPGIKGPAGIPGFPGMKGHRG |
| | FDGRNGEKGETGAPGLKGENGLPGENGAPGPMGPRGAPGERGRPGLPGAAGARGNDG |
| | ARGSDGQPGPPGPPGTAGFPGSPGAKGEVGPAGSPGSNGAPGQRGEPGPQGHAGAQG |
| | PPGPPGINGSPGGKGEMGPAGIPGAPGLMGARGPPGPAGANGAPGLRGGAGEPGKNG |
| | AKGEPGPRGERGEAGIPGVPGAKGEDGKDGSPGEPGANGLPGAAGERGAPGFRGPAG |
| | PNGIPGEKGPAGERGAPGPAGPRGAAGEPGRDGVPGGPGMRGMPGSPGGPGSDGKPG |
| | PPGSQGESGRPGPPGPSGPRGQPGVMGFPGPKGNDGAPGKNGERGGPGGPGPQGPPG |
| | KNGETGPQGPPGPTGPGGDKGDTGPPGPQGLQGLPGTGGPPGENGKPGEPGPKGDAG |
| | APGAPGGKGDAGAPGERGPPKTTKSAIAGIGGEKAGGFAPYYG |
| 1011 | DVKSGVAVGKTTKSGLAGYPGPAGPPGPPGPPGTSGHPGSPGSPGYQGPPGEPGQAG |
| | PSGPPGPPGAIGPSGPAGKDGESGRPGRPGERGLPGPPGIKGPAGIPGFPGMKGHRG |
| | FDGRNGEKGETGAPGLKGENGLPGENGAPGPMGPRGAPGERGRPGLPGAAGARGNDG |
| | ARGSDGQPGPPGPPGTAGFPGSPGAKGEVGPAGSPGSNGAPGQRGEPGPQGHAGAQG |
| | PPGPPGINGSPGGKGEMGPAGIPGAPGLMGARGPPGPAGANGAPGLRGGAGEPGKNG |
| | AKGEPGPRGERGEAGIPGVPGAKGEDGKDGSPGEPGANGLPGAAGERGAPGFRGPAG |
| | PNGIPGEKGPAGERGAPGPAGPRGAAGEPGRDGVPGGPGMRGMPGSPGGPGSDGKPG |
| | PPGSQGESGRPGPPGPSGPRGQPGVMGFPGPKGNDGAPGKNGERGGPGGPGPQGPPG |
| | KNGETGPQGPPGPTGPGGDKGDTGPPGPQGLQGLPGTGGPPGENGKPGEPGPKGDAG |
| | APGAPGGKGDAGAPGERGPPKTTKSAIAGIGGEKAGGFAPYYG |
| 1012 | DVKSGVAVGKTTKSGLAGYPGPAGPPGPPGPPGTSGHPGSPGSPGYQGPPGEPGQAG |
| | PSGPPGPPGAIGPSGPAGKDGESGRPGRPGAAGLPGPPGIKGPAGIPGFPGMKGHRG |
| | FDGRNGEKGETGAPGLKGENGLPGAAGAPGPMGPRGAPGAAGRPGLPGAAGARGNDG |
| | ARGSDGQPGPPGPPGTAGFPGSPGAKGEVGPAGSPGSNGAPGQRGEPGPQGHAGAQG |
| | PPGPPGINGSPGGKGEMGPAGIPGAPGLMGARGPPGPAGANGAPGLRGGAGEPGKNG |
| | AKGEPGPRGERGEAGIPGVPGAKGEDGKDGSPGEPGANGLPGAAGERGAPGFRGPAG |
| | PNGIPGEKGPAGERGAPGPAGPRGAAGEPGRDGVPGGPGMRGMPGSPGGPGSDGKPG |
| | PPGSQGESGRPGPPGPSGPRGQPGVMGFPGPKGNDGAPGKNGERGGPGGPGPQGPPG |
| | KNGETGPQGPPGPTGPGGDKGDTGPPGPQGLQGLPGTGGPPGENGKPGEPGPKGDAG |
| | APGAPGGKGDAGAPGAAGPPKTTKSAIAGIGGEKAGGFAPYYG |
| 1013 | DVKSGVAVGGLAGYPGPAGPPGPPGPPGTSGHPGSPGSPGYQGPPGEPGQAGPSGPP |
| | GPPGAIGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEEGKRGARGEAGSAGPPGPPGLRGAPGFRGLPGADGRA |
| | GVMGPPGERGAPGPAGVRGAAGDAGRPGEPGLMGPRGLPGSPGGPGPAGKEGPVGLP |
| | GIDGRPGPIGPAGARGEPGVMGFPGPKGNDGDPGKNGDKGHAGLAGARGAPGPDGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |
| 1014 | DVKSGVAVGGMGGYPGPAGPPGPPGPPGVSGHPGAPGAPGYQGPPGEPGQAGPAGPP |
| | GPPGAMGPAGPAGKDGESGRPGRPGERGFPGPPGIKGPAGMPGFPGMKGHRGFDGRN |
| | GEKGDTGAPGLKGENGLPGENGAPGPMGPRGAPGERGRPGLPGAAGARGNDGARGSD |
| | GQPGPPGPPGTAGFPGSPGAKGEVGPAGAPGPNGAPGQRGEPGLQGQAGAPGPPGPP |
| | GINGSPGGKGEMGPAGIPGAPGLIGARGPPGPPGANGVPGQRGAAGEPGKNGAKGDP |
| | GARGERGEAGIPGIAGPKGEDGKDGSPGEPGANGLPGAPGERGPPGFRGAPGANGIP |
| | GEKGPPGERGGPGPAGPRGVAGEPGRDGLPGGPGLRGIPGSPGGPGSDGKPGPPGSQ |
| | GESGRPGPPGSPGPRGQPGVMGFPGPKGNDGAPGKNGERGGPGGPGLPGPAGKNGET |
| | GPQGPPGPTGPAGDKGDAGPPGPPGLQGLPGTGGPPGENGKPGEPGPKGDVGAPGIP |
| | GGKGDAGAPGERGPPAIAGIGGEKAGGFAPYYG |
| 1015 | DVKSGVAVGGLAGYPGPAGPPGPPGPPGTSGHPGSPGSPGYQGPPGEPGQAGPAGPP |
| | GPIGNVGPSGPAGKDGESGRPGRPGERGLPGPPGIKGPAGIPGFPGMKGHRGFDGRN |
| | GEKGETGAPGLKGENGLPGENGAPGPMGPRGAPGERGRPGLPGAAGARGNDGARGSD |
| | GQPGPPGPPGTAGFPGSPGAKGEVGPAGSPGSNGAPGQRGEPGPQGHAGAQGPPGPP |
| | GINGSPGGKGEMGPAGIPGAPGLMGARGPPGPAGANGAPGLRGGAGEPGKNGAKGEP |
| | GPRGERGEAGIPGVPGAKGEDGKDGSPGEPGANGLPGAAGERGAPGFRGPAGPNGIP |
| | GEKGPAGERGAPGPAGPRGAAGEPGRDGVPGGPGMRGMPGSPGGPGSDGKPGPPGSQ |
| | GESGRPGPPGPSGPRGQPGVMGFPGPKGNDGAPGKNGERGGPGGPGPQGPPGKNGET |
| | GPQGPPGPTGPGGDKGDTGPPGPQGLQGLPGTGGPPGENGKPGEPGPKGDAGAPGAP |
| | GGKGDAGAPGERGPPAIAGIGGEKAGGFAPYYG |

In some embodiments, the collagen fragment sequence variant described herein can have an amino acid sequence that is encoded by a nucleic acid sequence set forth below in Table 5.

TABLE 5

| SEQ ID NO. | Sequences |
|---|---|
| 1016 | GACGTCAAATCTGGTGTTGCTGTCGGAGGACTTGCAGGATATCCAGGACC |
| | TGCCGGACCACCTGGACCACCAGGCCCACCAGGCACCTCAGGCCATCCA |
| | GGCTCCCCAGGATCACCTGGATATCAGGGACCACCAGGCGAGCCAGGAC |
| | AGGCCGGCCCAAGTGGACCACCTGGACCACCAGGCGCCATAGGCCCTTC |
| | AGGCCCAGCCGGAAAAGACGGAGAGAGTGGCCGTCCAGGAAGGCCAGG |
| | AGAAAGGGGACTGCCAGGACCACCTGGCATCAAAGGCCCTGCCGGAATA |
| | CCAGGATTCCCAGGCATGAAAGGACATCGTGGATTCGACGGAAGGAATG |
| | GAGAGAAAGGCGAGACCGGCGCCCCAGGACTCAAAGGCGAGAATGGCC |
| | TGCCTGGAGAGAATGGAGCCCCAGGACCAATGGGCCCTCGTGGAGCCCC |
| | AGGCGAAAGGGGACGTCCTGGACTGCCAGGAGCCGCAGGCGCCCGTGGC |
| | AATGACGGCGCCAGGGGAAGTGACGGCCAGCCTGGACCACCAGGCCCAC |
| | CAGGAACAGCTGGATTCCCAGGATCCCCAGGCGCCAAAGGCGAGGTAGG |
| | ACCAGCAGGATCACCAGGCAGTAATGGCGCTCCAGGACAGCGTGGAGAG |
| | CCAGGACCACAAGGACATGCCGGCGCCCAGGGCCCACCAGGACCACCAG |
| | GAATCAATGGCAGTCCAGGCGGCAAAGGAGAGATGGGCCCAGCCGGAAT |
| | CCCAGGAGCCCCAGGATTGATGGGAGCTCGTGGCCCACCTGGACCTGCT |
| | GGCGCCAATGGCGCCCCAGGATTGAGGGGCGGCGCAGGCGAACCAGGCA |
| | AAAATGGCGCTAAAGGAGAACCAGGACCTCGAGGCGAGCGTGGCGAAG |
| | CCGGCATCCCTGGCGTACCTGGAGCCAAAGGAGAGGACGGAAAAGACGG |
| | AAGTCCAGGAGAGCCAGGCGCAAATGGACTGCCTGGAGCCGCAGGAGA |
| | GCGTGGCGCTCCAGGATTCAGGGGACCAGCCGGACCTAATGGAATCCCT |
| | GGAGAGAAAGGCCCAGCCGGAGAACGAGGCGCCCCTGGACCAGCAGGA |
| | CCACGTGGAGCCGCCGGAGAGCCAGGACGTGACGGAGTCCCAGGAGGCC |
| | CTGGAATGCGTGGAATGCCAGGAAGTCCTGGAGGACCTGGAAGTGACGG |
| | AAAACCTGGACCACCAGGAAGTCAGGGAGAGAGTGGCAGGCCTGGCCCA |
| | CCAGGACCTTCAGGCCCAAGGGGCCAACCAGGCGTCATGGGATTCCCAG |
| | GCCCAAAAGGAAATGACGGCGCCCCAGGCAAAAATGGAGAGAGGGGCG |
| | GACCAGGAGGACCAGGACCACAAGGCCCACCAGGAAAAAATGGCGAGA |
| | CAGGACCACAAGGACCACCTGGACCAACAGGACCAGGCGGCGACAAAG |
| | GAGACACCGGACCACCAGGCCCTCAGGGACTCCAGGGACTGCCAGGCAC |
| | CGGCGGCCCACCTGGAGAGAATGGAAAACCAGGAGAGCCTGGCCCTAAA |
| | GGCGACGCTGGCGCACCAGGAGCCCCTGGAGGAAAAGGCGACGCCGGC |
| | GCTCCAGGCGAGCGAGGACCACCAGCAATTGCTGGTATTGGTGGAGAAA |
| | AGGCCGGCGGTTTCGCTCCATATTATGGT |
| 1017 | GACGTCAAATCTGGTGTTGCTGTTGGAGGTTTAGCAGGCTACCCTGGTCC |
| | CGCAGGGGCTCCAGGTCCGCCGGGTCCGCCCGGAGCTTCAGGTCATCCCG |
| | GAAGCCCTGGTGCTCCAGGTTATCAGGGACCGCCCGGAGCTCCTGGACA |
| | AGCTGGTCCCTCCGGAGCTCCTGGTCCACCAGGTGCTATTGGAGCTAGTG |
| | GTCCTGCCGGAAAAGACGGTGCTTCCGGTAGACCTGGTAGACCCGGCGA |
| | AAGGGGTTTACCAGGTCCTCCCGGAGCTAAGGGTCCAGCCGGTATACCC |
| | GGTGCTCCTGGGATGAAGGGTCACAGAGGATTTGATGGTAGAAACGGAG |
| | AGAAAGGCGCTACCGGTGCTCCCGGACTGAAGGGTGCTAACGGTCTTCCT |
| | GGTGAGAACGGCGCTCCTGGACCTATGGGTCCACGTGGTGCTCCAGGAG |
| | AAAGAGGCAGACCAGGAGCTCCTGGTGCAGCTGGTGCTAGAGGTGCTGA |
| | TGGTGCCCGTGGTTCCGATGGAGCTCCCGGGCCACCCGGCCCTCCAGGTG |
| | CTGCTGGATTTCCTGGAAGCCCTGGTGCTAAGGGGGAGGTTGGTCCGGCT |
| | GGTGCTCCCGGAAGTAACGGTGCCCCAGGTGCTAGAGGCGAACCAGGCC |
| | CTCAGGGTGCTGCAGGAGCACAGGGACCGCCTGGTGCTCCTGGTATTAAT |
| | GGTTCGCCTGGAGCTAAAGGTGAAATGGGGCCCGCAGGAGCTCCCGGTG |
| | CGCCTGGTCTTATGGGTGCCAGGGGTCCTCCAGGCCCGGCCGGTGCAAAT |
| | GGTGCTCCCGGATTACGAGGAGCTGCTGGTGAACCTGGTAAAAACGGTG |
| | CCAAAGGAGAACCAGGTCCTCGTGGAGCTCGTGGTGAAGCTGGCATTCC |
| | CGGTGCTCCTGGTGCAAAAGGTGAGGACGGTGCTGACGGTTCCCCTGGTG |
| | AGCCAGGTGCGAACGGACTGCCAGGTGCAGCCGGAGCTCGAGGAGCTCC |
| | AGGATTCAGGGGAGCTGCCGGTCCTAACGGCATTCCTGGTGCTAAAGGG |
| | CCCGCCGGTGAAAGGGGAGCTCCCGGTCCAGCAGGCCCTCGTGGAGCAG |
| | CTGGTGAGCCTGGACGTGACGGTGCTCCAGGAGGGCCAGGTATGAGGGG |
| | TGCTCCCGGGTCCCCTGGCGGTCCTGGAGCTGATGGAAAACCAGGGCCA |
| | CCAGGTGCTCAGGGTGAAAGCGGACGTCCAGGCGCTCCCGGCCCTTCAG |
| | GTCCAAGGGGTGCTCCTGGTGTCATGGGTTTCCCCGGTGCTAAGGGTAAT |
| | GACGGAGCACCGGGTGCTAATGGTGAACGTGGTGGCCCAGGTGCTCCAG |
| | GACCCCAAGGTCCACCAGGAGCTAACGGTGAGACAGGTCCTCAAGGAGC |
| | TCCAGGACCTACCGGTCCTGGAGGAGCTAAGGGAGATACGGGACCGCCA |
| | GGAGCTCAAGGATTGCAAGGTTTGCCTGGTGCTGGAGGCCCTCCCGGAG |
| | AAAATGGTGCTCCTGGAGAGCCAGGACCAAAAGGCGCTGCTGGAGCCCC |
| | AGGTGCACCCGGAGCTAAGGGAGACGCCGGTGCTCCGGGTGAGCGTGGT |
| | CCTCCGGCAATTGCTGGTATTGGTGGAGAAAAGGCCGGCGGTTTCGCTCC |
| | ATATTATGGT |
| 1018 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGAACCAGGACTGATGGGTCCTAGG |
| | GGTCTGCCCGGGTCCCCTGGCGGTCCTGGACCTGCAGGAAAAGAAGGGC |
| | CAGTGGGTCTGCCTGGTATTGACGGACGTCCAGGCCCAATTGGCCCTGCA |
| | GGTGCAAGGGGTGAACCTGGTGTCATGGGTTTCCCCGGTCCAAAGGGTA |
| | ATGACGGAGACCCGGGTAAAAATGGTGACAAGGGTCACGCAGGTCTGGC |
| | AGGAGCAAGGGGTGCACCAGGACCTGACGGTGAGACAGGTGCACAAGG |
| | ACCTCCAGGACCTCAAGGTGTCCAAGGAGGAAAGGGAGAACAAGGACC |
| | GGCAGGACCTCCTGGATTTCAAGGTTTGCCTGGTACAGGAGGCCCTGCAG |
| | GAGAAGTCGGTAAGCCTGGAGAGCAAGGACTGCACGGCGAGTTTGGACT |
| | GCCAGGTCCCGCAGGACCTCGTGGAGAGCGTGGTGCTCCGGGTGAGCGT |
| | GGTCCTCCGGCAATTGCTGGTATTGGTGGAGAAAAGGCCGGCGGTTTCGC |
| | TCCATATTATGGT |
| 1019 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAAGAGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTAGAGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACA |
| | GAGGTGCCCGTGGTTCCGATGGACAACCCGGGCCACCCGGCCCTCCAGG |
| | TACCGCTGGATTTCCTGGAAGCCCTGGTGCTAAGGGGAGAGTTGGTCCGG |
| | CTGGTAGTCCCGGAAGTAACGGTGCCCCAGGTCAAAGAGGCGAACCAGG |
| | CCCTCAGGGTCACGCAGGAGCACAGGGACCGCCTGGTCCTCCTGGTATTA |
| | ATGGTTCGCCTGGAGGAAAAGGTGAAATGGGGCCCGCAGGAATCCCCGG |
| | TGCGCCTGGTCTTATGGGTGCCAGGGGTCCTCCAGGCCCGGCCGGTGCAA |
| | ATGGTGCTCCCGGATTACGAGGAGGAGCTGGTAGACCTGGTAAAAACGG |
| | TGCCAAAGGAGAACCAGGTCCTCGTGGAGAGCGTGGTAGAGCTGGCATT |
| | CCCGGTGTGCCTGGTGCAAAAGGTGAGCGTGGTAAGGACGGTTCCCCTG |
| | GTGAGCCAGGTGCGAACGGACTGCCAGGTGCAGCCGGAGAGCGAGGAG |
| | CTCCAGGATTCAGGGGACCAGCCGGTCCTAACGGCATTCCTGGTGAAAA |
| | AGGGCCCGCCGGTGAAAGGGGAGCTCCCGGTCCAGCAGGCCCTCGTGGA |
| | GCAGCTGGTGAGCCTGGACGTAGAGGTGTCCCAGGAGGGCCAGGTATGA |
| | GGGGTATGCCCGGGTCCCCTGGCGGTCCTGGATCGAGAGGAAAACCAGG |
| | GCCACCAGGTTCGCAGGGTGAAAGCGGACGTCCAGGCCCACCCGGCCCT |
| | TCAGGTCCAAGGGGTCAGCCTGGTGTCATGGGTTTCCCCGGTCCAAAGGG |
| | TAATGACGGAGCACCGGGTAAAAATGGTGAACGTGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAAGAAAGGGAGATACGGGACC |
| | GCCAGGACCTCAAGGATTGCAAGGTTTGCCTGGTACAGGAGGCCCTCCC |
| | GGAGAAAATGGTAAGCCTGGAAGACCAGGACCAAAAGGCGATGCTGGA |
| | GCCCCAGGTGCACCCGGAGGTAAGGGACGTGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1020 | GACGTCAAATCTGGTGTTGCTGTTGGAGGTTTAGCAGGCTACCCTGGTCC |
| | CGCAGGGCCCCCAGGTCCGCCGGGTCCGCCCGGAGCATCAGGTCATCCC |
| | GGAAGCCCTGGTTCACCAGGTTATCAGGGACCGCCCGGAGAGCCTGGAC |
| | AAGCTGGTCCCGCAGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAGCAGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | GCAGCTGGATTTCCTGGAGCACCTGGTGCTAAGGGGGAGGTTGGTCCGG |
| | CTGGTAGTCCCGGAAGTAACGGTGCCCCAGGTCAAAGAGGCGAACCAGG |
| | CCCTCAGGGTCACGCAGGAGCACAGGGACCGCCTGGTCCTCCTGGTATTA |
| | ATGGTTCGCCTGGAGGAAAAGGTGAAATGGGGCCCGCAGGAATCCCCGG |
| | TGCGCCTGGTCTTATGGGTGCCAGGGGTCCTCCAGGCCCGGCCGGTGCAA |
| | ATGGTGCTCCCGGATTACGAGGAGGAGCTGGTGAACCTGGTAAAAACGG |
| | TGCCAAAGGAGAACCAGGTCCTCGTGGAGAGCGTGGTGAAGCTGGCATT |
| | CCCGGTGTGCCTGGTGCAAAAGGTGAGGACGGTAAGGACGGTTCCCCTG |
| | GTGAGCCAGGTGCGAACGGACTGCCAGGTGCAGCCGGAGAGCGAGGAG |
| | CTCCAGGATTCAGGGGACCAGCCGGTCCTAACGGCATTCCTGGTGAAAA |
| | AGGGCCCGCCGGTGAAAGGGGAGCTCCCGGTCCAGCAGGCCCTCGTGGA |
| | GCAGCTGGTGAGCCTGGACGTGACGGTGTCCCAGGAGGGCCAGGTATGA |
| | GGGGTATGCCCGGGTCCCCTGGCGGTCCTGGATCGGATGGAAAACCAGG |
| | GCCACCAGGTGCACAGGGTGAAAGCGGACGTCCAGGCCCACCCGGCCCT |
| | TCAGGTCCAAGGGGTCAGCCTGGTGTCATGGGTTTCCCCGGTCCAAAGGG |
| | TAATGACGGAGCACCGGGTAAAAATGGTGAACGTGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAGATAAGGGAGATACGGGACC |
| | GCCAGGACCTCAAGGATTGCAAGGTTTGCCTGGTACAGGAGGCCCTCCC |
| | GGAGAAAATGGTAAGCCTGGAGAGCCAGGACCAAAAGGCGATGCTGGA |
| | GCCCCAGGTGCACCCGGAGGTAAGGGAGACGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1021 | GACGTCAAATCTGGTGTTGCTGTTGGAGGTTTAGCAGGCTACCCTGGTCC |
| | CGCAGGGCCCCCAGGTCCGCCGGGTCCGCCCGGAAACTCAGGTCATCCC |
| | GGAAGCCCTGGTTCACCAGGTTATCAGGGACCGCCCGGAGAGCCTGGAC |
| | AAGCTGGTCCCAACGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAAACGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | AACGCTGGATTTCCTGGAAACCCTGGTGCTAAGGGGGAGGTTGGTCCGG |
| | CTGGTAGTCCCGGAAGTAACGGTGCCCCAGGTCAAAGAGGCGAACCAGG |
| | CCCTCAGGGTCACGCAGGAGCACAGGGACCGCCTGGTCCTCCTGGTATTA |
| | ATGGTTCGCCTGGAGGAAAAGGTGAAATGGGGCCCGCAGGAATCCCCGG |
| | TGCGCCTGGTCTTATGGGTGCCAGGGGTCCTCCAGGCCCGGCCGGTGCAA |
| | ATGGTGCTCCCGGATTACGAGGAGGAGCTGGTGAACCTGGTAAAAACGG |
| | TGCCAAAGGAGAACCAGGTCCTCGTGGAGAGCGTGGTGAAGCTGGCATT |
| | CCCGGTGTGCCTGGTGCAAAAGGTGAGGACGGTAAGGACGGTTCCCCTG |
| | GTGAGCCAGGTGCGAACGGACTGCCAGGTGCAGCCGGAGAGCGAGGAG |
| | CTCCAGGATTCAGGGGACCAGCCGGTCCTAACGGCATTCCTGGTGAAAA |
| | AGGGCCCGCCGGTGAAAGGGGAGCTCCCGGTCCAGCAGGCCCTCGTGGA |
| | GCAGCTGGTGAGCCTGGACGTGACGGTGTCCCAGGAGGGCCAGGTATGA |
| | GGGGTATGCCCGGGTCCCCTGGCGGTCCTGGATCGGATGGAAAACCAGG |
| | GCCACCAGGTAACCAGGGTGAAAGCGGACGTCCAGGCCCACCCGGCCCT |
| | TCAGGTCCAAGGGGTCAGCCTGGTGTCATGGGTTTCCCCGGTCCAAAGGG |
| | TAATGACGGAGCACCGGGTAAAAATGGTGAACGTGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAGATAAGGGAGATACGGGACC |
| | GCCAGGACCTCAAGGATTGCAAGGTTTGCCTGGTACAGGAGGCCCTCCC |
| | GGAGAAAATGGTAAGCCTGGAGAGCCAGGACCAAAAGGCGATGCTGGA |
| | GCCCCAGGTGCACCCGGAGGTAAGGGAGACGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1022 | GACGTCAAATCTGGTGTTGCTGTTGGAGGTTTAGCAGGCTACCCTGGTCC |
| | CGCAGGGGCTCCAGGTCCGCCGGGTCCGCCCGGAGCTTCAGGTCATCCCG |
| | GAAGCCCTGGTGCTCCAGGTTATCAGGGACCGCCCGGAGCTCCTGGACA |
| | AGCTGGTCCCTCCGGAGCTCCTGGTCCACCAGGTGCTATTGGAGCTAGTG |
| | GTCCTGCCGGAAAAGACGGTGCTTCCGGTAGACCTGGTAGACCCGGCGA |
| | AAGGGGTTTACCAGGTCCTCCCGGAGCTAAGGGTCCAGCCGGTATACCC |
| | GGTGCTCCTGGGATGAAGGGTCACAGAGGATTTGATGGTAGAAACGGAG |
| | AGAAAGGCGCTACCGGTGCTCCCGGACTGAAGGGTGCTAACGGTCTTCCT |
| | GGTGAGAACGGCGCTCCTGGACCTATGGGTCCACGTGGTGCTCCAGGAG |
| | AAAGAGGCAGACCAGGAGCTCCTGGTGCAGCTGGTGCTAGAGGTGCTGA |
| | TGGTGCCCGTGGTTCCGATGGAGCTCCCGGGCCACCCGGCCCTCCAGGTG |
| | CTGCTGGATTTCCTGGAAGCCCTGGTGCTAAGGGGGAGGTTGGTCCGGCT |
| | GGTGCTCCCGGAAGTAACGGTGCCCCAGGTGCTAGAGGCGAACCAGGCC |
| | CTCAGGGTGCTGCAGGAGCACAGGGACCGCCTGGTGCTCCTGGTATTAAT |
| | GGTTCGCCTGGAGCTAAAGGTGAAATGGGGCCCGCAGGAGCTCCCGGTG |
| | CGCCTGGTCTTATGGGTGCCAGGGGTCCTCCAGGCCCGGCCGGTGCAAAT |
| | GGTGCTCCCGGATTACGAGGAGCTGCTGGTGAACCTGGTAAAAACGGTG |
| | CCAAAGGAGAACCAGGTCCTCGTGGAGCTCGTGGTGAAGCTGGCATTCC |
| | CGGTGCTCCTGGTGCAAAAGGTGAGGACGGTGCTGACGGTTCCCCTGGTG |
| | AGCCAGGTGCGAACGGACTGCCAGGTGCAGCCGGAGCTCGAGGAGCTCC |
| | AGGATTCAGGGGAGCTGCCGGTCCTAACGGCATTCCTGGTGCTAAAGGG |
| | CCCGCCGGTGAAAGGGGAGCTCCCGGTCCAGCAGGCCCTCGTGGAGCAG |
| | CTGGTGAGCCTGGACGTGACGGTGCTCCAGGAGGGCCAGGTATGAGGGG |
| | TGCTCCCGGGTCCCCTGGCGGTCCTGGAGCTGATGGAAAACCAGGGCCA |
| | CCAGGTGCTCAGGGTGAAAGCGGACGTCCAGGCGCTCCCGGCCCTTCAG |
| | GTCCAAGGGGTGCTCCTGGTGTCATGGGTTTCCCCGGTGCTAAGGGTAAT |
| | GACGGAGCACCGGGTGCTAATGGTGAACGTGGTGGCCCAGGTGCTCCAG |
| | GACCCCAAGGTCCACCAGGAGCTAACGGTGAGACAGGTCCTCAAGGAGC |
| | TCCAGGACCTACCGGTCCTGGAGGAGCTAAGGGAGATACGGGACCGCCA |
| | GGAGCTCAAGGATTGCAAGGTTTGCCTGGTGCTGGAGGCCCTCCCGGAG |
| | AAAATGGTGCTCCTGGAGAGCCAGGACCAAAAGGCGCTGCTGGAGCCCC |
| | AGGTGCACCCGGAGCTAAGGGAGACGCCGGTGCTCCGGGTGAGCGTGGT |
| | CCTCCGGCAATTGCTGGTATTGGTGGAGAAAAGGCCGGCGGTTTCGCTCC |
| | ATATTATGGT |
| 1023 | GACGTCAAATCTGGTGTTGCTGTCGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGTCCCAGGAGGGCCAGGTATGAGG |
| | GGTATGCCCGGGTCCCCTGGCGGTCCTGGATCGGATGGAAAACCAGGGC |
| | CACCAGGTTCGCAGGGTGAAAGCGGACGTCCAGGCCCACCCGGCCCTTC |
| | AGGTCCAAGGGGTCAGCCTGGTGTCATGGGTTTCCCCGGTCCAAAGGGTA |
| | ATGACGGAGCACCGGGTAAAAATGGTGAACGTGGTGGCCCAGGTGGTCC |
| | AGGACCCCAAGGTCCACCAGGAAAAAACGGTGAGACAGGTCCTCAAGGA |
| | CCTCCAGGACCTACCGGTCCTGGAGGAGATAAGGGAGATACGGGACCGC |
| | CAGGACCTCAAGGATTGCAAGGTTTGCCTGGTACAGGAGGCCCTCCCGG |
| | AGAAAATGGTAAGCCTGGAGAGCCAGGACCAAAAGGCGATGCTGGAGC |
| | CCCAGGTGCACCCGGAGGTAAGGGAGACGCCGGTGCTCCGGGTGAGCGT |
| | GGTCCTCCGGCAATTGCTGGTATTGGTGGAGAAAAGGCCGGCGGTTTCGC |
| | TCCATATTATGGT |
| 1024 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | GCTATTGGACCATCTGGTCCTGCTGGAAAGGATGGAGAGTCAGGTCGTCC |
| | AGGTCGTCCTGGAGAAAGGGGCTTGCCTGGACCTCCCGGAATTAAGGGA |
| | CCAGCTGGCATTCCCGGCTTCCCCGGTATGAAAGGACATAGAGGCTTCGA |
| | CGGTCGTAATGGTGAGAAAGGTGAGACCGGAGCACCAGGTTTGAAAGGA |
| | GAGAACGGATTACCTGGAGAAAATGGCGCCCCTGGACCAATGGGACCTC |
| | GTGGAGCTCCTGGAGAACGTGGTAGGCCTGGATTACCAGGAGCAGCCGG |
| | CGCAAGAGGCAATGATGGTGCACGTGGTTCCGACGGACAGCCTGGTCCA |
| | CCAGGACCCCCCGCAATTGCTGGTATTGGTGGAGAAAAGGCCGGCGGTT |
| | TCGCTCCATATTATGGT |
| 1025 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | GCTATTGGACCATCTGGTCCTGCTGGAAAGGATGGAGAGTCAGGTCGTCC |
| | AGGTCGTCCTGGAGAAAGGGGCTTGCCTGGACCTCCCGGAATTAAGGGA |
| | CCAGCTGGCATTCCCGGCTTCCCCGGTATGAAAGGACATAGAGGCTTCGA |
| | CGGTCGTAATGGTGAGAAAGGTGAGACCGGAGCACCAGGTTTGAAAGGA |
| | GAGAACGGATTACCTGGAGAAAATGGCGCCCCTGGACCAATGGGACCTC |
| | GTGGAGCTCCTGGAGAACGTGGTAGGCCTGGATTACCAGGAGCAGCCGG |
| | CGCAAGAGGCAATGATGGTGCACGTGGTTCCGACGGACAGCCTGGTCCA |
| | CCAGGACCCCCCGCAATTGCTGGTATTGGTGGAGAAAAGGCCGGCGGTT |
| | TCGCTCCATATTATGGT |
| 1026 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | GCTATTGGTCCATCTGGGCCTGCTGGGAAGGATGGTGAATCGGGTCGGCC |
| | TGGTCGGCCTGGTGAACGTGGCCTTCCGGGTCCTCCCGGTATTAAAGGAC |
| | CGGCTGGTATTCCCGGTTTTCCTGGTATGAAAGGTCATAGAGGTTTTGAT |
| | GGTAGAAATGGCGAAAAGGGTGAAACTGGTGCTCCTGGATTGAAAGGTG |
| | AGAATGGTCTGCCTGGGGAGAATGGAGCGCCTGGTCCAATGGGTCCAAG |
| | AGGTGCTCCTGGTGAAAGAGGTAGACCGGGACTTCCCGGGGCAGCTGGC |
| | GCTCGAGGTAATGATGGTGCTAGAGGGTCTGATGGTCAACCGGGTCCAC |
| | CCGGTCCGCCAGGTGCAATAGGTCCTAGCGGACCGGCAGGCAAAGACGG |
| | GGAATCAGGTAGACCAGGAAGGCCCGGGGAGAGAGGGCTACCTGGCCCC |
| | CCCGGGATTAAGGGACCTGCAGGAATCCCTGGCTTCCCAGGCATGAAGG |
| | GTCATAGGGGCTTTGATGGAAGAAACGGAGAAAAGGGTGAAACGGGCG |
| | CGCCTGGCCTTAAAGGGGAAAACGGGCTGCCTGGGGAGAACGGAGCTCC |
| | GGGCCCCATGGGGCCGAGAGGCGCCCCAGGTGAAAGAGGCAGACCGGG |
| | ATTGCCAGGGGCTGCTGGCGCACGTGGTAACGACGGGGCAAGAGGGAGC |
| | GACGGTCAACCTGGCCCGCCGGGTCCACCAGCAATTGCTGGTATTGGTGG |
| | AGAAAAGGCCGGCGGTTTCGCTCCATATTATGGT |
| 1027 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGTCCCAGGAGGGCCAGGTATGAGG |
| | GGTATGCCCGGGTCCCCTGGCGGTCCTGGATCGGATGGAAAACCAGGGC |
| | CACCAGGTTCGCAGGGTGAAAGCGGACGTCCAGGCCCACCCGGCCCTTC |
| | AGGTCCAAGGGGTCAGCCTGGTGTCATGGGTTTCCCCGGTCCAAAGGGTC |
| | ACCGTGGATTTCCGGGTAAAAATGGTGAACGTGGTGGCCCAGGTGGTCC |
| | AGGACCCCAAGGTCCACCAGGAAAAAACGGTGAGACAGGTCCTCAAGGA |
| | CCTCCAGGACCTACCGGTCCTGGAGGAGATAAGGGAGATACGGGACCGC |
| | CAGGACCTCAAGGATTGCAAGGTTTGCCTGGTACAGGAGGCCCTCCCGG |
| | AGAAAATGGTAAGCCTGGAGAGCCAGGACCAAAAGGCGATGCTGGAGC |
| | CCCAGGTGCACCCGGAGGTAAGGGAGACGCCGGTGCTCCGGGTGAGCGT |
| | GGTCCTCCGGCAATTGCTGGTATTGGTGGAGAAAAGGCCGGCGGTTTCGC |
| | TCCATATTATGGT |
| 1028 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | CCGCTGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGCCGCTGGCGCTCCTGGACCTATGGGTCCACGTGGTGCTCCAGGAG |
| | CCGCTGGCAGACCAGGATTGCCTGGTGCAGCTGGTGCTAGAGGTAACGA |
| | TGGTGCCCGTGGTTCCGATGGACAACCCGGGCCACCCGGCCCTCCAGGTA |
| | CCGCTGGATTTCCTGGAAGCCCTGGTGCTAAGGGGGAGGTTGGTCCGGCT |
| | GGTAGTCCCGGAAGTAACGGTGCCCCAGGTCAAAGAGGCGAACCAGGCC |
| | CTCAGGGTCACGCAGGAGCACAGGGACCGCCTGGTCCTCCTGGTATTAAT |
| | GGTTCGCCTGGAGGAAAAGGTGAAATGGGGCCCGCAGGAATCCCCGGTG |
| | CGCCTGGTCTTATGGGTGCCAGGGGTCCTCCAGGCCCGGCCGGTGCAAAT |
| | GGTGCTCCCGGATTACGAGGAGGAGCTGGTGAACCTGGTAAAAACGGTG |
| | CCAAAGGAGAACCAGGTCCTCGTGGAGAGCGTGGTGAAGCTGGCATTCC |
| | CGGTGTGCCTGGTGCAAAAGGTGAGGACGGTAAGGACGGTTCCCCTGGT |
| | GAGCCAGGTGCGAACGGACTGCCAGGTGCAGCCGGAGAGCGAGGAGCTC |
| | CAGGATTCAGGGGACCAGCCGGTCCTAACGGCATTCCTGGTGAAAAAGG |
| | GCCCGCCGGTGAAAGGGGAGCTCCCGGTCCAGCAGGCCCTCGTGGAGCA |
| | GCTGGTGAGCCTGGACGTGACGGTGTCCCAGGAGGGCCAGGTATGAGGG |
| | GTATGCCCGGGTCCCCTGGCGGTCCTGGATCGGATGGAAAACCAGGGCC |
| | ACCAGGTTCGCAGGGTGAAAGCGGACGTCCAGGCCCACCCGGCCCTTCA |
| | GGTCCAAGGGGTCAGCCTGGTGTCATGGGTTTCCCCGGTCCAAAGGGTAA |
| | TGACGGAGCACCGGGTAAAAATGGTGAACGTGGTGGCCCAGGTGGTCCA |
| | GGACCCCAAGGTCCACCAGGAAAAAACGGTGAGACAGGTCCTCAAGGAC |
| | CTCCAGGACCTACCGGTCCTGGAGGAGATAAGGGAGATACGGGACCGCC |
| | AGGACCTCAAGGATTGCAAGGTTTGCCTGGTACAGGAGGCCCTCCCGGA |
| | GAAAATGGTAAGCCTGGAGAGCCAGGACCAAAAGGCGATGCTGGAGCCC |
| | CAGGTGCACCCGGAGGTAAGGGAGACGCCGGTGCTCCGGGTGCCGCTGG |
| | TCCTCCGAAGCAATTGCTGGTATTGGTGGAGAAAAGGCCGGCGGTTTCGC |
| | TCCATATTATGGTTAATCAAGAGGATGTCAGAATGCCATTTGCC |
| 1029 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAAACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAAAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCAAAGGGTGCTCCAGGA |
| | GAAAGAGGCAAACCAGGATTGCCTGGTGCAGCTGGTGCTAAGGGTAACG |
| | ATGGTGCCAAAGGTTCCGATGGACAACCCGGGCCACCCGGCCCTCCAGG |
| | TACCGCTGGATTTCCTGGAAGCCCTGGTGCTAAGGGGGAGGTTGGTCCGG |
| | CTGGTAGTCCCGGAAGTAACGGTGCCCCAGGTCAAAAAGGCGAACCAGG |
| | CCCTCAGGGTCACGCAGGAGCACAGGGACCGCCTGGTCCTCCTGGTATTA |
| | ATGGTTCGCCTGGAGGAAAAGGTGAAATGGGGCCCGCAGGAATCCCCGG |
| | TGCGCCTGGTCTTATGGGTGCCAAGGGTCCTCCAGGCCCGGCCGGTGCAA |
| | ATGGTGCTCCCGGATTAAAAGGAGGAGCTGGTGAACCTGGTAAAAACGG |
| | TGCCAAAGGAGAACCAGGTCCTAAAGGAGAGAAGGGTGAAGCTGGCATT |
| | CCCGGTGTGCCTGGTGCAAAAGGTGAGGACGGTAAGGACGGTTCCCCTG |
| | GTGAGCCAGGTGCGAACGGACTGCCAGGTGCAGCCGGAGAGAAAGGAG |
| | CTCCAGGATTCAAGGGACCAGCCGGTCCTAACGGCATTCCTGGTGAAAA |
| | AGGGCCCGCCGGTGAAAAAGGAGCTCCCGGTCCAGCAGGCCCTAAGGGA |
| | GCAGCTGGTGAGCCTGGAAAAGACGGTGTCCCAGGAGGGCCAGGTATGA |
| | AAGGTATGCCCGGGTCCCCTGGCGGTCCTGGATCGGATGGAAAACCAGG |
| | GCCACCAGGTTCGCAGGGTGAAAGCGGAAAACCAGGCCCACCCGGCCCT |
| | TCAGGTCCAAAAGGTCAGCCTGGTGTCATGGGTTTCCCCGGTCCAAAGGG |
| | TAATGACGGAGCACCGGGTAAAAATGGTGAAAAGGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAGATAAGGGAGATACGGGACC |
| | GCCAGGACCTCAAGGATTGCAAGGTTTGCCTGGTACAGGAGGCCCTCCC |
| | GGAGAAAATGGTAAGCCTGGAGAGCCAGGACCAAAAGGCGATGCTGGA |
| | GCCCCAGGTGCACCCGGAGGTAAGGGAGACGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1030 | GATGTCAAGTCAGGTGTGGCCGTGGGTGGCCTAGCCGGCTACCCAGGCC |
| | CAGCTGGTCCACCCGGCCCACCTGGCCCCCCCGGTACCTCAGGACATCCT |
| | GGCTCCCCTGGTTCTCCAGGTTACCAAGGCCCACCCGGTGAGCCTGGCCA |
| | GGCAGGCCCTTCCGGCCCTCCAGGACCCCCTGGAGCCATTGGACCTTCAG |
| | GTCCAGCTGGAAAAGATGGCGAGTCAGGAAGACCCGGCCGTCCAGGAGA |
| | ACGTGGCCTACCCGGTCCCCCCGGTATCAAGGGCCCCGCAGGTATTCCCG |
| | GTTTTCCTGGAATGAAGGGACACCGTGGATTTGATGGTAGGAACGGTGA |
| | AAAGGGCGAGACAGGTGCACCAGGTCTTAAGGGCGAGAACGGTTTGCCC |
| | GGTGAGAACGGCGCTCCAGGTCCTATGGGTCCCCGTGGAGCTCCTGGCG |
| | AGCGTGGAAGACCTGGTTTGCCTGGTGCTGCCGGTGCCAGGGGAAATGA |
| | TGGAGCACGTGGTTCTGATGGCCAGCCTGGACCCCCCGGACCCCCTGGAA |
| | CTGCAGGATTCCCCGGAGCTGTGGGCGCAAAAGGCGAGGCAGGTCCCCA |
| | AGGTCCCAGAGGTAGTGAGGGACCTCAGGGTGTCAGAGGAGAGCCTGGT |
| | CCTCCTGGCCCCGCAGGAGCTGCAGGCCCAGCCGGACCACCCGGAGCCG |
| | ATGGCCAACCCGGTGCCAAGGGTGCTATGGGTGCTCCCGGTATCGCTGGT |
| | GCCCCTGGTTTTCCAGGTGCACGTGGACCACCCGGACCCCAAGGCGCCGG |
| | TGGTCCCCCTGGACCCAAGGGTGGCGCCGGTGAACCCGGCGCCCCTGGA |
| | GCTAAAGGAGATACTGGCGCCAAGGGAGAGAGAGGCCCCGTCGGAGTGC |
| | AGGGCCCACCCGGACCCGCCGGTGAAGAGGGAAAGAGGGGAGCCAGGG |
| | GTGAACCCGGCCCAACTGGCTTACCAGGTCCACCAGGTGAAAGGGGTGG |
| | ACCTGGTTTTCGTGGCTTCCCTGGTGCCGATGGAATCCCAGGAGAGAAGG |
| | GACCCGCAGGAGAGCGTGGCGCACCCGGACCTGCAGGTCCAAGGGGCGC |
| | CGCCGGAGAACCAGGTAGAGATGGTGTCCCCGGCGGACCCGGTATGAGA |
| | GGCATGCCCGGTTCCCCCGGCGGACCTGGCTCAGATGGCAAGCCAGGTC |
| | CCCCCGGCAGTCAAGGTGAATCAGGAAGACCTGGACCCCCAGGCCCTAG |
| | TGGACCTCGTGGTCAGCCAGGAGTGATGGGCTTTCCTGGTCCTAAGGGAA |
| | ATGACGGCGCTCCCGGCAAAAATGGTGAGAGAGGAGGACCCGGAGGCCC |
| | AGGACCACAGGGCCCACCAGGCAAGAACGGAGAAACAGGTCCTCAGGG |
| | CCCCCCAGGACCCACTGGTCCCGGAGGCGATAAAGGCGATACTGGTCCT |
| | CCTGGTCCTCAAGGCCTACAAGGATTGCCAGGAACTGGAGGCCCCCCAG |
| | GAGAGAACGGAAAACCCGGTGAACCAGGTCCCAAGGGAGACGCAGGTG |
| | CTCCAGGTGCACCCGGTGGTAAGGGAGACGCAGGCGCACCTGGTGAGAG |
| | AGGACCTCCTGCAATAGCCGGCATTGGAGGTGAGAAAGCCGGTGGTTTT |
| | GCTCCATACTATGGT |
| 1031 | GACGTCAAATCTGGTGTTGCTGTTGGAGGTATTGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTTTAGGACCAAGT |
| | GGTCCTGCCGGAAAAGACGGTGAATCCGGTAGACCTGGTAGACCCGGCG |
| | AAAGGGGTGTTCCAGGTCCTCCCGGAGTTAAGGGTCCAGCCGGTTTACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGTTGGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTTTGAA |
| | TGGTTCGCCTGGAGGAAAAGGTGAAATGGGGCCCGCAGGAGTTCCCGGT |
| | GCGCCTGGTATTATGGGTGCCAGGGGTCCTCCAGGCCCGGCCGGTGCAA |
| | ATGGTGCTCCCGGAATACGAGGAGGAGCTGGTGAACCTGGTAAAAACGG |
| | TGCCAAAGGAGAACCAGGTCCTCGTGGAGAGCGTGGTGAAGCTGGCTTA |
| | CCCGGTTTACCTGGTGCAAAAGGTGAGGACGGTAAGGACGGTTCCCCTG |
| | GTGAGCCAGGTGCGAACGGAGTTCCAGGTGCAGCCGGAGAGCGAGGAGC |
| | TCCAGGATTCAGGGGACCAGCCGGTCCTAACGGCTTGCCTGGTGAAAAA |
| | GGGCCCGCCGGTGAAAGGGGAGCTCCCGGTCCAGCAGGCCCTCGTGGAG |
| | CAGCTGGTGAGCCTGGACGTGACGGTATTCCAGGAGGGCCAGGTATGAG |
| | GGGTATGCCCGGGTCCCCTGGCGGTCCTGGATCGGATGGAAAACCAGGG |
| | CCACCAGGTTCGCAGGGTGAAAGCGGACGTCCAGGCCCACCCGGCCCTT |
| | CAGGTCCAAGGGGTCAGCCTGGTTTGATGGGTTTCCCCGGTCCAAAGGGT |
| | AATGACGGAGCACCGGGTAAAAATGGTGAACGTGGTGGCCCAGGTGGTC |
| | CAGGACCCCAAGGTCCACCAGGAAAAAACGGTGAGACAGGTCCTCAAGG |
| | ACCTCCAGGACCTACCGGTCCTGGAGGAGATAAGGGAGATACGGGACCG |
| | CCAGGACCTCAAGGAGTTCAAGGTTTGCCTGGTACAGGAGGCCCTCCCG |
| | GAGAAAATGGTAAGCCTGGAGAGCCAGGACCAAAAGGCGATGCTGGAG |
| | CCCCAGGTGCACCCGGAGGTAAGGGAGACGCCGGTGCTCCGGGTGAGCG |
| | TGGTCCTCCGGCAATTGCTGGTATTGGTGGAGAAAAGGCCGGCGGTTTCG |
| | CTCCATATTATGGT |
| 1032 | GACGTCAAATCTGGTGTTGCTGTTGGAGGTTTAGCAGGCTACCCTGGTCC |
| | CGCAGGGCCCCCAGGTCCGCCGGGTCCGCCCGGAGCAGCAGGTCATCCC |
| | GGAGCACCTGGTGCACCAGGTTATCAGGGACCGCCCGGAGAGCCTGGAC |
| | AAGCTGGTCCCGCTGGACCCCCTGGTCCACCAGGTGCTATTGGACCAGCT |
| | GGTCCTGCCGGAAAAGACGGTGAAGCAGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAGATGGAG |
| | AGAAAGGCGAAGCAGGTGCTCCCGGACTGAAGGGTGAAGATGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTGATG |
| | ATGGTGCCCGTGGTGCTGATGGACAACCCGGGCCACCCGGCCCTCCAGGT |
| | GCAGCTGGATTTCCTGGAGCTCCTGGTGCTAAGGGGGAGGTTGGTCCGGC |
| | TGGTGCTCCCGGAGCAGATGGTGCCCCAGGTCAAAGAGGCGAACCAGGC |
| | CCTCAGGGTCACGCAGGAGCACAGGGACCGCCTGGTCCTCCTGGTATTGA |
| | TGGTGCTCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAGA |
| | TGGTGCTCCCGGATTACGAGGAGGAGCTGGTGAACCTGGTAAAGATGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTGCTCCTGG |
| | TGAGCCAGGTGCGGATGGACTGCCAGGTGCAGCCGGAGAGCGAGGAGCT |
| | CCAGGATTCAGGGGACCAGCCGGTCCTGATGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGTCCCAGGAGGGCCAGGTATGAGG |
| | GGTATGCCCGGGGCCCCTGGCGGTCCTGGAGCTGATGGAAAACCAGGGC |
| | CACCAGGTGCTCAGGGTGAAGCTGGACGTCCAGGCCCACCCGGCCCTGC |
| | TGGTCCAAGGGGTCAGCCTGGTGTCATGGGTTTCCCCGGTCCAAAGGGTG |
| | ATGACGGAGCACCGGGTAAAGATGGTGAACGTGGTGGCCCAGGTGGTCC |
| | AGGACCCCAAGGTCCACCAGGAAAAGATGGTGAGGCTGGTCCTCAAGGA |
| | CCTCCAGGACCTGCTGGTCCTGGAGGAGATAAGGGAGATGCTGGACCGC |
| | CAGGACCTCAAGGATTGCAAGGTTTGCCTGGTGCTGGAGGCCCTCCCGGA |
| | GAAGATGGTAAGCCTGGAGAGCCAGGACCAAAAGGCGATGCTGGAGCCC |
| | CAGGTGCACCCGGAGGTAAGGGAGACGCCGGTGCTCCGGGTGAGCGTGG |
| | TCCTCCGGCAATTGCTGGTATTGGTGGAGAAAAGGCCGGCGGTTTCGCTC |
| | CATATTATGGT |
| 1033 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCGAACCA |
| | GGAAGACCCGAGGGAGAGAAAGGAGGAGGTCGTGGTGGTGCTCCAGGC |
| | GCTCCAGGAAGAGGAGCAGGCAACGGTGAGGGAGCCCGTCCACCCCCTT |
| | TAATGGGTGGTGCAGGCGGTAACAAGGCTGGACCACTAGCTGCCCCAAT |
| | TCCCGGTGTGCCTGGTGCAAAAGGTGAGGACGGTAAGGACGGTTCCCCT |
| | GGTGAGCCAGGTGCGAACGGACTGCCAGGTGCAGCCGGAGAGCGAGGA |
| | GCTCCAGGATTCAGGGGACCAGCCGGTCCTAACGGCATTCCTGGTGAAA |
| | AAGGGCCCGCCGGTGAAAGGGGAGCTCCCGGTCCAGCAGGCCCTCGTGG |
| | AGCAGCTGGTGAGCCTGGACGTGACGGTGTCCCAGGAGGGCCAGGTATG |
| | AGGGGTATGCCCGGGTCCCCTGGCGGTCCTGGATCGGATGGAAAACCAG |
| | GGCCACCAGGTTCGCAGGGTGAAAGCGGACGTCCAGGCCCACCCGGCCC |
| | TTCAGGTCCAAGGGGTCAGCCTGGTGTCATGGGTTTCCCCGGTCCAAAGG |
| | GTAATGACGGAGCACCGGGTAAAAATGGTGAACGTGGTGGCCCAGGTGG |
| | TCCAGGACCCCAAGGTCCACCAGGAAAAAACGGTGAGACAGGTCCTCAA |
| | GGACCTCCAGGACCTACCGGTCCTGGAGGAGATAAGGGAGATACGGGAC |
| | CGCCAGGACCTCAAGGATTGCAAGGTTTGCCTGGTACAGGAGGCCCTCCC |
| | GGAGAAAATGGTAAGCCTGGAGAGCCAGGACCAAAAGGCGATGCTGGA |
| | GCCCCAGGTGCACCCGGAGGTAAGGGAGACGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1034 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAAGAGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTAGAGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACA |
| | GAGGTGCCCGTGGTTCCGATGGACAACCCGGGCCACCCGGCCCTCCAGG |
| | TACCGCTGGATTTCCTGGAAGCCCTGGTGCTAAGGGGAGAGTTGGTCCGG |
| | CTGGTAGTCCCGGAAGTAACGGTGCCCCAGGTCAAAGAGGCGAACCAGG |
| | CCCTCAGGGTCACGCAGGAGCACAGGGACCGCCTGGTCCTCCTGGTATTA |
| | ATGGTTCGCCTGGAGGAAAAGGTGAAATGGGGCCCGCAGGAATCCCCGG |
| | TGCGCCTGGTCTTATGGGTGCCAGGGGTCCTCCAGGCCCGGCCGGTGCAA |
| | ATGGTGCTCCCGGATTACGAGGAGGAGCTGGTAGACCTGGTAAAAACGG |
| | TGCCAAAGGAGAACCAGGTCCTCGTGGAGAGCGTGGTGAAGCTGGCATT |
| | CCCGGTGTGCCTGGTGCAAAAGGTGAGGACGGTAAGGACGGTTCCCCTG |
| | GTGAGCCAGGTGCGAACGGACTGCCAGGTGCAGCCGGAGAGCGAGGAG |
| | CTCCAGGATTCAGGGGACCAGCCGGTCCTAACGGCATTCCTGGTGAAAA |
| | AGGGCCCGCCGGTGAAAGGGGAGCTCCCGGTCCAGCAGGCCCTCGTGGA |
| | GCAGCTGGTGAGCCTGGACGTAGAGGTGTCCCAGGAGGGCCAGGTATGA |
| | GGGGTATGCCCGGGTCCCCTGGCGGTCCTGGATCGGATGGAAAACCAGG |
| | GCCACCAGGTTCGCAGGGTGAAAGCGGACGTCCAGGCCCACCCGGCCCT |
| | TCAGGTCCAAGGGGTCAGCCTGGTGTCATGGGTTTCCCCGGTCCAAAGGG |
| | TAATGACGGAGCACCGGGTAAAAATGGTGAACGTGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAGATAAGGGAGATACGGGACC |
| | GCCAGGACCTCAAGGATTGCAAGGTTTGCCTGGTACAGGAGGCCCTCCC |
| | GGAGAAAATGGTAAGCCTGGAGAGCCAGGACCAAAAGGCGATGCTGGA |
| | GCCCCAGGTGCACCCGGAGGTAAGGGAGACGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1035 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAAGAGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTAGAGGTAGAAACGGAG |
| | AGAAAGGCAGAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACA |
| | GAGGTGCCCGTGGTTCCAGAGGACAACCCGGGCCACCCGGCCCTCCAGG |
| | TACCGCTGGATTTCCTGGAAGCCCTGGTGCTAAGGGGAGAGTTGGTCCGG |
| | CTGGTAGTCCCGGAAGTAACGGTGCCCCAGGTCAAAGAGGCAGACCAGG |
| | CCCTCAGGGTCACGCAGGAGCACAGGGACCGCCTGGTCCTCCTGGTATTA |
| | ATGGTTCGCCTGGAGGAAAAGGTAGAATGGGGCCCGCAGGAATCCCCGG |
| | TGCGCCTGGTCTTATGGGTGCCAGGGGTCCTCCAGGCCCGGCCGGTGCAA |
| | ATGGTGCTCCCGGATTACGAGGAGGAGCTGGTAGACCTGGTAAAAACGG |
| | TGCCAAAGGAAGACCAGGTCCTCGTGGAGAGCGTGGTAGAGCTGGCATT |
| | CCCGGTGTGCCTGGTGCAAAAGGTAGACGTGGTAAGAGAGGTTCCCCTG |
| | GTGAGCCAGGTGCGAACGGACTGCCAGGTGCAGCCGGAGAGCGAGGAG |
| | CTCCAGGATTCAGGGGACCAGCCGGTCCTAACGGCATTCCTGGTGAAAA |
| | AGGGCCCGCCGGTGAAAGGGGAGCTCCCGGTCCAGCAGGCCCTCGTGGA |
| | GCAGCTGGTAGACCTGGACGTAGAGGTGTCCCAGGAGGGCCAGGTATGA |
| | GGGGTATGCCCGGGTCCCCTGGCGGTCCTGGATCGAGAGGAAAACCAGG |
| | GCCACCAGGTTCGCAGGGTGAAAGCGGACGTCCAGGCCCACCCGGCCCT |
| | TCAGGTCCAAGGGGTCAGCCTGGTGTCATGGGTTTCCCCGGTCCAAAGGG |
| | TAATAGAGGAGCACCGGGTAAAAATGGTAGACGTGGTGGCCCAGGTGGT |
| | CCAGGACCCCAAGGTCCACCAGGAAAAAACGGTGAGACAGGTCCTCAAG |
| | GACCTCCAGGACCTACCGGTCCTGGAGGAAGAAAGGGAAGAACGGGACC |
| | GCCAGGACCTCAAGGATTGCAAGGTTTGCCTGGTACAGGAGGCCCTCCC |
| | GGAGAAAATGGTAAGCCTGGAAGACCAGGACCAAAAGGCGATGCTGGA |
| | GCCCCAGGTGCACCCGGAGGTAAGGGACGTGCCGGTGCTCCGGGTGAGC |
| | GTGGTCCTCCGGCAATTGCTGGTATTGGTGGAGAAAAGGCCGGCGGTTTC |
| | GCTCCATATTATGGT |
| 1036 | GACGTCAAATCTGGTGTTGCTGTTGGAGGTATGGGTGGCTACCCTGGTCC |
| | CGCAGGGCCCCCAGGTCCGCCGGGTCCGCCCGGAGTTTCAGGTCATCCCG |
| | GAGCACCTGGTGCACCAGGTTATCAGGGACCGCCCGGAGAGCCTGGACA |
| | AGCTGGTCCCGCAGGACCCCCTGGTCCACCAGGTGCTATGGGACCAGCA |
| | GGTCCTGCCGGAAAAGACGGTGAATCCGGTAGACCTGGTAGACCCGGCG |
| | AAAGGGGTTTTCCAGGTCCTCCCGGAATTAAGGGTCCAGCCGGTATGCCC |
| | GGTTTTCCTGGGATGAAGGGTCACAGAGGATTTGATGGTAGAAACGGAG |
| | AGAAAGGCGATACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTGCTCCCGGACCAAACGGTGCCCCAGGTCAAAGAGGCGAACCAGGC |
| | CTTCAGGGTCAGGCAGGAGCACCGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATCGGTGCCAGGGGTCCTCCAGGCCCGCCGGGTGCAAA |
| | TGGTGTGCCCGGACAGCGAGGAGCTGCTGGTGAACCTGGTAAAAACGGT |
| | GCCAAAGGAGACCCAGGTGCTCGTGGAGAGCGTGGTGAAGCTGGCATTC |
| | CCGGTATTGCAGGTCCCAAAGGTGAGGACGGTAAGGACGGTTCCCCTGG |
| | TGAGCCAGGTGCGAACGGACTGCCAGGTGCACCAGGAGAGCGAGGACCA |
| | CCAGGATTCAGGGGAGCCCCTGGTGCTAACGGCATTCCTGGTGAAAAAG |
| | GGCCCCCAGGTGAAAGGGGAGGACCCGGTCCAGCAGGCCCTCGTGGAGT |
| | CGCTGGTGAGCCTGGACGTGACGGTTTGCCAGGAGGGCCAGGTTTGAGG |
| | GGTATTCCCGGGTCCCCTGGCGGTCCTGGATCGGATGGAAAACCAGGGC |
| | CACCAGGTTCGCAGGGTGAAAGCGGACGTCCAGGCCCACCCGGCTCACC |
| | TGGTCCAAGGGGTCAGCCTGGTGTCATGGGTTTCCCCGGTCCAAAGGGTA |
| | ATGACGGAGCACCGGGTAAAAATGGTGAACGTGGTGGCCCAGGTGGTCC |
| | AGGATTGCCCGGTCCAGCAGGAAAAAACGGTGAGACAGGTCCTCAAGGA |
| | CCTCCAGGACCTACCGGTCCTGCAGGAGATAAGGGAGATGCAGGACCGC |
| | CAGGACCTCCTGGATTGCAAGGTTTGCCTGGTACAGGAGGCCCTCCCGGA |
| | GAAAATGGTAAGCCTGGAGAGCCAGGACCAAAAGGCGATGTCGGAGCCC |
| | CAGGTATTCCCGGAGGTAAGGGAGACGCCGGTGCTCCGGGTGAGCGTGG |
| | TCCTCCGGCAATTGCTGGTATTGGTGGAGAAAAGGCCGGCGGTTTCGCTC |
| | CATATTATGGT |
| 1037 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCGTGGCAGGTCCTAAAG |
| | GGCCCGCCGGTGAAAGGGGAGCTCCCGGTCCAGCAGGCCCTAAAGGAGC |
| | AGCTGGTGAGGCAGGACGTCCCGGTGAAGCAGGACTGCCAGGTGCAAAA |
| | GGTATGCCCGGGTCCCCTGGCGGTCCTGGACCTGATGGAAAACCAGGGC |
| | CACCAGGTCCAGCAGGTCAGGATGGACGTCCAGGCCCACCCGGCCCTCC |
| | GGGTGCAAGGGGTCAGGCAGGTGTCATGGGTTTCCCCGGTCCAAAGGGT |
| | GCAGCAGGAGAACCGGGTAAAGCAGGTGAACGTGGTGTCCCAGGTCCGC |
| | CAGGAGCAGTCGGTCCAGCAGGAAAAGATGGTGAGGCAGGTGCACAAG |
| | GACCTCCAGGACCTGCAGGTCCTGCAGGAGAGAGGGGAGAGCAGGGACC |
| | GGCAGGACCTCAAGGATTTCAAGGTTTGCCTGGTCCTGCAGGCCCTCCCG |
| | GAGAAGCAGGTAAGCCTGGAGAGCAAGGAGTCCCCGGCGATTTGGGAGC |
| | CCCAGGTGCACCCGGAGCAAGGGGAGAGAGGGGTGCTCCGGGTGAGCGT |
| | GGTCCTCCGGCAATTGCTGGTATTGGTGGAGAAAAGGCCGGCGGTTTCGC |
| | TCCATATTATGGT |
| 1038 | GACGTCAAATCTGGTGTTGCTGTCGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGTCCCAGGAGGGCCAGGTATGAGG |
| | GGTATGCCCGGGTCCCCTGGCGGTCCTGGATCGGATGGAAAACCAGGGC |
| | CACCAGGTTCGCAGGGTGAAAGCGGACGTCCAGGCCCACCCGGCCCTTC |
| | AGGTCCAAGGGGTCAGCCTGGTGTCATGGGTTTCCCCGGTCCAAAGGGTA |
| | ATGACGGAGCACCGGGTAAAAATGGTGAACGTGGTGGCCCAGGTGGTCC |
| | AGGACCCCAAGGTCCACCAGGAAAAAACGGTGAGACAGGTCCTCAAGGA |
| | CCTCCAGGACCTACCGGTCCTGGAGGAGATAAGGGAGATACGGGACCGC |
| | CAGGACCTCAAGGATTGCAAGGTTTGCCTGGTACAGGAGGCCCTCCCGG |
| | AGAAAATGGTAAGCCTGGAGAGCCAGGACCAAAAGGCGATGCTGGAGC |
| | CCCAGGTGCACCCGGAGGTAAGGGAGACGCCGGTGCTCCGGGTGAGCGT |
| | GGTCCTCCG |
| 1039 | GACGTCAAATCTGGTGTTGCTGTTGGAAAAACAACAAAATCAGGTTTAGC |
| | AGGCTACCCTGGTCCCGCAGGGCCCCCAGGTCCGCCGGGTCCGCCCGGA |
| | ACATCAGGTCATCCCGGAAGCCCTGGTTCACCAGGTTATCAGGGACCGCC |
| | CGGAGAGCCTGGACAAGCTGGTCCCTCCGGACCCCCTGGTCCACCAGGT |
| | GCTATTGGACCAAGTGGTCCTGCCGGAAAAGACGGTGAATCCGGTAGAC |
| | CTGGTAGACCCGGCGAAAGGGGTTTACCAGGTCCTCCCGGAATTAAGGG |
| | TCCAGCCGGTATACCCGGTTTTCCTGGGATGAAGGGTCACAGAGGATTTG |
| | ATGGTAGAAACGGAGAGAAAGGCGAAACCGGTGCTCCCGGACTGAAGG |
| | GTGAAAACGGTCTTCCTGGTGAGAACGGCGCTCCTGGACCTATGGGTCCA |
| | CGTGGTGCTCCAGGAGAAAGAGGCAGACCAGGATTGCCTGGTGCAGCTG |
| | GTGCTAGAGGTAACGATGGTGCCCGTGGTTCCGATGGACAACCCGGGCC |
| | ACCCGGCCCTCCAGGTACCGCTGGATTTCCTGGAAGCCCTGGTGCTAAGG |
| | GGGAGGTTGGTCCGGCTGGTAGTCCCGGAAGTAACGGTGCCCCAGGTCA |
| | AAGAGGCGAACCAGGCCCTCAGGGTCACGCAGGAGCACAGGGACCGCCT |
| | GGTCCTCCTGGTATTAATGGTTCGCCTGGAGGAAAAGGTGAAATGGGGC |
| | CCGCAGGAATCCCCGGTGCGCCTGGTCTTATGGGTGCCAGGGGTCCTCCA |
| | GGCCCGGCCGGTGCAAATGGTGCTCCCGGATTACGAGGAGGAGCTGGTG |
| | AACCTGGTAAAAACGGTGCCAAAGGAGAACCAGGTCCTCGTGGAGAGCG |
| | TGGTGAAGCTGGCATTCCCGGTGTGCCTGGTGCAAAAGGTGAGGACGGT |
| | AAGGACGGTTCCCCTGGTGAGCCAGGTGCGAACGGACTGCCAGGTGCAG |
| | CCGGAGAGCGAGGAGCTCCAGGATTCAGGGGACCAGCCGGTCCTAACGG |
| | CATTCCTGGTGAAAAAGGGCCCGCCGGTGAAAGGGGAGCTCCCGGTCCA |
| | GCAGGCCCTCGTGGAGCAGCTGGTGAGCCTGGACGTGACGGTGTCCCAG |
| | GAGGGCCAGGTATGAGGGGTATGCCCGGGTCCCCTGGCGGTCCTGGATC |
| | GGATGGAAAACCAGGGCCACCAGGTTCGCAGGGTGAAAGCGGACGTCCA |
| | GGCCCACCCGGCCCTTCAGGTCCAAGGGGTCAGCCTGGTGTCATGGGTTT |
| | CCCCGGTCCAAAGGGTAATGACGGAGCACCGGGTAAAAATGGTGAACGT |
| | GGTGGCCCAGGTGGTCCAGGACCCCAAGGTCCACCAGGAAAAAACGGTG |
| | AGACAGGTCCTCAAGGACCTCCAGGACCTACCGGTCCTGGAGGAGATAA |
| | GGGAGATACGGGACCGCCAGGACCTCAAGGATTGCAAGGTTTGCCTGGT |
| | ACAGGAGGCCCTCCCGGAGAAAATGGTAAGCCTGGAGAGCCAGGACCAA |
| | AAGGCGATGCTGGAGCCCCAGGTGCACCCGGAGGTAAGGGAGACGCCGG |
| | TGCTCCGGGTGAGCGTGGTCCTCCGAAAACAACAAAATCAGCAATTGCT |
| | GGTATTGGTGGAGAAAAGGCCGGCGGTTTCGCTCCATATTATGGT |
| 1040 | GACGTCAAATCTGGTGTTGCTGTTGGAAAAACAACAAAATCAGGTTTAGC |
| | AGGCTACCCTGGTCCCGCAGGGCCCCCAGGTCCGCCGGGTCCGCCCGGA |
| | ACATCAGGTCATCCCGGAAGCCCTGGTTCACCAGGTTATCAGGGACCGCC |
| | CGGAGAGCCTGGACAAGCTGGTCCCTCCGGACCCCCTGGTCCACCAGGT |
| | GCTATTGGACCAAGTGGTCCTGCCGGAAAAGACGGTGAATCCGGTAGAC |
| | CTGGTAGACCCGGCGAAAGGGGTTTACCAGGTCCTCCCGGAATTAAGGG |
| | TCCAGCCGGTATACCCGGTTTTCCTGGGATGAAGGGTCACAGAGGATTTG |
| | ATGGTAGAAACGGAGAGAAAGGCGAAACCGGTGCTCCCGGACTGAAGG |
| | GTGAAAACGGTCTTCCTGGTGAGAACGGCGCTCCTGGACCTATGGGTCCA |
| | CGTGGTGCTCCAGGAGAAAGAGGCAGACCAGGATTGCCTGGTGCAGCTG |
| | GTGCTAGAGGTAACGATGGTGCCCGTGGTTCCGATGGACAACCCGGGCC |
| | ACCCGGCCCTCCAGGTACCGCTGGATTTCCTGGAAGCCCTGGTGCTAAGG |
| | GGGAGGTTGGTCCGGCTGGTAGTCCCGGAAGTAACGGTGCCCCAGGTCA |
| | AAGAGGCGAACCAGGCCCTCAGGGTCACGCAGGAGCACAGGGACCGCCT |
| | GGTCCTCCTGGTATTAATGGTTCGCCTGGAGGAAAAGGTGAAATGGGGC |
| | CCGCAGGAATCCCCGGTGCGCCTGGTCTTATGGGTGCCAGGGGTCCTCCA |
| | GGCCCGGCCGGTGCAAATGGTGCTCCCGGATTACGAGGAGGAGCTGGTG |
| | AACCTGGTAAAAACGGTGCCAAAGGAGAACCAGGTCCTCGTGGAGAGCG |
| | TGGTGAAGCTGGCATTCCCGGTGTGCCTGGTGCAAAAGGTGAGGACGGT |
| | AAGGACGGTTCCCCTGGTGAGCCAGGTGCGAACGGACTGCCAGGTGCAG |
| | CCGGAGAGCGAGGAGCTCCAGGATTCAGGGGACCAGCCGGTCCTAACGG |
| | CATTCCTGGTGAAAAAGGGCCCGCCGGTGAAAGGGGAGCTCCCGGTCCA |
| | GCAGGCCCTCGTGGAGCAGCTGGTGAGCCTGGACGTGACGGTGTCCCAG |
| | GAGGGCCAGGTATGAGGGGTATGCCCGGGTCCCCTGGCGGTCCTGGATC |
| | GGATGGAAAACCAGGGCCACCAGGTTCGCAGGGTGAAAGCGGACGTCCA |
| | GGCCCACCCGGCCCTTCAGGTCCAAGGGGTCAGCCTGGTGTCATGGGTTT |
| | CCCCGGTCCAAAGGGTAATGACGGAGCACCGGGTAAAAATGGTGAACGT |
| | GGTGGCCCAGGTGGTCCAGGACCCCAAGGTCCACCAGGAAAAAACGGTG |
| | AGACAGGTCCTCAAGGACCTCCAGGACCTACCGGTCCTGGAGGAGATAA |
| | GGGAGATACGGGACCGCCAGGACCTCAAGGATTGCAAGGTTTGCCTGGT |
| | ACAGGAGGCCCTCCCGGAGAAAATGGTAAGCCTGGAGAGCCAGGACCAA |
| | AAGGCGATGCTGGAGCCCCAGGTGCACCCGGAGGTAAGGGAGACGCCGG |
| | TGCTCCGGGTGAGCGTGGTCCTCCGAAAACAACAAAATCAGCAATTGCT |
| | GGTATTGGTGGAGAAAAGGCCGGCGGTTTCGCTCCATATTATGGT |
| 1041 | GACGTCAAATCTGGTGTTGCTGTTGGAAAAACAACAAAATCAGGTTTAGC |
| | AGGCTACCCTGGTCCCGCAGGGCCCCCAGGTCCGCCGGGTCCGCCCGGA |
| | ACATCAGGTCATCCCGGAAGCCCTGGTTCACCAGGTTATCAGGGACCGCC |
| | CGGAGAGCCTGGACAAGCTGGTCCCTCCGGACCCCCTGGTCCACCAGGT |
| | GCTATTGGACCAAGTGGTCCTGCCGGAAAAGACGGTGAATCCGGTAGAC |
| | CTGGTAGACCCGGCGCCGCTGGTTTACCAGGTCCTCCCGGAATTAAGGGT |
| | CCAGCCGGTATACCCGGTTTTCCTGGGATGAAGGGTCACAGAGGATTTGA |
| | TGGTAGAAACGGAGAGAAAGGCGAAACCGGTGCTCCCGGACTGAAGGGT |
| | GAAAACGGTCTTCCTGGTGCCGCTGGCGCTCCTGGACCTATGGGTCCACG |
| | TGGTGCTCCAGGAGCCGCTGGCAGACCAGGATTGCCTGGTGCAGCTGGT |
| | GCTAGAGGTAACGATGGTGCCCGTGGTTCCGATGGACAACCCGGGCCAC |
| | CCGGCCCTCCAGGTACCGCTGGATTTCCTGGAAGCCCTGGTGCTAAGGGG |
| | GAGGTTGGTCCGGCTGGTAGTCCCGGAAGTAACGGTGCCCCAGGTCAAA |
| | GAGGCGAACCAGGCCCTCAGGGTCACGCAGGAGCACAGGGACCGCCTGG |
| | TCCTCCTGGTATTAATGGTTCGCCTGGAGGAAAAGGTGAAATGGGGCCCG |
| | CAGGAATCCCCGGTGCGCCTGGTCTTATGGGTGCCAGGGGTCCTCCAGGC |
| | CCGGCCGGTGCAAATGGTGCTCCCGGATTACGAGGAGGAGCTGGTGAAC |
| | CTGGTAAAAACGGTGCCAAAGGAGAACCAGGTCCTCGTGGAGAGCGTGG |
| | TGAAGCTGGCATTCCCGGTGTGCCTGGTGCAAAAGGTGAGGACGGTAAG |
| | GACGGTTCCCCTGGTGAGCCAGGTGCGAACGGACTGCCAGGTGCAGCCG |
| | GAGAGCGAGGAGCTCCAGGATTCAGGGGACCAGCCGGTCCTAACGGCAT |
| | TCCTGGTGAAAAAGGGCCCGCCGGTGAAAGGGGAGCTCCCGGTCCAGCA |
| | GGCCCTCGTGGAGCAGCTGGTGAGCCTGGACGTGACGGTGTCCCAGGAG |
| | GGCCAGGTATGAGGGGTATGCCCGGGTCCCCTGGCGGTCCTGGATCGGA |
| | TGGAAAACCAGGGCCACCAGGTTCGCAGGGTGAAAGCGGACGTCCAGGC |
| | CCACCCGGCCCTTCAGGTCCAAGGGGTCAGCCTGGTGTCATGGGTTTCCC |
| | CGGTCCAAAGGGTAATGACGGAGCACCGGGTAAAAATGGTGAACGTGGT |
| | GGCCCAGGTGGTCCAGGACCCCAAGGTCCACCAGGAAAAAACGGTGAGA |
| | CAGGTCCTCAAGGACCTCCAGGACCTACCGGTCCTGGAGGAGATAAGGG |
| | AGATACGGGACCGCCAGGACCTCAAGGATTGCAAGGTTTGCCTGGTACA |
| | GGAGGCCCTCCCGGAGAAAATGGTAAGCCTGGAGAGCCAGGACCAAAA |
| | GGCGATGCTGGAGCCCCAGGTGCACCCGGAGGTAAGGGAGACGCCGGTG |
| | CTCCGGGTGCCGCTGGTCCTCCGAAAACAACAAAATCAGCAATTGCTGGT |
| | ATTGGTGGAGAAAAGGCCGGCGGTTTCGCTCCATATTATGGT |
| 1042 | GACGTCAAATCTGGTGTTGCTGTTGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCCTCCGGACCCCCTGGTCCACCAGGTGCTATTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGAGGGTAAGCGAGGTGCACGAGG |
| | TGAGGCAGGTTCGGCGGGACCTCCAGGTCCTCCAGGATTACGAGGAGCT |
| | CCAGGATTCAGGGGATTACCAGGTGCAGATGGCAGGGCAGGTGTGATGG |
| | GGCCCCCTGGTGAAAGGGGAGCTCCCGGTCCAGCAGGCGTGCGTGGAGC |
| | AGCTGGTGATGCAGGACGTCCTGGTGAACCAGGATTAATGGGTCCTAGG |
| | GGTTTACCCGGGTCCCCTGGCGGTCCTGGACCTGCAGGAAAAGAAGGGC |
| | CAGTGGGTTTACCTGGTATTGATGGACGTCCAGGCCCAATTGGCCCTGCA |
| | GGTGCAAGGGGTGAACCTGGTGTCATGGGTTTCCCCGGTCCAAAGGGTA |
| | ATGACGGAGATCCGGGTAAAAATGGTGACAAAGGTCACGCAGGTTTAGC |
| | AGGAGCAAGGGGTGCACCAGGACCCGATGGTGAGACAGGTCCTCAAGGA |
| | CCTCCAGGACCTACCGGTCCTGGAGGAGATAAGGGAGATACGGGACCGC |
| | CAGGACCTCAAGGATTGCAAGGTTTGCCTGGTACAGGAGGCCCTCCCGG |
| | AGAAAATGGTAAGCCTGGAGAGCCAGGACCAAAAGGCGATGCTGGAGC |
| | CCCAGGTGCACCCGGAGGTAAGGGAGACGCCGGTGCTCCGGGTGAGCGT |
| | GGTCCTCCGGCAATTGCTGGTATTGGTGGAGAAAAGGCCGGCGGTTTCGC |
| | TCCATATTATGGT |
| 1043 | GACGTCAAATCTGGTGTTGCTGTTGGAGGTATGGGTGGCTACCCTGGTCC |
| | CGCAGGGCCCCCAGGTCCGCCGGGTCCGCCCGGAGTTTCAGGTCATCCCG |
| | GAGCACCTGGTGCACCAGGTTATCAGGGACCGCCCGGAGAGCCTGGACA |
| | AGCTGGTCCCGCAGGACCCCCTGGTCCACCAGGTGCTATGGGACCAGCA |
| | GGTCCTGCCGGAAAAGACGGTGAATCCGGTAGACCTGGTAGACCCGGCG |
| | AAAGGGGTTTTCCAGGTCCTCCCGGAATTAAGGGTCCAGCCGGTATGCCC |
| | GGTTTTCCTGGGATGAAGGGTCACAGAGGATTTGATGGTAGAAACGGAG |
| | AGAAAGGCGATACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTGCTCCCGGACCAAACGGTGCCCCAGGTCAAAGAGGCGAACCAGGC |
| | CTTCAGGGTCAGGCAGGAGCACCGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATCGGTGCCAGGGGTCCTCCAGGCCCGCCGGGTGCAAA |
| | TGGTGTGCCCGGACAGCGAGGAGCTGCTGGTGAACCTGGTAAAAACGGT |
| | GCCAAAGGAGACCCAGGTGCTCGTGGAGAGCGTGGTGAAGCTGGCATTC |
| | CCGGTATTGCAGGTCCCAAAGGTGAGGACGGTAAGGACGGTTCCCCTGG |
| | TGAGCCAGGTGCGAACGGACTGCCAGGTGCACCAGGAGAGCGAGGACCA |
| | CCAGGATTCAGGGGAGCCCCTGGTGCTAACGGCATTCCTGGTGAAAAAG |
| | GGCCCCCAGGTGAAAGGGGAGGACCCGGTCCAGCAGGCCCTCGTGGAGT |
| | CGCTGGTGAGCCTGGACGTGACGGTTTGCCAGGAGGGCCAGGTTTGAGG |
| | GGTATTCCCGGGTCCCCTGGCGGTCCTGGATCGGATGGAAAACCAGGGC |
| | CACCAGGTTCGCAGGGTGAAAGCGGACGTCCAGGCCCACCCGGCTCACC |
| | TGGTCCAAGGGGTCAGCCTGGTGTCATGGGTTTCCCCGGTCCAAAGGGTA |
| | ATGACGGAGCACCGGGTAAAAATGGTGAACGTGGTGGCCCAGGTGGTCC |
| | AGGATTGCCCGGTCCAGCAGGAAAAAACGGTGAGACAGGTCCTCAAGGA |
| | CCTCCAGGACCTACCGGTCCTGCAGGAGATAAGGGAGATGCAGGACCGC |
| | CAGGACCTCCTGGATTGCAAGGTTTGCCTGGTACAGGAGGCCCTCCCGGA |
| | GAAAATGGTAAGCCTGGAGAGCCAGGACCAAAAGGCGATGTCGGAGCCC |
| | CAGGTATTCCCGGAGGTAAGGGAGACGCCGGTGCTCCGGGTGAGCGTGG |
| | TCCTCCGGCAATTGCTGGTATTGGTGGAGAAAAGGCCGGCGGTTTCGCTC |
| | CATATTATGGT |
| 1044 | GACGTCAAATCTGGTGTTGCTGTCGGAGGTTTAGCAGGCTACCCTGGTCC |
| | CGCAGGGCCCCCAGGTCCGCCGGGTCCGCCCGGAACATCAGGTCATCCC |
| | GGAAGCCCTGGTTCACCAGGTTATCAGGGACCGCCCGGAGAGCCTGGAC |
| | AAGCTGGTCCAGCTGGTCCACCAGGTCCAATTGGTAACGTTGGACCAAGT |
| | GGTCCTGCCGGAAAAGACGGTGAATCCGGTAGACCTGGTAGACCCGGCG |
| | AAAGGGGTTTACCAGGTCCTCCCGGAATTAAGGGTCCAGCCGGTATACCC |
| | GGTTTTCCTGGGATGAAGGGTCACAGAGGATTTGATGGTAGAAACGGAG |
| | AGAAAGGCGAAACCGGTGCTCCCGGACTGAAGGGTGAAAACGGTCTTCC |
| | TGGTGAGAACGGCGCTCCTGGACCTATGGGTCCACGTGGTGCTCCAGGA |
| | GAAAGAGGCAGACCAGGATTGCCTGGTGCAGCTGGTGCTAGAGGTAACG |
| | ATGGTGCCCGTGGTTCCGATGGACAACCCGGGCCACCCGGCCCTCCAGGT |
| | ACCGCTGGATTTCCTGGAAGCCCTGGTGCTAAGGGGGAGGTTGGTCCGGC |
| | TGGTAGTCCCGGAAGTAACGGTGCCCCAGGTCAAAGAGGCGAACCAGGC |
| | CCTCAGGGTCACGCAGGAGCACAGGGACCGCCTGGTCCTCCTGGTATTAA |
| | TGGTTCGCCTGGAGGAAAAGGTGAAATGGGGCCCGCAGGAATCCCCGGT |
| | GCGCCTGGTCTTATGGGTGCCAGGGGTCCTCCAGGCCCGGCCGGTGCAAA |
| | TGGTGCTCCCGGATTACGAGGAGGAGCTGGTGAACCTGGTAAAAACGGT |
| | GCCAAAGGAGAACCAGGTCCTCGTGGAGAGCGTGGTGAAGCTGGCATTC |
| | CCGGTGTGCCTGGTGCAAAAGGTGAGGACGGTAAGGACGGTTCCCCTGG |
| | TGAGCCAGGTGCGAACGGACTGCCAGGTGCAGCCGGAGAGCGAGGAGCT |
| | CCAGGATTCAGGGGACCAGCCGGTCCTAACGGCATTCCTGGTGAAAAAG |
| | GGCCCGCCGGTGAAAGGGGAGCTCCCGGTCCAGCAGGCCCTCGTGGAGC |
| | AGCTGGTGAGCCTGGACGTGACGGTGTCCCAGGAGGGCCAGGTATGAGG |
| | GGTATGCCCGGGTCCCCTGGCGGTCCTGGATCGGATGGAAAACCAGGGC |
| | CACCAGGTTCGCAGGGTGAAAGCGGACGTCCAGGCCCACCCGGCCCTTC |
| | AGGTCCAAGGGGTCAGCCTGGTGTCATGGGTTTCCCCGGTCCAAAGGGTA |
| | ATGACGGAGCACCGGGTAAAAATGGTGAACGTGGTGGCCCAGGTGGTCC |
| | AGGACCCCAAGGTCCACCAGGAAAAAACGGTGAGACAGGTCCTCAAGGA |
| | CCTCCAGGACCTACCGGTCCTGGAGGAGATAAGGGAGATACGGGACCGC |
| | CAGGACCTCAAGGATTGCAAGGTTTGCCTGGTACAGGAGGCCCTCCCGG |
| | AGAAAATGGTAAGCCTGGAGAGCCAGGACCAAAAGGCGATGCTGGAGC |
| | CCCAGGTGCACCCGGAGGTAAGGGAGACGCCGGTGCTCCGGGTGAGCGT |
| | GGTCCTCCGGCAATTGCTGGTATTGGTGGAGAAAAGGCCGGCGGTTTCGC |
| | TCCATATTATGGT |

### C. Compositions Comprising Collagen Fragments and Sequence Variants Thereof

In some embodiments, the present disclosure provides a composition comprising one or more recombinant collagen fragments disclosed herein, and at least one pharmaceutically acceptable or cosmetically acceptable excipient. In particular embodiments, the composition can comprise a recombinant collagen fragment according to SEQ ID NO: 1 or SEQ TD NO: 986, and at least one pharmaceutically acceptable or cosmetically acceptable excipient. In further embodiments, the composition can comprise a recombinant collagen fragment having at least about 70%, at least about 7500 at least about 80%, at least about 8500, at least about 87.5%, at least about 90%, at least about 92.5%, at least about 95%, at least about 97.5%, at least about 98%, at least about 99% or 100% sequence identity, or similarity to, SEQ ID NO: 1 or SEQ ID NO: 986, and at least one pharmaceutically acceptable or cosmetically acceptable excipient. In still further embodiments, the composition can comprise a recombinant collagen fragment having about 85%, about 86%, about 87%, about 88%, about 89%, about 90%, about 91%, about 92%, about 93%, about 94%, about 95%, about 96%, about 97%, about 98%, about 99%, or 100% identity, or similarity, to SEQ ID NO: 1 or SEQ ID NO: 986, and at least one pharmaceutically acceptable or cosmetically acceptable excipient. In still further embodiments, the composition can comprise a recombinant collagen fragment having about 85%, about 86%, about 87%, about 88%, about 89%, about 90%, about 91%, about 92%, about 93%, about 94%, about 95%, about 96%, about 97%, about 98%, about 99%, or 100% sequence identity, or similarity, to SEQ ID NO: 1 or SEQ ID NO: 986, and at least one excipient that is suitable for use in a dietary supplement, e.g., a nutritional supplement.

In some embodiments, the composition can comprise a hydrolysis product of a collagen fragment, wherein the hydrolysis product can have a sequence that is a portion of SEQ ID NO: 1 or SEQ ID NO: 986, and at least one pharmaceutically acceptable or cosmetically acceptable excipient. In some embodiments, the composition comprises a hydrolysis product with a sequence according to one or more of SEQ ID NOs: 2-972, and at least one pharmaceutically acceptable or cosmetically acceptable excipient. In some embodiments, the composition can comprise any of the hydrolysis products set forth in SEQ ID NOs: 2-972, and at least one pharmaceutically acceptable or cosmetically acceptable excipient. In some embodiments, the composition can comprise a recombinant collagen fragment having at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, one of SEQ ID NOs: 2-972, and at least one pharmaceutically acceptable or cosmetically acceptable excipient.

In yet another embodiment, the composition can comprise a recombinant collagen fragment according to SEQ ID NO: 1 or SEQ ID NO: 986, a hydrolysis product having a sequence according to one of SEQ ID NOs: 2-972, and at least one pharmaceutically acceptable or cosmetically acceptable excipient. In yet a further embodiment, the composition can comprise a recombinant collagen fragment according to SEQ ID NO: 1 or SEQ ID NO: 986, a plurality of hydrolysis products having sequences, that can be the same or different, according to any of SEQ ID NOs: 2-972, and at least one pharmaceutically acceptable or cosmetically acceptable excipient. In certain embodiments, the number of hydrolysis products present in the plurality of hydrolysis products in the composition can increase with time, with temperature, pH, or as a result of other conditions that would typically cause a recombinant collagen fragment, such as a recombinant collagen fragment according to SEQ ID NO: 1 or SEQ ID NO: 986, to hydrolyze or otherwise breakdown. In other embodiments, the composition can be stabilized with one or more stabilizers so that the concentrations of the recombinant collagen fragment according to SEQ ID NO: 1 or SEQ ID NO: 986 and the concentrations each of the fragments in the plurality of fragments, remain substantially constant (i.e. vary by no more than +5% by HPLC over a given time period) or remain constant. In certain embodiments, the recombinant collagen fragment, such as a recombinant collagen fragment according to SEQ ID NO: 1 or SEQ ID NO: 986, can be hydrolyzed, such that less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of non-hydrolyzed recombinant fragment remains in the composition as measured by HPLC. In other embodiments, the composition can comprise a mixture of a recombinant collagen fragment (e.g., a recombinant collagen fragment according to SEQ ID NO: 1 or SEQ ID NO: 986) and a plurality of hydrolyzed products of that recombinant collagen fragment (e.g., a plurality of collagen fragments according to any of SEQ ID NOs: 2-972), such that the weight of the hydrolyzed products in the composition is less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of the weight of the collagen-related protein in the composition.

In some embodiments, the present disclosure provides a composition comprising one or more recombinant collagen fragment sequence variants disclosed herein, and at least one pharmaceutically acceptable or cosmetically acceptable excipient. In particular embodiments, the composition can comprise a recombinant collagen fragment sequence variant according to any one of SEQ ID NOs: 987-1015, and at least one pharmaceutically acceptable or cosmetically acceptable excipient. In further embodiments, the composition can comprise a recombinant collagen fragment sequence variant having at least about 70%, at least about 75%, at least about 80%, at least about 85%, at least about 87.5%, at least about 90%, at least about 92.5%, at least about 95%, at least about 97.5%, at least about 98%, or at least about 99% sequence identity, or similarity to SEQ ID NO: 986, and at least one pharmaceutically acceptable or cosmetically acceptable excipient. In still further embodiments, the composition can comprise a recombinant collagen fragment sequence variant having about 85%, about 86%, about 87%, about 88%, about 89%, about 90%, about 91%, about 92%, about 93%, about 94%, about 95%, about 96%, about 97%, about 98%, or about 99%, identity, or similarity, to SEQ ID NO: 986, and at least one pharmaceutically acceptable or cosmetically acceptable excipient. In still further embodiments, the composition can comprise a recombinant collagen fragment sequence variant having about 85%, about 86%, about 87%, about 88%, about 89%, about 90%, about 91%, about 92%, about 93%, about 94%, about 95%, about 96%, about 97%, about 98%, or about 99% sequence identity, or similarity SEQ ID NO: 986, and at least one excipient that is suitable for use in a dietary supplement, e.g., a nutritional supplement.

In some embodiments, the composition can comprise a recombinant collagen fragment having an amino acid chain length from about 350 amino acids to about 600 amino acids that overlaps with the amino acid sequence in SEQ ID NO: 1 or SEQ ID NO: 986. In some embodiments, the composition comprises a recombinant collagen fragment having a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids. In a particular embodiment, the composition comprises a recombinant collagen fragment having an amino acid chain length of 528 amino acids. In a particular embodiment, the composition comprises a recombinant collagen fragment having an amino acid chain length of 546 amino acids.

In some embodiments, the composition can comprise a recombinant collagen fragment sequence variant according to any one of SEQ ID NOs: 987-1015, having a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids. In a particular embodiment, the composition comprises a recombinant collagen fragment having an amino acid chain length of 528 amino acids. In a particular embodiment, the composition comprises a recombinant collagen fragment having an amino acid chain length of 546 amino acids.

In some embodiments, the composition can comprise a recombinant collagen fragment or sequence variant thereof having an amino acid chain length from about 350 amino acids to about 600 amino acids and can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to SEQ ID NO: 1 or SEQ ID NO: 986. In some embodiments, the such a collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the composition can comprise a hydrolysis product of a collagen fragment, wherein the hydrolysis product can have an amino acid chain length from about 10 amino acids to about 75 amino acids. In some embodiments, the composition can comprise a hydrolysis product of a collagen fragment having an amino acid chain length from about 20 amino acids to about 50 amino acids. In some embodiments, the hydrolysis product described herein can have a length of about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, or about 75 amino acids.

In some embodiments the composition can comprise from about 5 ppm to about 500 ppm of a recombinant collagen fragment or sequence variant thereof. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment sequence variant. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment with a sequence according to SEQ ID NO: 1 or SEQ ID NO: 986. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of one or more hydrolysis products of a recombinant collagen fragment. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of one or more hydrolysis products of a recombinant collagen fragment with a sequence according to SEQ ID NO: 1 or SEQ ID NO: 986. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen sequence variant according to any one of SEQ ID NOs: 987-1015. In some embodiments, the composition can comprise a mixture of a recombinant collagen fragment or variant thereof at a concentration from about 5 ppm to about 500 ppm, and one or more hydrolyzed products of that recombinant collagen fragment at a concentration from about 5 ppm to about 500 ppm. In some embodiments, the composition can comprise a mixture of a recombinant collagen fragment with a sequence according to SEQ ID NO: 1 or SEQ ID NO: 986, or a variant thereof, at a concentration from about 5 ppm to about 500 ppm, and one or more hydrolyzed products of that recombinant collagen fragment at a concentration from about 5 ppm to about 500 ppm.

In some embodiments, the composition can be prepared with about 0.1% to about 20% by volume of an about 0.5% to about 25% by weight recombinant collagen fragment solution. In some embodiments, the composition can be prepared with about 0.1% to about 20% by volume of an about 0.5% to about 25% by weight recombinant collagen fragment sequence variant solution. In some of these embodiments, the composition can comprise about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by volume of a recombinant collagen fragment solution or recombinant collagen fragment sequence variant solution. In some of these embodiments, the recombinant collagen fragment solution comprises about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of the recombinant collagen fragment or about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of the recombinant collagen fragment sequence variant. In some of these embodiments, the recombinant collagen fragment solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment with a sequence according to SEQ ID NO: 1 or SEQ ID NO: 986. In some of these embodiments, the recombinant collagen fragment solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolyzed products of a recombinant collagen fragment. In some of these embodiments, the recombinant collagen fragment solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolyzed products of a recombinant collagen fragment with sequences according to one or more of SEQ ID NOs: 2-972. In some of these embodiments, the recombinant collagen fragment sequence variant solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or recombinant collagen fragment sequence variants with sequences according to one or more of SEQ ID NOs: 987-1015. In some of these embodiments, the composition can be prepared using about 0.1% to about 20% by volume of an about 0.5% to about 25% by weight of a mixture of a recombinant collagen fragment and one or more hydrolyzed products of that recombinant collagen fragment. In some of these embodiments, the composition can be prepared using about 0.1% to about 20% by volume of an about 0.5% to about 25% by weight of a mixture of a recombinant collagen fragment with a sequence according to SEQ ID NO: 1 or SEQ ID NO: 986 and one or more hydrolyzed products of that recombinant collagen fragment.

In some embodiments, the composition can be prepared using about 0.1% to about 20% by volume of an about 0.5% to about 25% by weight solution comprising a mixture of recombinant collagen fragments with a sequence according to SEQ ID NO: 1 or SEQ ID NO: 986 and one or more hydrolyzed products of either recombinant collagen fragment. In some embodiments, the composition can be prepared using about 0.1% to about 20% by volume of an about 0.5% to about 25% by weight solution comprising a mixture of a recombinant collagen fragment sequence variants with sequences according to any one of SEQ ID NOs: 987-1015. In some embodiments, the composition can be prepared with about 0.1% to about 20% by volume of an about 0.5% to about 25% by weight solution comprising a mixture of a recombinant collagen fragment with a sequence according to SEQ ID NO: 1 or SEQ ID NO: 986 and one or more hydrolyzed products of either recombinant collagen fragment and a recombinant collagen fragment sequence variant solution comprising sequence variants with sequences according to any one of SEQ ID NOs: 987-1015

In some of these embodiments, the recombinant collagen fragment and recombinant collagen fragment sequence variant solution comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolyzed products of a recombinant collagen fragment with sequences according to one or more of SEQ ID NOs: 2-972.

In some embodiments, the composition can comprise from about 0.0005% to about 25% by weight of a recombinant collagen fragment, from about 0.001% to about 25% by weight of a recombinant collagen fragment, from about 0.01% to about 25% by weight of a recombinant collagen fragment, from about 0.1% to about 25% by weight of a recombinant collagen fragment, from about 0.5% to about 20% by weight of a recombinant collagen fragment, from about 0.7% to about 17% by weight of recombinant collagen fragment, from about 1% to about 15% by weight of recombinant collagen fragment, from about 2% to about 12% by weight of recombinant collagen fragment, from about 2% to about 10% by weight of recombinant collagen fragment, from about 3% to about 9% by weight of recombinant collagen fragment, from about 4% to about 8% by weight of recombinant collagen fragment, or from about 5% to about 7% by weight of recombinant collagen fragment.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment with a sequence according to SEQ ID NO: 1 or SEQ ID NO: 986. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment with sequences according to one or more of SEQ ID NOs: 2-972. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment and one or more hydrolysis products of that recombinant collagen fragment. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment with a sequence according to SEQ ID NO: 1 or SEQ ID NO: 986 and one or more hydrolysis products of that recombinant collagen fragment.

In some embodiments, the composition can comprise from about 0.0005% to about 25% by weight of a recombinant collagen fragment sequence variant, from about 0.001% to about 25% by weight of a recombinant collagen fragment sequence variant, from about 0.01% to about 25% by weight of a recombinant collagen fragment sequence variant, from about 0.1% to about 25% by weight of a recombinant collagen fragment sequence variant, from about 0.5% to about 20% by weight of a recombinant collagen fragment sequence variant, from about 0.7% to about 17% by weight of recombinant collagen fragment sequence variant, from about 1% to about 15% by weight of recombinant collagen fragment sequence variant, from about 2% to about 12% by weight of recombinant collagen fragment sequence variant, from about 2% to about 10% by weight of recombinant collagen fragment sequence variant, from about 3% to about 9% by weight of recombinant collagen fragment, from about 4% to about 8% by weight of recombinant collagen fragment sequence variant, or from about 5% to about 7% by weight of recombinant collagen fragment sequence variant.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a recombinant collagen fragment sequence variant. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment sequence variant with a sequence according to any one of SEQ ID NOs: 987-1015. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment and one or more recombinant collagen fragment sequence variants. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment with a sequence according to SEQ ID NO: 1 or SEQ ID NO: 986 and one or more collagen fragment sequence variants with a sequence according to any one of SEQ ID NOs: 987-1015.

### Skin Care

In some embodiments, the compositions described herein can be used to improve the aesthetic appearance of the skin and/or its appendages, for example, the surface appearance and/or texture of the skin. In some embodiments, the compositions described herein can be formulated for use on the body and face, hands, and feet, including as treatment for the eye area, for nails, and hair. The term “surface appearance” means the visual and/or tactile irregularities in the skin and/or in the scalp, including wrinkles and fine lines, expression lines on the forehead and in the space between the eyebrows, wrinkles and/or fine lines around the mouth, and/or slackening in the area around the lips and the top lip area (area located between the top lip and the nose), heterogeneity of the skin tone (liver spots, actinic lentigos), appearance and/or visibility of the pores, papery appearance of the skin, defects in the skin microrelief such as chicken pox or acne scars, imperfections of greasy skin (shiny appearance, etc.). The term “skin texture” can mean slack, flabby, less firm, less elastic skin, and/or skin that has sagged.

In some embodiments, the compositions described herein can be used to improve the aesthetic appearance of the skin, including improvement in the appearance of expression lines. Expression lines are produced by the effect of the stress exerted on the skin by the underlying muscles. Age and environmental factors such as exposure to sunlight, can deepen expression lines and make them permanent. Expression lines are characterized by the presence of grooves in the area around the orifices formed by the nose (nasal grooves), the mouth (perioral lines and so-called bitterness lines) and the eyes (crow's feet wrinkles), around which the skin muscles are located, and also between the eyebrows (glabella or lion wrinkles) and on the forehead.

In some embodiments, the compositions described herein can be used to improve the aesthetic appearance of the skin and/or visibility of the pores. Visibility of the pores can be due to an excess of sebum, aging, loss of firmness, slackening, stress, fatigue, unsuitable hygiene, climatic factors, or any combination thereof. The compositions described herein can tighten the pores, making them less visible.

In some embodiments, the compositions described herein can be used to improve a papery appearance of the skin and the behavior of the skin to the touch. Specifically, older skin can visually take on the appearance of cigarette paper, giving it an appearance similar to that of a sheet of papyrus. The papery appearance of the skin can be seen on the face and on the back of the hands of the elderly.

In some embodiments, the compositions described herein can be a composition for protecting, treating or caring for the face, for the hands, for the feet, or for the body, for example, day creams, night creams, makeup remover creams, anti-sun compositions, body milks for skin protection or care, after-sun milks, skincare lotions, gels, foams, artificial tanning compositions, and aftershave compositions. In some embodiments, the compositions described herein can be formulated, for example, as solutions, suspensions, lotions, creams, serums, gels, balms, gels, oils, oil in creams, micellar waters, face mists, face essences, blemish balm or complexion corrector formulas, toners (water and/or alcohol based), paints, polishes, sticks, pencils, sprays, aerosols, ointments, cleansing liquid washes, solid bars, shampoos, hair conditioners, hair styling products, pastes, foams, powders, mousses, balms, shaving creams, wipes, strips, patches, wound dressings, adhesive bandages, hydrogels, film-forming products, facial and skin masks, cosmetics (e.g. foundations, eye liners, eye shadows), exfoliators, deodorants and anti-perspirants, and the like. Exemplary formulations are provided herein.

In some embodiments, the composition described herein can be a cosmetic composition and the at least one excipient can be a cosmetically acceptable excipient. Cosmetically acceptable excipients are excipients suitable for use in a cosmetic product. Exemplary cosmetically acceptable excipients are described below.

In some embodiments, the cosmetic composition described herein can comprise a hydrolysis product of a recombinant collagen fragment and at least one excipient, e.g., a cosmetically acceptable excipient. In some embodiments, the cosmetic compositions described herein can comprise a recombinant collagen fragment, one or more hydrolyzed products of that recombinant collagen fragment, and at least one excipient, e.g., a cosmetically acceptable excipient.

In some embodiments, the cosmetic composition can comprise ingredients commonly used in cosmetic, e.g., skin care, eye care, nail care, and hair care products. These ingredients can include, but are not limited to, soaps, antimicrobials, anti-inflammatories, moisturizers, waxy alcohols, hydration agents, moisturizers, penetration enhancers, emulsifiers, natural or synthetic oils, solvents, fats, surfactants, detergents, gelling agents, emollients, antioxidants, fragrances, paints, polishes, fillers, thickeners, waxes, odor absorbers, dyestuff, coloring agents, powders, viscosity-controlling agents, anesthetics, anti-itch agents, botanical extracts, conditioning agents, darkening or whitening agents, humectants, mica, minerals, polyphenols, silicones or silicone derivatives such as dimethicone, sun blocks, vitamins, phytomedicinals, alcohols, such as denatured alcohols and ethanol, polyols, polyolethers, and other ingredients listed in the International Cosmetic Ingredient Dictionary and Handbook, 13th Ed. (2009), the entirety of which is incorporated herein by reference. In certain embodiments, a given ingredient can perform more than one function and can belong to more than one class.

In some embodiments, the composition described herein can be a therapeutic composition and the at least one excipient can be a therapeutically acceptable excipient. Therapeutic compositions can be useful for treating one or more conditions such as reducing or preventing the formation of scar tissue, promoting healing, promoting tissue regeneration, minimizing local inflammation, minimizing tissue rejection, and/or enhancing skin and/or hair graft integration. Therapeutically acceptable excipients are excipients that can serve as a vehicle or medium for an active substance and include excipients commonly used in therapeutic compositions, i.e., compositions useful for treating one or more conditions. Exemplary therapeutically acceptable excipients are described below.

In some embodiments, the composition described herein can be a dietary composition or dietary supplement, and the at least one excipient can be, for example, a food or drink additive. A “dietary supplement” is a preparation intended to supplement the diet and can be useful for providing nutrients or additives that may be missing or may not be consumed in sufficient quantities in a person's diet. In some embodiments, the dietary supplement can be provided in the form of any commonly used solid or liquid dosage form for oral administration including, without limitation, a capsule, a tablet, a pill, a powder, a granule or powder, a soft and hard gelatin capsule, and/or as a gummy. Suitable excipients include, but are not limited to, lactose or milk sugar as well as high molecular weight polyethyleneglycols, and the like. Exemplary dietary compositions are described below.

The compositions described herein can also include one or more of the following additional components. Exemplary contemplated additional ingredients are set forth below; however, this disclosure is not limited to these exemplary additional ingredients.

In some embodiments, the composition described herein can further comprise one or more anti-wrinkle agents. An anti-wrinkle agent is a compound which produces an increase in the synthesis and/or in the activity of certain enzymes of the skin, when the composition is brought into contact with an area of wrinkled skin, e.g., on the body or face, including the eye area, which reduces the outward appearance of the wrinkles and/or fine lines. Exemplary anti-wrinkle agents include, but are not limited to, desquamating agents, antiglycation agents, nitric oxide synthase inhibitors, muscle relaxants and/or dermo-decontracting agents, agents for combating free radicals, and mixtures thereof.

Additional exemplary anti-wrinkle agents that can be included in the composition described herein include, but are not limited to, adenosine and its derivatives, retinol and its derivatives (e.g., retinyl palmitate), ascorbic acid and its derivatives (e.g., magnesium ascorbyl phosphate and ascorbyl glucoside), tocopherol and its derivatives (e.g., tocopheryl acetate), nicotinic acid and its precursors (e.g., nicotinamide), ubiquinone, glutathione and its precursors (e.g., L-2-oxothiazolidine-4-carboxylic acid), C-glycoside compounds (also known as C-glycosyl compounds) and their derivatives (e.g., a 3-C-xylosyl derivative with the trade name PRO-XYLANE, plant extracts (e.g., rock samphire extracts and olive leaf extracts), plant proteins and their hydrolysates (e.g., rice or soybean protein hydrolysates), algal extracts (e.g., laminarian extracts), bacterial extracts, sapogenins (e.g., diosgenin), Dioscorea extracts (e.g., wild yam extracts), α-hydroxy acids, β-hydroxy acids (e.g., salicylic acid and 5-(n-octanoyl) salicylic acid), oligopeptides and pseudodipeptides and their acylated derivatives (e.g., {2-[acetyl (3-(trifluoromethyl) phenyl) amino]-3-methylbutyrylamino}acetic acid), lipopeptides e.g. MATRIXYL 3000 available from Croda), lycopene, manganese, magnesium salts (e.g., gluconates), and combinations of any of the foregoing.

Exemplary adenosine derivatives include, but are not limited to, 2′-deoxyadenosine; 2′,3′-iso-propylideneadenosine; toyocamycin, 1-methyladenosine; N-6-methyladenosine, adenosine N-oxide, 6-methyl-mercaptopurine riboside, and 6-chloropurine riboside. Other adenosine derivatives comprise adenosine receptor agonists, including phenylisopropyladenosine (“PIA”)/1-methylisoguanosine, Ns-cyclohexyladenosine (CHA), N6-cyclopentyladenosine (CPA), 2-chloro-Ns-cyclopentyladenosine, 2-chloroadenosine, N6-phenyladenosine, 2-phenylaminoadenosine, MECA, Ne-phenethyladenosine, 2-p-(2-carboxyethyl) phenethylamino-5′-N-ethylcarboxamido-adenosine (CGS-21680), (N-ethylcarboxamido) adenosine-S-(NECA), 5′-(N-cyclopropylcarboxamido) adenosine, DPMA (PD 129,944), and metrifudil.

In some embodiments, the composition described herein can comprise one or more adenosine derivatives that increase the intracellular concentration of adenosine, such as erythro-9-(2-hydroxy-3-nonyl) adenine (“EHNA”), iodotubercidin, or combinations thereof. Additional adenosine derivatives contemplated herein include adenosine salts and alkyl esters of adenosine.

In some embodiments, the composition can further comprise one or more pearlescent agents. Pearlescent agents are iridescent particles of any shape produced in particular by certain shellfish in their shells. Alternatively, pearlescent agents can be synthesized, i.e. man made. The pearlescent agents can be chosen from white pearlescent agents such as, but not limited to, mica covered with titanium oxide or with bismuth oxychloride, colored pearlescent agents such as, but not limited to, pearlescent agents based on bismuth oxychloride, titanium oxide-coated mica covered with iron oxides, titanium oxide-coated mica covered with in particular ferric blue or chromium oxide, or titanium oxide-coated mica covered with an organic pigment.

In some embodiments, the composition can further comprise one or more hydroxy acids. Examples of hydroxy acids include beta hydroxy acids such as salicylic acid, acetylsalicylic acid, and the like. Additional exemplary hydroxy acids suitable for use in the composition include citric acid, glycolic acid, hydroxycaproic acid, hydroxycaprylic acid, lactic acid, malic acid, tartaric acid, polyhydroxy acids including gluconolactone, and any combination thereof.

In some embodiments, the composition can further comprise one or more emulsifiers. An emulsifier keeps unlike ingredients (such as oil and water) from separating in an emulsion. Suitable emulsifiers include but are not limited to: polysorbates, laureth-4, potassium cetyl sulfate, glyceryl caprylate, and any combinations thereof.

In some embodiments, the composition can further comprise one or more chelating agents. Chelating agents bind with metal ions and prevent them from chemically reacting with other substances in a formulation. Suitable chelating agents include but are not limited to: sodium phytate, disodium EDTA, tetrasodium EDTA, tetrasodium glutamate diacetate, and trisodium ethylenediamine disuccinate.

In addition to a chelating agent, tetrasodium glutamate diacetate can also act as a stabilizer in any of the compositions described herein.

In some embodiments, the composition can further comprise one or more antimicrobials. Suitable antimicrobials include but are not limited to: caprylyl glyceryl ether, benzalkonium chloride, benzethonium chloride, and chloroxylenol (PCMX), tea tree oil, witch hazel, rosemary oil, lemon oil, and any combination thereof.

In some embodiments, the composition can further comprise one or more humectants (water-retaining agents) to improve the level of moisture in the skin. Non-limiting examples of suitable humectants for use in the compositions described herein are described in WO 98/22085, WO 98/18444, and WO 97/01326 and include: amino acids and derivatives thereof such as proline and arginine aspartate, 1,3-butylene glycol, propylene glycol, pentylene glycol, water, codium tomentosum extract, creatinine, diglycerol, biosaccharide gum-1, glucamine salts, glucuronic acid salts, glutamic acid salts, polyethylene glycol ethers of glycerine (e. g. glycereth 20), glycerine, glycerol monopropoxylate, glycogen, hexylene glycol, honey, hydrogenated starch hydrolysates, hydrolyzed mucopolysaccharides (such as xanthan gum and biosaccharide gum-1), inositol, keratin amino acids, glycosaminoglycans, methoxy PEG 10, methyl gluceth-10 and -20, methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), propanediol, pentaerythitol, 1,2 pentanediol, PPG-1 glyceryl ether, 2-pyrrolidone-5-carboxylic acid (including salts and esters thereof), saccharide isomerate, sericin, silk amino acids, sodium acetylhyaluronate, sodium hyaluronate, sodium poly-aspartate, sodium polyglutamate, caprylyl glycol, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, and yeast extract, and mixtures thereof.

Additional humectants suitable for use herein include polyhydric alcohols selected from the group consisting of glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

In some embodiments, the composition can further comprise a gelling agent comprising a polyacrylamide-based polymer. The polyacrylamide-based polymer can, aside from being polyacrylamide itself, be a derivative thereof, and can be a mixture of a plurality of types of polymers, and can also be a copolymer with acrylamide and its derivatives as monomers. A gelling agent can be used to provide a good appearance to the skin, provide a sense of coolness to the skin, and provide a sense of refreshment without stickiness to the skin. In some embodiments, a gelling agent can include one or more pigments, or one or more fillers, including inorganic pigments, including extender pigments, coloring pigments, and whitening pigments, organic pigments, pearlescent gloss pigments, macromolecular powders, functional pigments, talc, mica, kaolin, calcium carbonate, magnesium carbonate, silicic anhydride, aluminum silicate, magnesium silicate, calcium silicate, aluminum oxide, barium sulfate, red iron oxide, yellow iron oxide, black iron oxide, chrome oxide, ultramarine blue, prussian blue, carbon black, zinc oxide, mica titanium, fish scale flakes, bismuth oxychloride, boron nitride, nylon powder, silk powder, carbomer, tar pigments, natural pigments and titanium oxide, such as amorphous or rutile type and/or anatase type crystals.

In some embodiments, the composition can comprise a pigment and/or filler that is resistant to water and an oil and can further include any conventionally used water-repellent and/or oil-repellent agent to confer water repellence and oil repellence to pigments, for example, fluorine compounds. Representative fluorine compounds which are conventionally used and can act as water-repellent and oil-repellent agents include compounds having perfluoroalkyl groups such as perfluororalkyl phosphates, perfluoroalkyl silanes, perfluoroalkyl silazanes, polyhexafluoropropylene oxides, perfluoroalkyl-group-containing organosiloxanes, per-fluoropolyethers, perfluoro alcohols, perfluoroalkylacrylate polymers, and derivatives thereof. Perfluoroalkyl phosphates can provide a uniform and stable dispersement of pigments within the formulation of a gel composition, and perfluoroalkyl silanes can have exceptional compatibility with other cosmetic ingredients. Additionally, the perfluoroalkyl phosphate-diethanol amine salt marketed by Asahi Glass as AsahiGuard AG530, and perfluoroalkyl silane coupling agents, such as LP-IT and LP-4T of Shin-Etsu Silicone, can be used.

Representative gelling agents include, but are not limited to, those marketed by Seppic under the trade names Sepigel 305, Sepigel 501, and Sepigel 600. Sepigel 305 is a mixture containing approximately 40% polyacrylamide, approximately 24% —C₁₃-C₁₄ isoparaffin and approximately 6% Laureth-7 (here, Laureth-7 is a non-ionic surfactant having the formula C₁₂H₂₅—(OCH₂CH₂)ₙ—OH, wherein n has an average value of 7). Sepigel 600 is a mixture of a acrylamide/acrylamide-2-propane sulfonate copolymer, isohexadecane and polysorbate 80 (polyoxyethylene sorbitan mono-oleate (20 EO)). A suitable gelling agent comprising a polyacrylamide-based polymer is disclosed in EP 0 503 853 (Scott Bader Company Ltd.), the disclosure of which is incorporated by reference herein.

In some embodiments, the composition can further comprise hyaluronic acid (HA). In some embodiments, the HA can be in an uncrosslinked state. In some embodiments, the HA can be in a crosslinked state. Like collagen, HA is an important structural component of human tissues. Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronan present in the skin decreases.

In some embodiments, the composition described herein can be a dermal filler composition, e.g., an injectable dermal filler composition. Dermal fillers are generally made of collagens and can optionally comprise HA. Dermal filler compositions can be suitable for use on the face and body, including, e.g., around the eyes, on or around the cheeks, on or around the decolletage, on or around the hands, on or around the nails, on or around the ears, including on the earlobes, on or around the legs, and on or around the feet.

In some embodiments, the composition described herein can be used with a microneedle array, such as an array included in a sheet or patch. Microneedle arrays can comprise a plurality of microneedles that are of a length sufficient to penetrate the skin across the stratum corneum and into the viable epidermis. It some embodiments it can be desirable to deliver polypeptides to the area of epidermal/dermal junction for cosmetic or therapeutic purposes.

In some embodiments, the composition described herein can be used with microneedle sheets or patches. Microneedles and microneedle patches are suitable for delivering collagen into the epidermis and dermis of human skin on the face and body, including, e.g., the eyes, the cheeks, the lips, the decolletage, and the hands. In some embodiments, the microneedles for delivering compositions into the epidermis and dermis in a targeted manner are injectable microneedles, drug coated metal microneedles, or microneedles having dissolvable tips. Exemplary methods and disclosures regarding microneedles can be found in, for example, Aditya et al., Kinetics of collagen microneedle drug delivery system, Journal of Drug Delivery Science and Technology, vol. 52, pp. 618-623 (August 2019) and Sun et al., Transdermal Delivery of Functional Collagen Via Polyvinylpyrrolidone Microneedles, Ann. Biomed. Eng., 43(12):2978-2990 (2015), each of which is incorporated by reference in its entirety.

In some embodiments, the composition can further comprise a waxy lipid, e.g., a ceramide. Ceramides help create a barrier to prevent permeability, which helps prevent dryness and irritation and can also protect the epidermis from environmental damage.

In some embodiments, the composition can further comprise vitamin A or a vitamin A derivative. Examples of vitamin derivatives include, but are not limited to, retinoids such as retinal, retinoic acid, retinoate, retinyl ester, retinol, tretinoin, isotretinoin, adapalene, tazarotene, and the like. The term “retinoids” includes cis and trans derivatives of retinoids (e.g. all-trans-retinoic acid, 13-cis-retinoic acid, 13-trans retinoic acid, and 9-cis-retinoic acid).

In some embodiments, the composition can further comprise vitamin C or its derivatives, e.g., ascorbic acid, ascorbate (e.g. tetrahexyldecyl ascorbate), and the like.

In some embodiments, the composition can further comprise vitamin B, e.g., biotin, (i.e., vitamin B7), niacinamide, and the like.

In some embodiments, the composition can further comprise vitamin E, e.g. α-, β-, γ-, and α-tocopherols and their related corresponding tocotrienols), and the like.

In some embodiments, the composition can further comprise vitamin K and derivatives thereof.

Any vitamin, vitamin analog, or derivative thereof that can be suitably formulated as a topical composition is contemplated for the present disclosure.

In some embodiments, the composition disclosed herein can further comprise one or more thickening agents. A thickening agent, i.e., structure builder, is able to suspend pigments and/or build viscosity in the composition. Thickeners and/or structure builders suitable for the present compositions include, but are not limited to, organically modified clays, fumed silica, trihydroxystearin, silicone gels or silicone elastomers, ammonium acryloyldimethyltaurate/VP copolymer, acrylates/C10-30 alkyl acrylate crosspolymer, and mixtures thereof.

Suitable organically modified clays include, but are not limited to, organically modified versions of hectorite, bentonite, smectite and montmorillonite clay (such as those sold under tradename BENTONE® from Elementis Specialties, TIXO-GEL® from Sud-Chemie, and CLAYTONE® from Southern Clay Products). Hydrophilically modified fumed silicas include, but are not limited to, WACKER HDK® N20 and T30 grades (Wacker-Chemie AG), and hydrophilic grades under tradename of AEROSIL® (Evonik). Silicone gels or silicone elastomers include, but are not limited to, the “KSG” thickening series (KSG-15, KSG-16. KSG-18, KSG-41, KSG-42, KSG-43, KSG-44) from Shin-Etsu Silicones, DOW CORNING®9040, 9041, 9045, and 9546 silicone elastomer blends from Dow Corning, SFE839™, and Velvesil™ silicone gels from Momentive Performance Materials, and WACKER-BELSIL® RG-100 from Wacker-Chemie AG.

In some embodiments, the compositions disclosed herein can further comprise one or more lipo-soluble/lipo-dispersible film-forming agent. Lipo-soluble/lipo-dispersible film-forming agents suitable for use herein include, but are not limited to, organic silicone resins (e.g., trimethylsiloxysilicate such as SRI 000 from GE Silicones) and copolymers of organic silicone resins (e.g., diisostearyl trimethylolpropane siloxy silicate such as SF1318 from GE Silicones); fluorinated silicone resins; acrylic and/or vinyl based polymers or copolymers, including silicone and/or fluorinated versions (e.g., the “KP” series of silicone acrylates from Shin-Etsu Silicones, and 3M™ Silicones “Plus” Polymer VS70 and SA70); polyurethanes (e.g., the hydroxyester triglyceride derived Poly derm® series from Alzo International); polyesters (e.g., the Lexorez® series of polymeric polyesters from Inolex Chemical Company); and mixtures thereof.

In some embodiments, the composition disclosed herein can further comprise one or more coloring agents. Coloring agents suitable for use herein include all inorganic and organic colors/pigments, including mineral or pearl pigments suitable for use in cosmetic compositions. Such coloring agents include those either with or without a surface coating or treatment. Coloring agents can intensify coloration, and/or light scattering, and/or light reflecting effects of the composition.

In some embodiments, the composition disclosed herein can further comprise one or more sunscreens, e.g., mineral and/or physical sunscreens. Sunscreens can block UVA and/or UVB radiation. Exemplary UVA sunscreen agents include, but are not limited to, avobenzone, terephthalylidene dicamphor sulfonic acid, bis-disulizole disodium, disodium phenyl dibenzimidazole tetrasulfonate, diethylamino hydroxybenzoyl hexylbenzoate, bis-diethylamino hydroxybenzoyl benzoate, bis-benzoxazolylphenyl ethylhexylamino triazine, and combinations thereof.

Exemplary UVB sunscreen agents include, but are not limited to, octocrylene, octinoxate, octisalate, homosalate, ensulizole, ethylhexyl triazone, enzacamene, amiloxate, diethylhexyl butamido triazine, benzylidene malonate polysiloxane, padimate-O, trolamine salicylate, cinoxate, p-aminobenzoic acid and derivatives thereof, and combinations thereof.

Exemplary sunscreen agents that absorb both UVA and UVB radiation such as, for example, oxybenzone, meradimate, titanium dioxide, zinc oxide, bis-octrizole, bemotrizinol, drometrizole trisiloxane, sulisobenzone, dioxybenzone, or combinations thereof.

Specific Suitable sunscreens include but are not limited to p-aminobenzoic acid, its salts and its derivatives (ethyl, isobutyl, glyceryl esters, p-dimethylaminobenzoic acid, anthranilates (i.e., o-aminobenzoates, methyl, menthyl, phenyl, benzyl, phenylethyl, linallyl, terpinyl, and cyclohexenyl esters), salicylates (amyl, phenyl, benzyl, menthyl, glyceryl, and dipropylene glycol esters), cinnamic acid derivatives (methyl and benzyl esters, alpha-phenyl cinnamonitrile, butyl cinnamoyl pyruvate), dihydroxycinnamic acid derivatives (umbelliferone, methylumbelliferone, methylaceto umbelliferone), trihydroxycinnamic acid derivatives (esculetin, methyl eSculletin, daphnetin, and the glucosides, esculin and daphnin), hydrocarbons (diphenylbutadiene, stilbene), dibenzalacetone and benzalacetophenone, naphtholsulfonates (sodium salts of 2-naphthol-3,3-disulfonic and of 2-naphthol-6,8-disulfonic acids), cihydroxynaphthoic acid and its salts, o- and p-hydroxybiphenyldisulfonates, coumarin derivatives (7 hydroxy, 7-methyl, 3-phenyl), diazoles (2-acetyl-3-bromoindazole, phenyl benzoxazole, methylnaphthoxalole, various arylbenzothiazoles), quinine salts (bisulfate, sulfate, chloride, oleate, and tannate), quinoline derivatives (8-hydroxyquinoline salts, 2-phenylquinoline), hydroxy- or methoxy substituted benzophenones, uric and vilouric acids, tannic acid and its derivatives (e.g., hexaethylether), (butyl carbityl) (6-propyl piperonyl) ether, hydroquinone, benzophenones (oxybenzene, sulisobenzone, dioxybenzone, benzoresorcinol, 2,2,4,4′-tetrahydroxybenzophenone, 2,2′-dihydroxy4,4′-dimethoxybenzophenone, octabenzone, 4-isopropyhldibenzoylmethane, butylmethoxydibenzoylmethane, etocrylene, and 4-isopropyl-di-benzoylmethane, titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-bis(hydroxypropyl)aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less). In some embodiments, the composition disclosed herein can further comprise D-aspartic acid and/or D-alanine and any salts thereof. As used herein, the term “derivatives” of D-aspartic acid and D-alanine indicates D-aspartic acid and D-alanine molecules that are covalently bound to any organic group via their amino groups, carboxyl groups, or side chains, provided that the effect on promoting collagen production of D-aspartic acid and D-alanine is not impaired. Exemplary organic groups include, but are not limited to, protective groups, such as N-phenylacetyl group, and 4,4′-dimethoxytrityl (DMT) group; biopolymers, such as a protein, a peptide, a saccharide, a lipid, and a nucleic acid; synthetic polymers, such as a polystyrene, a polyethylene, a polyvinyl, a polypropylene, and a polyester; and functional groups such as an ester group. The ester group may comprise, for example, an aliphatic ester, such as methyl ester, and ethyl ester; and an aromatic ester.

In some embodiments, the composition can further comprise one or more general skin care additives such as, e.g., conditioning agents such silicones. In some embodiments, the composition can further comprise one or more shark liver oils, e.g., squalane and/or squalene. In some embodiments, the composition can further comprise one or more polysaccharides produced by microalgae, e.g., alguronic acid.

In some embodiments, the composition can further comprise at least one preservative. In some embodiments, the at least one preservative can be quaternary ammonium compounds, halogenated phenols, sorbic acid, potassium sorbate, benzoic acid, sodium benzoate, sodium citrate, sodium anisate, caprylhydroxamic acid, sodium levulinate, phenoxyethanol, or combinations thereof.

The compositions described herein can have a pH in the range of about 4 to about 8, from about 4.7 to about 5.5, from about 5 to about 7, from about 6 to about 7, from about 6.1 to about 6.8, or from about 6.4 to 6.6.

### Soaps

In some embodiments, the cosmetic composition described herein can be a cleansing composition or soap, including traditional soaps in the form of solid bar and liquid soaps in the form of cleaners, makeup removers, body washes, milks, creams, foams cream gels, or gels that can be packaged in tubes, bottles, pump bottles, aerosol shower foams or foam pump bottles. Soaps can be used in a cosmetic process for cleaning the dirt residues of human keratinous materials in the presence of water, massed to form a foam and the formed foam and the soil residues are removed by rinsing with water, and can be used on any part of the body and face disclosed herein, including, for example, on the skin of the body, face, hands, lips, eyelids, nails, hair, eyelashes and/or eyebrows.

Traditionally, solid soaps include alkali metal fatty acid salts and potassium fatty acid soaps, and liquid soaps include four main families of detergent formulation: (1) those based on lauryl sulfate; those based on alpha-olefin sulfonate, (3) those based on a mixture of synthetic anionic, amphoteric and/or nonionic surfactants; (4) mixed formulations based on soaps and synthetic surfactants. Liquid soaps generally contain a thickening system chosen, for example, from electrolytes such as sodium chloride, potassium chloride or potassium sulphate; alkanolamides such as cocamide DEA or cocamide MWA; esters of polyethylene glycol and monoacid or stearic acid such as polyethylene glycol distearate 6000 or mixtures thereof, and are contained in a cosmetically acceptable aqueous medium. However, both solid and liquid soaps can comprise any suitable additional ingredients such as those listed below herein, in any combination.

In some embodiments, the cleansing composition or soap described herein can further comprise one or more cellulosic compounds, or polysaccharide compound having in its structure chains of glucose residues linked by β-1,4 bonds, one or more fatty acids comprising a linear or branched, saturated or unsaturated alkyl chain having from 6 to 30 carbon atoms or 12 to 22 carbon atoms, one or more fatty acids, including lauric acid, myristic acid, palmitic acid and stearic acid, linolenic acid, and mixtures thereof, and one or more mineral bases, including alkali metal hydroxides (sodium hydroxide and potassium hydroxide), metal hydroxides or ammonia or organic bases such as triethanolamine, monethanolamine, monoisopropanolmaine, N-methylglucamine, lysine and arginine.

In some embodiments, the composition described herein can further comprise one or more anionic surfactants or salts, including alkali metal salts such as sodium salts, ammonium salts, amine salts, aminoalcohol salts or salts. alkaline earth metals, for example, magnesium, of the following types: alkyl sulphates, alkyl ether sulphates, alkyl amido ether sulphates, alkyl aryl polyether sulphates, monoglyceride sulphates; alkylsulfonates, alkylamidesulfonates, alkylarylsulphonates, α-olefin-sulfonates, paraffin-sulfonates; alkylsulfosuccinates, alkylethersulfosuccinates, alkylamide-sulfosuccinates; alkylsulfoacetates; acylsarcosinates; and the acylglutamates, the alkyl and acyl groups of all these compounds having from 6 to 24 carbon atoms and the aryl group denoting a phenyl or benzyl group, C₆-C₂₄ alkyl esters of polyglycoside carboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all these compounds having from 12 to 20 carbon atoms, and/or acyllactylates whose acyl group contains from 8 to 20 carbon atoms and their mixtures. In some embodiments, alkyl-D-galactoside uronic acids, polyoxyalkylenated (C₆-C₂₄) ether carboxylic acids, polyoxyalkylenated (C₆-C₂₄) aryl (C₆-C₂₄) polyoxyalkylenated ether carboxylic acids, polyoxyalkylenated (C₆-C₂₄) alkyl amidoether carboxylic acids, in particular those containing from 2 to 50 ethylene oxide groups; and their alkali metal, ammonium, amine, aminoalcohol or alkaline earth metal salts can also be suitable.

Suitable C₆-C₂₄ alkyl ether sulphate salts containing from 1 to 30 ethylene oxide groups, include alkali metals or alkaline earth metals, ammonium, amine or amino alcohol salts, sodium salts and oxyethylenated (C₁₂-C₁₄) alkyl ethersulfates having an average number of ethylene oxide groups of between 1 and 4 and including sodium laureth sulfate (CTFA name) such as the commercial product sold under the name TEXAPON AOS 225 UP TEXAPON N702 TEXAPON NSW marketed by COGNIS or EMPICOL ESB3/FL2, EMPICOL ESB3/FL3, EMPICOL ESB70/FL2 sold by the company Huntsman.

Suitable amphoteric surfactants include, but are not limited to, derivatives of secondary or tertiary aliphatic amines, in which the aliphatic group is a linear or branched chain comprising from 8 to 22 carbon atoms. The amphoteric surfactants can contain at least one water-soluble anionic group such as a carboxylate, sulfonate, sulfate, phosphate or phosphonate group, (C₈-C₂₀) alkylbetaines, sulphobetaines, (C₈-C₂₀) alkylamido (C₆-C₈) alkylbetaines or (C₈-C₂₀) alkylamidoalkyl (C₆-C₈) sulfobetaines, and mixtures thereof.

Suitable amine derivatives include the products sold under the name MIRANOL®, as described in patents U.S. Pat. Nos. 2,528,378 and 2,781,354 and filed in the CTFA dictionary, 3rd edition, 1982, under the terms Amphocarboxy-glycinate and Amphocarboxypropionate denominations. Additional suitable amine derivatives include those classified in the CTFA dictionary, 5th edition, 1993, under the names cocoamphodiacetate disodium, lauroamphodiacetate disodium, caprylamphodiacetate disodium, capryloamphodiacetate disodium, cocoamphodipropionate disodium, lauroamphodipropionate disodium, caprylamphodipropionate disodium, capryloamphodipropionate disodium, acid lauroamphodipropionic, cocoamphodipropionic acid, and cocoamphodiacetate sold under the trade name MIRANOL® C2M concentrated by Rhodia. Suitable alkyl (C₈-C₂₀) betaines include cocamidopropyl betaine and cocobetaine such as the commercial products MIRATAINE BB/FLA from RHODIA or EMPIGEN BB/FL from Huntsman.

In some embodiments, the cleansing compositions or soaps described herein can be sulfate-free, and can include a sulfate-free surfactant system.

In some embodiments, the cleansing composition or soap described herein can further comprise one or more thickeners of the nonionic cellulosic compound type. Suitable cellulosic compounds include, but are not limited to, nonionic cellulose ethers, including methylcelluloses and ethylcelluloses; hydroxyalkylcelluloses such as hydroxymethylcelluloses, hydroxyethylcelluloses and hydroxypropylcelluloses; mixed hydroxyalkyl-alkylcellulose celluloses such as hydroxypropyl-methylcelluloses, hydroxyethyl-methylcelluloses, hydroxyethylethylcelluloses and hydroxybutyl-methylcelluloses and hydroxyalkylcelluloses modified with an alkyl chain. Suitable hydroxypropyl methylcelluloses include the commercial products METHOCEL E, F, J and K sold by Dow Corning and even more particularly METHOCEL E 4MQG or METHOCEL F 4M. Suitable cellulosic ingredients can be in a crystalline form, a microcrystalline form, or a mixture thereof.

In some embodiments, the cleansing composition or soap described herein can further comprise one or more additional thickeners including electrolytes such as sodium chloride, potassium chloride or potassium sulphate; alkanolamides such as cocamide DEA or cocamide MEA; esters of polyethylene glycol and monoacid or stearic acid such as polyethylene glycol distearate 6000 or mixtures thereof, polysaccharide biopolymers such as xanthan gum, guar gum, alginates, synthetic polymers such as polyacrylics such as CARBOPOL 980, CARBOPOL 1382 marketed by NOVEON, acrylate/acrylonitrile copolymers such as HYPAN SS201 marketed by KINGSTON, clays such as smectites, modified or unmodified hectorites such as BENTONE products marketed by Rheox, LAPONITE products marketed by Southern Clay Products, VEEGUM HS product marketed by RT Vanderbilt, and mixtures thereof.

In some embodiments, the cleansing composition or soap described herein can further comprise one or more nonionic surfactants. These are well-known compounds (see regard “Handbook of Surfactants” by MR PORTER, Blackie & Son editions (Glasgow and London), 1991, pp 116-178), and can be chosen from alcohols, alpha-diols, (C₁-C₂₀) alkyl phenols or polyethoxylated, polypropoxylated or polyglycerolated fatty acids, having a fatty chain comprising, for example, from 8 to 18 atoms, the number of ethylene oxide groups or propylene oxide may range from 2 to 50 and the number of glycerol groups may range from 2 to 30, copolymers of ethylene oxide and propylene, condensates of ethylene oxide and propylene oxide on fatty alcohols; polyethoxylated fatty amides having from 2 to 30 moles of ethylene oxide, polyglycerolated fatty amides comprising on average 1 to 5 glycerol groups; polyethoxylated fatty amines having 2 to 30 moles of ethylene oxide, ethoxylated sorbitan fatty acid esters having 2 to 30 moles of ethylene oxide; sucrose fatty acid esters, polyethylene glycol fatty acid esters, (C 6-C 24) alkyl polyglycosides, N-alkyl (C₆-C₂₄) glucamine derivatives, amine oxides such as that the oxides of alkyl (C₁₀-C₁₄) amines or the oxides of N-acyl (C₁₀-C₁₄)-aminopropylmorpholine, and mixtures thereof.

Additional suitable nonionic surfactants include, but are not limited to, alkyl polyglucosides (APG), maltose esters, polyglycerolated fatty alcohols, glucamine derivatives, for instance 2-ethylhexyloxycarbonyl-N-methylglucamine, and mixtures thereof. Suitable alkylpolyglucosides include those that contain an alkyl group comprising from 6 to 30 carbon atoms, and a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UP® by the company Cognis.

Suitable maltose derivatives include those described in document EP-A-566 438, such as O-octanoyl-6′-D-maltose or O-dodecanoyl-6′-D-maltose described in document FR-2 739 556.

In some embodiments, the cleansing composition or soap can be formulated in a cosmetically acceptable aqueous medium. Suitable cosmetically acceptable aqueous media can include, in addition to water, one or more solvents such as lower alcohols containing from 1 to 6 carbon atoms, such as ethanol; polyols such as glycerine; glycols such as butylene glycol, isoprene glycol, propylene glycol, polyethylene glycols such as PEG-8, sorbitol, sugars such as glucose, fructose, maltose, lactose, sucrose, and mixtures thereof. The amount of solvent(s) in the composition disclosed herein can range from 0.1 to 95% by weight.

In some embodiments, the cleansing composition or soap described herein can further comprise one or more cationic polymers of the polyquaternium type, which can provide softness and lubricity to a foaming composition. Suitable cationic polymers include Polyquaternium 5 such as the product MERQUAT 5 marketed by the company CALGON, Polyquaternium 6 such as the product SALCARE SC 30 marketed by the company CIBA, and the product MERQUAT 100 marketed by the company CALGON, Polyquaternium 7 such as the MERQUAT S, MERQUAT 2200 and MERQUAT 550 products marketed by the company CALGON, and the SALCARE SC 10 product marketed by the company CIBA, Polyquaternium 10 such as the product Polymer JR400 marketed by the company Amerchol, Polyquaternium 11 such as GAFQUAT 755, GAFQUAT 755N and GAFQUAT 734 products marketed by ISP, Polyquaternium 15 such as the product ROHAGIT KF 720 F marketed by the company ROHM, Polyquaternium 16 such as LUVIQUAT FC905, LUVIQUAT FC370, LUVIQUAT HM552 and LUVIQUAT FC550 products marketed by BASF, Polyquaternium 22 such as the product Merquat 280 sold by the company Calgon, Polyquaternium 28 such as the product STYLEZE CC10 marketed by the company ISP, Polyquaternium 39 such as the MERQUAT PLUS 3330 product marketed by Calgon, Polyquaternium 44 such as the product LUVIQUAT CARE sold by the company BASF, Polyquaternium 46 such as the product LUVIQUAT HOLD marketed by the company BASF, Polyquaternium 47 such as the product MERQUAT 2001 marketed by Calgon, and cationic guars such as the product Jaguar marketed by the company Rhodia can also be used as cationic polymer.

In some embodiments, the cleansing composition or soap described herein can further comprise one or more adjuvants or additives used in cosmetic compositions. Suitable adjuvants or additives include but are not limited to: oils, active agents, perfumes, preservatives, sequestering agents, pearlescent or opacifying agents, pigments, pearlescent agents, mineral or organic fillers such as talc, kaolin, silica powders or of polyethylene, soluble dyes, or any combination thereof.

Examples of oils include vegetable oils (jojoba, avocado, sesame, sunflower, corn, soy, safflower, grape seed), mineral oils (vaseline, isoparaffins optionally hydrogenated), synthetic oils (isopropyl myristate, cetearyl octanoate, polyisobutylene, ethyl hexyl palmitate, alkyl benzoates), volatile or non-volatile silicone oils such as polydimethylsiloxanes (PDMS) and cyclodimethylsiloxanes or cyclomethicones, and fluorinated or fluorosilicone oils and mixtures thereof.

Exemplary active agents include sunscreens, desquamating agents, moisturizing agents, depigmenting agents, pro-pigmentants, alpha-hydroxy acids, antibacterial agents, antiradical agents, anti-pollution agents, anti-inflammatories, retinoids, extracts of algae, mushrooms, vegetables, yeasts, bacteria, hydrolysed, partially hydrolyzed or unhydrolyzed proteins, enzymes, hormones, vitamins and their derivatives, flavonoids and isoflavones, and mixtures thereof.

The cleansing composition or soap described herein can have a pH ranging from 6 to 10 depending on the application chosen. The adjustment of the pH to the desired value can be done conventionally by adding a base (organic or inorganic) in the composition, for example ammonia or a primary, secondary or tertiary (poly) amine such as monoethanolamine, diethanolamine, triethanolamine, isopropanolamine or 1,3-propanediamine, or by addition of a mineral or organic acid, such as a carboxylic acid, for example, citric acid. In the context of shower gels, the pH can vary from 8 to 10.

In some embodiments, the cleansing composition described herein can further comprise optional additives such as colorants, fragrances, antibacterials, preservatives, antioxidants, beads (fragrance, exfoliating or moisturizing), mica, glitter, shea butter, shea butter beads, opacifying agents, pearlizing agents and other such ingredients. In some embodiments, the composition has high clarity (about 2 to about 25 NTU's), a targeted viscosity (about 4,000 to about 10,000 centipoise) for ease of dispensing from an orifice in the range of about 1% to about 1%8, and a yield value (about 3 to about 15 Pascals) that allows the composition to suspend a variety of additives with a uniformity of distribution and enhanced stability (for example, about 8 months at 120° F. (49° C.), and any mixture thereof.

In some embodiments, the cleansing composition or soap can further comprise one or more moisturizers/emollients. Moisturizers can be included in bar or liquid soap compositions to provide conditioning benefits to the skin. The term “moisturizer” describes a material which imparts a smooth and soft feeling to the skin surface.

There are two ways of reducing water loss from the stratum corneum. One is to deposit on the surface of the skin an occlusive layer which reduces the rate of evaporation. The second method is to add nonocclusive hygroscopic substances to the stratum corneum which will retain water, and make this water available to the stratum corneum to alter its physical properties and produce a cosmetically desirable effect. Nonocclusive moisturizers also function by improving the lubricity of the skin.

Both occlusive and nonocclusive moisturizers are contemplated for use in the compositions described herein. Exemplary moisturizers include long chain fatty acids, liquid water-soluble polyols, glycerin, propylene glycol, sorbitol, polyethylene glycol, ethoxylated/propoxylated ethers of methyl glucose (e.g., methyl gluceth-20), ethoxylated/propoxylated ethers of lanoline alcohol (e.g., Solulan-75® available from the Amerchol Co.) coconut and tallow fatty acids, liquid water-soluble polyols (e.g., glycerin, propylene glycol, butylene glycol, hexylene glycol, polypropylene glycol and polyethylene glycol).

Nonocclusive moisturizers can naturally occur in the stratum corneum of the skin, such as sodium pyrrolidone carboxylic acid, lactic acid, urea, L-proline, guanidine and pyrrolidone. Examples of other nonocclusive moisturizers include hexadecyl, myristyl, isodecyl or isopropyl esters of adipic, lactic, oleic, stearic, isostearic, myristic or linoleic acids, as well as many of their corresponding alcohol esters (sodium isostearoyl-2-lactylate, sodium capryl lactylate), hydrolyzed protein and other collagen-derived proteins, aloe vera gel and acetamide MEA (N-acetyl ethanolamine). Other examples of both occlusive and nonocclusive types of moisturizers are disclosed in “Emollients—A Critical Evaluation,” by J. Mausner, Cosmetics & Toiletries, May 1981, incorporated herein by reference.

Exemplary occlusive moisturizers include petrolatum, mineral oil, beeswax, silicones, lanolin and oil-soluble lanolin derivatives, saturated and unsaturated fatty alcohols such as behenyl alcohol, squalene and squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

### Hair Care

In some embodiments, the composition be a hair care composition comprising ingredients commonly used for hair care products. These ingredients can include, but are not limited to, cleansing agents, lathering agents, hydration agents, surfactants, detergents, gelling agents, fragrances, botanical extracts, conditioning agents, humectants, silicones or silicone derivatives, thickening agents, sun blocks, vitamins, alcohols, polyols, polyolethers, and other commonly used ingredients in shampoos, conditioners, and styling agents. Hair care products generally include one or more surfactants, one or more viscosity adjusting agents, one or more preservatives, and one or more fragrances, and any of the ingredients listed below or combinations thereof.

Generally, hair can be damaged and embrittled by the action of external atmospheric agents such as light and bad weather, but also by mechanical or chemical treatments, such as brushing, combing, dyeing, bleaching, permanent-waving and/or relaxing, and over time, hair can become dry, coarse, dull, and/or fragile. To overcome these drawbacks, it is common practice to use care compositions that condition the hair appropriately, giving it satisfactory cosmetic properties, especially in terms of smoothness, sheen, softness, suppleness, lightness, a natural feel and good disentangling properties. These hair care compositions may be, for example, shampoos, conditioning shampoos, hair conditioners, masks, sera, gels, hair lotions and creams that can be rinsed-out or leave-in compositions. In various embodiments, these compositions generally comprise a combination of cationic conditioning agents such as cationic surfactants, cationic polymers, silicones and/or fatty substances, such as fatty alcohols, in order to give the hair satisfactory cosmetic properties, in terms of softness, smoothness and suppleness. Exemplary compositions can comprise silicones, which are known to improve the cosmetic properties of hair in terms of smoothness and flexibility (as shown in U.S. Pat. No. 5,374,421, each of which is incorporated herein by reference).

In some embodiments, the hair care composition can further comprise one or more non-amino polyalkylsiloxanes, one or more oxyethylenated polymers in the presence of fatty alcohols, one or more non-amino polyalkylsiloxanes comprising at least one alkyl chain having at least 12 carbon atoms, one or more oxyethylenated polymers, and/or one or more fatty alcohols. Exemplary oxyethylenated polymers can have a weight-average molecular mass greater than or equal to 10⁶. In some embodiments, the oxyethylenated polymers can be chosen from the compounds having the formula H(OCH₂CH₂)_{z}OH, wherein, z is an integer greater than or equal to 30,000. In certain embodiments, z can range from 30,000 to 120,000, or from 40,000 to 95,000. In some embodiments, the oxyethylenated polymer can be PEG-45M (z=45,000) such as the product sold under the name Polyox WSR N 60 K by the company Amerchol, and PEG-90M (z=90000), and mixtures thereof.

In some embodiments, the hair care composition can further comprise one or more fatty alcohols. The term “fatty alcohol” means any saturated or unsaturated, linear or branched alcohol comprising at least 8 carbon atoms and which is not oxyalkylenated. Exemplary fatty alcohols are solid at room temperature (25° C.) and at atmospheric pressure (1.013*10₅ Pa). Exemplary fatty alcohols include cetyl alcohol, stearyl alcohol, oleyl alcohol, behenyl alcohol, linoleyl alcohol, palmitoleyl alcohol, arachidonyl alcohol, erucyl alcohol, cetylstearyl (or cetearyl) alcohol, and mixtures thereof.

In some embodiments, the hair care composition can further comprise one or more conditioning agents, including cationic surfactants, cationic polymers and mixtures thereof. The term “cationic surfactant” means a surfactant that is positively charged when it is contained in the composition described herein. Suitable cationic surfactants can be chosen from primary, secondary or tertiary fatty amines, optionally polyoxyalkylenated, or salts thereof, and quaternary ammonium salts, and mixtures thereof. An exemplary fatty amine is stearamidopropyl dimethylamine. Exemplary quaternary ammonium salts include tetraalkylammonium salts, including dialkyldimethylammonium or alkyltrimethylammonium salts in which the alkyl group contains approximately from 16 to 22 carbon atoms, in particular behenyltrimethylammonium, distearyldimethylammonium, cetyltrimethylammonium or benzyldimethylstearylammonium salts, or, on the other hand, the palmitylamidopropyltrimethylammonium salt, the stearamidopropyltrimethylammonium salt, the stearamidopropyldimethylcetearylammonium salt, or the stearamidopropyldimethyl(myristyl acetate)ammonium salt sold under the name CERAPHYL 70 by the company Van Dyk.

The term “cationic polymer” means any polymer containing cationic groups and/or groups that can be ionized to cationic groups, which can be non-siliceous. Exemplary cationic polymers include any known for styling the hair, for example, those described in patent application EP-A-0 337 354 and in French patent applications FR-A-2 270 846, 2 383 660, 2 598 611, 2 470 596 and 2 519 863, each of which is incorporated by reference in its entirety. Additional exemplary cationic polymers include those containing units comprising primary, secondary, tertiary and/or quaternary amine groups that may either form part of the main polymer chain or may be borne by a side substituent directly connected thereto. Suitable cationic polymers can have a weight-average molecular mass of greater than 10⁵, including polymers of polyamine, polyaminoamide and polyquaternary ammonium type, including those described in French patents 2 505 348 and 2 542 997, each of which is incorporated by reference in its entirety.

In some embodiments, the hair care composition can further comprise a “non-sulfate” cleansing agent, lathering agent, or surfactant agent. Suitable “non-sulfate” agents include but are not limited to: sodium lauroyl methyl isethionate propanediol, sodium methyl oleoyl taurate, and sodium cocoyl isethionate.

In some embodiments, the hair care composition can further comprise any of the following ingredients or mixtures thereof: quaternary ammonium compound synthetically derived from rapeseed, quaternary polymers of vinylpyrrolidone and/or of vinylimidazole, for instance the products sold under the names LUVIQUAT FC 905, FC 550 and FC 370 and LUVIQUAT Excellence by the company BASF, Cationic polysaccharides, including cationic celluloses, including cellulose ether derivatives comprising quaternary ammonium groups, cationic cellulose copolymers or cellulose derivatives grafted with a water-soluble quaternary ammonium monomer, and cationic galactomannan gums. Exemplary cellulose ether derivatives comprising quaternary ammonium groups are described in French patent 1 492 597. These polymers are also defined in the CTFA dictionary as quaternary ammoniums of hydroxyethylcellulose that have reacted with an epoxide substituted with a trimethylammonium group. Cationic cellulose copolymers or the cellulose derivatives grafted with a water-soluble quaternary ammonium monomer are described in U.S. Pat. No. 4,131,576, such as hydroxyalkyl celluloses, for instance hydroxymethyl, hydroxyethyl or hydroxypropyl celluloses grafted especially with a methacryloylethyltrimethylammonium, methacrylamidopropyltrimethylammonium or dimethyldiallylammonium salt. Suitable associative celluloses such as alkylhydroxyethylcelluloses quaternized with C₈-C₃₀ fatty chains, such as the product QUATRISOFT LM 200, sold by the company Amerchol/Dow Chemical (INCI name Polyquaternium-24) and the products CRODACEL QM (INCI name PG-Hydroxyethylcellulose cocodimonium chloride), CRODACEL QL (C₁₂ alkyl) (INCI name PG-Hydroxyethylcellulose lauryldimonium chloride) and CRODACEL QS (Cis alkyl) (INCI name PG-Hydroxyethylcellulose stearyldimonium chloride) sold by the company Croda. Other suitable fatty-chain hydroxyethylcellulose derivatives include the commercial products SOFTCAT Polymer SL such as SL-100, SL-60, SL-30 and SL-5 from the company Amerchol/Dow chemical of INCI name Polyquaternium-67. Suitable cationic galactomannan gums are described in U.S. Pat. No. 3,589,578 and 4 031 307. Suitable cellulosic ingredients can be in a crystalline form, a microcrystalline form, or a mixture thereof.

In some embodiments, the hair care composition can further comprise one or more cationic proteins or cationic protein hydrolysates, polyalkyleneimines, including polyethyleneimines, polymers containing vinylpyridine or vinylpyridinium units, condensates of polyamines and of epichlorohydrin, quaternary polyureylenes and chitin derivatives, animal protein hydrolysates bearing trimethylbenzylammonium groups, such as the products sold under the name Crotein BTA by the company Croda and referred to in the CTFA dictionary as Benzyltrimonium hydrolyzed animal protein, protein hydrolysates bearing quaternary ammonium groups on the polypeptide chain, the said ammonium groups comprising at least one alkyl radical having from 1 to 18 carbon atoms.

In some embodiments, the hair care composition can further comprise one or more quaternized plant proteins such as from wheat, corn or soybean proteins, for example, quaternized wheat proteins, including those sold by the company Croda under the names Hydrotriticum WQ or QM, referred to in the CTFA dictionary as Cocodimonium hydrolysed wheat protein, Hydrotriticum QL, referred to in the CTFA dictionary as Laurdimonium hydrolysed wheat protein, or else Hydrotriticum QS, referred to in the CTFA dictionary as Steardimonium hydrolysed wheat protein.

In some embodiments, the hair care composition can further comprise one or more polyamines such as POLYQUART R H sold by Cognis, referred to under the name polyethylene glycol tallow polyamine in the CTFA dictionary. Additional suitable polymers include those sold especially under the name Lupamin by the company BASF, and the products sold under the names Lupamin 9095, Lupamin 5095, Lupamin 1095, Lupamin 9030 and Lupamin 9010.

In some embodiments, the hair care composition can further comprise one or more fatty substances that are liquid at room temperature (25° C.) and at atmospheric pressure (1.013*10⁵ Pa). The term “fatty substance” means an organic compound that is insoluble in water at ordinary temperature (25° C.) and at atmospheric pressure (1.013*10⁵ Pa) (solubility of less than 5%, less than 1%, or less than 0.1%). Fatty substances are generally soluble in organic solvents under the same temperature and pressure conditions, for example, in chloroform, dichloromethane, carbon tetrachloride, ethanol, benzene, toluene, tetrahydrofuran (THF), liquid petroleum jelly or decamethylcyclopentasiloxane. The liquid fatty substances of the present disclosure can be nonpolyoxyethylenated and nonpolyglycerolated. The term “oil” means a “fatty substance” that is liquid at room temperature (25° C.) and at atmospheric pressure (1.013*10⁵ Pa). The term “non-silicone oil” means an oil not containing any silicon atoms (Si) and the term “silicone oil” means an oil containing at least one silicon atom. The liquid fatty substances can be chosen from non-silicone oils such as in particular C₆-C₁₆ liquid hydrocarbons, liquid hydrocarbons containing more than 16 carbon atoms, non-silicone oils of animal origin, triglycerides of plant or synthetic origin, fluoro oils, liquid fatty acid and/or fatty alcohol esters other than triglycerides, and mixtures thereof. The liquid hydrocarbons can be linear, branched or optionally cyclic, including hexane, cyclohexane, undecane, dodecane, tridecane or isoparaffins, such as isohexadecane, isodecane or isododecane, and mixtures thereof. Suitable linear or branched liquid hydrocarbons of mineral or synthetic origin containing more than 16 carbon atoms can be chosen from liquid paraffins, petroleum jelly, liquid petroleum jelly, mineral oil, polydecenes and hydrogenated polyisobutene such as PARLEAM and mixtures thereof hydrocarbon-based oils of animal origin, such as perhydrosqualene, can be used.

Exemplary triglycerides of vegetable or synthetic origin can be chosen from liquid fatty acid triglycerides comprising from 6 to 30 carbon atoms, for instance heptanoic or octanoic acid triglycerides, or alternatively, more particularly from those present in plant oils, for instance coconut oil, sunflower oil, corn oil, soybean oil, marrow oil, grapeseed oil, sesame seed oil, hazelnut oil, apricot oil, macadamia oil, arara oil, castor oil, avocado oil, jojoba oil, shea butter oil or synthetic caprylic/capric acid triglycerides, such as those sold by the company Stearineries Dubois or those sold under the names MIGLYOL 810, 812 and 818 by the company Dynamit Nobel, and mixtures thereof. Suitable fluoro oils include perfluoromethylcyclopentane and perfluoro-1,3-dimethylcyclohexane, such as FLUTEC PCl and FLUTEC PC3 by the company BNFL Fluorochemicals; perfluoro-1,2-dimethylcyclobutane; perfluoroalkanes such as dodecafluoropentane and tetradecafluorohexane, sold under the names PF 5050 and PF 5060 by the company 3M, or bromoperfluorooctyl sold under the name FORALKYL by the company Atochem; nonafluoromethoxybutane and nonafluoroethoxyisobutane; perfluoromorpholine derivatives such as 4-trifluoromethyl perfluoromorpholine sold under the name PF 5052 by the company 3M.

Suitable monoesters include dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate, and mixtures thereof.

In some embodiments, the hair care composition can further comprise diethyl sebacate, diisopropyl sebacate, diisopropyl adipate, di(n-propyl) adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and polyethylene glycol distearates, and mixtures thereof.

In some embodiments, the hair care composition can further comprise one or more fatty esters, one or more sugar esters, and/or one or more diesters of C₆-C₃₀, such as C₁₂-C₂₂ fatty acids. The term “sugar” means oxygen-bearing hydrocarbon-based compounds containing several alcohol functions, with or without aldehyde or ketone functions, and which comprise at least 4 carbon atoms. Suitables sugars can include monosaccharides, oligosaccharides or polysaccharides, for example, sucrose (or saccharose), glucose, galactose, ribose, fucose, maltose, fructose, mannose, arabinose, xylose and lactose, and derivatives thereof, such as alkyl derivatives, such as methyl derivatives, for instance methylglucose. Suitable esters can include oleates, laurates, palmitates, myristates, behenates, cocoates, stearates, linoleates, linolenates, caprates, arachidonates or mixtures thereof, such as, oleate/palmitate, oleate/stearate or palmitate/stearate mixed esters. Suitable monoesters and diesters also include mono- or di-oleate, -stearate, -behenate, -oleopalmitate, -linoleate, -linolenate or -oleostearate of sucrose, of glucose or of methylglucose, including the product sold under the name GLUCATE DO by the company Amerchol, which is a methylglucose dioleate. Additional exemplary esters or mixtures of esters of sugar and of fatty acid that may also be mentioned include: the products sold under the names F160, F140, F 110, F90, F70 and SL40 by the company Crodesta, respectively denoting sucrose palmitate/stearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose mono laurate; the products sold under the name Ryoto Sugar Esters, for example reference B370 and corresponding to sucrose behenate formed from 20% monoester and 80% diester-triester-polyester; the sucrose mono-dipalmitate/stearate sold by the company Goldschmidt under the name TEGOSOFT PSE.

In some embodiments, the hair care composition can further comprise a pH modifying agent such as citric acid and/or sodium hydroxide. Any commonly used pH-modifying agent for hair care compositions is contemplated for use herein.

In various embodiments, the hair care composition can further comprise any of the following ingredients, and/or mixtures thereof: sodium lauroyl methyl isethionate (a cleansing and lathering agent), liquid fatty substances including silicone oils different from the non-amino polyalkylsiloxanes discussed above herein, and organomodified polysiloxanes comprising at least one functional group chosen from amino groups, aryl groups and alkoxy groups. Organopolysiloxanes are defined in greater detail in Walter Noll's Chemistry and Technology of Silicones (1968), Academic Press, the entirety of which is hereby incorporated by reference. They may be volatile or non-volatile. Suitable cyclic polydialkylsiloxanes include octamethylcyclotetrasiloxane sold under the name VOLATILE SILICONE 7207 by Union Carbide or SILBIONE 70045 V2 by Rhodia, decamethylcyclopentasiloxane sold under the name VOLATILE SILICONE 7158 by Union Carbide, and SILBIONE 70045 V5 by Rhodia, and mixtures thereof. Cyclocopolymers of the dimethylsiloxane/methylalkylsiloxane type, such as VOLATILE SILICONE FZ 3109 sold by the company Union Carbide are also suitable. Exemplary cyclic polydialkylsiloxanes with organosilicon compounds include octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane, linear volatile polydialkylsiloxanes such as those sold under the name SH 200 by the company Toray Silicone. Silicones coming within this category are also described in the paper published in Cosmetics and Toiletries, Vol. 91, January 76, pp. 27-32, Todd & Byers, “Volatile Silicone Fluids for Cosmetics,” which is incorporated by reference herein.

Exemplary suitable non-volatile polydialkylsiloxanes include polydimethylsiloxanes having trimethylsilyl end groups such as the SILBIONE oils of the 47 and 70 047 series or the MIRASIL oils sold by Rhodia, such as, for example, the oil 70 047 V 500 000; the oils of the MIRASIL series sold by Rhodia; the oils of the 200 series from the company Dow Corning, such as DC200 with a viscosity of 60 000 mm²/s; the VISCASIL oils from General Electric and certain oils of the SF series (SF 96, SF 18) from General Electric, the polydimethylsiloxanes having dimethylsilanol end groups known under the name dimethiconol (CTFA), such as the oils of series 48 from the company Rhodia.

Exemplary organomodified silicones include polyalkylarylsiloxanes, and products sold under the following names: SILBIONE oils of the 70 641 series from Rhodia; the oils of the RHODORSIL 70 633 and 763 series from Rhodia; the oil Dow Corning 556 Cosmetic Grade Fluid from Dow Corning; the silicones of the PK series from Bayer, such as the product PK20; the silicones of the PN and PH series from Bayer, such as the products PN1000 and PH1000, certain oils of the SF series from General Electric, such as SF 1023, SF 1154, SF 1250 and SF 1265, the products sold under the names GP 4 Silicone Fluid and GP 7100 by Genesee or the products sold under the names Q2 8220 and Dow Corning 929 or 939 by the company Dow Corning.

In various embodiments, the hair care composition can further comprise one or more additional surfactants different from the cationic surfactants described above, including anionic surfactants, amphoteric or zwitterionic surfactants, nonionic surfactants and mixtures thereof. Exemplary anionic surfactants include alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfo succinates, alkylsulfoacetates, acylsarcosinates, acylglutamates, alkylsulfosuccinamates, acylisethionates and N—(C₁-C₄)alkyl N-acyltaurates, salts of alkyl monoesters and of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds (unless otherwise mentioned) generally comprising from 6 to 24 carbon atoms and the aryl group generally denoting a phenyl group. Anionic surfactants in salt form can include alkali metal salts such as the sodium or potassium salt, the sodium salt, ammonium salts, amine salts and amino alcohol salts or alkaline-earth metal salts such as the magnesium salt. Exemplary amino alcohol salts include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Suitable anionic surfactants also include mild anionic surfactants, i.e. anionic surfactants without a sulfate function, including polyoxyalkylenated alkyl ether carboxylic acids; polyoxyalkylenated alkylaryl ether carboxylic acids; polyoxyalkylenated alkylamido ether carboxylic acids, in particular those comprising 2 to 50 ethylene oxide groups; alkyl-D-galactoside uronic acids; acylsarcosinates, acylglutamates; and alkylpolyglycoside carboxylic esters such as those sold under the name AKYPO RLM 45 CA from Kao.

Exemplary suitable amphoteric or zwitterionic surfactant(s) can be secondary or tertiary aliphatic amine derivatives, optionally quaternized, in which the aliphatic group is a linear or branched chain containing from 8 to 22 carbon atoms, where the amine derivatives contain at least one anionic group, for example a carboxylate, sulfonate, sulfate, phosphate or phosphonate group, such as (C₈-C₂₀)alkylbetaines, sulfobetaines, (C₈-C₂₀)alkylamido(C₃-C₈)alkylbetaines or (C₈-C₂₀)alkylamido(C₆-C₈)alkylsulfobetaines. Any suitable secondary or tertiary aliphatic amine derivative can be present in coconut oil or in hydrolysed linseed oil, or the like. Representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, the cocoamphodiacetate sold by the company Rhodia under the trade name MIRANOL C2M Concentrate, and sodium diethylaminopropyl cocoaspartamide and sold by the company Chimex under the name Chimexane HB are suitable for use in the disclosed compositions.

Suitable nonionic surfactants are described in the Handbook of Surfactants by M. R. Porter, published by Blackie & Son (Glasgow and London), 1991, pp. 116-178, and include fatty alcohols, fatty α-diols, fatty (C₁-C₂₀)alkylphenols and fatty acids, which can be ethoxylated, propoxylated or glycerolated and contain at least one fatty chain comprising from 8 to 18 carbon atoms, the number of ethylene oxide or propylene oxide groups ranging from 1 to 200, and the number of glycerol groups ranging from 1 to 30. Condensates of ethylene oxide and of propylene oxide with fatty alcohols, ethoxylated fatty amides having from 1 to 30 ethylene oxide units, polyglycerolated fatty amides comprising on average from 1 to 5 glycerol groups, and in particular from 1.5 to 4, ethoxylated fatty acid esters of sorbitan containing from 1 to 30 ethylene oxide units, fatty acid esters of sucrose, fatty acid esters of polyethylene glycol, (C₆-C₂₄)alkylpolyglycosides, oxyethylenated plant oils, N—(C₆-C₂₄)alkylglucamine derivatives, amine oxides such as (C₁₀-C₁₄)alkylamine oxides or N—(C₁₀-C₁₄)acylaminopropylmorpholine oxides are also suitable for use in the compositions disclosed herein.

Additional suitable nonionic surfactants include alkyl polyglucosides (APG), maltose esters, polyglycerolated fatty alcohols, glucamine derivatives, for instance 2-ethylhexyloxycarbonyl-N-methylglucamine, and mixtures thereof. Alkylpolyglucosides that are those containing an alkyl group comprising from 6 to 30 carbon atoms, and containing a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UP® by the company Cognis.

Suitable maltose derivatives include those described in document EP-A-566 438, such as O-octanoyl-6′-D-maltose or O-dodecanoyl-6′-D-maltose described in document FR-2 739 556. Each of these documents is incorporated by reference in its entirety.

In some embodiments, the hair care composition can be formulated in a cosmetically acceptable medium. The term “cosmetically acceptable medium” means a medium that is compatible with human keratin fibers, such as the hair. A cosmetically acceptable medium can be formed from water or from a mixture of water and one or more cosmetically acceptable solvents chosen from lower alcohols, such as ethanol and isopropanol; polyols and polyol ethers, including 2-butoxyethanol, propylene glycol, propylene glycol monomethyl ether, diethylene glycol monoethyl ether and monomethyl ether, and mixtures thereof.

In some embodiments, the hair care composition can further comprise any of the following additives, or mixtures thereof: solid fatty substances different from fatty alcohols such as waxes, anionic, nonionic or amphoteric polymers or mixtures thereof, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners different from oxyethylenated polymers, opacifiers or nacreous agents, antioxidants, hydroxy acids, fragrances and/or preserving agents.

### Nail Care

In some embodiments, the cosmetic composition can comprise ingredients commonly used in nail care products. Nail care products include, but are not limited to, nail treatments including nail strengtheners, top coats, and base coats, nail polishes, nail polish removers, hands skincare, feet skincare, drying agents, and corrector pens including nail polish removers.

In some embodiments, the nail care composition can be a nail treatment composition. A nail treatment composition can comprise a composition for treating ingrown nails or nail deformities, a composition for topical treatment of nail infections, including fungal infections, nail strengtheners, top coats, base coats, polish removers, or any combination thereof. In some embodiments, a nail treatment composition can be formulated as a topical nail lacquer or polish, creams, solutions, suspensions, lotions, serums, gels, balms, gels, oils, oil in creams, and/or scrubs for treating the hands and/or the feet.

In some embodiments, the nail treatment composition can be nail strengthener. Nail strengtheners can treat fingernails and toenails to both harden, strengthen, and promote growth of the nails, to prevent or minimize breaking, cracking, splitting and peeling, and can comprise any known composition used to prevent and heal quarter cracks while increasing the growth of horse hooves, which have a similar protein consistency to human fingernails and toenails, including: lanolin, butter, beeswax, rosin, copper acetate, and turpentine. In some embodiments, the nail treatment composition can further comprise titanium Dioxide for example, TI-PURE R900 from E.I. DuPont.

In some embodiments, the nail treatment composition can be a base coat. In some embodiments, a base coat can be a liquid composition comprising at least one polymer which provides adhesion, for example a polymer co-polymerized from methyl methacrylate (MMA) and methacrylic acid (MAA) to form a polymer composed of polymethyl methacrylate (PMMA) and polymethacrylic acid (PMAA), in which the MAA monomer fraction may vary from 0 to 100%. In some embodiments, suitable polymers for use as a base coat include hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, and mixtures thereof. In some aspects, a base coat can further comprise a non-reactive, solvent-dissolvable, film-forming polymer such as a cellulose ester, for example cellulose acetate alkylate, cellulose acetate butyrate, or cellulose acetate propionate. The above exemplary ingredients are not limiting.

In some embodiments, the nail treatment composition can be a top coat, for example a quick drying top coat. Top coats can include a base resin which is cellulose acetate butyrate, a film former which is a methacrylate polymer, thermally curable or photocurable monomers which are monofunctional methacrylates and cross-linkers (i.e., difunctional and trifunctional methacrylates), a thermal initiator or photoinitiator(s) together in a solution of aliphatic esters and alcohol, and a photoreactive coating. Suitable solvents include acetates and alcohols, particularly ethyl acetate, butyl acetate, and isopropyl alcohol. The photoreactive coating can include photoreactive monomers including methacrylate monomers such as: cyclohexyl methacrylate, n-decyl methacrylate, 2-ethyl hexyl methacrylate, ethyl methacrylate, hydroxy propyl methacrylate, isobornyl methacrylate, 2-methoxy ethyl methacrylate; difunctional and trifunctional methacrylate monomers, cross-linking agents such as diurethane dimethacrylate, ethylene glycol dimethacrylate, 1,10 decanediol dimethacrylate, 1,6-hexanediol dimethacrylate, and trimethylolpropane trimethacrylate. Commercially available photoinitiators suitable for use include but are not limited to: benzoin methyl ether, 2-hydroxy-2-methyl-1-phenyl-1-propanone (“Darocur 1173”), diethoxyacetophenone, and benzyl diketal. Photoinitiators are oligomeric mixtures of phenyl propanones such as a mixture of 2,4,6-trimethylbenzophenone and 4-methylbenzophenone and a mixture of oligo-[2-hydroxy-2-methyl-1[4-(1-methylvinyl)phenyl]propanone] and 2-hydroxy-2-methyl-phenyl propanone that are sold by Sartomer under the names “Esacure KIP 100F” and “Esacure TZT Photoinitiator.”

In some embodiments, the nail care composition can be a nail polish or nail enamel composition. In some embodiments, the nail polish or nail enamel composition can further comprise a nitrocellulose film former, a latex film former, a polycarbodiimide film former, a low volatile organic compound (VOC), and polycarbodiimide film formers. Polycarbodiimides can include polymers with a plurality of carbodiimide groups appended to the polymer backbone. For example, U.S. Pat. No. 5,352,400 (the disclosure of which is incorporated by reference herein) discloses polymers and co-polymers derived from alpha-methylstyryl-isocyanates. Suitable polycarbodiimide compounds include, but are not limited to, those commercially sold by the suppliers Nisshinbo (including those known by the name under the CARBODILITE series, V-02, V02-L2, SV-02, E-02, V-10, SW-12G, E-03A), Picassian, and 3M.

In some embodiments, the nail polish or nail enamel composition can further comprise one or more latex polymers, including carboxyl functional acrylate latex polymers, carboxyl functional polyurethane latex polymers, carboxyl functional silicone latex polymers, carboxyl functional non-acrylate latex polymers and mixtures thereof. In various embodiments, suitable latex polymers can be film-forming latex polymers or non film-forming latex polymers. In some embodiments, the latex polymers can be carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers The term “(meth)acryl” and variations thereof, as used herein, means acryl or methacryl. The (meth)acrylic monomers may be chosen from, for example, acrylic acid, methacrylic acid, citraconic acid, itaconic acid, maleic acid, fumaric acid, crotonic acid, and maleic anhydride. The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth)acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, .alpha.-methylstyrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers herein is not limiting, and it should be understood that it is possible to use any monomer known to those skilled in the art which includes acrylic and/or vinyl monomers (including monomers modified with a silicone chain).

In some non-limiting exemplary embodiments, carboxyl functional acrylate latex polymers can be chosen from aqueous dispersions of Methacrylic Acid/Ethyl Acrylate copolymer (INCI: Acrylates Copolymer, such as LUVIFLEX SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), Acrylates Copolymers, such as those known under the tradenameDermacryl AQF (Akzo Nobel), under the tradename LUVIMER MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In some embodiments, the nail care composition can comprise a nail polish remover. Nail polish removers can include a polyhydric alcohol compound including glycerin, glycols, polyglycerin, esters of polyhydric alcohols, and mixtures thereof. The glycol can contain from 2 to 12 carbon atoms, such as, for example, glycerin, propylene glycol, butylene glycol, propane diol, hexylene glycol, polyglycerin, dipropylene glycol and diethylene glycol. Suitable esters of polyhydric alcohol include liquid esters of saturated or unsaturated, linear or branched C1-C26 polyhydric alcohols. Examples of suitable esters of polyhydric alcohol include, but are not limited to, esters of dihydroxy, trihydroxy, tetrahydroxy or pentahydroxy alcohols. The ester of polyhydric alcohol may be a glyceryl ester such as, glyceryl triglycolate, glyceryl tricitrate, glyceryl trilactate, glyceryl trilactate, glyceryl tributanoate, glyceryl triheptanoate, glyceryl trioctanoate, etc.

A nail polish remover can further comprise a low carbon alcohol, a containing from 1 to 8 carbon atoms. The low carbon alcohol may contain from 2 to 6 carbon atoms, such as from 2 to 5 carbon atoms. Examples of low carbon alcohols include, but are not limited to, ethanol, propanol, butanol, pentanol, isopropanol, isobutanol, and isopentanol. A nail polish remover can further comprise a high boiling point ester compound including, but are not limited to, carbonate esters, adipates, sebacates and succinates. Exemplary high boiling point ester compounds include, but are not limited to, alkylene carbonates such as propylene carbonate, dimethyl succinate, diethyl succinate, dimethyl glutarate, diethyl glutarate, dimethyl sebacate, diethyl sebacate, diisopropyl sebacate, bis(2-ethylhexyl) sebacate, dimethyl adipate, diisopropyl adipate, di-n-propyl adipate, dioctyl adipate, bis(2-ethylhexyl) adipate, diisostearyl adipate, ethyl maleate, bis(2-ethylhexyl) maleate, triisopropyl citrate, triisocetyl citrate, triisostearyl citrate, trioctyldodecyl citrate and trioleyl citrate.

A nail polish remover can further comprise a thickening agent including but not limited to: nonionic, anionic, cationic, amphiphilic, and amphoteric polymers, and other known rheology modifiers, such as cellulose-based thickeners such as hydroxyethylcellulose, hydroxypropylcellulose, methylcellulose, and ethylhydroxyethylcellulose. Certain notable cellulose derivatives include hydroxyl-modified cellulose polymers such as Hydroxyethylcellulose, e.g., those having a molecular weight over 500,000 daltons such as NATROSOL 250 HHR and Hydroxypropyl cellulose, e.g., KLUCEL MF—both available from Ashland of Covington, Ky. The thickening agent can be a polysaccharide such as fructans, glucans, galactans and mannans or heteropolysaccharides such as hemicellulose, pullulan or branched polysaccharides such as gum arabic and amylopectin, or mixed polysaccharides such as starch. The thickening agent can be an acrylic thickening agent (acrylic thickener) or an acrylamide thickening agent (acrylamide thickener). The thickening agent can comprise at least one monomer performing a weak acid function such as acrylic acid, methacrylic acid, itaconic acid, crotonic acid, maleic acid and/or fumaric acid. The thickening agent can comprise a monomer performing a strong acid function such as monomers having a function of the sulfonic acid type or phosphonic acid type, such as 2-acrylamido-2-methylpropane sulfonic acid (AMPS). The thickening agent can comprise a crosslinking agent such as methylene bisacrylamide (MBA), ethylene glycol diacrylate, polyethylene glycol dimethacrylate, diacrylamide, cyanomethacrylate, vi nyloxyethacrylate or methacrylate, formaldehyde, glyoxal, and compositions of the glycidylether type such as ethyleneglycol diglycidylether, or epoxides. Suitable acrylic thickeners are disclosed in U.S. patent application publication nos. 2004/0028637 and 2008/0196174, both of which are incorporated herein by reference. In some embodiments, the thickening agent can comprise an organoclay (hydrophobically treated clay) or a hydrophilic clay.

In some embodiments, the thickening agent can comprise an abrasive compound (abrasive system). An “abrasive compound” is a compound capable of providing abrasion or mechanical exfoliation. The abrasive particles can comprise perlite, pumice, zeolites, hydrated silica, calcium carbonate, dicalcium phosphate dihydrate, calcium pyrophosphate, alumina, sodium bicarbonate, polylactic acid, as well as synthetic polymeric materials such as polyethylene, polypropylene, polyethylene terephthalate, polymethyl methacrylate or nylon. In certain embodiments a moderately hard abrasive includes perlite, such as a cosmetic grade perlite available from Imerys under the name IMERCARE 270P-Scrub. In certain embodiments a soft abrasive is a sugar, a ground fruit kernel or shell powders such as apricot kernel, coconut husk, or spherical waxes (for example, carnauba jojoba); argan shell powder, and the like.

In some embodiments, the nail care composition can further comprise an additive commonly used in cosmetic compositions and known to a person skilled include solvents, preservatives, fragrances, oils, waxes, surfactants, antioxidants, agents for combating free radicals, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

### Therapeutic Compositions

In some embodiments, the composition described herein can be a therapeutic composition that is useful for treating one or more conditions. For example, and in some embodiments, application of the compositions described herein can promote wound healing, reduce or prevent the formation of scar tissue, promote tissue regeneration, minimize local inflammation, minimize tissue rejection, and/or enhance graft integration. In some embodiments, the composition can be formulated as an injectable material, e.g., a hydrogel. Collagen hydrogels present a large, uniform surface area, and can serve as a delivery system for collagen and, optionally, one or more additional therapeutic agents. Injectable collagen materials can also form scaffolds or networks capable of both replacing tissue function and supporting tissue regeneration. In certain embodiments, the composition can be topically applied. In certain embodiments, the composition can be dermally, intradermally, or subcutaneously injected.

In some embodiments, the therapeutic compositions described herein can comprise one or more additional therapeutic agents and/or prophylactic agents—other than the collagen fragments described elsewhere herein. The one or more additional therapeutic and/or prophylactic agents can be a small molecule active agent or a biomolecule, such as an enzyme or protein, polypeptide, or nucleic acid.

Non-limiting examples of additional therapeutic and/or prophylactic agents include anti-cancer agents, antimicrobial agents (including anti-viral agents, antibacterial agents, anti-fungal agents, and anti-parasitic agents), antioxidants, analgesics, local anesthetics, anti-inflammatory agents, cytokines, immunosuppressant agents, anti-allergenic agents, essential nutrients, growth factors (such as fibroblast growth factor, hepatocyte growth factor, platelet-derived growth factor, vascular endothelial cell growth factor, and insulin-like growth factor), and combinations thereof. Specific dosages of the additional therapeutic and/or prophylactic agents can be readily determined by those of skill in the art. See Ansel, Howard C. et al., Pharmaceutical Dosage Forms and Drug Delivery Systems (6th ed.) Williams and Wilkins, Malvern, PA (1995).

In other embodiments, the recombinant collagen fragment composition described herein can be used in combination with cell delivery, for example, the delivery of stem cells, pluripotent cells, somatic cells, and combinations thereof.

Although the present disclosure contemplates that the therapeutic and/or prophylactic agents will be an integral part of the composition described herein, in some embodiments, the therapeutic and/or prophylactic agents can be administered prior to, in conjunction with, or subsequent to administration of the therapeutic composition described herein. In other words, in some embodiments, the one or more additional therapeutic and/or prophylactic agents may not be integrally incorporated into the present compositions, but be provided separately from the present compositions.

Non-limiting examples of suitable local anesthetics capable of being included in the present compositions include, but are not limited to, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxy tetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof.

Non-limiting examples of suitable antiviral agents include ganciclovir and acyclovir. Non-limiting examples of suitable antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines. Additional exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

Non-limiting examples of suitable anti-inflammatory agents include steroidal active agents include glucocorticoids, progestins, mineralocorticoids, and corticosteroids. Exemplary non-steroidal anti-inflammatory drug include ketorolac, ibuprofen, nepafenac, diclofenac, aspirin, and naproxen. Other exemplary anti-inflammatory agents include triamcinolone acetonide, fluocinolone acetonide, prednisolone, dexamethasone, loteprendol, fluorometholone, and dipotassium glycyrrhizate.

In some embodiments, the composition can further comprise one or more additional pharmaceutically active agents. Exemplary pharmaceutical agents can include non-steroidal anti-inflammatory agents (NSAIDs), e.g., flurbiprofen, ibuprofen, naproxen, indomethacin and related compounds. In some embodiments, the composition can further comprise one or more anti-mitotic drugs including colchicine, taxol and related compounds. In some embodiments, the composition can further comprise one or more topical antiseptics such as, e.g., benzoyl peroxide. In some embodiments, the composition can further comprise one or more polysaccharides produced by microalgae, e.g., alguronic acid.

In some embodiments, the composition can further comprise one or more immune-modulating drugs. Exemplary immune-modulating drugs include imiquimod, cyclosporine, tacrolimus, and rapamycin.

In some embodiments, the composition can further comprise one or more cytokines. Exemplary suitable cytokines include, but are not limited to, IL-10, TGF-β, IL-25, and IL-35. In certain embodiments, the cytokines can induce Treg activation (e.g. IL-25) and suppress Th17 activation (e.g. IL-10) in order to minimize rejection.

In some embodiments, the compositions described herein further comprises at least one eukaryotic cell type. Some exemplary eukaryotic cell types include stem cells, mesenchymal stem cells, keratinocytes, fibroblasts, melanocytes, adipocytes, immune cells such as T lymphocytes, B lymphocytes, natural killer cells, and dendritic cells, or combinations thereof. In some embodiments, the stem cells can be adipose-derived mesenchymal stem cells. Functional characteristics of mesenchymal stem cells that can benefit wound healing include their ability to migrate to the site of injury or inflammation, participate in regeneration of damaged tissues, stimulate proliferation and differentiation of resident progenitor cells, promote recovery of injured cells through growth factor secretion and matrix remodeling, and exert unique immunomodulatory and anti-inflammatory effects (See e.g., Phinney O G et al., Stem Cells, 25:2896-2902 (2007); Chamberlain G et al, Stem Cells, 25:2739-2749(2007); Dazzi F et al., Curr Opin Oncol. 19:650-655 (2007)). Each of these references is incorporated by reference in its entirety.

In some embodiments, the eukaryotic cell can be responsible for increasing the structural integrity of connective tissue and/or promote healing. In some embodiments, the eukaryotic cell, such as a fibroblast, can be responsible for enhancing or promoting the growth or connection of cells or tissues.

In some embodiments, the recombinant collagen composition can be in contact with 1) an implanted hair graft and 2) tissue into which the hair graft is implanted. Recipient sites for hair grafts include the scalp, the facial region, the armpit or chest region or the pubic region. Specific areas of the facial region include eyebrows, eyelid, mustache, sideburn, chin and cheeks. The recipient site can be any area of skin wherein the appearance of hair is desired by the subject. In some embodiments, the contact between the composition described herein and the implanted hair with the surrounding tissues of the recipient site promotes nutritional perfusion from the surrounding tissues into the graft and increases the survival rate of the graft compared to a graft implanted without the use of the composition described herein. In certain embodiments, the contact between the composition described herein and the implanted hair, and between the composition described herein and the surrounding tissues of the recipient site promotes vascularization around the implanted grafts, such that the survival rate of the graft is increased compared to a graft implanted without the use of the composition described herein.

### Dietary Compositions

In some embodiments, the composition described herein can be a dietary composition useful for useful for providing collagen to a subject in need thereof. For example, and in some embodiments, consumption of the dietary compositions described herein can provide health and/or skin benefits, such as increasing collagen intake, relieving joint pain, and improving skin health. In certain embodiments, the composition can be in the form of a powder, a capsule, a liquid, or any other suitable form.

In some embodiments, the dietary compositions can comprise one or more nutritional ingredients such as: ascorbic acid, biotin, chromium nicotinate, copper citrate, D-calcium pantothenate, cyanocobalamin, flax seed, Linum usitatissimum, folic acid, fructooligosaccharide (fiber), magnesium oxide, manganese citrate, maltodextrin, medium chain triglycerides, flavor, niacinamide, potassium citrate potassium iodide, riboflavin, sugar cane (Saccharum officinarum), sodium molybdate dihydrate, sodium selenate (selenium), soy protein isolate, Stevia leaf extract/Stevia rebaudiana, thiamin HCl, tricalcium phosphate, vitamin a palmitate, vitamin D3, xanthan gum, zinc citrate, cellulose gum, guar gum, pyridoxine hydrochloride, salt, tocopherol, antioxidants, e.g., resveratrol, CoQ10, acai berry, lycopene and pomegranate, natural or artificial sweeteners, e.g., glucose, sucrose, fructose, saccharides, cyclamates, aspartamine, sucralose, aspartame, acesulfame K, or sorbitol, flavoring, such as a flavored extract, volatile oil, chocolate flavoring (e.g., non-caffeinated cocoa or chocolate, chocolate substitutes such as carob), peanut butter flavoring, cookie crumb, vanilla, or any commercially available flavoring, and any combination thereof.

In some embodiments, as mentioned above, the composition described herein can be in the form of an alcohol- or water-based toner. Exemplary toner formulations are set forth below.

### Alcohol-Based Toner

| INGREDIENTS: | % WT/WT |
|---|---|
| Water (Aqua) | Remainder Ingredient |
| Recombinant collagen fragment | about 0.0005% to about 25% |
| disclosed herein | |
| Alcohol Denat. | 10-20% |
| Pentylene Glycol | 5-10% |
| Glycerin | 1-5% |
| Gluconolactone | 0.1-1% |
| Dipotassium Glycyrrhizate | 0.1-1% |
| Sodium Citrate | 0.1-1% |
| Sodium Benzoate | 0.1-1% |

### Water-Based Toner

| INGREDIENTS: | % WT/WT |
|---|---|
| Water (Aqua) | Remainder Ingredient |
| Recombinant collagen fragment | about 0.0005% to about 25% |
| disclosed herein | |
| Niacinamide | 1-5% |
| Pentylene Glycol | 1-5% |
| Propanediol | 1-5% |
| Glycerin | 1-5% |
| Biosaccharide Gum-1 | 0.1-1% |
| Glyceryl Caprylate | 0.1-1% |
| Sodium Anisate | 0.1-1% |
| Sodium Hydroxide | 0.1-1% |
| Caprylhydroxamic Acid | 0.1-1% |
| Acrylates/C10-30 Alkyl Acrylate | 0.1-1% |
| Crosspolymer | |
| Sodium Levulinate | 0.1-1% |
| Caprylyl Glycol | 0.1-1% |

In some embodiments as mentioned above, the composition described herein can be in the form of a cream, a gel, or a serum. Exemplary cream, gel, and serum formulations are set forth below.

### Cream

| INGREDIENTS: | % WT/WT |
|---|---|
| Water (Aqua) | Remainder Ingredient |
| Recombinant collagen | about 0.0005% to about 25% |
| fragment disclosed herein | |
| Cetearyl Alcohol | 5-10% |
| Glycerin | 1-5% |
| Squalane | 1-5% |
| Butyrospermum Parkii (Shea) | 1-5% |
| Butter | |
| Glyceryl Caprylate | 1-5% |
| Microcrystalline Cellulose | 1-5% |
| Glyceryl Stearate Citrate | 0.1-1% |
| Tocopheryl Acetate | 0.1-1% |
| Cetearyl Glucoside | 0.1-1% |
| Sodium Stearoyl Glutamate | 0.1-1% |
| Cellulose Gum | 0.1-1% |
| Xanthan Gum | 0.1-1% |
| Caprylhydroxamic Acid | 0.1-1% |
| Sodium Phytate | 0.01-0.1% |

### Gel

| INGREDIENTS: | % WT/WT |
|---|---|
| Water (Aqua) | Remainder Ingredient |
| Recombinant collagen fragment | about 0.0005% to about 25% |
| disclosed herein | |
| Sodium Phytate | 0.1-1% |
| Sodium Hydroxide | 0.1-1% |
| Carbomer | 0.1-1% |
| Phenoxyethanol | 0.1-1% |

| INGREDIENTS: | % WT/WT |
|---|---|
| Water (Aqua) | Remainder Ingredient |
| Recombinant collagen fragment | about 0.0005% to about 25% |
| disclosed herein | |
| Pentylene Glycol | 1-5% |
| Niacinamide | 1-5% |
| Dimethicone | 1-5% |
| Propanediol | 1-5% |
| Tocopherol | 0.1-1% |
| Sodium Hyaluronate | 0.1-1% |
| Linoleic Acid | 0.1-1% |
| Ammonium | 0.1-1% |
| Acryloyldimethyltaurate/VP | |
| Copolymer | |
| Acrylates/C10-30 Alkyl Acrylate | 0.1-1% |
| Crosspolymer | |
| Caprylyl Glyceryl Ether | 0.1-1% |
| Tetrasodium Glutamate Diacetate | 0.01-0.1% |
| Sodium Hydroxide | 0.01-0.1% |
| Phenoxyethanol | 0.01-0.1% |
| Linolenic Acid | 0.001-0.0 |

### Serum

In some embodiments as mentioned above, the composition described herein can be a shampoo or conditioner. Exemplary shampoo or conditioner formulations are set forth below.

### Shampoo

| INGREDIENTS: | % WT/WT |
|---|---|
| Water (Aqua) | Remainder Ingredient |
| Recombinant collagen fragment | about 0.0005% to about 25% |
| disclosed herein | |
| Cocamidopropyl Betaine | 5-10% |
| Sodium Lauroyl Methyl Isethionate | 5-10% |
| Propanediol | 1-5% |
| Sodium Methyl Oleoyl Taurate | 1-5% |
| Sodium Cocoyl Isethionate | 1-5% |
| Trisodium Ethylenediamine | 0.1-1% |
| Disuccinate | |
| Caprylhydroxamic Acid | 0.1-1% |
| Panthenol | 0.1-1% |
| Citric Acid | 0.1-1% |
| Caprylyl Glycol | 0.1-1% |
| Sodium Benzoate | 0.1-1% |

### Conditioner

| INGREDIENTS: | % WT/WT |
|---|---|
| Water (Aqua) | Remainder Ingredient |
| Recombinant collagen fragment | about 0.0005% to about 25% |
| disclosed herein | |
| Cetearyl Alcohol | 5-10% |
| Glyceryl Caprylate | 1-5% |
| Behentrimonium Methosulfate | 1-5% |
| Glycerin | 1-5% |
| Caprylhydroxamic Acid | 0.1-1% |
| Panthenol | 0.1-1% |
| Hydroxyethylcellulose | 0.1-1% |
| Cocos Nucifera (Coconut) Oil | 0.1-1% |
| Citric Acid | 0.01-0.1% |

### D. Yeast Strains

The present disclosure can use yeast to produce the collagen fragments described herein. In some embodiments, modified yeast can be used to produce collagen fragments. Suitable yeast include those of the genus Pichia, Candida, Komatagaella, Hansenula, Cryptococcus, Saccharomyces and combinations thereof. In some embodiments, the yeast can be from the genus Pichia. The yeast can be modified or hybridized. Hybridized yeast can be prepared by breeding different strains of the same species, different species of the same genus, or strains of different genera. Examples of yeast strains that are suitable to produce the collagen fragments disclosed herein include Pichia pastoris, Pichia membranfaciens, Pichia deserticola, Pichia cephalocereana, Pichia eremophila, Pichia myanmarensis, Pichia anomala, Pichia nakasei, Pichia siamensis, Pichia heedii, Pichia barkeri, Pichia norvegensis, Pichia thermomethanolica, Pichia stipites, Pichia subpelliculosa, Pichia exigua, Pichia occidentalis, Pichia cactophila, and the like.

In one embodiment, the Pichia pastoris strains can be engineered to express a codon-optimized polynucleotide encoding a collagen fragment.

In some embodiments, a collagen fragment encoded by a yeast host cell is fused to a polypeptide sequence that facilitates its secretion from the yeast. For example, a vector can encode a chimeric gene comprising a coding sequence for a collagen fragment fused to a sequence encoding a secretion peptide. Secretion sequences which can be used for this purpose include Saccharomyces alpha mating factor Prepro sequence, Saccharomyces alpha mating factor Pre sequence, PHO1 secretion signal, α-amylase signal sequence from Aspergillus niger, Protein with internal repeats 1 signal sequence, Glucoamylase signal sequence from Aspergillus awamori, Serum albumin signal sequence from Homo sapiens, Inulinase signal sequence from Kluyveromcyes maxianus, Invertase signal sequence from Saccharomyces cerevisiae, Killer protein signal sequence from Saccharomyces cerevisiae and Lysozyme signal sequence from Gallus gallus. Other secretion sequences known in the art can also be used.

In some embodiments one or more of the following yeast promoters can be incorporated into a vector to promoter transcription of mRNA encoding the protein of interest (e.g., a collagen fragment). Promoters are known in the art and include pAOX1, pDas1, pDas2, pPMP20, pCAT, pDF, pGAP, pFDH1, pFLD1, pTAL1, pFBA2, pAOX2, pRKI1, pRPE2, pPEX5, pDAK1, pFGH1, pADH2, pTPI1, pFBP1, pTAL1, pPFK1, pGPM1, and pGCW14.

In some embodiments a yeast terminator sequence is incorporated into a vector to terminate transcription of mRNA encoding the protein of interest (e.g., a collagen fragment). Terminators include but are not limited to AOX1 TT, Das1 TT, Das2 TT, AOD TT, PMP TT, Cat1 TT, TPI TT, FDH1 TT, TEF1 TT, FLD1 TT, GCW14 TT, FBA2 TT, ADH2 TT, FBP1 TT, and GAP TT.

In some embodiments, the recombinant collagen fragment described herein is produced in a genetically engineered strain of yeast. In some embodiments, the yeast is Pichia pastoris.

In some embodiments, the yeast was transformed with a plasmid (vector) comprising the nucleic acid sequence set forth in SEQ ID NO: 973. In some embodiments, to improve recombinant protein expression, the yeast was transformed again (i.e., doubly transformed) with a second plasmid. In some embodiments, the yeast was doubly transformed with a plasmid comprising the nucleic acid sequence set forth in SEQ ID NO: 974.

In some embodiments, the recombinant collagen fragment described herein is produced by a method comprising:
(i) fermenting a yeast strain in a fermentation broth;
(ii) recovering from fermentation broth, recombinant collagen fragments secreted by the yeast strain; and
(iii) optionally, purifying the recombinant collagen fragments.

In some embodiments, the recombinant collagen fragment can then undergo ex vivo hydroxylation.

In some embodiments, the recombinant collagen fragment can be produced in a genetically engineered strain of yeast. In some embodiments, the yeast can be Pichia pastoris. In some embodiments, the yeast can be transformed with a plasmid comprising the nucleic acid sequence set forth in SEQ ID NO: 973. In some embodiments, the yeast can be doubly transformed with a plasmid comprising the nucleic acid sequence set forth in SEQ ID NO: 974.

In some embodiments, the recombinant collagen fragment sequence variant described herein can be produced by a method comprising:
(i) fermenting a yeast strain in a fermentation broth;
(ii) recovering from fermentation broth, recombinant collagen fragment sequence variants secreted by the yeast strain; and
(iii) optionally, purifying the recombinant collagen fragment sequence variants.

In some embodiments, the recombinant collagen fragment sequence variants. can then undergo ex vivo hydroxylation.

In some embodiments, the recombinant collagen fragment variant can be produced in a genetically engineered strain of yeast. In some embodiments, the yeast can be Pichia pastoris. In some embodiments, the yeast can be transformed with a plasmid comprising the nucleic acid sequence set forth in any one of SEQ ID NOs: 1045-1073. In some embodiments, the yeast can be doubly transformed with a nucleic acid sequence set forth in any one of SEQ ID NOs: 1045-1073.

DNA can be introduced into the yeast strain by electroporation. Transformants can be selected, for example, using host yeast cells that are auxotrophic for leucine, tryptophan, uracil, or histidine together with selectable marker genes such as LEU2, TRP1, URA3, HIS3, or LEU2-D. The DNA sequence for the collagen fragment can be introduced into the yeast via a vector, e.g., by electroporation. DNA can be inserted into a vector. Suitable vectors include, but are not limited to, pHTX1-BiDi-P4HA-Pre-P4HB hygro, pHTX1-BiDi-P4HA-PHO1-P4HB hygro, pGCW14-pGAP1-BiDi-P4HA-Prepro-P4HB G418, pGCW14-pGAP1-BiDi-P4HA-PHO1-P4HB Hygro, pDF-Col3A1 modified Zeocin, pCAT-Col3A1 modified Zeocin, pDF-Col3A1 modified Zeocin with AOX1 landing pad, pHTX1-BiDi-P4HA-Pre-Pro-P4HB hygro. The vectors typically included at least one restriction site for linearization of DNA. Once within a yeast strain, DNA can be inserted into the yeast genome and used to produce collagen fragments.

In some embodiments, the present disclosure provides a strain of yeast, e.g., Pichia pastoris, genetically engineered to produce the recombinant collagen fragment described herein, wherein the strain of yeast comprises a vector comprising a DNA sequence encoding the recombinant collagen fragment. In some embodiments, the vector can comprise a nucleic acid sequence comprising the DNA sequence set forth in SEQ ID NO: 973. In some embodiments, the vector can comprise a nucleic acid sequence comprising the DNA sequence set forth in SEQ ID NO: 974. In some embodiments, the vector is inserted into the yeast through electroporation.

In some embodiments, the present disclosure provides a vector comprising a DNA sequence for producing a collagen fragment, wherein the vector comprises the nucleic acid sequence set forth in SEQ ID NO: 973.

In some embodiments, the present disclosure provides a vector comprising a DNA sequence for producing a collagen fragment, wherein the vector comprises the nucleic acid sequence set forth in SEQ ID NO: 974.

In some embodiments, the present disclosure provides a vector comprising a DNA sequence for producing a collagen fragment, wherein the vector comprises the nucleic acid sequence set forth in any one of SEQ ID NOs: 1045-1073.

In some embodiments, the present disclosure provides a strain of collagen fragment-producing yeast comprising a vector comprising a DNA sequence for producing the recombinant collagen fragment described herein.

A promoter can improve the production of a recombinant protein and can be included in a vector comprising sequences encoding the protein of interest (e.g., the collagen fragments described herein). Suitable promoters for use in preparing the collagen fragments disclosed herein include, but are not limited to, AOX1 methanol induced promoter, pDF de-repressed promoter, pCAT de-repressed promoter, Das1-Das2 methanol induced bi-directional promoter, pHTX1 constitutive Bi-directional promoter, pGCW14-pGAP1 constitutive Bi-directional promoter and combinations thereof. Suitable methanol induced promoters include but are not limited to AOX2, Das 1, Das 2, pDF, pCAT, pPMP20, pFDH1, pFLD1, pTAL2, pFBA2, pPEX5, pDAK1, pFGH1, pRKI1, pREP2, and combinations thereof.

A terminator can be placed at the end of each open reading frame utilized in the vectors incorporated into the yeast. The DNA sequence for the terminator can be inserted into the vector. For replicating vectors, an origin of replication is necessary to initiate replication. The DNA sequence for the origin of replication is inserted into the vector. One or more DNA sequences containing homology to the yeast genome can be incorporated into the vector to facilitate recombination and incorporation into the yeast genome or to stabilize the vector once transformed into the yeast cell.

A vector can also generally include at least one selective marker that is used to select yeast cells that have been successfully transformed. The markers sometimes are related to antibiotic resistance and markers can also be related to the ability to grow with or without certain amino acids (auxotrophic markers). Suitable auxotrophic markers included, but are not limited to ADE, HIS, URA, LEU, LYS, TRP and combinations thereof. To provide for selection of yeast cells containing a recombinant vector, at least one DNA sequence for a selection marker can be incorporated into the vector.

The engineered yeast cells described above can be utilized as hosts to produce the collagen fragment described herein. In order to do so, the cells can be placed in media within a fermentation chamber and fed dissolved oxygen and a source of carbon (e.g., glycerol), under controlled pH conditions for a period of time ranging from twelve hours to 1 week. Suitable media include but are not limited to buffered glycerol complex media (BMGY), buffered methanol complex media (BMMY), and yeast extract peptone dextrose (YPD).

### E. Methods of Use

### Increasing Collagen Production in Cells

The present inventors have surprisingly discovered that the collagen fragments described in this disclosure can induce the production of Type I and Type III collagen in cells such as fibroblasts. Thus, when applied to cells such as fibroblasts, or to other appropriate cells, the collagen fragment described here (i.e., a collagen fragment having the amino acid sequence of SEQ ID NO: 1 or SEQ ID NO: 986, or a composition comprising such a fragment) can advantageously induce collagen formation in the cells on which it is applied.

Accordingly, in some embodiments, the present disclosure provide methods of increasing collagen production in cells, comprising administering a collagen fragment described herein to cells. Such administration can consist of directly exposing the cells to the collagen fragment in an amount sufficient to induce the cells to produce greater amounts of collagen. In some embodiments, the method comprises administering the collagen fragment as part of a composition comprising the collagen fragment and at least one pharmaceutically acceptable excipient. In some embodiments, the collagen fragment can be administered to cells in their natural context, such as in an organism or tissue. In some embodiments, the collagen fragment can be topically applied to skin. In some embodiments, the collagen fragment can be topically applied to human skin. In some embodiments, the collagen fragment can be administered to cultured cells as part of the culture media.

In some embodiments, the cells can be fibroblasts. In some embodiments, the cells can be fibroblasts in their native context (e.g., within human skin). In some embodiments, the cells can be primary human fibroblasts. In some embodiments, the cells can be muscle cells, transformed human cells, cardiomyocytes, endothelial cells, stem cells, or induced pluripotent stem cells.

In some embodiments, the recombinant collagen fragments disclosed herein, or compositions comprising one or more fragments, can be applied to a wound, such as a cut, laceration, gash, tear, scrape, abrasion, or scratch, in order to increase collagen production at the wound site and/or its surrounding tissues. As part of the wound healing process, fibroblasts migrate to a wound site and produce collagen that is necessary for wound repair. The cells eventually fill the wound cavity with a network of interlacing threads of collagen which in due time, arrange themselves in firm bands and form the permanent new tissue. Accordingly, in some embodiments, the recombinant collagen fragment according to SEQ ID NO: 1 or SEQ ID NO: 986 or compositions comprising the fragment can be applied to a wound, such as a cut, laceration, gash, tear, scrape, abrasion, or scratch, in order to increase collagen production at the wound site and/or its surrounding tissues. In some embodiments, one or more hydrolysis products of the recombinant collagen fragment according to SEQ ID NO: 1 or SEQ ID NO: 986, or compositions comprising such hydrolysis products, can be applied to a wound, such as a cut, laceration, gash, tear, scrape, abrasion, or scratch, in order to increase collagen production at the wound site and/or its surrounding tissues. In some embodiments, compositions comprising a mixture of the recombinant collagen fragment according to SEQ ID NO: 1 or SEQ ID NO: 986 and one or more hydrolysis products thereof can be applied to a wound, such as a cut, laceration, gash, tear, scrape, abrasion, or scratch, in order to increase collagen production at the wound site and/or its surrounding tissues.

Therefore, in certain embodiments, the present disclosure provides methods of applying compositions comprising a recombinant collagen fragment, hydrolysis products of a recombinant collagen fragment, or combinations thereof to a wound, such as a cut, laceration, gash, tear, scrape, abrasion, or scratch. In certain embodiments, the wound can be a wound in a human subject. In some embodiments, the method comprises providing a composition for and promoting collagen production and wound healing in the skin of a subject in need thereof, wherein the composition comprises a recombinant collagen fragment described herein (e.g., a fragment comprising SEQ ID NO: 1 or SEQ ID NO: 986). In some embodiments, the method comprises providing a composition for and promoting collagen production and wound healing in the skin of a subject in need thereof, wherein the composition comprises one or more hydrolyzed products of a recombinant collagen fragment described herein (e.g., hydrolyzed products comprising one or more of SEQ ID NOs: 2-972). In some embodiments, the method comprises providing a composition for and promoting collagen production and wound healing in the skin of a subject in need thereof, wherein the composition comprises a mixture of a recombinant collagen fragment and one or more hydrolyzed products of that recombinant collagen fragment, as described herein (e.g., a mixture of a fragment comprising SEQ ID NO: 1 or SEQ ID NO: 986, and hydrolyzed products comprising SEQ ID NOs: 2-972).

In some embodiments, the method can comprise administering a collagen fragment with the amino acid sequence of SEQ ID NO: 1 or SEQ ID NO: 986 to cells. In some embodiments, the method can comprise administering a hydrolysis product resulting from the hydrolysis of a collagen fragment with the amino acid sequence of SEQ ID NO: 1 or SEQ ID NO: 986 to cells (e.g., administering one or more hydrolysis products with sequences according to SEQ ID NOs: 2-972). In some embodiments the method comprises administering a recombinant collagen fragment described herein and one or more hydrolysis products of that recombinant collagen fragment to cells. In some embodiments the method can comprise administering a collagen fragment with the amino acid sequence of SEQ ID NO: 1 or SEQ ID NO: 986, and one or more hydrolysis products of that fragment that have sequences according to SEQ ID NOs: 2-972 to cells.

In some embodiments, the method can increase the production of Type I collagen. In some embodiments, the method can increase the production of Type III collagen. In some embodiments, the method can increase the production of both Type I and Type III collagen.

### Skincare

In some embodiments, the composition described herein can be a skincare composition that can be used to treat an area of the skin by topically applying the skincare composition to the area of skin.

In some embodiments, the composition described herein can be a skincare composition that can be used to treat the skin, hair, and nails, by consuming the composition as a dietary supplement.

In some embodiments, administration of the composition described herein to the skin can improve or maintain the quality of skin and reduce or eliminate signs of aging. Signs of aging include, but are not limited to, all outward visibly and tactilely perceptible manifestations as well as any other macro or micro effects due to skin aging. Such signs may be induced or caused by intrinsic factors or extrinsic factors (such as chronological aging and/or environmental damage). These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin.

In some embodiments, a composition as described herein can be suitable for use as a dermal filler. A dermal filler composition can replace lost endogenous matrix polymers, or enhance/facilitate the function of existing matrix polymers, in order to treat skin conditions due to aging or injury. The dermal filler composition can fill wrinkles, lines, folds, scars, and to enhance dermal tissue, such as, e.g., to plump thin lips, or fill-in sunken eyes or shallow cheeks. Earlier dermal filler products generally were made of collagens. One common matrix polymer used in modern dermal filler compositions is hyaluronan. Because hyaluronan is natural to the human body, it is a generally well tolerated and a fairly low risk treatment for a wide variety of skin conditions.

In some embodiments, a composition as described herein can be suitable for use with a microneedle sheet or patch. Microneedles can provide the composition described herein to any area of the face and body to achieve a cosmetic benefit or wound healing benefit.

In some embodiments, the composition described herein can be a therapeutic composition that can be used to reduce or prevent the formation of scar tissue, promoting healing, promoting tissue regeneration, minimizing local inflammation, minimizing tissue rejection, and/or enhance skin and/or hair graft integration.

In some embodiments, the disclosure provides a method of treating a dermatological condition comprising administering an effective amount of the composition comprising a recombinant collagen fragment to a subject in need thereof. In some embodiments, the dermatological condition can be fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

In some embodiments, the composition can be topically administered to an area of skin. In some embodiments, the area of skin can be selected from the group consisting of a facial surface, scalp, neck, ears, shoulders, chest (including breasts and/or the decolletage), arms, hands, legs, stomach, buttocks, groin, back, feet, and combinations thereof.

In some embodiments, the composition can be topically administered to a facial surface. In some embodiments, the facial surface can be selected from the group consisting of forehead, eyes, a perioral surface, a chin surface, a periorbital surface, a nasal surface, a cheek skin surface, and combinations thereof.

In some embodiments, the disclosure provides a method for improving collagen production in the skin, comprising administering an effective amount of a composition comprising a recombinant collagen fragment.

In some embodiments, the disclosure provides a skincare product comprising the composition described herein for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

### EMBODIMENTS

E1. A recombinant collagen fragment having a molecular weight of about 50 kDa and a sequence identity of at least about 85% to the amino acid sequence set forth in SEQ ID NO: 986.

E2. The recombinant collagen fragment of embodiment 1, wherein the recombinant collagen fragment is unhydroxylated.

E3. The recombinant collagen fragment of embodiment 1, wherein the recombinant collagen fragment is hydroxylated.

E4. The recombinant collagen fragment of embodiment 1, wherein the collagen fragment has the amino acid sequence set forth in SEQ ID NO: 986.

E5. A sequence variant of the recombinant collagen fragment of embodiment 1, wherein the sequence variant comprises the amino acid sequence set forth in any one of SEQ ID NOs: 987-1015.

E6. The sequence variant of embodiment 5, wherein the sequence variant is unhydroxylated.

E7. The sequence variant of embodiment 5, wherein the sequence variant is hydroxylated.

E8. A composition comprising the recombinant collagen fragment of any one of embodiments 1-4.

E9. A composition comprising the sequence variant of any one of embodiments 5-7.

E10. The composition of embodiment 8, wherein the composition further comprises one or more peptides formed from the hydrolyzation of the collagen fragment having the amino acid sequence set forth in SEQ ID NO: 986.

E11. The composition of embodiment 10, wherein at least one of the one or more peptides formed from the hydrolyzation of the collagen fragment having the amino acid sequence set forth in SEQ ID NO: 986 has an amino acid sequence according to one of SEQ ID NOs: 2-972.

E12. The composition of any one of embodiments 8-11, further comprising a pharmaceutically acceptable or cosmetically acceptable excipient.

E13. A method of producing the recombinant collagen fragment of any one of embodiments 1-4, comprising producing the recombinant collagen fragment in a genetically engineered strain of yeast.

E14. The method of embodiment 13, wherein the yeast is Pichia pastoris.

E15. The method of embodiment 13 or 14, wherein the yeast has been transformed with a plasmid comprising the nucleic acid sequence set forth in SEQ ID NO: 973.

E16. The method of any one of embodiments 13-15, wherein the yeast is a yeast that has been transformed with a plasmid comprising the nucleic acid sequence set forth in SEQ ID NO: 974.

E17. The method of any one of embodiments 13-16, wherein the method comprises:
(i) fermenting a genetically engineered yeast in a fermentation broth;
(ii) recovering from the fermentation broth recombinant collagen fragments secreted by the genetically engineered yeast; and
(iii) optionally, purifying the recombinant collagen fragments.

E18. The method of embodiment 17, further comprising hydroxylating the recombinant collagen fragment ex vivo.

E19. A method of producing the sequence variant of any one of embodiments 5-7, comprising producing the recombinant collagen fragment in a genetically engineered strain of yeast.

E20. The method of embodiment 19, wherein the yeast is Pichia pastoris.

E21. The method of embodiment 19 or 20, wherein the yeast has been transformed with a plasmid comprising the nucleic acid sequence set forth in any one of SEQ ID NO: 1045-1073.

E22. The method of any one of embodiments 19-21, wherein the method comprises:
(i) fermenting a genetically engineered yeast in a fermentation broth;
(ii) recovering from the fermentation broth recombinant collagen fragment sequence variants secreted by the genetically engineered yeast; and
(iii) optionally, purifying the recombinant collagen fragments.

E23. The method of embodiment 22, further comprising hydroxylating the recombinant collagen sequence variant ex vivo.

E24. A strain of yeast genetically engineered to produce the recombinant collagen fragment of any one of embodiments 1-4, wherein the strain of yeast comprises a vector comprising a DNA sequence encoding the recombinant collagen.

E25. A strain of yeast genetically engineered to produce the sequence variant of any one of embodiments 5-7, wherein the strain of yeast comprises a vector comprising a DNA sequence encoding the variant.

E26. The strain of yeast of embodiment 24, wherein the vector comprises a nucleic acid sequence comprising the DNA sequence set forth in SEQ ID NO: 973.

E27. The strain of yeast of embodiment 26, further comprising a second vector comprising a nucleic acid sequence comprising the DNA sequence set forth in SEQ ID NO: 974.

E28. The strain of yeast of embodiment 25, wherein the vector comprises a nucleic acid sequence comprising the DNA sequence set forth in any one of SEQ ID NO: 1045-1073.

E29. The strain of yeast of any one of embodiments 24-28, wherein the strain of yeast is a Pichia pastoris.

E30. A method of treating a dermatological condition comprising administering an effective amount of the recombinant collagen fragment of any one of embodiments 1-4, or the sequence variant of any one of embodiments 5-7, to a subject in need thereof.

E31. A method of treating a dermatological condition comprising administering an effective amount of the composition of any one of embodiments 8-12 to a subject in need thereof.

E32. The method of embodiment 31, wherein the dermatological condition comprises fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

E33. The method of embodiment 31 or 32, wherein the composition is topically administered to an area of skin.

E34. The method of embodiment 33, wherein the area of skin is selected from the group consisting of a facial surface, scalp, neck, ears, shoulders, chest (including breasts and/or the decolletage), arms, hands, legs, stomach, buttocks, groin, back, feet, and combinations thereof.

E35. A method of increasing collagen production in cells, comprising administering an effective amount of the recombinant collagen fragment of any one of embodiments 1-4, or the sequence variant of any one of embodiments 5-7, to the cells.

E36. The method of embodiment 35, wherein the method increases the production of Type I collagen.

E37. The method of embodiment 34 or 35, wherein the method increases the production of Type III collagen.

E38. The method of any one of embodiments 35-37, wherein the cells are fibroblasts.

E39. The method of any one of embodiments 35-38 wherein the cells are cultured cells.

E40. The method of any one of embodiments 35-39, wherein the fragment or variant is formulated in a composition.

E41. The method of any one of embodiments 35-41, wherein the fragment has an amino acid sequence set forth in SEQ ID NO: 986.

E42. The method of any one of embodiments 35-41, wherein the sequence variant has an amino acid sequence set forth in any one of SEQ ID NOs: 987-1015.

E43. A skincare product comprising the composition of any one of embodiments 8-12, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

E44. A method of treating a wound in a human subject in need thereof, the method comprising applying the composition of any one of embodiments 8-12 to the wound on the subject, wherein applying the recombinant collagen fragment induces the production of human Type I collagen, human Type III collagen, or a combination thereof.

E45. The method of embodiment 44, wherein the collagen fragment is topically applied to the wound.

E46. A recombinant collagen fragment having a molecular weight of about 50 kDa and a sequence identity of at least about 85% to the amino acid sequence set forth in SEQ ID NO: 1.

E47. The recombinant collagen fragment of embodiment 46, wherein the recombinant collagen fragment is unhydroxylated.

E48. The recombinant collagen fragment of embodiment 46, wherein the recombinant collagen fragment is hydroxylated.

E49. The recombinant collagen fragment of embodiment 48, wherein the collagen fragment has the amino acid sequence set forth in SEQ ID NO: 1.

E50. A recombinant collagen fragment comprising an amino acid sequence according to any one of SEQ ID NOs: 2-972.

E51. A composition comprising the recombinant collagen fragment of any one of embodiments 46-50.

E52. The composition of embodiment 51, wherein the composition further comprises one or more peptides formed from the hydrolyzation of the collagen fragment having the amino acid sequence set forth in SEQ ID NO: 1.

E53. The composition of embodiment 52, wherein at least one of the one or more peptides formed from the hydrolyzation of the collagen fragment having the amino acid sequence set forth in SEQ ID NO: 1 has an amino acid sequence according to one of SEQ ID NOs: 2-972.

E54. The composition of any one of embodiments 51-53, further comprising a pharmaceutically acceptable or cosmetically acceptable excipient.

E55. A method of producing the recombinant collagen fragment of any one of embodiments 46-50, comprising producing the recombinant collagen fragment in a genetically engineered strain of yeast.

E56. The method of embodiment 55, wherein the yeast is Pichia pastoris.

E57. The method of embodiment 55 or 56, wherein the yeast has been transformed with a plasmid comprising the nucleic acid sequence set forth in SEQ ID NO: 973.

E58. The method of any one of embodiments 55-57, wherein the yeast is a yeast that has been transformed with a plasmid comprising the nucleic acid sequence set forth in SEQ ID NO: 974.

E59. The method of any one of embodiments 55-58, wherein the method comprises:
(i) fermenting a genetically engineered yeast in a fermentation broth;
(ii) recovering from the fermentation broth recombinant collagen fragments secreted by the genetically engineered yeast; and
(iii) optionally, purifying the recombinant collagen fragments.

E60. The method of embodiment 59, further comprising hydroxylating the recombinant collagen fragment ex vivo.

E61. A strain of yeast genetically engineered to produce the recombinant collagen fragment of any one of embodiments 46-50, wherein the strain of yeast comprises a vector comprising a DNA sequence encoding the recombinant collagen.

E62. The strain of yeast of embodiment 61, wherein the vector comprises a nucleic acid sequence comprising the DNA sequence set forth in SEQ ID NO: 973.

E63. The strain of yeast of embodiment 62, further comprising a second vector comprising a nucleic acid sequence comprising the DNA sequence set forth in SEQ ID NO: 974.

E64. The strain of yeast of any one of embodiments 61-63, wherein the strain of yeast is a Pichia pastoris.

E65. A method of treating a dermatological condition comprising administering an effective amount of the recombinant collagen fragment of any one of embodiments 46-50 to a subject in need thereof.

E66. A method of treating a dermatological condition comprising administering an effective amount of the composition of any one of embodiments 51-54 to a subject in need thereof.

E67. The method of embodiment 66, wherein the dermatological condition comprises fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

E68. The method of embodiment 66 or 67, wherein the composition is topically administered to an area of skin.

E69. The method of embodiment 68, wherein the area of skin is selected from the group consisting of a facial surface, scalp, neck, ears, shoulders, chest (including breasts and/or the decolletage), arms, hands, legs, stomach, buttocks, groin, back, feet, and combinations thereof.

E70. A method of increasing collagen production in cells, comprising administering an effective amount of the recombinant collagen fragment of any one of embodiments 46-50 to the cells.

E71. The method of embodiment 70, wherein the method increases the production of Type I collagen.

E72. The method of embodiment 70 or 71, wherein the method increases the production of Type III collagen.

E73. The method of any one of embodiments 70-72, wherein the cells are fibroblasts.

E74. The method of any one of embodiments 70-73, wherein the cells are cultured cells.

E75. The method of any one of embodiments 70-74, wherein the fragment is formulated in a composition.

76. The method of any one of embodiments 70-75, wherein the fragment has an amino acid sequence set forth in SEQ ID NO: 1.

E77. A skincare product comprising the composition of any one of embodiments 51-54, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, or any combination thereof.

E78. A method of treating a wound in a human subject in need thereof, the method comprising applying the composition of any one of embodiments 51-54 to the wound on the subject, wherein applying the recombinant collagen fragment induces the production of human Type I collagen, human Type III collagen, or a combination thereof.

E79. The method of embodiment 78, wherein the collagen fragment is topically applied to the wound.

### EXAMPLES

### Example 1: Generation of Pichia Strain Producing Truncated Human Collagen III Fragments

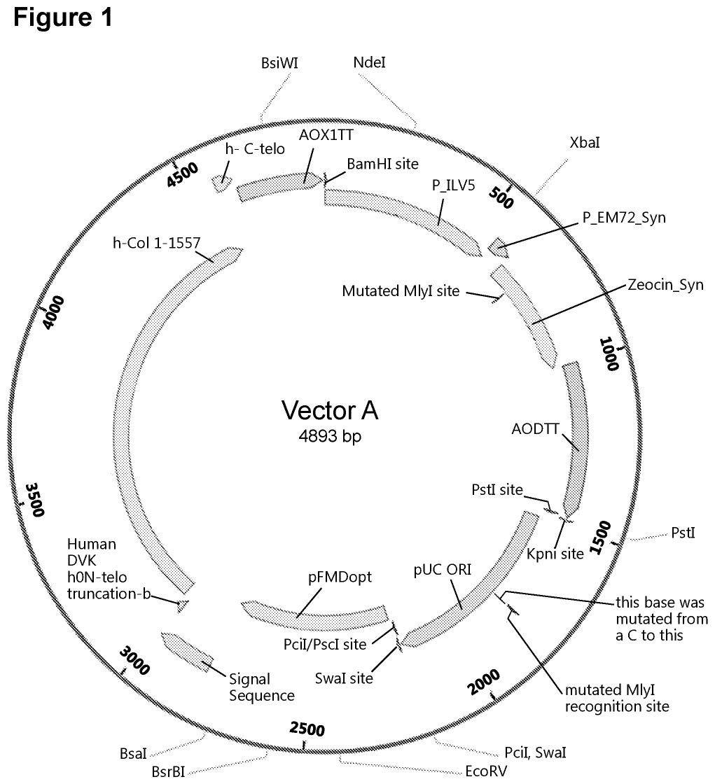

A plasmid encoding secretion signal-secreted 50 kDa human collagen having an amino acid sequence according to SEQ ID NO: 986 (the “50 kDa human collagen fragment”) with zeocin resistance, named Vector A, and shown in FIG. 1, was constructed as follows. To generate the secretion signal fragment, the vector of SEQ ID NO: 984 was amplified via polymerase chain reaction (PCR) with the primers 1 (SEQ ID NO: 975) and 2 (SEQ ID NO: 976). PCR was performed with PHUSION PLUS DNA POLYMERASE using an initial denaturation of 2 min at 95° C. followed by 25 cycles of 95° C. for 30 seconds, 60° C. for 30 seconds, and 72° C. for 15 seconds, and a final extension at 72° C. for 5 minutes. To generate a 50 kDa collagen fragment comprising an amino acid according to SEQ ID NO: 986, the vector of SEQ ID NO: 985 was amplified via PCR with the primers 3 and 4 (SEQ ID NO: 981) and (SEQ ID NO: 977), respectively. PCR was performed with an initial denaturation of 2 minutes at 95° C. followed by 25 cycles of 95° C. for 30 s, 60° C. for 30 s, and 72° C. for 1 minute, and a final extension at 72° C. for 5 minutes. The MMV132 vector (see U.S. 2019/0040400, the entirety of which is incorporated herein by reference) backbone was digested with Mly I restriction endonuclease. All DNA fragments were purified by agarose gel electrophoresis. Fragments were assembled using Gibson Assembly® Master Mix (New England BioLabs).

The resulting circular plasmid DNA was transformed into DH5a E. coli. Zeocin-resistant transformants were obtained after overnight growth at 37° C. 50 kDa collagen fragment constructs were verified by Sanger sequencing. Plasmid DNA from positive clones was purified by PureLink™ HiPure Plasmid Midiprep Kit (Invitrogen). The DNA was linearized by Swa I digestion.

For expression of the 50 kDa collagen fragment, the linearized plasmid was transformed into the PP97 Pichia pastoris (Komagataella phaffii) strain by electroporation with the BIO-RAD GENE PULSER XCELL Total System. Transformants were grown at 30° C. on YPD agar containing 1M sorbitol and 500 μg/mL zeocin. The resulting strain, containing the 50 kDa human collagen fragment under zeocin resistance, was designated as Pichia Strain 1.

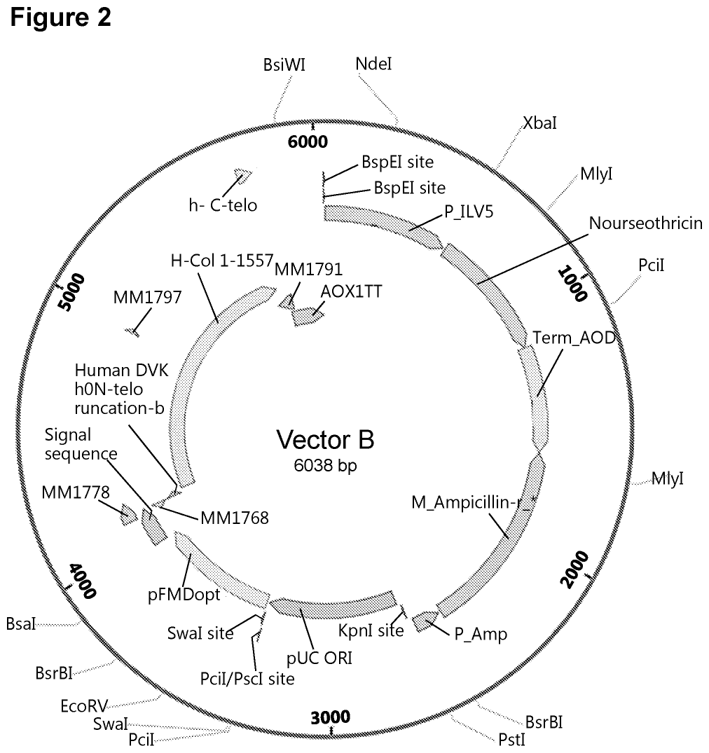

To further improve the recombinant protein expression, a second transformation of the 50 kDa human collagen fragment construct was performed with a nourseothricin N-acetyl transferase (NAT) marker. First, molecular cloning was performed to generate the necessary plasmid DNA. A new vector encoding signal sequence, the 50 kDa human collagen fragment, NAT, and beta-lactamase, named Vector B was created as follows (see FIG. 2). Vector A (FIG. 1) was digested with the Xba I and Eco RV restriction enzymes. The vector of SEQ ID NO: 983, containing NAT resistance, was digested with the Nde I and Bsa I restriction enzymes. The vector fragments containing the 50 kDa human collagen fragment and NAT resistance, respectively, were purified from an agarose gel. Fragments were joined via Gibson assembly and transformed into DH5a competent Escherichia coli (E. coli). For carbenicillin-resistant transformants, the 50 kDa human collagen fragment construct was verified by Sanger sequencing. Plasmid DNA was purified via midiprep, linearized with the Swa I restriction enzyme, and transformed into Pichia strain 1. The resulting doubly transformed strain was designated as Pichia strain 2.

To identify Pichia clones with high expression levels, small-scale cultures were grown for 40 hours in 96 deep well blocks. Cells were pelleted by centrifugation and the supernatant was harvested. Recombinant protein expression was assessed via sodium dodecyl sulphate-polyacrylamide gel electrophoresis (SDS-PAGE). Western blotting was performed with a custom-made primary antibody against the C-telopeptide of collagen (Abmart) and the IRDye® 800CW Goat anti-Mouse IgG secondary antibody (LI-COR).

Screening for collagen production was performed by sandwich enzyme-linked immunosorbent assay (ELISA). This assay utilizes the aforementioned custom-made C-telo antibody as a capture antibody (Abmart) and a commercially available detection antibody, anti-COL3A horseradish peroxidase-conjugate (sc-271249, Santa Cruz). The signal was generated with 3,3′,5,5′-tetramethylbenzidine (TMB) and the reaction was stopped with 2M sulfuric acid. The signal was detected by measuring absorbance at 450 nm with a spectrophotometer. Using these methods, Pichia strain 3 was selected as the best clone.

### Example 2: Fermentation of Selected Clone

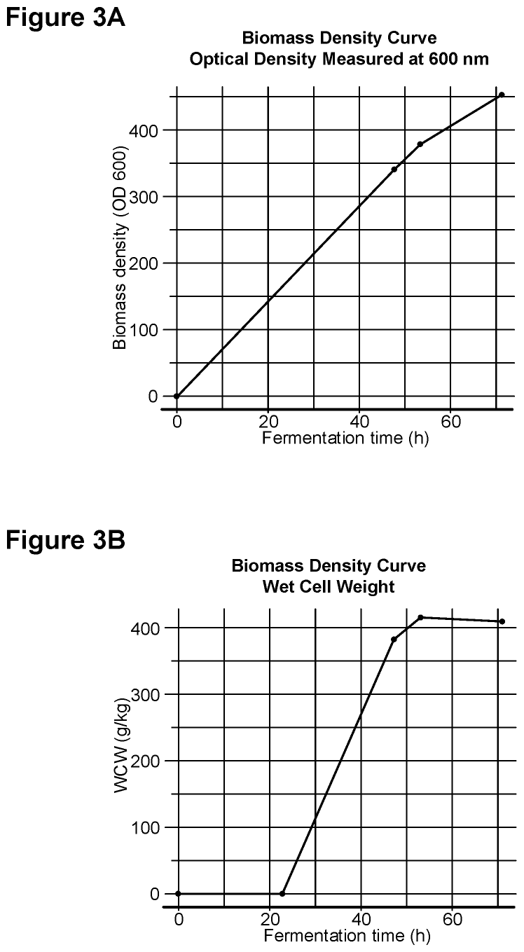

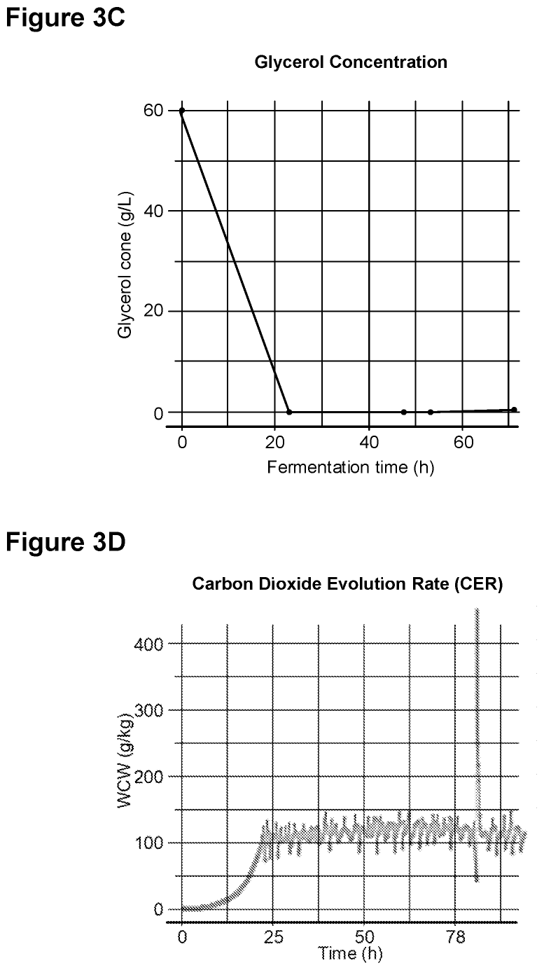

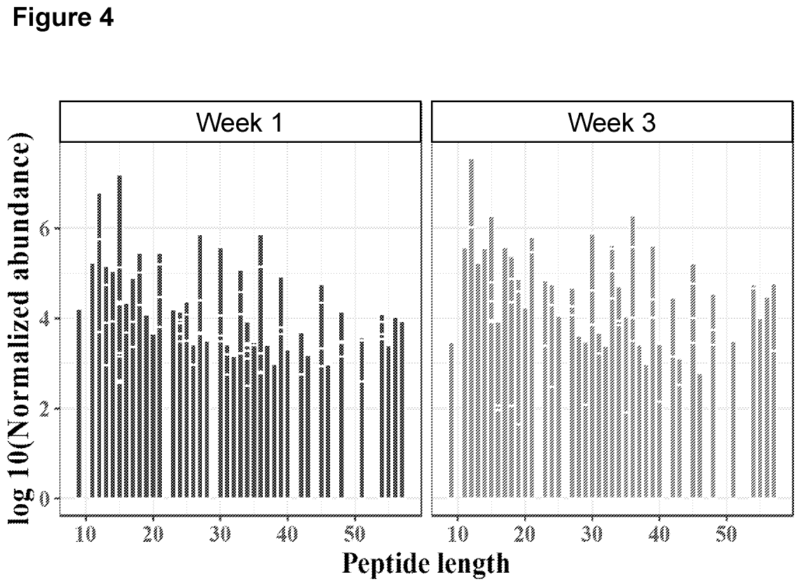

A three-step fermentation process was started by propagating Pichia strain 3 clone in shake flasks, followed by a seed fermentation lasting approximately 23-27 hours, which was then used to inoculate a production fermenter. The production fermenter was run for about 72 hours with a 24-hour batch phase followed by a 48 hour fed-batch phase. Glycerol is used as the carbon source, and a glycerol feed to maintain a constant max OTR value of 150 mmol per L per hour was begun at the end of batch phase. The OTR of 150 mmol per L per hour was maintained by a constant glycerol feeding and by adjusting initial pressure to 800 mbar overpressure, and 1 vvm aeration. The pH was controlled to 6±0.05 by automated addition of 25% ammonium hydroxide and the temperature and the DO were controlled to 32° C. and 25% (of saturation at 800 mbar overpressure) respectively. Samples were taken throughout the fermentation, and the OD600, wet cell weight, dry weight, residual nitrogen, and residual glycerol were measured. (FIGS. 3A-3D).

### Example 3: Purification of Truncated Human Collagen III Fragment

### Fermentation Supernatant Recovery

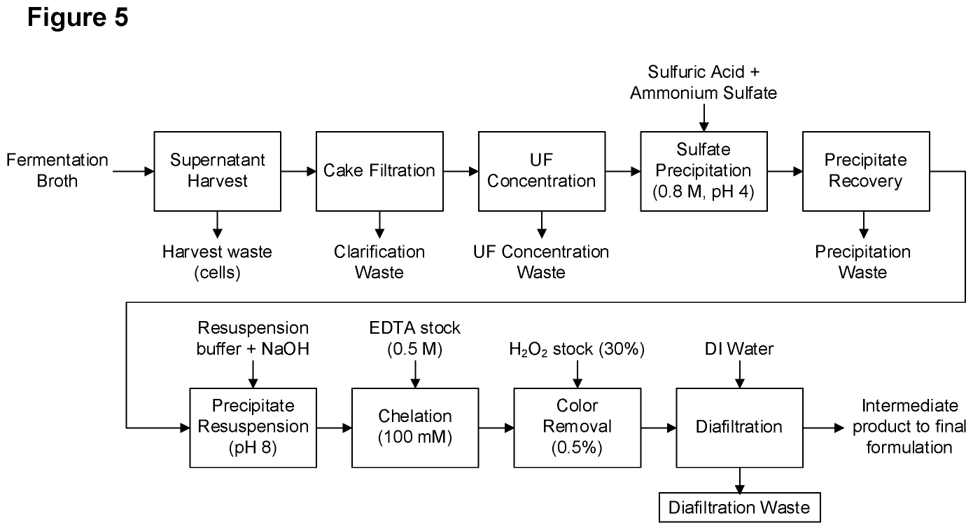

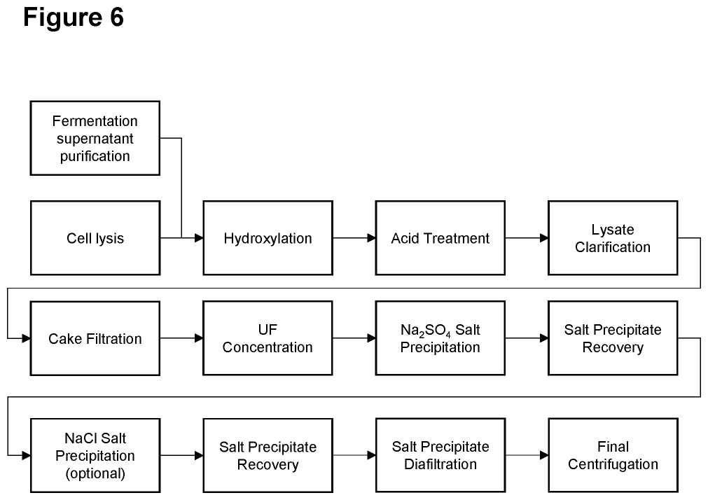

The fermentation broth included a supernatant containing the secreted 50 kDa human collagen having an amino acid sequence according to SEQ ID NO: 986 (the “50 kDa human collagen fragment”) that was first recovered through centrifugation. Depending on the process scale, the supernatant was recovered by batch centrifugation using a Sorvall centrifuge (10 min, 10° C., 17568×g) or using a disc stack centrifuge. For the recovery of the supernatant through disc stack centrifugation, the fermentation broth was diluted with DI water to a wet cell weight (WCW) of 200 g/kg. The disc stack centrifuge (GEA HFC-15) was operated at maximum bowl speed and a feed rate of 350 L/h. After the feed was passed through the centrifuge once, the ejected solids were diluted back to a WCW of 200 g/kg before a second pass. The supernatant obtained from both passes was pooled and continued to the secondary clarification. Flow charts showing the process of purifying the resulting 50 kDa human collagen fragment are shown in FIG. 5 and FIG. 6. FIG. 5 shows the purification process for preparing unhydroxylated 50 kDa human collagen, and FIG. 6 shows the purification process for preparing hydroxylated 50 kDa human collagen

### Cake Filtration

Cake filtration was performed as a secondary clarification step, using a Metchem 470 mm filter press. Per plate used, 0.379 kg of Celite 512 diatomaceous earth was mixed with 20 kg water and applied as a precoat on the filter press. 0.5 wt. % of Celite 512 diatomaceous earth and 0.5 wt. % of Celite SuperCel Fine DE were added to the supernatant and the material and passed through the filter press to remove residual particulate matter.

### UF Concentration

Prior to concentrating the collagen solution, the pH was adjusted to 4 by addition of 50% sulfuric acid. The volume of the resulting solution was decreased by a minimum of a factor 10 through cross flow filtration, using spiral wound PES membranes with a molecular weight cut-off of 10 kDa.

### Diafiltration

Diafiltration was performed to remove small impurities such as salts from the product. Before diafiltration, the pH was adjusted to 8 when necessary by addition of acid (50% sulfuric acid) or base (10 N sodium hydroxide). The diafiltration was performed either in a KrosFlo® KR2i filtration skid equipped with a 10 kDa mPES hollow fiber module or an Alfa Laval M20 filtration skid equipped with a 10 kDa PES spiral wound module. DI water was used as diafiltration medium and the diafiltration was continued until the conductivity of the solution plateaued.

### Preservative

The product of the diafiltration was diluted by addition of deionized (DI) water to reach a concentration of dissolved solids of 2% and during this dilution, 1,2-hexanediol and butylene glycol were added to reach effective concentrations of respectively 3% and 5%.

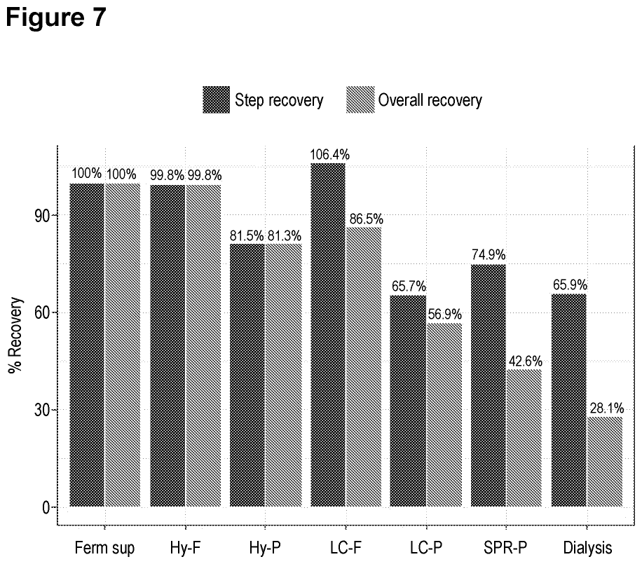

A chart depicting the percent recovery of the 50 kDa human collagen fragment during individual steps of its purification process, and overall during all of the steps up to those individual steps, is shown in FIG. 7.

### Example 4: Characterization of Truncated Human Collagen III Fragment

### Mass Spectrometry (MS) and Sequence Analyses

In solution digestion was performed using an S-Trap micro spin column (ProtiFi, Huntington, NY, USA) following a Strap protocol on as described previously with slight modification. The protein samples (30 μg) in 25 μL of 50 mM TEAB pH 8.5, 6 M Urea, 2 M Thiourea, 1% SDS, 10 mM DTT were reduced for 1 h at 34° C., followed by alkylation with 50 mM iodoacetamide for 45 min at room temperature in the dark and then quenched with a final concentration of 40 mM Dithiothreitol (DTT). After quenching, 12% phosphoric acid was added to a final concentration of 1.2%. Followed by 1:7 dilution (v/v) with 90% methanol, 0.1M Ambic pH 7.9. The samples were then placed into the S-Trap spin column and centrifuged at 4000×g for 30 sec. Then washed three times with 150 μL 90% methanol, 0.1 M Ambic 7.9. Digestion was performed with 125 L LysC at 120 ng/L (1:10 w/w) in 0.1 M Ambic added to the top of the spin column. LysC solution absorbed into the highly hydrophilic matrix, the spin columns and incubated overnight (16 hrs) at 37° C. Following incubation, the digested peptides were eluted off the S-trap column sequentially with 40 μL each of 50 mM TEAB pH 8.5 followed by 0.2% formic acid and finally, 50% acetonitrile-0.2% formic acid. Three eluted peptides washes were pooled together and evaporated to dryness by a Speedvac SC110 (Thermo Savant, Milford, MA). Half (15 μg) of the Lys-digested peptides were reconstituted and digested with 33 μL chymotrypsin at 60 ng/L in 50 mM Ambic (1:7.5 w/w) at 37° C. for 16 hrs. The digestion was stopped by adding 10 μL 5% FA and evaporated to dryness.

The double digested peptides were reconstituted in 150 μL of 0.5% formic acid (“FA”) and 2.5 μL were injected to the nanoLC-MS/MS system. The LysC-chymotryptic peptides were eluted in a 60 min gradient of 5% to 33% ACN-0.1% FA at 300 nL/min. The operation of Orbitrap Fusion was basically same as described above, with an additional EThcD MS2 scan in ion trap for 3-7 charged ions that was toggled with the CID MS2 scan for 2-3 charged ions in 3-second “Top Speed” workflow. The calibrated ETD parameters (reaction time and maximal reagent injection time) were applied for ETD supplemented with an additional 20% HCD activation energy to yield EThcD MS2 spectra.

By combining the results for both the LysC and chymotrypsin sequential digestion and the trypsin single digestion, 97.1% sequence coverage of the target sequence was obtained. The nucleotides in the reconstructed sequence were confirmed as 100% identical to the relevant portion of the human collagen III protein.

### Example 5: Ex-Vivo Hydroxylation Reaction and Analysis Reaction

Fermentation supernatant containing unhydroxylated 50 kDa human collagen having an amino acid sequence according to SEQ ID NO: 986 (the unhydroxylated “50 kDa human collagen fragment”) was partially purified using salt precipitation. Ammonium sulfate was added to the precipitate as salt to a final concentration of 800 mM, the pH was adjusted to 4±0.05 with 50% sulfuric acid the supernatant was then incubated at room temperature with mixing for 30 minutes. The 50 kDa human collagen fragment was recovered from the supernatant by centrifuging at 17000×g and the resulting pellet was resuspended in half the volume of DI water.

The PP547 cell mass was resuspended separately in the Lysis Buffer (50 mM sodium phosphate) to a 30-45% w/w concentration. The pH of the lysis feed was adjusted to 9±0.05 with 2M Citric Acid and/or 2M Sodium Hydroxide. This feed was then lysed in a bead meal according to standard lysis times, volumes and cleaning methods.

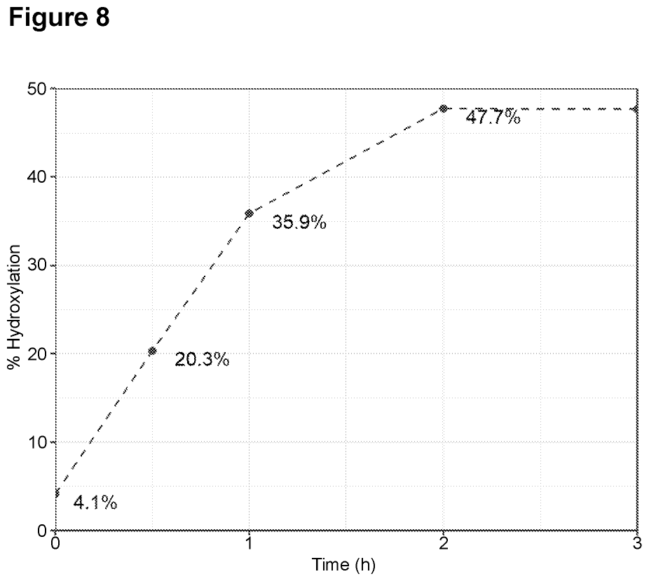

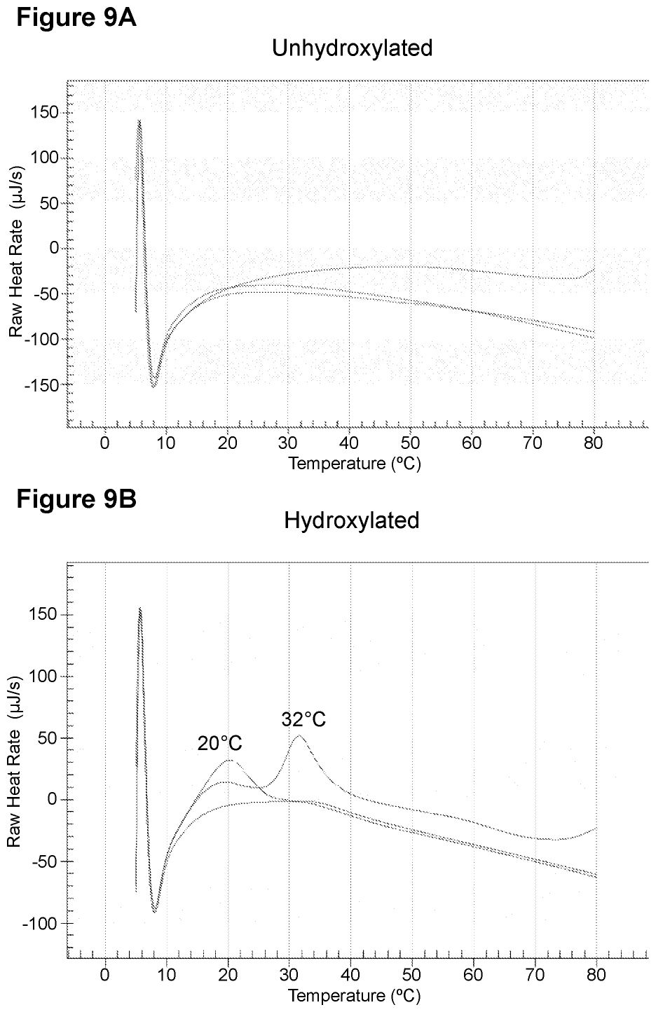

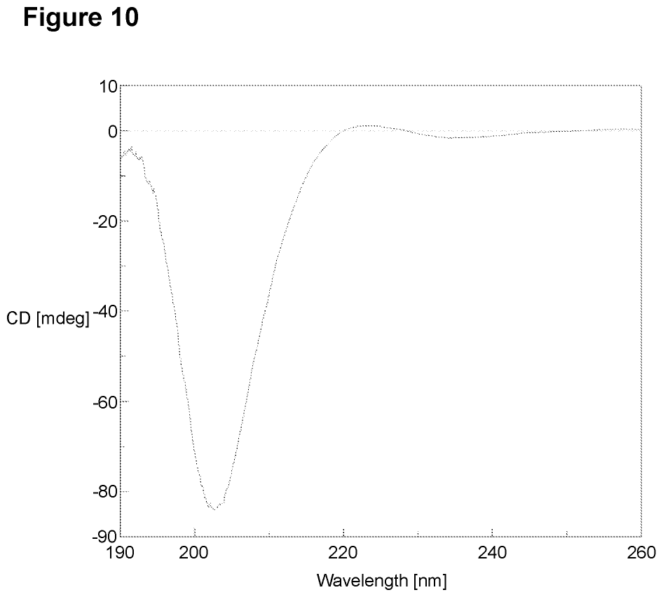

The lysis product was mixed together with the purified supernatant containing the 50 kDa human collagen fragment on a magnetic stirrer with gentle stirring and then 25-65 mM AKG, 25-65 mM DTT, and 2-5 mM Ascorbic acid were added as dry powders. During addition of AKG, DTT, and Ascorbate, the pH was constantly measured and adjusted to avoid going below pH 6.5±0.05 (pH<6.5 inactivates the P4H enzyme). Subsequently 0.1-0.25 mM ferrous sulfate was added from a 0.5M freshly prepared stock solution. This reaction feed was then transferred to a reaction vessel outfitted with pH control, temperature control, mixing control, and aeration control. Once the reaction feed was added to the vessel, aeration was set to 1-2 vvm, mixing to 150-500 rpm depending on tank OTR configuration, temperature control to 26-32° C. and pH control to 7.5±0.05. After 5 hours of reaction, the vessel pH was adjusted to 4±0.05 through automated addition of 2M citric acid, the temperature of the reaction vessel was adjusted to 20-26° C. and nitrogen was sparged through the system to maintain zero dissolved oxygen (DO). After 16 hours of incubation at the aforementioned conditions, primary clarification was performed by centrifuging the reaction product at 17000×g. Following primary clarification, diatomaceous earth was used in a cake filtration system to perform secondary clarification. The clarified reaction product was cooled to 12° C. and concentrated in an ultrafiltration system with a 10 kDa cutoff to reduce the volume by a factor of 10-15. Hydroxylated 50 kDa collagen fragment is then precipitated from the solution using 800 mM sodium sulfate. In some cases, further precipitation can be performed using 1M sodium chloride. Precipitated protein was then resuspended in DI water and the residual salt was removed by diafiltration using a 10 kDa cutoff using one of 0.01 N HCl, 0.001 N HCl, or DI water, depending on the final application of the 50 kDa human collagen fragment. As a final step, the diafiltration product was clarified by further centrifuging at 17,000×g for 10 minutes to remove all particulate material. FIG. 8 shows the percent hydroxylation of the 50 kDa human collagen fragment achieved over time during this ex vivo hydroxylation reaction. FIG. 9A and FIG. 9B show thermograms of the 50 kDa human collagen fragment before and after hydroxylation, respectively.

### Hydrolyzed Peptide Analysis

The final purified product contains native proteases that are co-purified with the 50 kDa human collagen fragment. Incubation at room temperature (T=25° C.) allows the proteases to further hydrolyze the 50 kDa human collagen fragment to produce short collagen peptides. The final product was incubated at room temperature for three weeks to study the rate and extent of hydrolysis. The samples were analyzed with mass spectrometry after one and three weeks of incubation at room temperate (T=25° C.). (FIG. 4). No enzymatic digestion was performed in order to check the relative abundance and sequence of the short collagen peptides. MS spectra revealed that the peptide distribution was unchanged after the first week with nearly 70% of short peptides (1-6 kDa) being of 1 to 1.5 kDa in length. Peptides larger than 6 kDa were not measured in this analysis. Mapping the peptides revealed that the detected peptides covered 97% of the sequence of the initial recombinant collagen amino acid sequence (SEQ ID NO: 986). The peptides identified by mass spectrometry after one week of incubation are provided in Table 1 above. The peptides identified by mass spectrometry after three weeks of incubation are provided in Table 2 above.

### Example 6: Stimulation of Collagen I and Collagen III Synthesis by Truncated Human Collagen III in Fibroblast Cells

A fibroblast cell culture model was used to assess the ability of the test materials to exert an effect on collagen synthesis. This study also assessed the viability of the cells after exposure to the test materials.

### MTT Assay

Changes in cell number can be assessed via an MTT assay. The MTT assay is a colorimetric analysis of the metabolic activity of the cell, which is a reflection of the number of viable cells. Reduction of MTT by mitochondria results in the formation of insoluble purple formazin crystals that are extracted from the cells with isopropanol and quantified spectrophotometrically. The intensity of the purple color is directly proportional to the metabolic activity of the cells and inversely proportional to the toxicity of the test material.

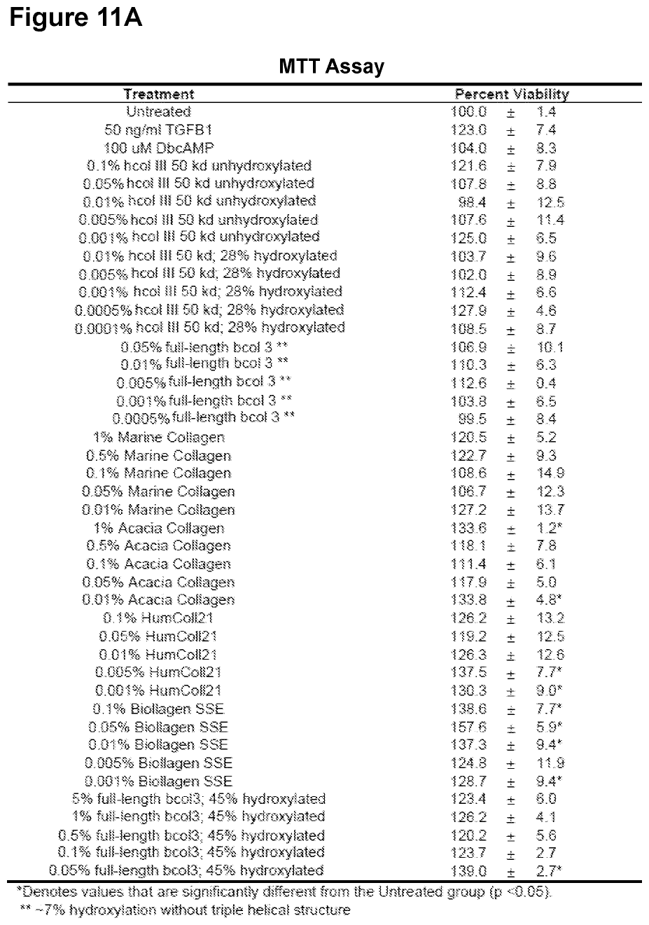

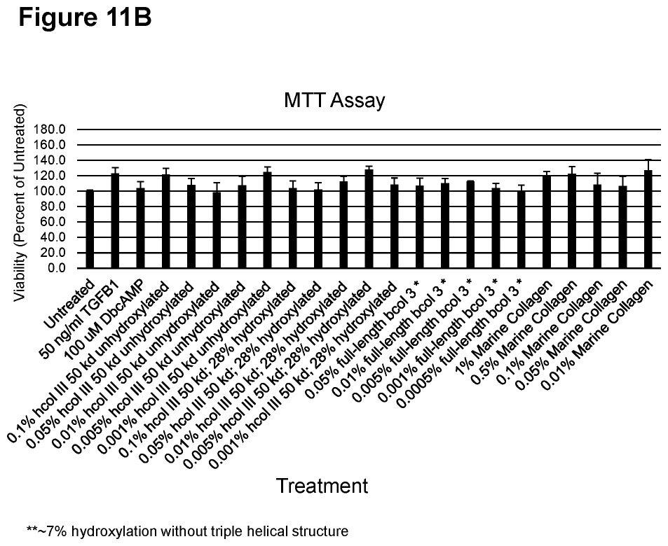

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials at the specified concentrations (see FIG. 11A and FIG. 11B) dissolved in FGM with 1.5% FBS. TGF-B (50 ng/ml) was used as a positive control for inducing collagen expression. Untreated cells (negative controls) just received DMEM with 1.5% FBS. As a negative control, cells were treated with 100 μM bDcAMP in FGM with 1.5% FBS. The cells were incubated for 48 hours and at the end of the incubation period cell culture medium was collected and either stored frozen (−75° C.) or assayed immediately. Materials were tested in triplicate.

The samples tested were an unhydroxylated 50 kDa fragment of type III human collagen (SEQ ID NO: 986) (“hcol III 50 kDa unhydroxylated”), a hydroxylated (28%) 50 kDa fragment of type III human collagen (also SEQ ID NO: 986) (“hcol III 50 kDa; 28% hydroxylated”), full-length bovine collagen 3 with hydroxylation (7%) (“full-length bcol 3”), marine collagen (Ashland), acacia collagen (Lipoid Kosmetik AG), recombinant human collagen 21 (Geltor) (“HumColl21”), BIOLLAGEN (Jland Biotech), and full length type III bovine collagen with hydroxylation (45%) (“full-length bcol3”). Each collagen sample was diluted at a range of percent concentrations in the tissue culture media, FGM with 1.5% FBS (see FIG. 11A and FIG. 11B). The unhydroxylated 50 kDa fragment of type III human collagen (SEQ ID NO: 986) was tested at 0.1%, 0.05%, 0.01%, 0.005%, and 0.001% by weight in the culture media. The hydroxylated 50 kDa fragment of type III human collagen (SEQ ID NO: 986) was tested at 0.01%, 0.005%, 0.001%, 0.0005%, and 0.0001% by weight in the culture media. The full length type III bovine collagen with 7% hydroxylation was tested at 0.05%, 0.01%, 0.005%, 0.001%, and 0.0005% by weight in the culture media. The marine collagen was tested at 1%, 0.5%, 0.01%, 0.05%, and 0.01% by weight in the culture media. The acacia collagen was tested at 1%, 0.5%, 0.01%, 0.05%, and 0.01% by weight in the culture media. The human collagen 21 was tested at 0.1%, 0.05%, 0.01%, 0.005%, and 0.001% by weight in the culture media. The human collagen 21 was tested at 0.1%, 0.05%, 0.01%, 0.005%, and 0.001% by weight in the culture media. The human collagen 21 was tested at 0.1%, 0.05%, 0.01%, 0.005%, and 0.001% by weight in the culture media. The full length bovine type III collagen with 45% hydroxylation was tested at 0.015%, 0.003%, 0.0015%, 0.0003%, and 0.00015% by weight in the culture media.

After the 2-day incubation, the cell culture medium was removed (see above) and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts were transferred to a 96-well plate and the plate was read at 540 nm using isopropyl alcohol as a blank. The mean MTT absorbance value for the negative control cells was calculated and used to represent 100% cell viability. The individual MTT values from the cells undergoing the various treatments were then divided by the mean value for the negative control cells and expressed as a percent to determine the change in cell viability caused by each treatment.

The results of the MTT assay are shown in FIG. 11A and FIG. 11B. As shown, none of the tested collagens were observed to impact cell viability. Each treated sample of cells were at least as viable as untreated control cells.

### Type I Collagen Assay

Fibroblasts are the main source of the extracellular matrix peptides, including collagen. Procollagen is a large peptide synthesized by fibroblasts in the dermal layer of the skin and is the precursor for collagen. As procollagen is processed to form a mature collagen protein, a propeptide portion is cleaved off in the form of a type I C-peptide. Both the mature collagen protein and the type I C-peptide fragment are then released into the extracellular environment. As collagen is synthesized the type I C-peptide fragment accumulates in the tissue culture medium. Since there is a 1:1 stoichiometric ratio between the two parts of the procollagen peptide, assaying for type I C-peptide reflects the amount of collagen synthesized. To measure the effects of different forms of collagen on collagen synthesis and secretion, Type 1 C-peptide was assayed via an ELISA based method.

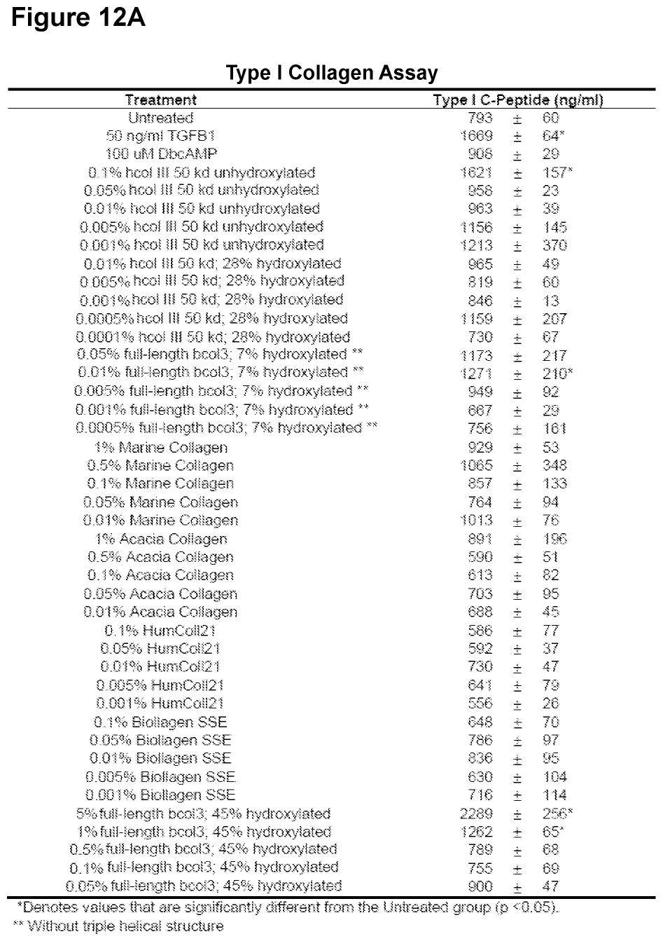

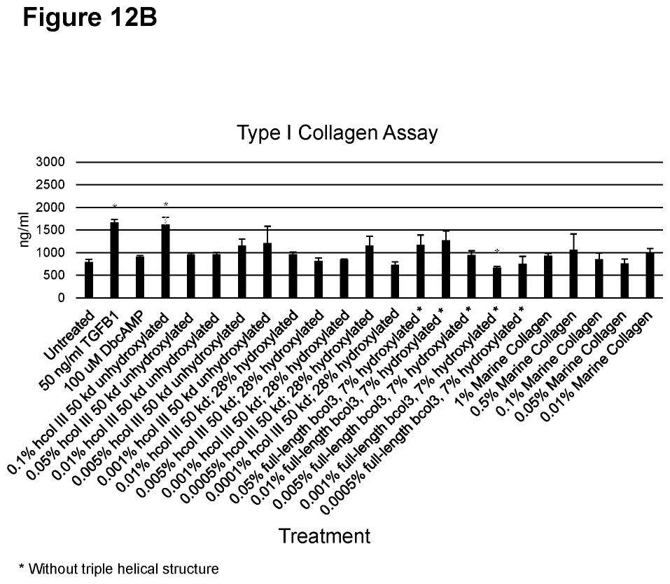

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials described below at the specified concentrations (see FIGS. 12A and 12B) dissolved in FGM with 1.5% FBS. TGF-B (50 ng/ml) was used as a positive control for inducing collagen expression. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate.

The samples tested were an unhydroxylated 50 kDa fragment of type III human collagen (SEQ ID NO: 986), a hydroxylated (28%) 50 kDa fragment of type III human collagen (also SEQ ID NO: 986), full-length bovine collagen 3 with hydroxylation (7%), marine collagen (Ashland), acacia collagen (Lipoid Kosmetik AG), recombinant human collagen 21 (Geltor), BIOLLAGEN (Jland Biotech), and full length type III bovine collagen with hydroxylation (45%). Each collagen sample was diluted at a range of percent concentrations in the tissue culture media, FGM with 1.5% FBS (see FIG. 11A and FIG. 11B). The unhydroxylated 50 kDa fragment of type III human collagen (SEQ ID NO: 986) was tested at 0.1%, 0.05%, 0.01%, 0.005%, and 0.001% by weight in the culture media. The hydroxylated 50 kDa fragment of type III human collagen (SEQ ID NO: 986) was tested at 0.01%, 0.005%, 0.001%, 0.0005%, and 0.0001% by weight in the culture media. The full length type III bovine collagen with 7% hydroxylation was tested at 0.05%, 0.01%, 0.005%, 0.001%, and 0.0005% by weight in the culture media. The marine collagen was tested at 1%, 0.5%, 0.01%, 0.05%, and 0.01% by weight in the culture media. The acacia collagen was tested at 1%, 0.5%, 0.01%, 0.05%, and 0.01% by weight in the culture media. The human collagen 21 was tested at 0.1%, 0.05%, 0.01%, 0.005%, and 0.001% by weight in the culture media. The human collagen 21 was tested at 0.1%, 0.05%, 0.01%, 0.005%, and 0.001% by weight in the culture media. The human collagen 21 was tested at 0.1%, 0.05%, 0.01%, 0.005%, and 0.001% by weight in the culture media. The full length bovine type III collagen with 45% hydroxylation was tested at 0.015%, 0.003%, 0.0015%, 0.0003%, and 0.00015% by weight in the culture media.

For the ELISA assay, a series of type I C-peptide standards was prepared ranging from 0 ng/ml to 640 ng/ml. Next, an ELISA microplate was prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard was then added to appropriate wells and the microplate was covered and allowed to incubate for 3±0.25 hours at 37° C.

After the incubation the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 15±5 minutes at room temperature. After the incubation 100 μl of stop solution (1 N sulfuric acid) was added to each well and the plate was read using a microplate reader at 450 nm. To quantify the amount of each substance present, a standard curve was generated using known concentrations of each substance. A regression analysis was performed to establish the line that best fits these data points. Absorbance values for the test materials and untreated samples were used to estimate the amount of each substance present in each sample. Treatment means were compared using an ANOVA, with an n=3 per treatment. Statistical significance was set at p<0.05.

The results of the type I collagen assay are shown in FIG. 12A and FIG. 12B. These results were obtained after treating cells with a range of percent by weight concentrations of each collagen solution. These data show that 0.1% of the 50 kDa fragment of human collagen (SEQ ID NO: 986) unexpectedly increased the amount of Type I collagen secreted by the treated fibroblasts. Treatment with 0.1% by weight (final concentration) of the 50 kDa fragment of human collagen increased collagen I expression by over 200% relative to untreated cells. While the effect was observed with the unhydroxylated 50 kDa fragment of human collagen (SEQ ID NO: 986) but not the hydroxylated collagen, this difference is attributable to the hydroxylated collagen not being tested at the highest concentration (0.1%). The cells were separately treated with equivalent or even higher concentrations of a range of different collagens. The cells were treated with a range of collagens of similar size to, or smaller than, the 50 kDa fragment of human collagen, particularly hydrolyzed marine collagen, acacia collagen, human collagen 21, and BIOLLAGEN. These collagens of similar size had no effect on collagen I production, even when those collagens were tested at up to 10-fold higher percent concentrations than the 50 kDa fragment. The cells were treated with full length bovine collagen III, both when it was approximately 7% hydroxylated and when it was approximately 45% hydroxylated. Full length bovine collagen III was able to induce collagen I expression, but only when it was highly hydroxylated (˜45%). Therefore, these data indicate that compositions comprising the 50 kDa fragment of human collagen are useful when increased production of type I collagen is desired.

### Type III Collagen Assays

Type III collagen is synthesized by dermal fibroblasts as a large pro-peptide. As the peptide is processed to form a mature type III collagen protein, a pro-peptide portion is cleaved off (type III N-peptide). Both the mature collagen protein and the type III N-peptide fragment are then released into the extracellular environment. As collagen is synthesized the type III N-peptide fragment accumulates in the tissue culture medium. Since there is a 1:1 stoichiometric ratio between the two parts of the procollagen peptide, assaying for type III N-peptide reflects the amount of collagen synthesized. To measure the effects of different forms of collagen on collagen synthesis and secretion, Type III N-peptide was assayed via an ELISA based method.

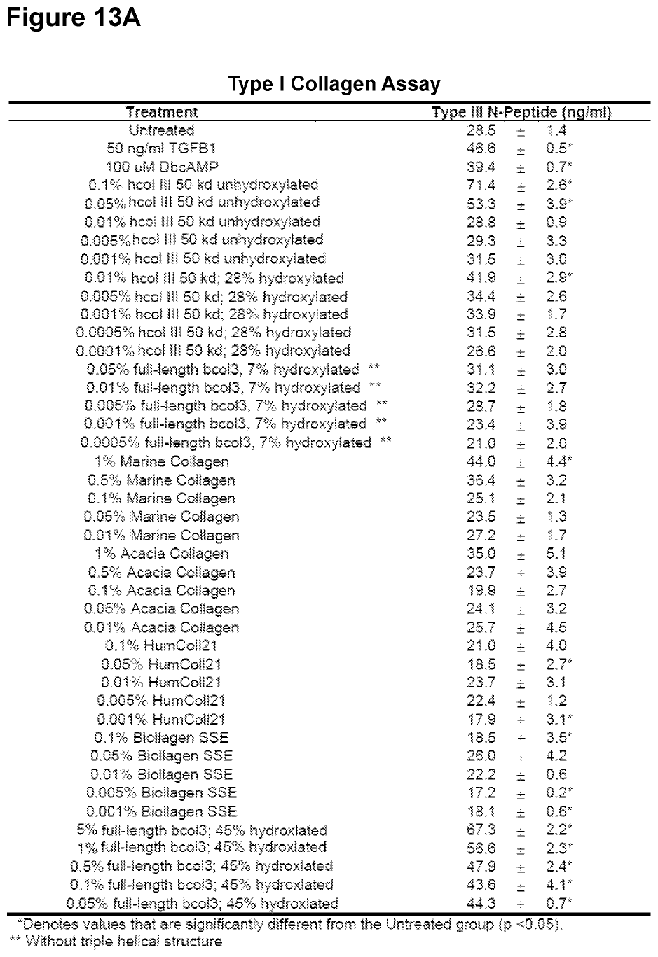

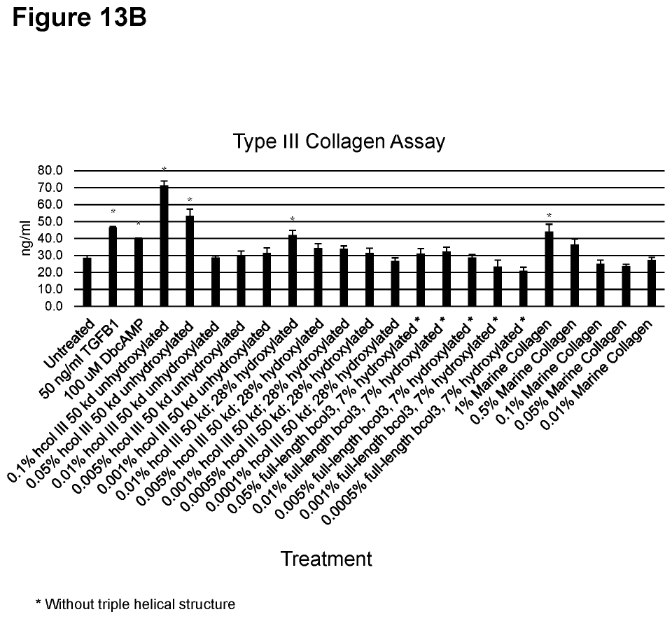

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials described below at the specified concentrations (see FIGS. 13A and 13B) dissolved in FGM with 1.5% FBS. TGF-B (50 ng/ml) was used as a positive control for inducing collagen expression. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate.

The samples tested were an unhydroxylated 50 kDa fragment of type III human collagen (SEQ ID NO: 986), a hydroxylated (28%) 50 kDa fragment of type III human collagen (SEQ ID NO: 986), full-length bovine collagen 3 with hydroxylation (7%), marine collagen (Ashland), acacia collagen (Lipoid Kosmetik AG), recombinant human collagen 21 (Geltor), BIOLLAGEN (Jland Biotech), and full length type III bovine collagen with hydroxylation (45%). Each collagen sample was diluted at a range of percent concentrations in the tissue culture media, FGM with 1.5% FBS (see FIG. 11A and FIG. 11B). The unhydroxylated 50 kDa fragment of type III human collagen (SEQ ID NO: 986) was tested at 0.1%, 0.05%, 0.01%, 0.005%, and 0.001% by weight in the culture media. The hydroxylated 50 kDa fragment of type III human collagen (SEQ ID NO: 986) was tested at 0.01%, 0.005%, 0.001%, 0.0005%, and 0.0001% by weight in the culture media. The full length type III bovine collagen with 7% hydroxylation was tested at 0.05%, 0.01%, 0.005%, 0.001%, and 0.0005% volume in the culture media. The marine collagen was tested at 1%, 0.5%, 0.01%, 0.05%, and 0.01% volume in the culture media. The acacia collagen was tested at 1%, 0.5%, 0.01%, 0.05%, and 0.01% volume in the culture media. The human collagen 21 was tested at 0.1%, 0.05%, 0.01%, 0.005%, and 0.001% volume in the culture media. The human collagen 21 was tested at 0.1%, 0.05%, 0.01%, 0.005%, and 0.001% volume in the culture media. The human collagen 21 was tested at 0.1%, 0.05%, 0.01%, 0.005%, and 0.001% volume in the culture media. The full length bovine type III collagen with 45% hydroxylation was tested at 0.015%, 0.003%, 0.0015%, 0.0003%, and 0.00015% volume in the culture media.

For the ELISA assay, a series of standards was prepared and 100 μl of these standards or samples were added to the wells of the type III collagen ELISA plates. The plates were then incubated at 37° C. for 1.5 hours. After this incubation the ELISA plates were then washed twice with wash buffer, followed by the application of 100 μl of detection antibody solution. The ELISA plates were then incubated for 1 hour at 37° C. After the incubation all of the ELISA plates were washed with wash solution followed by the addition of 100 μl of HRP conjugate solution and incubated at 37° C. for 30 minutes. After this incubation the ELISA plates were again washed and 100 μl of substrate solution was added to each well and the well-plates were incubated for 10-30 minutes at room temperature to allow the color generation reaction to occur. At the end of the color generation reaction 100 μl of stop solution was added to each well and the plates were read at 460 nm using a plate reader. To quantify the amount of each substance present, a standard curve was generated using known concentrations of each substance. A regression analysis was performed to establish the line that best fits these data points. Absorbance values for the test materials and untreated samples were used to estimate the amount of each substance present in each sample. Treatment means were compared using an ANOVA, with an n=3 per treatment. Statistical significance was set at p<0.05.

The results of the type III collagen assay are shown in FIG. 13A and FIG. 13B. These results were obtained after treating cells with a range of percent by weight concentrations of each collagen solution. These results show that treatment with 0.1% the 50 kDa fragment of human collagen (SEQ ID NO: 986) unexpectedly increased the amount of Type III collagen secreted by the treated fibroblasts by over 200% relative to untreated cells. A smaller but still significant effect was also observed after treatment with 0.01% of the 50 kDa fragment of human collagen (SEQ ID NO: 986; both hydroxylated and unhydroxylated). No effect on collagen III production was observed after treatment with bovine collagen III (7% hydroxylated), acacia collagen, human collagen 21, or BIOLLAGEN, even when those collagens were tested at up to 10-fold higher percent concentrations than the 50 kDa fragment. Full length bovine collagen III was able to induce collagen I expression, but only when it was highly hydroxylated (˜45%). The marine collagen induced an effect on collagen III expression only when tested at a 10-fold higher percent concentration than the 50 kDa fragment of human collagen. Therefore, these data indicate that compositions comprising the 50 kDa fragment of human collagen are useful when increased production of type III collagen is desired.

### Example 7: Additional Characterization of Truncated Human Collagen III Fragment

Additional assays were conducted on the 50 kDa fragment of human collagen (SEQ ID NO: 986) to further characterize the fragment.

### Physical Properties

The purified unhydroxylated and purified hydroxylated preparations of the 50 kDa fragment of human collagen (SEQ ID NO: 986) were evaluated with regards to their solubility and character in various solutions. These properties were compared to those of full length bovine collagen 3. The following samples were each dissolved at a 1% by weight concentration in deionized water, with 1% phenoxyethanol for preservation:
1. Unhydroxylated human collagen 3 50 kDa fragment
2. Hydroxylated (28%) human collagen 3 50 kDa fragment
3. Full-length bovine collagen 3 with 7% hydroxylation
4. Full-length bovine collagen 3 with 45% hydroxylation

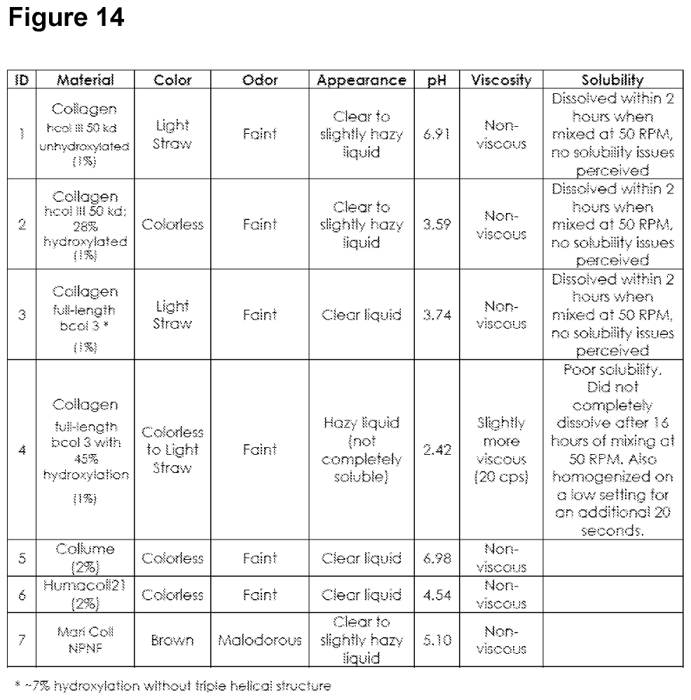

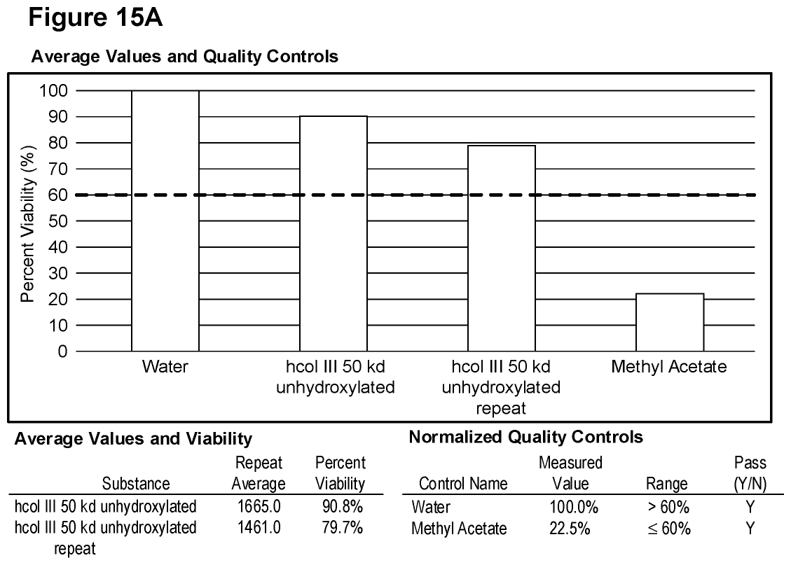

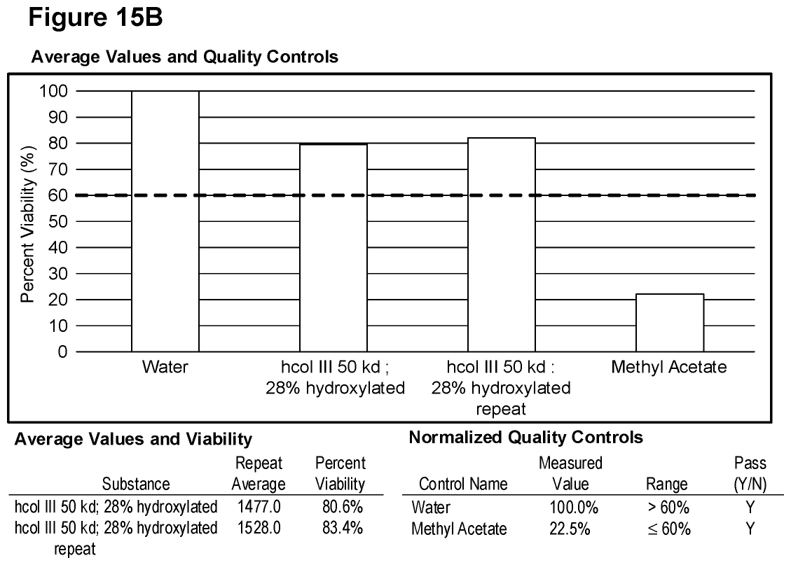

The dissolved collagen solutions were compared against COLLUME (2%; Geltor), human collagen 21 (2%; Geltor), and marine collagen NPNF (Tri-K), each treated in the same way. The results of the analysis are shown in FIG. 14. The tested 50 kDa collagen fragments were soluble and non-viscous, with little to no color or odor.

The four collagen samples were also added at 3% by weight into seven base formulations to test compatibility and performance. Those base solutions were a gel (carbomer system), a serum (ammonium acryloyldimethyltaurate/VP copolymer and acrylates/C10-30 alkyl acrylate crosspolymer system), a cream emulsion (oil/water), hair conditioner (containing cetrimonium chloride and behentrimonium methosulfate), Shampoo/Cleanser (Sulfate-Free), water-based toner, and alcohol-based toner. The unhydroxylated collagen was compatible with all base formulations tested, while the hydroxylated collagen was compatible with all base formulations except the alcohol-based toner. Moreover, the 50 kDa human collagen fragment displayed more compatibilities than the full length bovine collagen across the range of tested base formulations.

These results demonstrate that the 50 kDa fragment of human collagen is a viable material with potential applications in personal care formulas, provided that it is properly incorporated into the appropriate formulations. In general, it exhibited good color and odor and could easily be incorporated into the tested formulas. The characteristics of the collagen fragment provide possible benefits in skin care formulations in terms of enhanced elasticity, wrinkle reduction, smoothing, moisture-loss prevention, firming, and film forming. There was no observed upper limit on the use level of these materials in certain formula types in regards to aesthetics or compatibility. There was no negative skin feel observed for any of the 1% solutions at any included level.

### HRIPT Test for Skin Irritation

To determine the irritation and sensitization (contact allergy) potential of the 50 kDa human collagen fragment (SEQ ID NO: 986), a volunteer study was performed in which collagen solutions were repeatedly applied to human skin. The study involved testing of both hydroxylated and unhydroxylated collagen fragment solution.

Patches containing 7.5 mm paper discs were soaked in a solution containing 2% by weight of the 50 kDa human collagen fragment (SEQ ID NO: 986). The patches were then affixed directly to the skin of the intrascapular regions of the back, to the right of left of the midline and subjects were dismissed with instructions not to wet or expose the test area to direct sunlight. One patch was administered for each collagen solution being tested to each patient. Patches remained in place for 48 hours after the first application. Subjects were instructed not to remove the patches prior to their 48 hour scheduled visit. Thereafter, subjects were instructed to remove patches 24 hours after application for the remainder of the study. The procedure was repeated until a series of nine consecutive, 24 hour exposures had been made three times a week for three consecutive weeks. Prior to each reapplication, the test sites were evaluated by trained laboratory personnel. Following a 10-14 day rest period, a retest/challenge dose was applied once to a previously unexposed test site. Retest sites were evaluated by trained laboratory personnel 48 and 96 hours after application.

No adverse events of any kind were reported during the course of the study in any of the 55 test subjects, for either collagen solution. In comparison, administration of a positive control (2% sodium lauryl sulfate solution) resulted in one subject with a grade 4 reaction (erythema, induration, and bulae) and three subjects with a grade 1 reaction (erythema throughout at least three quarters of the patch area. Therefore, there was no indication of a potential to elicit dermal irritation or sensitization (contact allergy) noted for the 50 kDa human collagen fragment.

### Example 8: Application of Cosmetic Compositions

To characterize the effects of application of the 50 kDa human collagen fragment (SEQ ID NO: 986) on human skin, a volunteer study was performed in which collagen solutions were repeatedly applied to human skin. Two different formulations were applied, each of which comprised the 50 kDa human collagen fragment in a composition comprising 1.5% xantham gum (Kelltrol® CG-BT), 0.2% potassium sorbate, 0.3% sodium benzoate, and 0.05% citric acid, with a pH of 5.2. Formulation 1 comprised a final concentration of 0.05% of the 50 kDa human collagen fragment, while formulation 2 comprised a final concentration of 0.002% of the 50 kDa human collagen fragment solution. Twenty-five subjects applied formulation 1 on a daily basis for 12 weeks, while 24 subjects applied formulation 2 on a daily basis for 12 weeks.

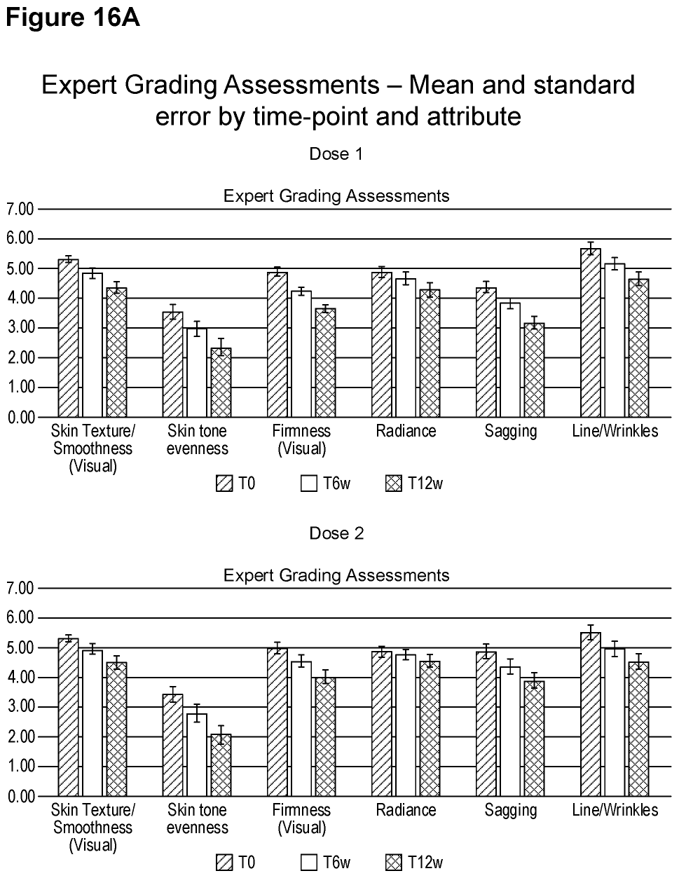

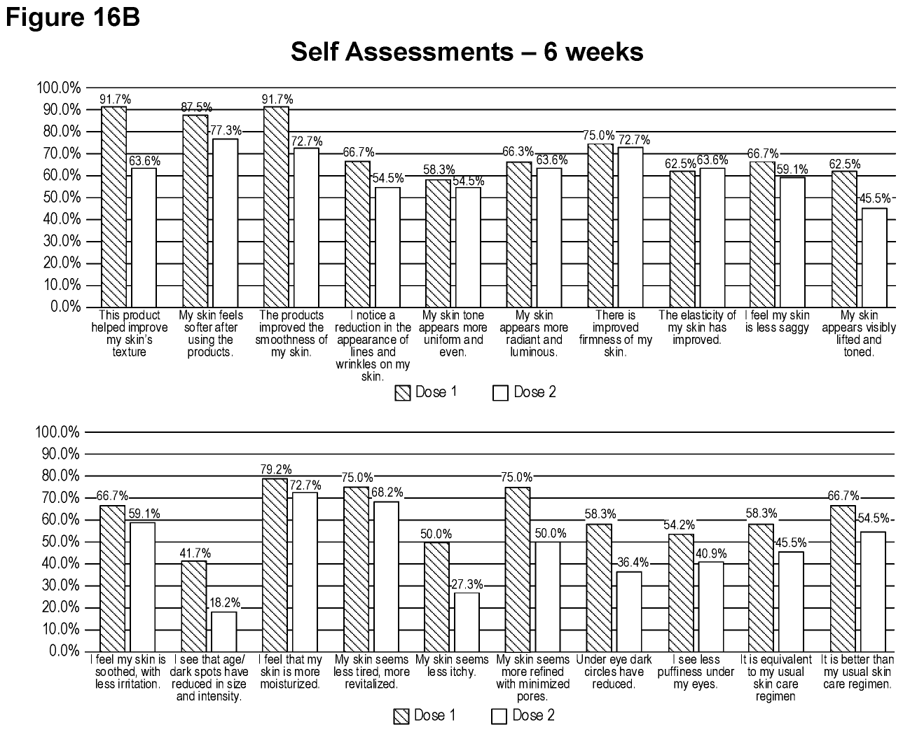

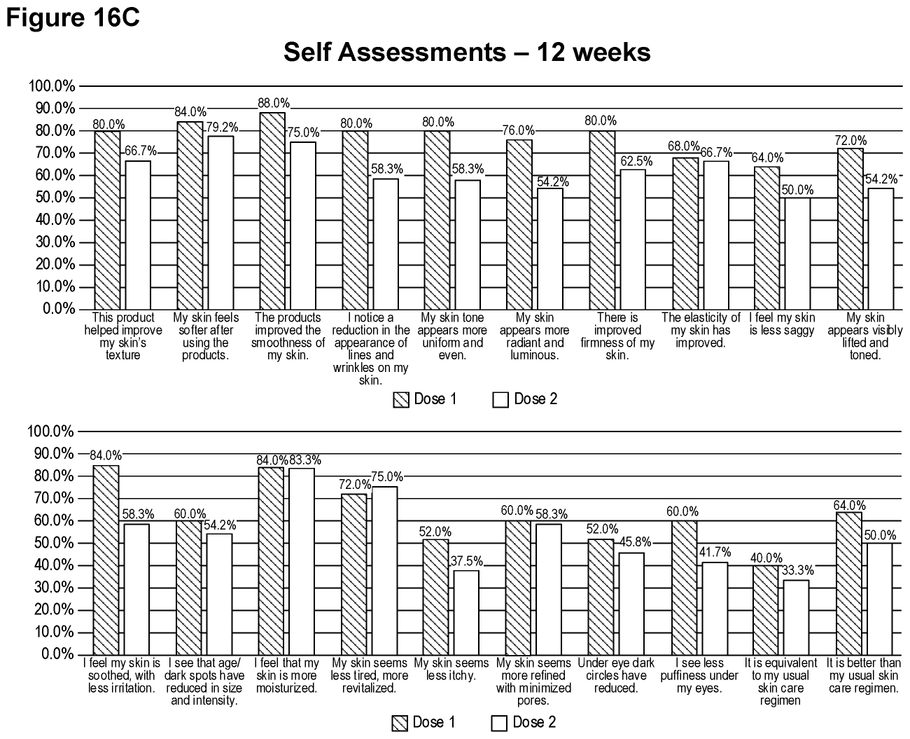

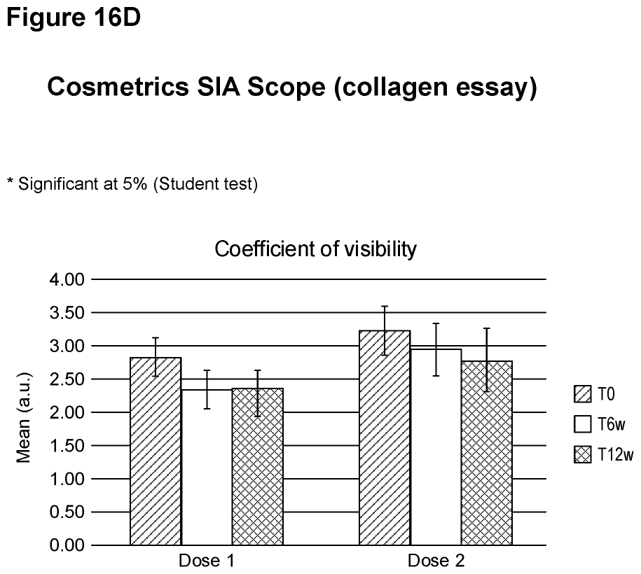

Objective clinical efficacy assessments were performed by an expert clinical grader both before the start of application, after 6 weeks, and after 12 weeks of application. The grader examined the skin for: texture and smoothness (visual), skin tone evenness, firmness (visual), radiance, sagging, and lines/wrinkles, using 10-Point Ordinal Scales on the Face. The results of these assessments are shown in FIG. 16A, with respect to the degree of improvement. A statistical significant improvement was observed after 6 and 12 weeks of product use of dose 1 and dose 2 for skin texture/smoothness (visual), skin tone evenness, firmness (visual), sagging and lines/wrinkles. For radiance, a statistically significant improvement was observed after 6 and 12 weeks of products use of dose, but ony after 12 weeks of products use of dose 2.

The subjects also performed subjective self-assessments after six and after 12 weeks of application. These assessments took the form of a series of questions presented to the subjects as listed in FIGS. 16B and 16C. Broadly, both formulation 1 and formulation 2 were consistently characterized as leading to improvements in skin condition with greater than 50% of patients noting improvements (i.e., agree or strongly agree) with respect to almost all questions.

Spectrophotometric Intracutaneous Analysis (SIA) was performed using a SIAscope® (Astron Clinica Ltd.) to determine the levels of collagen within the skin both before and after 6 and 12 weeks of application of either formulation 1 or formulation 2. As shown in FIG. 16D, application of both formulation 1 and formulation 2 lead to an 18% increase in collagen levels after six weeks of application and to a 25% increase in collagen after 12 weeks of application, with almost all patients having some improvement in collagen.

Cutometer MPA 580 (Courage+Khazaka, Germany) was used to measure the viscoelastic properties of the skin (firmness and elasticity) by applying suction to the skin surface. A statistically significant reduction of RO was observed after 12 weeks of product use compared to T0 for dose 1, indicating improvement of skin firmness. A statistically significant increase of elasticity was observed after 6 and 12 weeks of product use compared to T0 for Dose 1 and 2.

### Example 9: Generation of Truncated Human Collagen III Fragment Sequence Variants

Plasmids encoding secretion signal-secreted human collagen variants having amino acid sequences according to SEQ ID NO: 987-1015 were constructed. The plasmid DNA is set forth in the nucleic acid sequences according to SEQ ID NOs: 1045-1073.

Each plasmid was transformed into the PP97 Pichia pastoris (Komagataella phaffii) strain by electroporation with the BIO-RAD GENE PULSER XCELL Total System as in Example 1. The resulting strains were used to express the variants. In some cases, the strains were doubly transformed to improve the recombinant protein expression. Then, the three-step fermentation process of Example 2 was utilized.

### Example 10: Preparation and Analysis of Collagen Variant Samples

### Preparation of Samples

### Fermentation Harvest

The fermentation broth was removed from the fermenter and mixed broth at ˜200-400 RPM to ensure the solution was homogenous. Then, the cells were removed from the fermentation supernatant. The broth was transferred into 1 L Sorvall bottles and spun down at max speed (17,568 g) for 20 min at 20° C. The supernatant was decanted into a 1-5 L container and the pellets were discarded. The fermentation supernatant was stored in the −20° C. freezer, until ready to use.

### pH and Ammonium Sulfate Precipitation and Recovery

The fermentation supernatant was thawed in a room temperature water bath. 1 M ammonium sulfate was added to the fermentation supernatant, which was then mixed at ˜200-400 RPM to ensure the solution was homogenous. The amount of ammonium sulfate mass required to target a concentration of 1 M was calculated and added according to the equation below. Density=1.07 kg/L

$$Mass of salt ( kg ) = \frac{\frac{Desired Molarity ( \frac{mol}{L} ) * Mass Feed ( kg )}{Density ( \frac{kg}{L} )}}{\frac{1000}{Salt Molecular weight ( \frac{g}{mol} )} - \frac{Desired Molarity ( \frac{mol}{L} )}{Density ( \frac{kg}{L} )}} \\ Density = 1.07 kg / L$$

The pH was adjusted to 4±0.05 using 50% sulfuric acid. And the solution was incubated for 30 minutes while mixing at ˜200-400 RPM to ensure the solution was homogenous. The pH was checked again and adjusted back to pH 4±0.05, if necessary, by addition of 50% sulfuric acid or 10 N NaOH. The pH/salt precipitated pellet was recovered. The solution was transferred into 1 L Sorvall bottles and spun down at max speed (17,568 g) for 20 min at 4° C. Then, the supernatant was decanted into a beaker the waste was discarded. The precipitated protein pellet was resuspend in water and the fermentation supernatant was stored in the −20° C. freezer until ready to use.

### Ultra Diafiltration

The resuspended protein solution was thawed in a room temperature water bath. The system was set up with 10 kD hollow fiber filters and a water flux test was performed. The 0.1 N NaOH storage solution was drained from the membrane housing and disposed and the system was flushed with water. The clean water flux at a Transmembrane pressure (TMP) of approximately 3, 5, & 10 psi was performed. Then, 10× phosphate saline solution was added to the resuspended pellet and the pH was adjusted. The solution was mixed at ˜200-400 RPM until the solution was homogenous. The amount of 10×PBS was calculated and added to achieve a final concentration of 1× according to the equation below. *Assume density=1 g/mL

$$Volume of 10 X PBS to add = \frac{Mass solution}{9}$$

The pH was adjusted to 7.2, if necessary, by addition 10 N NaOH. Any insoluble particles were removed and the solution was transferred into 1 L Sorvall bottles and spun down at max speed (17,568 g) for 20 min at 4° C. The supernatant was decanted into a beaker for further processing and the pellet waste was discarded. The resuspended protein solution was concentrated by setting the pump to 1020 mL/min, applying back pressure to achieve a transmembrane pressure (TMP) of 20-25 psi, and concentrating until 200 mL.

The concentrated protein solution was diafiltered with 3 diavolumes of water by setting the pump to 1020 mL/min, applying back pressure to achieve a TMP of 20-25 psi, and diafiltering against water until 3 diavolumes was achieved. A check was performed to confirm the total solids were >1.5%. If the total solids were <1.5%, the protein solution was concentrated to minimal volume (˜75 mL). The product was retrieved from the system and stored in the −20° C. freezer until ready to use. A water flush was performed, the system underwent clean in place solution (CIP) with 1% CIP 100 solutions and the membrane was put in in 0.1 M Sodium hydroxide storage solutions.

### Formulation

The diafiltered protein solution was thawed in a room temperature water bath and 10× Phosphate Buffered Saline (PBS) was added. The solution was mixed at ˜200-400 RPM until the solution was homogenous. The amount of 10× Phosphate Buffered Saline (PBS) volume required to target a final concentration of 1× was calculated using the excel calculator.

$$Mass of PBS ( mL ) = \frac{Starting mass ( g )}{9} \\ * Assume density = 1 g / mL$$

The pH was adjusted to 7.2±0.1 (pH 7.1-7.3) using concentrated hydrochloric acid (HCl) or 10 N sodium hydroxide (NaOH). The final formulated product was vacuum sterile filtered with a 0.2 μm sterile bottle top filter in a biosafety cabinet and evaluated.

### Data Collection Methods

### SDS-PAGE of the Protein

The pH was adjusted to 7.2±0.1 (pH 7.1-7.3) using concentrated hydrochloric acid (HCl) or 10 N sodium hydroxide (NaOH). The final formulated product was vacuum sterile filtered with a 0.2 μm sterile bottle top filter in a biosafety cabinet and evaluated.

Protein quantification, monitoring and detection was provided by electrophoretic separation via Sodium dodecyl sulfate-Polyacrylamide gel electrophoresis (SDS-PAGE). Standards were gravimetrically prepared at a concentration of 0.25 g/L, 0.125 g/L and 0.0625 g/L to create a standard curve. Working solutions of each standard were stored at 4° C. Based on estimated concentrations, samples were diluted using DI water to fit within the standard curve. Lithium dodecyl sulfate (LDS) running buffer was added in a 1:1 solution with each individual standard and sample. It was heated to breakdown disulfide bonds and denature the protein so it could pass through the pores of the Tris acetate gel. The tris acetate gel was placed into a Midi Gel tank. Tris-acetate buffer (1×) was added to the gel box. In an SDS-PAGE, each gel contained the following: Molecular weight marker, standard curve points, samples, and positive control. Once standards and samples were loaded in the gel wells, the gel box was covered by the power cables connected to the Electrophoresis for one hour and twenty minutes at 125 Volts and 300 Watts.

The gel was removed from the plastic case and placed in the Licor incubation box to undergo the two-hour staining process using Page-Blue protein staining solution and placed on an orbital shaker with gentle agitation.

The Page-Blue solution was washed off and the gel begins the de-staining process using 10% methanol. The gel incubated for 60 minutes with gentle agitation on an orbital shaker. Then, the methanol was washed off and the gel continued to de-stain using Millipore water. The gel incubated for 60 minutes with gentle agitation on an orbital shaker.

Using the Image lab Densitometer, an image scan of the gel was taken. Samples positive for the expression of collagen protein had a strong band migrating at the appropriate molecular weight corresponding with the positive control. These bands were analyzed using the application “app v2.01 TT”. A CSV excel file was created and added to the corresponding “qSDS template”

Using statistical control charts, each gel underwent quality control checks to ensure all system suitability parameters were met before reporting data.

### Sequence Verification of Protein (LC/MS Analysis):

Before sequence verification by LC/MS, all protein samples were digested as described in the above section: Enzymatic digestion of protein (for LC/MS analysis). Following enzymatic digestion, the identified peptide sequences of digested peptides were matched against the theoretical protein sequence to calculate sequence coverage % (calculated by dividing the number of amino acids in all found peptides by the total number of amino acids in the entire protein sequence). The sequence coverage maps and detected maps for the target variant protein identified in each sample were generated using Byonic software (Protein Metrics Inc., San Carlos, CA) for visualization.

In addition to total sequence coverage %, an additional sequence verification calculation, potential sequence coverage %, was computed to address identification limitations of the LC/MS instrument. This calculation omits peptides present in the digested sample that were too small to be detected by the LC/MS instrument in its current settings. As such, potential sequence coverage % was calculated to be the total number of amino acids in all found peptides divided by the total number of amino acids in peptides that are possible to be detected by the LC/MS in its current acquisition mode. The % sequence unaccounted for was calculated as the complement of potential sequence coverage %, representing the portions of the protein sequence that, based upon tryptic digestion rules, should be seen post-digestion, but have no matched peptides.

### Glycosylation Analysis of Protein (LC/MS Analysis):

Before sequence verification by LC/MS, all protein samples were digested as described in the above section: Enzymatic digestion of protein (for LC/MS analysis). Following enzymatic digestion, the mass spectrum raw data, Agilent *.d files were processed using Byonic and Byologic software (Protein Metrics Inc., San Carlos, CA) and were searched against respective in-house variant protein sequences. Byonic search parameters were set to include peptides cleaved C-terminal to Arg and Lys residues, with fully-specific trypsin digestion, allowing up to two missed cleavages. Precursor mass tolerance was set to 10 ppm with fragment mass tolerance at 0.02 Da with a QTOF/HCD fragmentation type. Post-translational modifications considered within the search parameters included Carbamidomethylation (Cys; +57.021464 Da), Deamidation (Asn, Gln; +0.984016 Da), Gln->pyro-Glu (N-terminal Gln; −17.026549 Da), Oxidation (Met; +15.994915) and Acetylation (Protein N-terminal; +42.010565 Da). All modifications were considered variable, except for carbamidomethylation of Cys which was set as a fixed modification. Glycopeptides were identified in the samples following a separate, more focused search using the 20 fungal N-glycans and 14 fungal O-glycans databases. Data were filtered at 1% protein false discovery rate (FDR or 20% reverse count). The Byonic result files were imported into Byologic for further detailed analysis for result reporting. Following peptide mapping, peak areas of matched peptides were used to calculate the proportion of glycosylated to unglycosylated Ser (S) or Thr (T) residues in the peptide sequence. For each detected S/T, its individual glycosylation % was calculated as: (sum of XIC area of glycosylated peptide/(sum of XIC areas of glycosylated+unmodified peptide). The overall average % glycosylation for the sample was then calculated by averaging the individual S/T glycosylation % of all the detected (non-zero) Ser/Thr modifications.

### Example 11: Stimulation of Collagen I and Collagen III Synthesis by Truncated Human Collagen III Variants in Fibroblast Cells

A fibroblast cell culture model was used to assess the ability of the human collagen III variants having the amino acid sequences set forth in SEQ ID NOs: 987-1015 (the “second group of test materials”) to exert an effect on collagen synthesis. This study also assessed the viability of the cells after exposure to the second group of test materials.

### Summary of the Test Method

Fibroblasts are the main source of the extracellular matrix peptides, including the structural proteins type I collagen and type III collagen. In this study human dermal fibroblasts were treated with the second group of test materials for 48 hours, after which changes in the extracellular matrix (ECM) components were measured in the cell culture media using ELISA based methods.

### MTT Assay

In addition to the changes in ECM component production, the impact of the second group of test materials on cell viability was assessed using an MTT assay. The MTT assay on the second group of test materials was conducted as described for the test materials discussed in Example 6.

The samples were separated into batches 1-3 for testing. Batches 1-3, included each of the truncated human collagen III variants having the amino acid sequences set forth in SEQ ID NOs: 987-1015, provided herein. The relationship between the variant (identified by “variant letter”), their amino acid sequences, their nucleic acid sequence, the plasmid sequence used to prepare the Pichia strain for collagen expression, and identity to SEQ ID NO: 986 is provided below in Table 6.

TABLE 6

| | | | | % Protein |
|---|---|---|---|---|
| Variant | AA SEQ | Nucleic Acid | Plasmid SEQ | Sequence |
| Number | ID NO. | SEQ ID NO: | ID NO: | similarity |
| A | 987 | 1016 | 1045 | 100% |
| B | 988 | 1017 | 1046 | 92% |
| C | 989 | 1018 | 1047 | 90% |
| D | 990 | 1019 | 1048 | 98% |
| E | 991 | 1020 | 1049 | 90% |
| F | 992 | 1021 | 1050 | 96% |
| G | 993 | 1022 | 1051 | 90% |
| H | 994 | 1023 | 1052 | 100% |
| I | 995 | 1024 | 1053 | 77% |
| J | 996 | 1025 | 1054 | 77% |
| K | 997 | 1026 | 1055 | 68% |
| L | 998 | 1027 | 1056 | 99% |
| M | 999 | 1028 | 1057 | 92% |
| N | 1000 | 1029 | 1058 | 94% |
| O | 1001 | 1030 | 1059 | 90% |
| P | 1002 | 1031 | 1060 | 99% |
| Q | 1003 | 1032 | 1061 | 93% |
| R | 1004 | 1033 | 1062 | 90% |
| S | 1005 | 1034 | 1063 | 99% |
| T | 1006 | 1035 | 1064 | 96% |
| U | 1007 | 1036 | 1065 | 91% |
| V | 1008 | 1037 | 1066 | 90% |
| W | 1009 | 1038 | 1067 | 100% |
| X | 1010 | 1039 | 1068 | 98% |
| Y | 1011 | 1040 | 1069 | 98% |
| Z | 1012 | 1041 | 1070 | 96% |
| AA | 1013 | 1042 | 1071 | 90% |
| BB | 1014 | 1043 | 1072 | 91% |
| CC | 1015 | 1044 | 1073 | 99% |

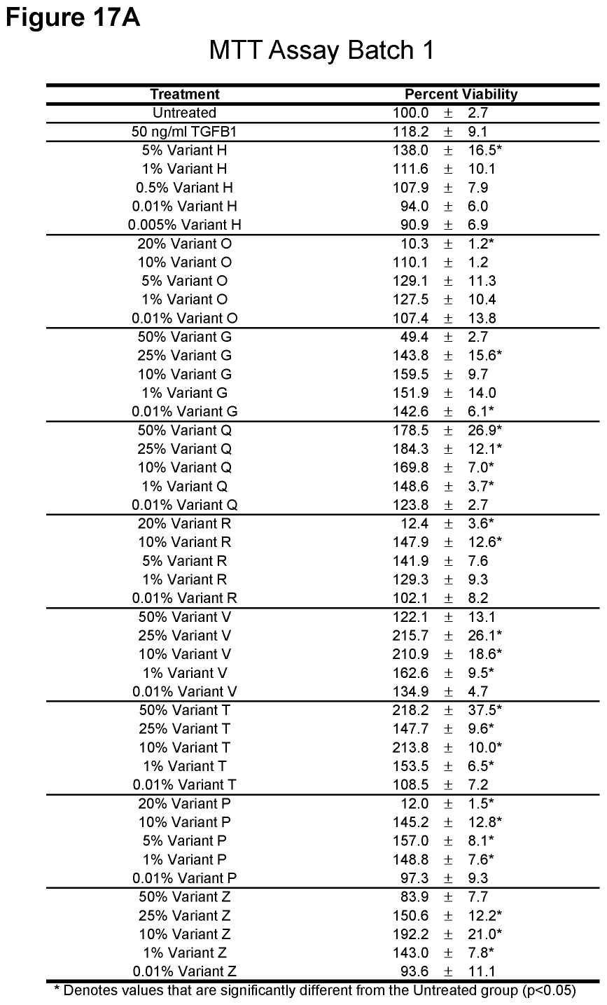

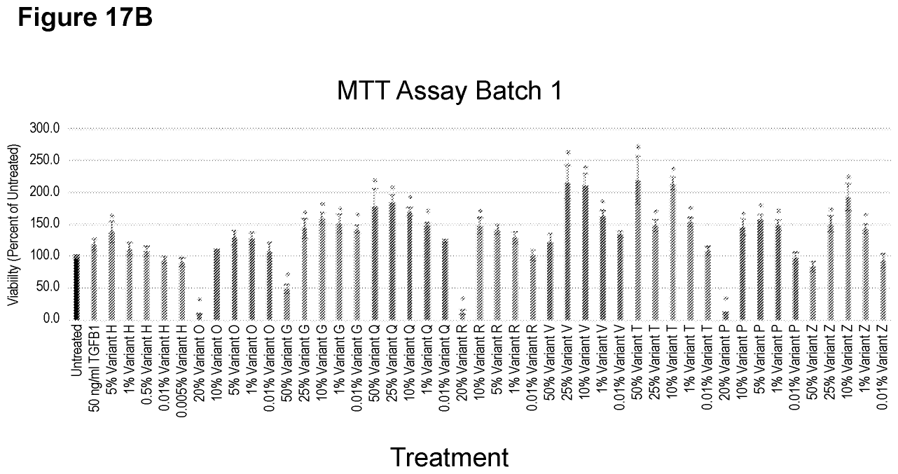

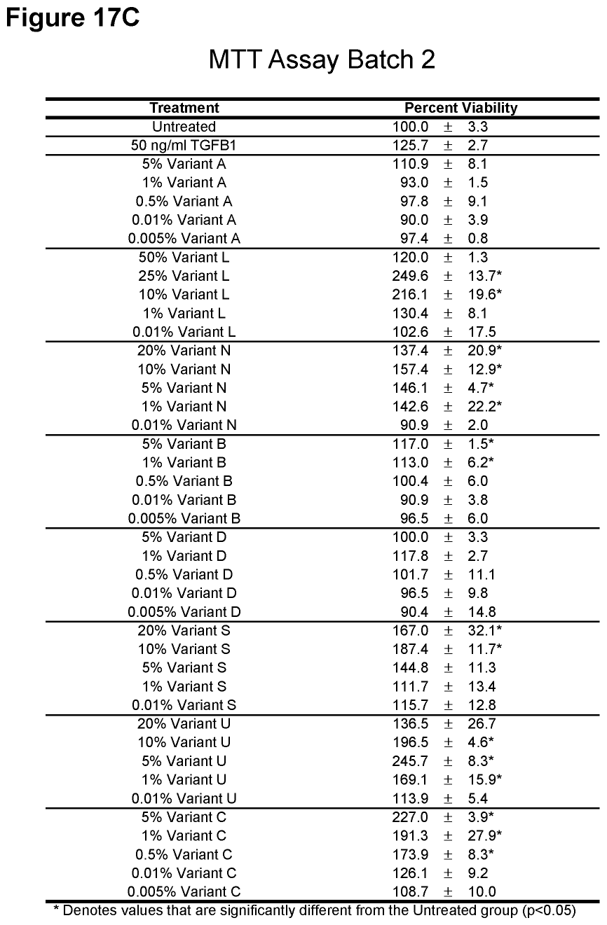

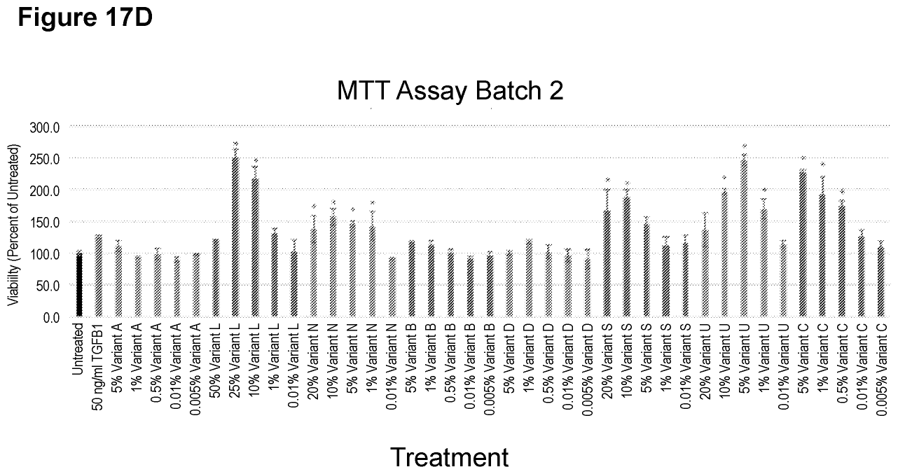

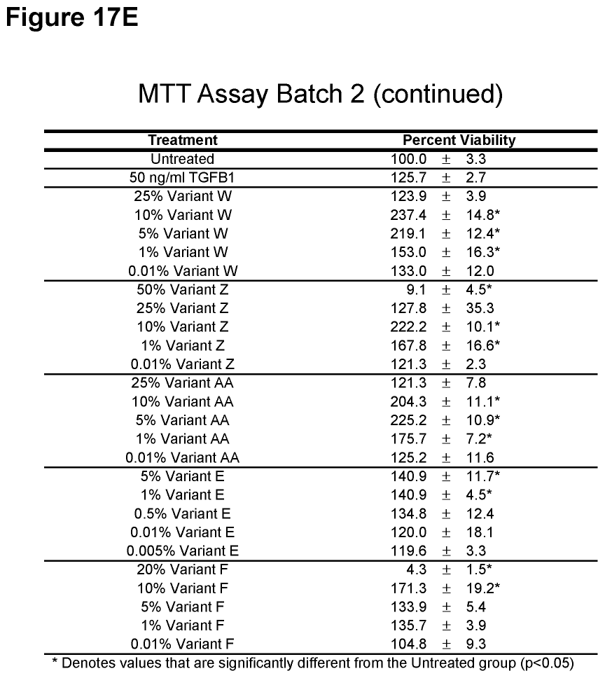

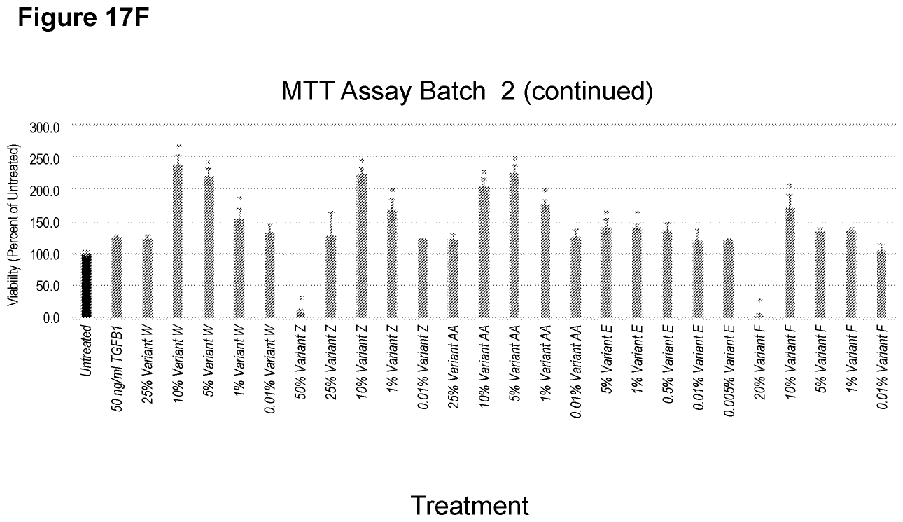

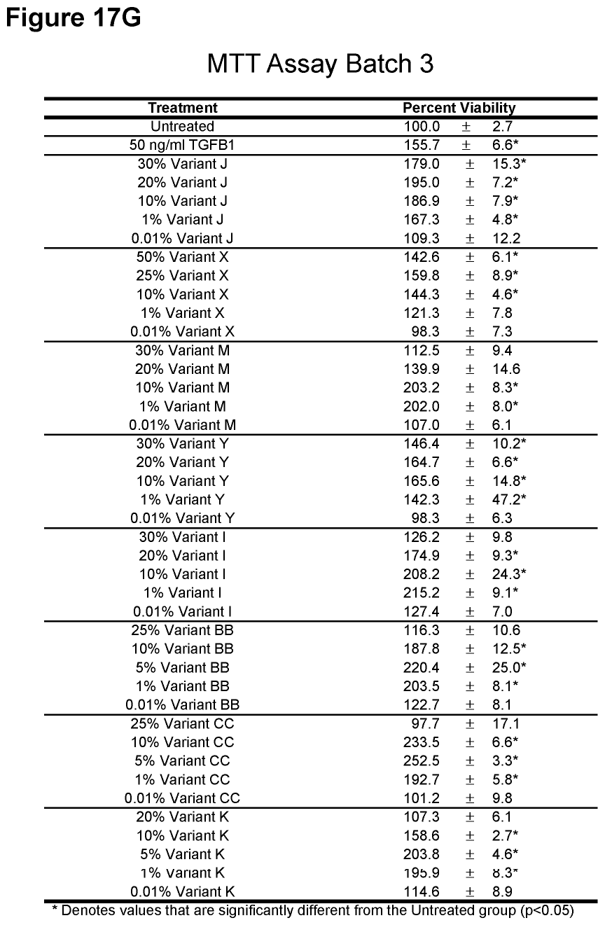

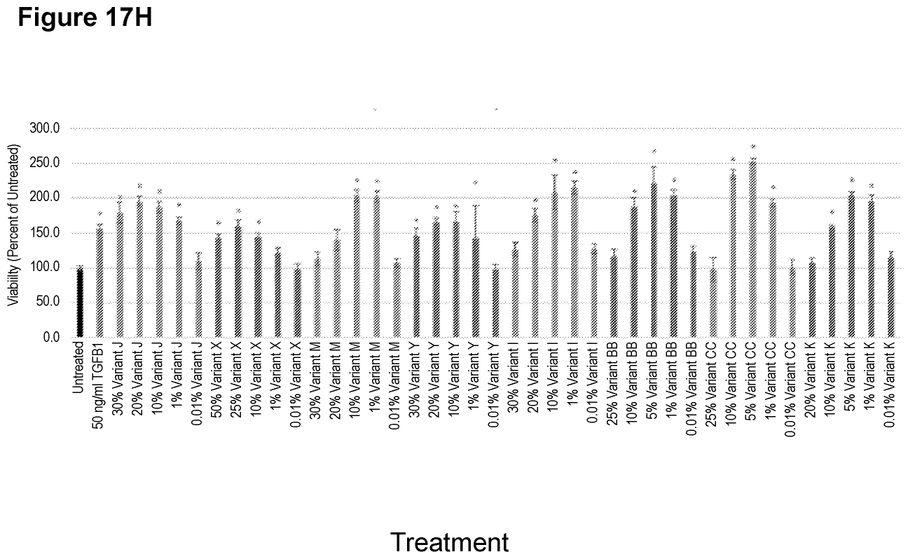

The variants in Batch 1 of the second group of test materials (“Batch 1 test materials”) include Variants H, O, G, Q, R, V, T, P, and Z. The Batch 1 test materials were prepared as actives dissolved in a solution. Each variant collagen sample was diluted at a range of percent concentrations in the tissue culture media, FGM with 1.5% FBS (see FIGS. 17A-FIG. 17B). The concentrations reported in the results represent the final concentration of the solution in the tissue culture media (v/v).

Variant H (SEQ ID NO: 994) was tested at 5%, 1%, 0.5%, 0.01%, and 0.005% of the original concentration, which corresponded to actual tested concentrations of 0.068%, 0.014%, 0.007%, 0.000%, and 0.000%, respectively.

Variants O (SEQ ID NO: 1001), R(SEQ ID NO: 1004), and P (SEQ ID NO: 1002), were tested at 20%, 10%, 5%, 1% and 0.01% of the original concentration, which corresponded to actual tested concentrations of 0.127%, 0.063%, 0.032%, 0.006%, and 0.000% (Variant O); 0.103%, 0.052%, 0.026%, 0.005%, and 0.000% (Variant R); and 0.081%, 0.041%, 0.020%, 0.004%, and 0.000% (Variant P).

Variants G (SEQ ID NO: 993), Q (SEQ ID NO: 1003), V (SEQ ID NO: 1008), T (SEQ ID NO: 1006), and Z (SEQ ID NO: 1012) were tested at 50%, 25%, 10%, 1%, and 0.01% of the original concentration, which corresponded to actual tested concentrations of 0.614%, 0.307%, 0.123%, 0.012%, and 0.000% (Variant G); 0.369%, 0.185%, 0.074%, 0.007%, and 0.000% (Variant Q); 0.307%, 0.153%, 0.061%, 0.006%, and 0.000% (Variant V); 0.019%, 0.009%, 0.004%, 0.000% (Variant 23); and 0.767%, 0.383%, 0.153%, 0.015%, and 0.000% (Variant Z).

The variants in Batch 2 of the second group of test materials (“Batch 2 test materials”) include Variants A, L, N, B, D, S, U, C, W, Z, AA, E, and F. The Batch 2 test materials were prepared as actives dissolved in a solution. Each variant collagen sample was diluted at a range of percent concentrations in the tissue culture media, FGM with 1.5% FBS (see FIGS. 17C-FIG. 17F). The concentrations reported in the results represent the final concentration of the solution in the tissue culture media (v/v).

Variants A (SEQ ID NO: 987), B (SEQ ID NO: 988), D (SEQ ID NO: 990), C (SEQ ID NO: 989), and E (SEQ ID NO: 991) were tested at 5%, 1%, 0.5%, 0.01% and 0.005% of the original concentration, which corresponded to actual tested concentrations of 0.040%, 0.008%, 0.004%, and 0.000% (Variant A); 0.043%, 0.009%, 0.004%, and 0.000% (Variant B); 0.025%, 0.005%, 0.0025%, 0.00005%, and 0.000025% (Variant B); 0.0.109%, 0.022%, 0.011%, 0.000% (Variant C); and 0.060%, 0.012%, 0.006%, 0.000% (Variant E).

Variant L (SEQ ID NO: 998) and Variant Z (SEQ ID NO: 1012) were tested at 50%, 25%, 10%, 1%, and 0.01% % of the original concentration, which corresponded to actual tested concentrations of 0.563%, 0.281%, 0.113%, 0.011%, and 0.000% (Variant L); and 0.767%, 0.383%, 0.153%, 0.015%, and 0.000% (Variant Z).

Variants N (SEQ ID NO: 1000), S (SEQ ID NO: 1005), U (SEQ ID NO: 1007) and F (SEQ ID NO: 992) were tested at 20%, 10%, 5%, 1%, 0.01% of the original concentration, which corresponded to actual tested concentrations of 0.125%, 0.063%, 0.031%, 0.006%, and 0.000% respectively (Variant N); 0.146%, 0.073%, 0.036%, 0.007%, and 0.000% respectively (Variant S); 0.026%, 0.013%, 0.006%, 0.001%, and 0.000% respectively (Variant U); and 0.155%, 0.078%, 0.039%, 0.008%, and 0.000% respectively (Variant F).

Variants W (SEQ ID NO: 1009) and AA (SEQ ID NO: 1013) were tested at 25%, 10%, 5%, 1%, and 0.01% % of the original concentration, which corresponded to actual tested concentrations of 0.126%, 0.051%, 0.025%, 0.005%, and 0.000% respectively (Variant W); 0.608%, 0.243%, 0.122%, 0.024%, and 0.000% respectively (Variant AA).

The variants in Batch 3 of the second group of test materials (“Batch 3 test materials”) include Variants I, J, K, M, X, Y, BB, and CC. The Batch 3 test materials were prepared as actives dissolved in a solution. Each variant collagen sample was diluted at a range of percent concentrations in the tissue culture media, FGM with 1.5% FBS (see FIGS. 17G-FIG. 17H). The concentrations reported in the results represent the final concentration of the solution in the tissue culture media (v/v).

Variants J (SEQ ID NO: 996), M (SEQ ID NO: 999), Y (SEQ ID NO: 1011), and I (SEQ ID NO: 995) were tested at 30%, 20%, 10%, 1%, and 0.01% of the original concentration, which corresponded to actual tested concentrations of 0.193%, 0.129%, 0.064%, 0.006%, and 0.000% respectively (Variant J); 0.180%, 0.120%, 0.060%, 0.006%, 0.000% respectively (Variant M); 0.080%, 0.053%, 0.027%, 0.003%, and 0.000% respectively (Variant Y); and 0.204%, 0.136%, 0.068%, 0.007%, and 0.000% respectively (Variant I).

Variant X (SEQ ID NO: 1010) was tested at 50%, 25%, 10%, 1%, and 0.01% of the original concentration, which corresponded to actual tested concentrations of 0.103%, 0.0510%, 0.021%, 0.002%, and 0.000%.

Variants BB (SEQ ID NO: 1014) and CC (SEQ ID NO: 1015) were tested at 25%, 10%, 5%, 1%, 0.01% of the original concentration, which corresponded to actual tested concentrations of 0.414%, 0.165%, 0.083%, 0.017%, and 0.000% respectively (Variant 41); and 0.471%, 0.188%, 0.094%, 0.019%, and 0.000% respectively (Variant 42).

Variant K (SEQ ID NO: 1009) was tested at 20%, 10%, 5%, 1%, and 0.01% of the original concentration, which corresponded to actual tested concentrations of 0.270%, 0.135%, 0.068%, 0.014%, and 0.000%, respectively.

The results of the MTT assay are shown in FIGS. 17A-H, and the results are summarized below in Tables 7A-7C. In the tables, “Increase” indicates that the treatment significantly increased the end point, while “Decrease” indicates that the treatment significantly decreased the end point.

### Type I Collagen Assay

Fibroblasts are the main source of the extracellular matrix peptides, including collagen. Procollagen is a large peptide synthesized by fibroblasts in the dermal layer of the skin and is the precursor for collagen. As procollagen is processed to form a mature collagen protein, a propeptide portion is cleaved off in the form of a type I C-peptide. Both the mature collagen protein and the type I C-peptide fragment are then released into the extracellular environment. As collagen is synthesized the type I C-peptide fragment accumulates in the tissue culture medium. Since there is a 1:1 stoichiometric ratio between the two parts of the procollagen peptide, assaying for type I C-peptide reflects the amount of collagen synthesized. To measure the effects of different forms of collagen on collagen synthesis and secretion, Type 1 C-peptide was assayed via an ELISA based method. The Type I Collagen assay on the second group of test materials was conducted as described for the test materials discussed in Example 6.

The samples were separated into batches 1-3 for testing as described above. Batches 1-3, included each of the truncated human collagen III variants having the amino acid sequences set forth in SEQ ID NOs: 987-1015, provided herein. The relationship between the variant (identified by “variant letter”), their amino acid sequences, and their identity to SEQ ID NO: 986 is provided above in Table 6.

The variants in each batch of the second group of test materials were tested at the concentrations described in the MTT assay section above.

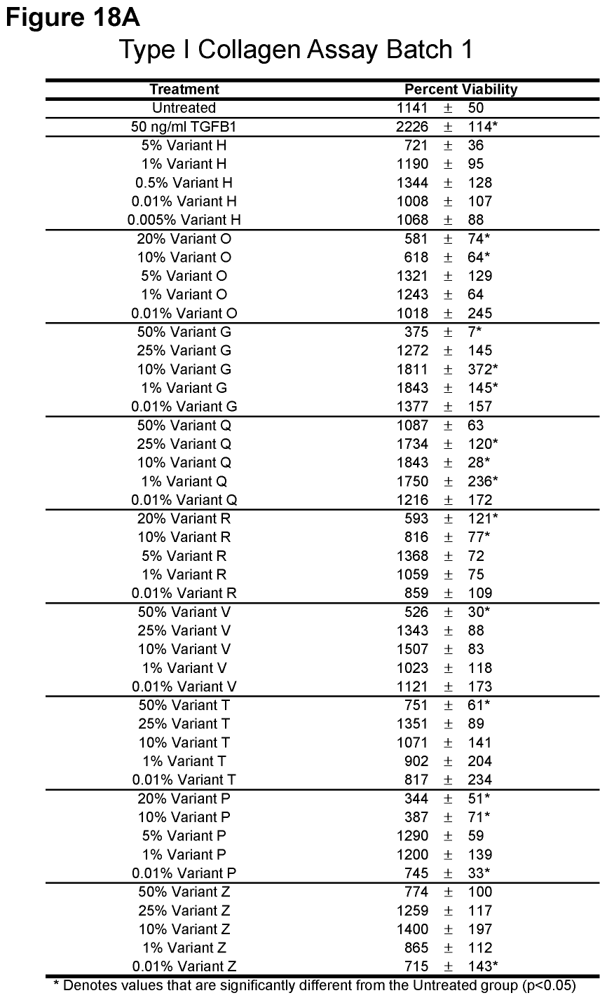

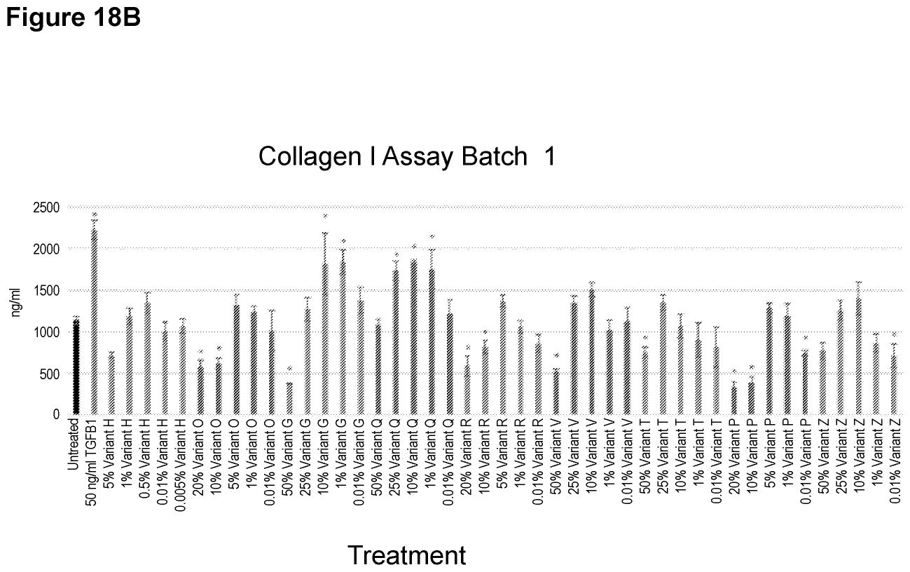

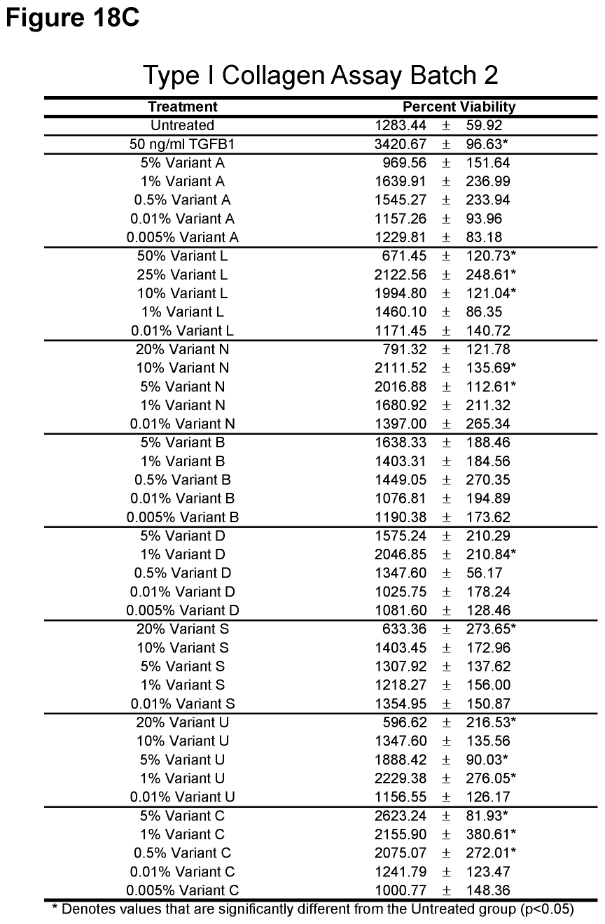

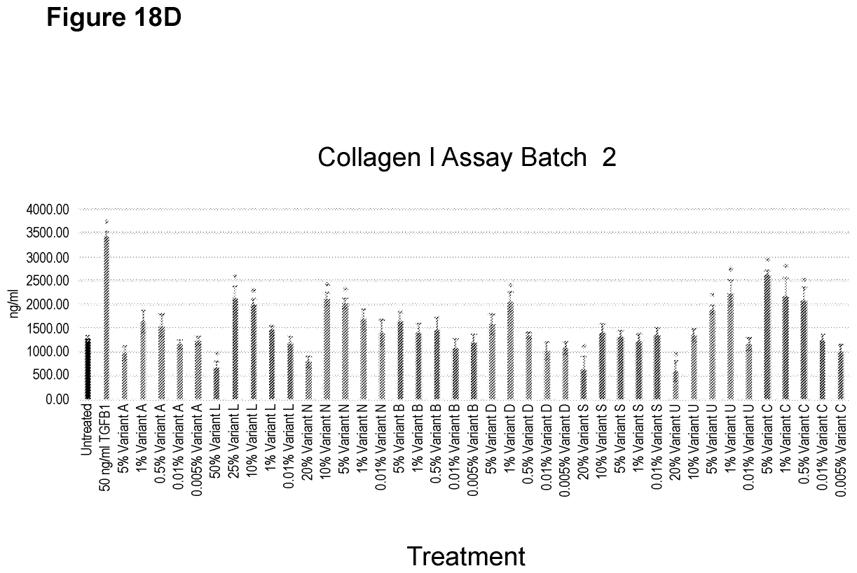

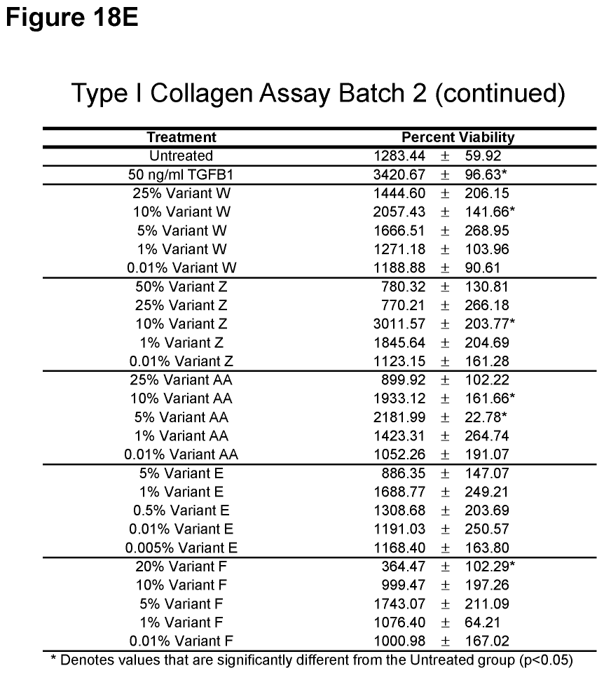

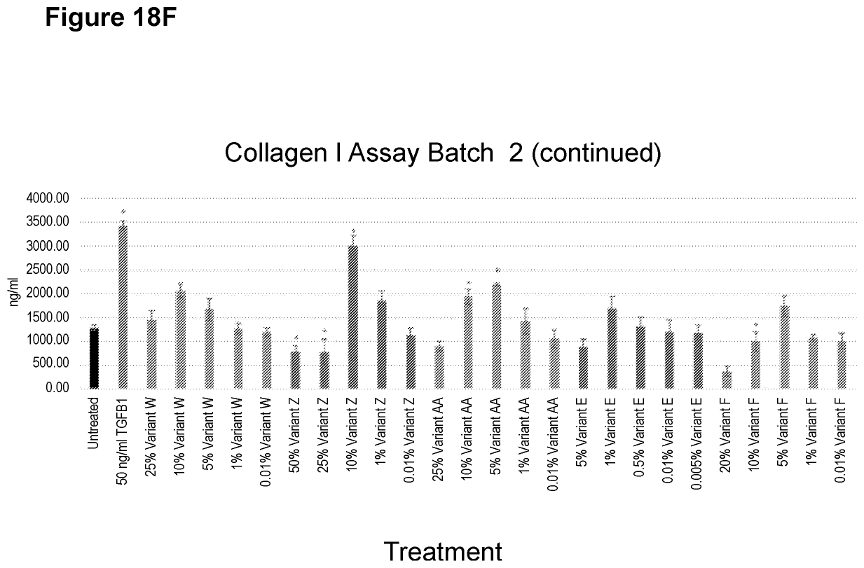

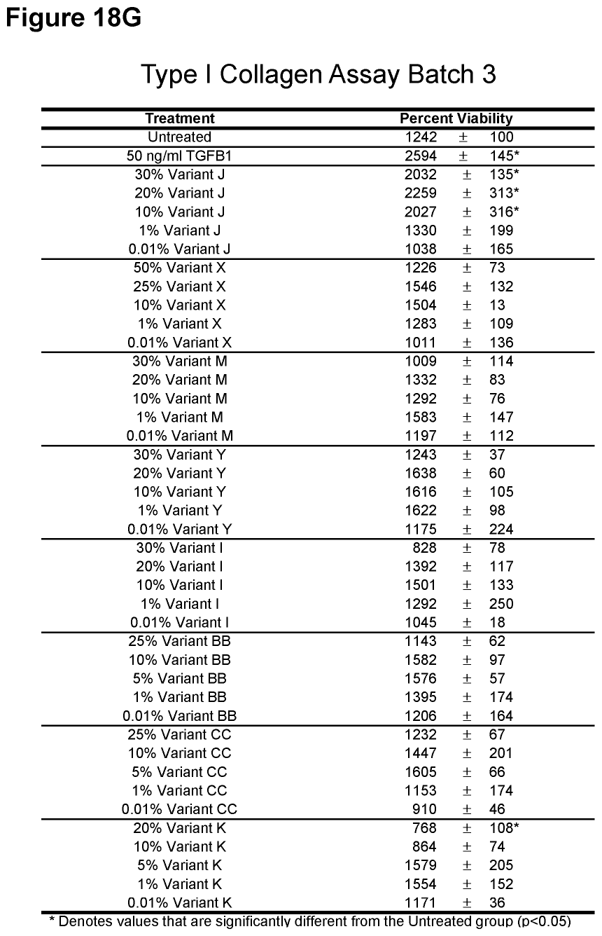

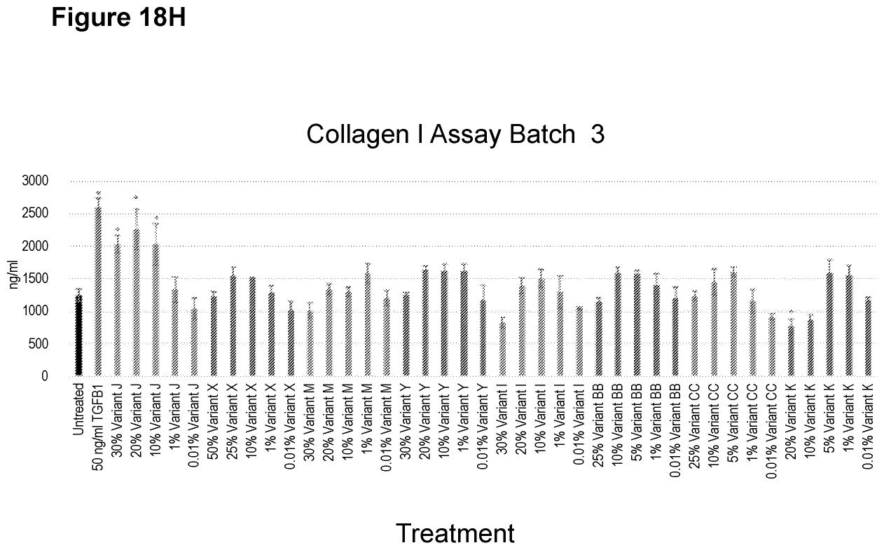

The results of the Type I Collagen assay are shown in FIGS. 18A-H, and the results are summarized below in Tables 7A-7C. In the tables, “Increase” indicates that the treatment significantly increased the end point, while “Decrease” indicates that the treatment significantly decreased the end point.

### Type III Collagen Assay

Type III collagen is synthesized by dermal fibroblasts as a large pro-peptide. As the peptide is processed to form a mature type III collagen protein, a pro-peptide portion is cleaved off (type III N-peptide). Both the mature collagen protein and the type III N-peptide fragment are then released into the extracellular environment. As collagen is synthesized the type III N-peptide fragment accumulates in the tissue culture medium. Since there is a 1:1 stoichiometric ratio between the two parts of the procollagen peptide, assaying for type III N-peptide reflects the amount of collagen synthesized. To measure the effects of different forms of collagen on collagen synthesis and secretion, Type III N-peptide was assayed via an ELISA based method. The Type III Collagen assay on the second group of test materials was conducted as described in Example 6.

The samples were separated into batches 1-3 for testing as described above. Batches 1-3, included each of the truncated human collagen III variants having the amino acid sequences set forth in SEQ ID NOs: 987-1015, provided herein. The relationship between the variant (identified by “variant number”), their amino acid sequences, and their identity to SEQ ID NO: 986 is provided above in Table 6.

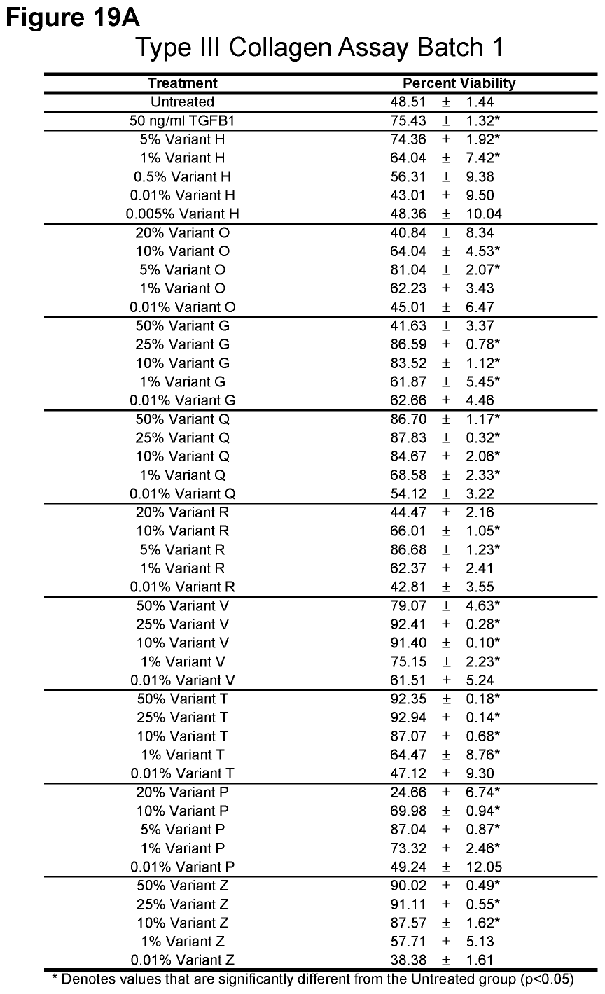

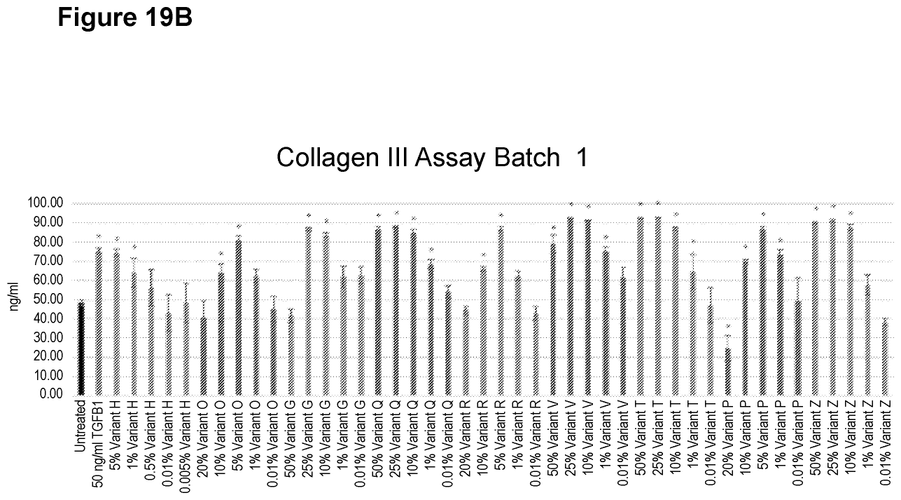

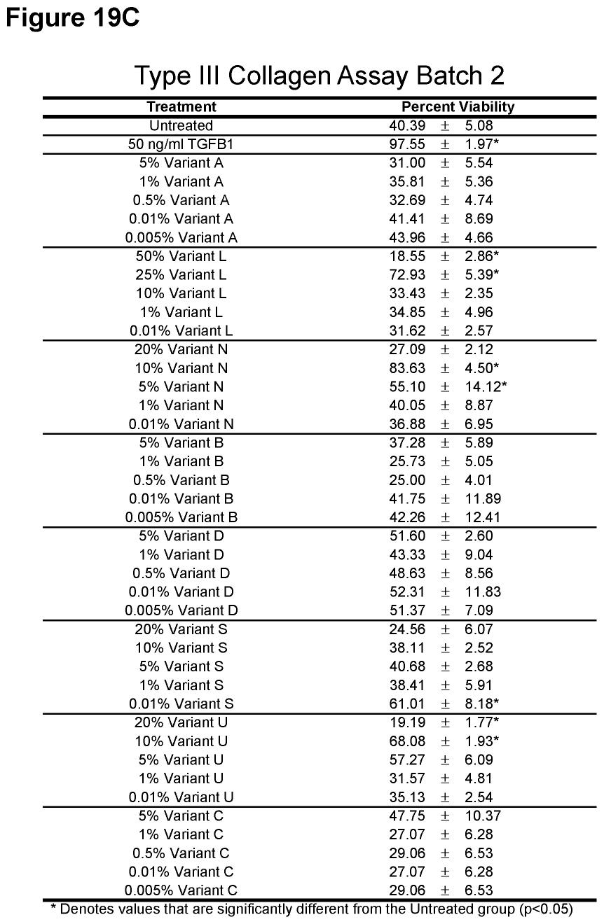

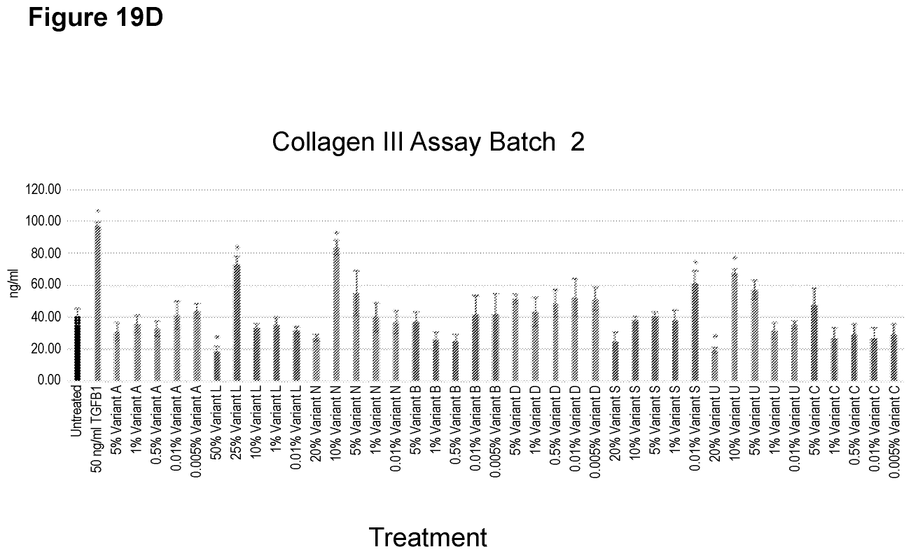

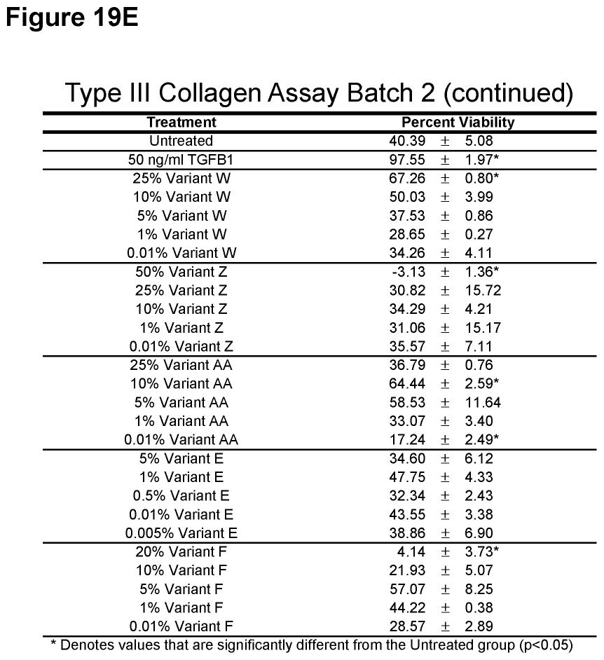

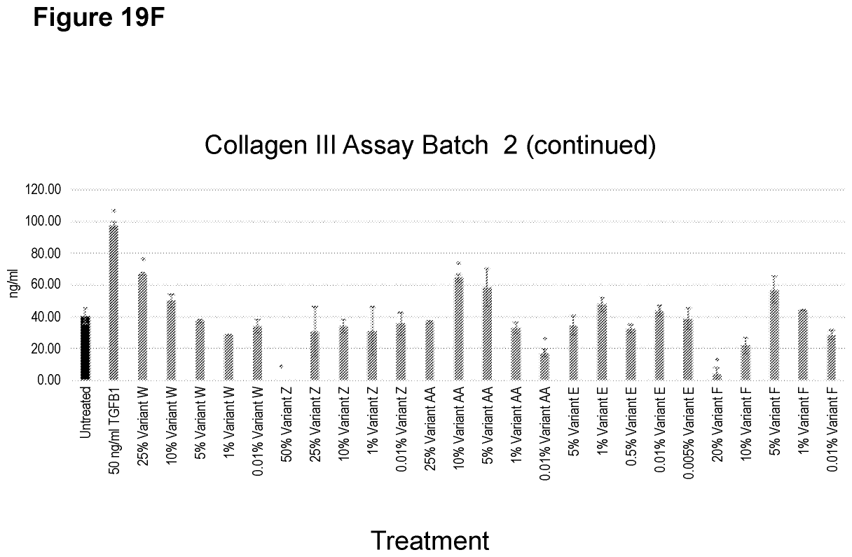

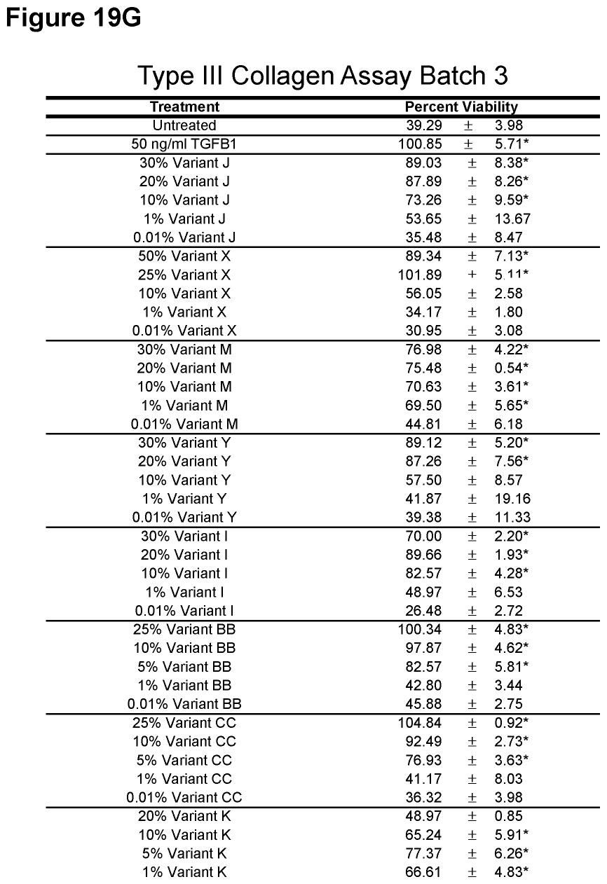

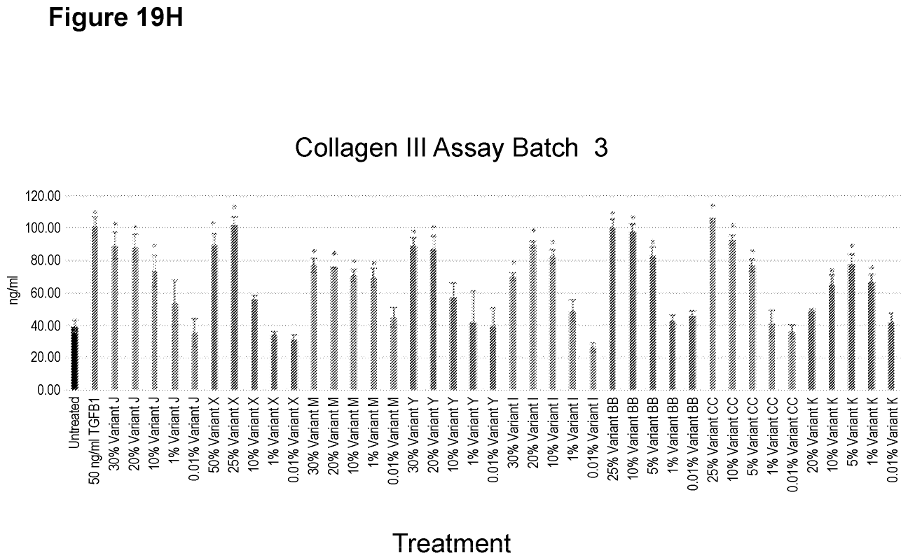

The variants in each batch of the second group of test materials were tested at the concentrations described in the MTT assay section above. Each variant collagen sample was diluted at a range of percent concentrations in the tissue culture media, FGM with 1.5% FBS (see FIGS. 19A-FIG. 19B). The concentrations reported in the results represent the final concentration of the solution in the tissue culture media (v/v).

The results of the Collagen III assay are shown in FIGS. 19A-19H, and the results are summarized below in Tables 7A-7C. In the tables, “Increase” indicates that the treatment significantly increased the end point, while “Decrease” indicates that the treatment significantly decreased the end point.

### Tables 7A-7C Summary of Effects—Batches 1-3

TABLE 7A

| Treatment | MTT | Type I Col | Type III Col |
|---|---|---|---|
| 5% Variant H | Increase | — | Increase |
| 1% Variant H | — | — | Increase |
| 0.5% Variant H | — | — | |
| 0.01% Variant H | — | — | — |
| 0.005% Variant H | — | — | — |
| 20% Variant O | Decrease | Decrease | — |
| 10% Variant O | — | Decrease | Increase |
| 5% Variant O | — | — | Increase |
| 1% Variant O | — | — | — |
| 0.01% Variant O | — | — | — |
| 50% Variant G | Decrease | Decrease | — |
| 25% Variant G | Increase | — | Increase |
| 10% Variant G | Increase | Increase | Increase |
| 1% Variant G | Increase | Increase | — |
| 0.01% Variant G | Increase | — | — |
| 50% Variant Q | Increase | — | Increase |
| 25% Variant Q | Increase | Increase | Increase |
| 10% Variant Q | Increase | Increase | Increase |
| 1% Variant Q | Increase | Increase | Increase |
| 0.01% Variant Q | — | — | — |
| 20% Variant R | Decrease | Decrease | — |
| 10% Variant R | Increase | Decrease | Increase |
| 5% Variant R | — | — | Increase |
| 1% Variant R | — | — | — |
| 0.01% Variant R | — | — | — |
| 50% Variant V | — | Decrease | Increase |
| 25% Variant V | Increase | — | Increase |
| 10% Variant V | Increase | — | Increase |
| 1% Variant V | Increase | — | Increase |
| 0.01% Variant V | — | — | |
| 50% Variant T | Increase | Decrease | Increase |
| 25% Variant T | Increase | — | Increase |
| 10% Variant T | Increase | — | Increase |
| 1% Variant T | Increase | — | Increase |
| 0.01% Variant T | — | — | — |
| 20% Variant P | Decease | Decrease | Decrease |
| 10% Variant P | Increase | Decrease | Increase |
| 5% Variant P | Increase | — | Increase |
| 1% Variant P | Increase | — | Increase |
| 0.01% Variant P | — | — | |
| 50% Variant Z | — | — | Increase |
| 25% Variant Z | Increase | — | Increase |
| 10% Variant Z | Increase | — | Increase |
| 1% Variant Z | Increase | — | — |
| 0.01% Variant Z | — | Decrease | — |
| 5% Variant A | — | — | — |
| 1% Variant A | — | — | — |
| 0.5% Variant A | — | — | — |
| 0.01% Variant A | — | — | — |
| 0.005% Variant A | — | — | — |

TABLE 7B

| Treatment | MTT | Type I Col | Type III Col |
|---|---|---|---|
| 50% Variant L | — | Decrease | Decrease |
| 25% Variant L | Increase | Increase | Increase |
| 10% Variant L | Increase | Increase | — |
| 1% Variant L | — | — | — |
| 0.01% Variant L | — | — | — |
| 20% Variant N | Increase | — | — |
| 10% Variant N | Increase | Increase | Increase |
| 5% Variant N | Increase | Increase | — |
| 1% Variant N | Increase | — | — |
| 0.01% Variant N | — | — | — |
| 5% Variant B | Increase | — | — |
| 1% Variant B | Increase | — | — |
| 0.5% Variant B | — | — | — |
| 0.01% Variant B | — | — | — |
| 0.005% Variant B | — | — | — |
| 5% Variant D | — | — | — |
| 1% Variant D | — | Increase | — |
| 0.5% Variant D | — | — | — |
| 0.01% Variant D | — | — | — |
| 0.005% Variant D | — | — | — |
| 20% Variant S | Increase | Decrease | — |
| 10% Variant S | Increase | — | — |
| 5% Variant S | — | — | — |
| 1% Variant S | — | — | — |
| 0.01% Variant S | — | — | Increase |
| 20% Variant U | — | Decrease | Decrease |
| 10% Variant U | Increase | — | Increase |
| 5% Variant U | Increase | Increase | — |
| 1% Variant U | Increase | Increase | — |
| 0.01% Variant U | — | — | — |
| 5% Variant C | Increase | Increase | — |
| 1% Variant C | Increase | Increase | — |
| 0.5% Variant C | Increase | Increase | — |
| 0.01% Variant C | — | — | — |
| 0.005% Variant C | — | — | — |
| 25% Variant W | — | — | Increase |
| 10% Variant W | Increase | Increase | — |
| 5% Variant W | Increase | — | — |
| 1% Variant W | Increase | — | — |
| 0.01% Variant W | — | — | — |
| 50% Variant Z | Decrease | — | Decrease |
| 25% Variant Z | — | — | — |
| 10% Variant Z | Increase | Increase | — |
| 1% Variant Z | Increase | — | — |
| 0.01% Variant Z | — | — | — |
| 25% Variant AA | — | — | — |
| 10% Variant AA | Increase | Increase | Increase |
| 5% Variant AA | Increase | Increase | — |
| 1% Variant AA | Increase | — | — |
| 0.01% Variant AA | — | — | Decrease |

TABLE 7C

| Treatment | MTT | Type I Col | Type III Col |
|---|---|---|---|
| 5% Variant E | Increase | — | — |
| 1% Variant E | Increase | — | — |
| 0.5% Variant E | — | — | — |
| 0.01% Variant E | — | — | — |
| 0.005% Variant E | — | — | — |
| 20% Variant F | Decrease | Decrease | Decrease |
| 10% Variant F | Increase | — | — |
| 5% Variant F | — | — | — |
| 1% Variant F | — | — | — |
| 0.01% Variant F | — | — | — |
| 30% Variant J | Increase | Increase | Increase |
| 20% Variant J | Increase | Increase | Increase |
| 10% Variant J | Increase | Increase | Increase |
| 1% Variant J | Increase | — | — |
| 0.01% Variant J | — | — | — |
| 50% Variant X | Increase | — | Increase |
| 25% Variant X | Increase | — | Increase |
| 10% Variant X | Increase | — | — |
| 1% Variant X | — | — | — |
| 0.01% Variant X | — | — | — |
| 30% Variant M | — | — | Increase |
| 20% Variant M | — | — | Increase |
| 10% Variant M | Increase | — | Increase |
| 1% Variant M | Increase | — | Increase |
| 0.01% Variant M | — | — | — |
| 30% Variant Y | Increase | — | Increase |
| 20% Variant Y | Increase | — | Increase |
| 10% Variant Y | Increase | — | — |
| 1% Variant Y | Increase | — | — |
| 0.01% Variant Y | — | — | — |
| 30% Variant I | — | — | Increase |
| 20% Variant I | Increase | — | Increase |
| 10% Variant I | Increase | — | Increase |
| 1% Variant I | Increase | — | — |
| 0.01% Variant I | — | — | — |
| 25% Variant BB | — | — | Increase |
| 10% Variant BB | Increase | — | Increase |
| 5% Variant BB | Increase | — | Increase |
| 1% Variant BB | Increase | — | — |
| 0.01% Variant BB | — | — | — |
| 25% Variant CC | — | — | Increase |
| 10% Variant CC | Increase | — | Increase |
| 5% Variant CC | Increase | — | Increase |
| 1% Variant CC | Increase | — | — |
| 0.01% Variant CC | — | — | — |
| 20% Variant K | — | Decrease | — |
| 10% Variant K | Increase | — | Increase |
| 5% Variant K | Increase | — | Increase |
| 1% Variant K | Increase | — | Increase |
| 0.01% Variant K | — | — | — |

The results of the MTT assay demonstrate that, at certain concentrations, Variants B, C, E, F, G, H, I, J, K, L, M, N, P, Q, R, S, T, U, V, W, X, Y, Z, AA, BB, and CC were at least as viable as untreated control cells or increased cell viability.

The results of the Collagen I assay demonstrate that, at certain concentrations, Variants C, D, G, J, L, N, Q, U, W, Z, and AA increase increased the amount of Type I collagen secreted by the treated fibroblasts. These data indicate that compositions comprising these recombinant collagen variants are useful when increased production of type I collagen is desired.

The results of the Collagen III assay demonstrate that, at certain concentrations, Variants G, H, L, N, O, P, Q, R, S, T, U, V, W, Z, AA increased the amount of Type III collagen secreted by the treated fibroblasts. These data indicate that compositions comprising these recombinant collagen variants are useful when increased production of type III collagen is desired.

The collagen variants described in this example were produced and purified by the same procedures. However, impurity levels can vary from sample to sample which led to different toxicity level results in the MTT assay. In general, lower titer is associated with higher impurity levels. As a consequence, some variants with low titers could not be tested at relatively high concentrations for collagen stimulation evaluation.

The breadth and scope of the present disclosure should not be limited by any of the above-described exemplary embodiments, but should be defined only in accordance with the following claims and their equivalents.

1. A recombinant collagen fragment having a molecular weight of about 50 kDa and a sequence identity of at least about 85% to the amino acid sequence set forth in SEQ ID NO: 986.

2. The recombinant collagen fragment of claim 1, wherein the recombinant collagen fragment is unhydroxylated.

3. The recombinant collagen fragment of claim 1, wherein the recombinant collagen fragment is hydroxylated.

4. The recombinant collagen fragment of claim 1, wherein the collagen fragment has the amino acid sequence set forth in SEQ ID NO: 986.

5. A sequence variant of the recombinant collagen fragment of claim 1, wherein the sequence variant comprises the amino acid sequence set forth in any one of SEQ ID NOs: 988-1015.

6-7. (canceled)

8. A composition comprising the recombinant collagen fragment of claim 1.

9. (canceled)

10. The composition of claim 8, wherein the composition further comprises one or more peptides formed from the hydrolyzation of the collagen fragment having the amino acid sequence set forth in SEQ ID NO: 986.

11. (canceled)

12. The composition of claim 8, further comprising a pharmaceutically acceptable or cosmetically acceptable excipient.

13. A method of producing the recombinant collagen fragment of claim 1, comprising producing the recombinant collagen fragment in a genetically engineered strain of yeast.

14. The method of claim 13, wherein the yeast is Pichia pastoris.

15-16. (canceled)

17. The method of claim 13, wherein the method comprises:
(i) fermenting a genetically engineered yeast in a fermentation broth;
(ii) recovering from the fermentation broth recombinant collagen fragments secreted by the genetically engineered yeast; and
(iii) purifying the recombinant collagen fragments.

18. The method of claim 17, further comprising hydroxylating the recombinant collagen fragment ex vivo.

19. A method of producing the sequence variant of claim 5, comprising producing the recombinant collagen fragment in a genetically engineered strain of yeast.

20-23. (canceled)

24. A strain of yeast genetically engineered to produce the recombinant collagen fragment of claim 1, wherein the strain of yeast comprises a vector comprising a DNA sequence encoding the recombinant collagen.

25. A strain of yeast genetically engineered to produce the sequence variant of claim 5, wherein the strain of yeast comprises a vector comprising a DNA sequence encoding the variant.

26. The strain of yeast of claim 24, wherein the vector comprises a nucleic acid sequence comprising the DNA sequence set forth in SEQ ID NO: 973.

27. The strain of yeast of claim 26, further comprising a second vector comprising a nucleic acid sequence comprising the DNA sequence set forth in SEQ ID NO: 974.

28. (canceled)

29. The strain of yeast of claim 24, wherein the strain of yeast is Pichia pastoris.

30. (canceled)

31. A method of treating a dermatological condition comprising topically administering an effective amount of the composition of claim 8 to an area of skin on a subject in need thereof, wherein the dermatological condition comprises fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

32-34. (canceled)

35. A method of increasing collagen production in cells, comprising administering an effective amount of the recombinant collagen fragment of claim 1 to the cells, wherein the method increases the production of Type I collagen, Type III collagen, or a combination thereof.

36-40. (canceled)

41. The method of claim 35, wherein the fragment has an amino acid sequence set forth in SEQ ID NO: 986.

42-43. (canceled)

44. A method of treating a wound in a human subject in need thereof, the method comprising applying the composition of claim 8 to the wound on the subject, wherein applying the recombinant collagen fragment induces the production of human Type I collagen, human Type III collagen, or a combination thereof.

45-79. (canceled)
